(19) United States
(12) Patent Application Publication
(10) Pub. No.: US 2020/0221778 A1
(43) Pub. Date: Jul. 16, 2020

(54) VAPORIZER DEVICE AND SYSTEM

(71) Applicant: MICHAEL TRZECIESKI, Toronto (CA)

(72) Inventor: MICHAEL TRZECIESKI, Toronto (CA)

(21) Appl. No.: 16/801,569

(22) Filed: Feb. 26, 2020

Related U.S. Application Data
(63) Continuation-in-part of application No. 16/207,275, filed on Dec. 3, 2018.
(60) Provisional application No. 62/810,588, filed on Feb. 26, 2019.
(60) Provisional application No. 62/810,588, filed on Feb. 26, 2019.
(60) Provisional application No. 62/876,474, filed on Jul. 19, 2019.
(60) Provisional application No. 62/593,906, filed on Dec. 2, 2017.

(51) Int. Cl. A24F 40/49; A24F 40/65; A24F 40/53; A24F 40/46; A24F 40/42; A24F 40/51; A24F 40/48; A24F 40/60; A24F 40/57; A24F 40/44; A24F 40/10; H04W 12/06; G10L 25/51; A61M 11/04; A61M 15/00; A61M 15/06

(52) U.S. Cl. CPC A24F 40/49; H04L 67/02; A24F 40/53; A24F 40/46; A24F 40/42; A24F 40/51; A24F 40/48; A24F 40/60; A24F 40/57; A24F 40/44; A24F 40/10; H04W 12/06; G10L 25/51; A61M 11/042; A61M 15/0021; A61M 15/06; A24F 40/65

(57) ABSTRACT

A vaporization device allow users to consume removable cartridges filled with vaporizable material. The vaporizer devices defines a receptacle shaped to receive a cartridge in a snug and compact nesting arrangement. The vaporizer device ensures that the installed cartridges are secured and provide a sealed fluid path. A system is disclosed for wirelessly provisioning of the vaporization device to connect to a data server.

### CROSS-REFERENCE TO RELATED APPLICATIONS

This application claims the benefit of U.S. Provisional Application No. 62/593,906, filed Dec. 2, 2017, and is a Continuation in Part of application Ser. No. 16/207,275 filed on Dec. 3, 2018 and claims the benefit of U.S. Provisional Application No. 62/810,588 filed on Feb. 26, 2019 and claims the benefit of U.S. Provisional Application No. 62/876,474 filed on Jul. 29, 2019, the entireties of which is incorporated herein by reference.

### FIELD OF THE INVENTION

This application relates generally to vaporization of phyto materials, and more specifically to devices for use with phyto material extracts and apparatuses and methods for filling cartridges usable with vaporizer devices.

### INTRODUCTION

The following is intended to introduce the reader to the detailed description that follows and not to define or limit the claimed subject matter.

Phyto materials extracts are used for various therapeutic and health applications. For instance, cannabis extracts are used to treat a variety of medical conditions, such as glaucoma, epilepsy, dementia, multiple sclerosis, gastrointestinal disorders and many others. Cannabis extracts have also been used for the general management of pain.

While interest in the therapeutic uses of cannabis is growing, there are a number of challenges associated with its safe and effective use. Challenges include establishing dosing regimens, standardizing the potency and efficacy of cannabis products, and monitoring the use of cannabis by individual patients. These challenges also relate to the various forms in which cannabis may be delivered (e.g. ingestion, smoking, vaporizing). While vaporization of phyto materials avoids some of the deleterious side effects of smoking, there is often still uncertainty in the dose provided by vaporization due to variability in factors such as vaporization temperature, duration and flow volume.

Additionally, the phyto material products themselves (e.g. loose leaf phyto material, extracts etc.) may vary in potency from batch to batch, resulting in different experiences for the patient when consuming different batches of even the same phyto material product. Furthermore, the type or potency of phyto material product that a user consumes may vary over time, as their therapeutic needs change.

### SUMMARY

The following introduction is provided to introduce the reader to the more detailed description to follow and not to limit or define any claimed or as yet unclaimed invention. One or more inventions may reside in any combination or sub-combination of the elements or process steps disclosed in any part of this document including its claims and figures.

In accordance with one aspect of this disclosure, which may be used alone or in combination with any other aspect, a vaporization device that allow users to consume removable cartridges filled with phyto material products is provided. The vaporizer devices may facilitate the consumption of varying types and/or potencies of phyto material products through the same vaporizer device. The vaporizer devices may provide a compact nesting arrangement for cartridges that enables the cartridges to be easily installed and removed. The vaporizer devices may also ensure that, once installed, the cartridges are secured and may provide a sealed fluid path through the device.

In accordance with this broad aspect, there is provided a vaporizer device comprising: a vaporizer body comprising: an elongated base extending from a first end to a second end, the elongated base including a pair of opposed sidewalls extending between the first end and the second end and a second end wall at the second end; a mouthpiece formed at the second end of the base, the mouthpiece comprising an inhalation aperture through the second end wall; an air intake manifold mounted to the base, the air intake manifold having a first manifold end and a second manifold end, the air intake manifold comprising an ambient air input port disposed between the first manifold end and the second manifold end, the ambient air input port being exposed to an external environment; a cartridge receptacle formed within the elongated base, wherein the cartridge receptacle is defined between the sidewalls, the second end wall and the second end of the air intake manifold; and a cartridge removably mountable in the cartridge receptacle, the cartridge comprising: a cartridge housing extending from a first cartridge end to a second cartridge end; an elongated storage compartment, the storage compartment being configured to store a vaporizable material, the storage compartment comprising an inner storage volume wherein the vaporizable material is storable in the inner storage volume, wherein the inner storage volume is enclosed by the cartridge housing; a heating assembly disposed at the first cartridge end, the heating assembly comprising a heating element and a wicking element, wherein the heating element thermally coupled to the wicking element, and wherein the wicking element is in fluid communication with the inner storage volume; and a fluid conduit extending through the cartridge housing, the fluid conduit having a fluid conduit inlet at the first cartridge end and a fluid conduit outlet at the second cartridge end, wherein the fluid conduit is in fluid communication with the wicking element; wherein when the cartridge is mounted within the cartridge receptacle, the fluid conduit inlet is fluidly connected to the air intake manifold and the fluid conduit outlet is fluidly connected to the mouthpiece, and a fluid flow passage is defined between the ambient air input port and the inhalation aperture, the fluid flow passage passing through the heating assembly whereby vaporized material is inhalable through the inhalation aperture.

In some embodiments, the fluid conduit outlet protrudes beyond the second cartridge end and is received by the mouthpiece when the cartridge is mounted within the cartridge receptacle.

In some embodiments, the cartridge includes a plurality of cartridge electrical contacts disposed at the first cartridge end; the device body includes a plurality of device electrical contacts disposed at the second end of the air intake manifold, the plurality of device electrical contacts engaging the plurality of cartridge electrical contacts when the cartridge is mounted within the cartridge receptacle.

In some embodiments, the device includes a cartridge lock unit, the cartridge lock unit configured to secure the cartridge in a mounted position within the cartridge receptacle, the cartridge lock unit being adjustable between a locked position and an unlocked position, where when the cartridge is mounted in the cartridge receptacle and the cartridge lock unit is in the locked position, the cartridge lock unit retains the cartridge in the cartridge receptacle and prevents removal of the cartridge, and when the cartridge is positioned in the cartridge receptacle and the cartridge lock unit is in the unlocked position, the cartridge unit is removable from the cartridge receptacle.

In some embodiments, the device includes an ejection actuator positioned within the base underlying the cartridge receptacle, the ejection actuator adjustable between an extended position in which the ejection actuator extends into the cartridge receptacle and a retracted position in which the actuator is retracted within the base. The ejection actuator may be biased to the extended position.

In some embodiments, the inner storage volume at least partially surrounds the fluid conduit.

In some embodiments, an outer surface of the elongated storage compartment is externally exposed when the cartridge is mounted within the cartridge receptacle.

In some embodiments, the elongated storage compartment includes a viewing region overlying at least a portion of the inner storage volume, the viewing region positioned on a portion of the exposed outer surface of the elongated storage compartment, where the viewing region is at least partially transparent such that vaporizable liquid positioned in the storage compartment is visible through the viewing region.

In some embodiments, the device body includes a plurality of display indicators proximate the first end of the base, the plurality of display indicators including a plurality of light emitting diodes.

In some embodiments, the vaporizer body includes: at least one energy storage member mounted to base; and a recharging port proximate the first end of the base.

In some embodiments, the center of gravity of the vaporizer device is closer to the first end of base than to the second end of the base.

In some embodiments, the vaporizer body has an elliptical cross section.

In some embodiments, the vaporizer body is tapered from the first end to the second end, such that a first surface area of the elliptical cross-section proximate the first end is greater than a second surface area of the elliptical cross-section proximate the second end.

In some embodiments, the base is formed using a metal material.

In some embodiments, the base has a unitary construction and in some embodiments the heating element assembly has a unitary construction from a porous ceramic material.

In some embodiments, the base defines a recess, the recess extending from the first end of the device body to the second end of the device body.

In some embodiments, the recess includes a plurality of recess sections, the plurality of recess sections including a first recess section and a second recess section, the first section extending from the first end of the base towards the second end of the base, and the second section defining the cartridge receptacle; and at least one of an energy storage member and a control circuit are mounted within the first recess section.

In some embodiments, the air intake manifold is mounted within a third recess section that is between the first recess section and the second recess section.

In some embodiments, the vaporizer body includes a body cover that is securable to the base, where the body cover overlies the first recess section.

In some embodiments, the body cover is formed using a non-conductive material.

In some embodiments, the vaporizer device includes a control circuit assembly that includes the control circuit mounted to a support assembly, the support assembly including a support member that extends through the first recess section to the first end of the base, where the support assembly includes a rubberized end cover member that frictionally engages the base and the body cover at the first end of the base and defines a first end of the vaporizer body at the first end of the base.

In some embodiments, the cartridge includes a plurality of cartridge electrical contacts disposed at a first cartridge end; the vaporizer body includes a plurality of device electrical contacts disposed at the second manifold end, the plurality of device electrical contacts engaging the plurality of cartridge electrical contacts when the cartridge is secured within the cartridge receptacle; and the vaporizer body includes a control circuit assembly having a wireless communication module and at least one energy storage member, and the control circuit assembly is electrically connected to the plurality of device electrical contacts.

In some embodiments, a flow sensor is disposed within the air intake manifold, the flow sensor operable to detect a mass of air entering the ambient air input port.

In some embodiments, the fluid flow sensor includes a mass airflow sensor.

In some embodiments, the fluid flow sensor includes a volumetric airflow sensor.

In some embodiments, the fluid flow sensor includes a barometric pressure sensor.

In some embodiments, the volumetric airflow sensor includes a microphone.

In some embodiments, a puff sensor is disposed within the air intake manifold, the puff sensor operable to detect air entering the ambient air input port.

In some embodiments, the device body includes a plurality of device electrical contacts disposed at the second end of the air intake manifold; the cartridge includes a plurality of cartridge electrical contacts disposed at the first cartridge end; and the elongated storage compartment includes at least one registration feature, the registration feature permitting the cartridge to engage the cartridge receptacle with the fluid conduit fluidly connected to the air intake manifold at the first cartridge end and the fluid conduit fluidly connected to the mouthpiece at the second cartridge end and with the plurality of device electrical contacts engaging the plurality of cartridge electrical contacts, and preventing the cartridge from being secured within the cartridge receptacle in any other orientation.

In some embodiments, the cartridge includes a filling aperture defined in the cartridge housing extending into the inner storage volume, the filling aperture configured to allow the vaporizable material to be deposited into the inner storage volume; and the filling aperture is sealable by heating the filling aperture to a melting temperature to seal the inner storage volume with the vaporizable material deposited therein.

In some embodiments, the vaporizer body includes an activation lock, the activation lock being adjustable between an activated state and a deactivated state, in the deactivated state the activation lock prevents the heating assembly from being energized, and in the activated state the activation lock enables energizing of the heating assembly, and the activation lock is set to the deactivated state by default.

In some embodiments, the vaporizer body includes an activation lock input, the activation lock input being usable to adjust the activation lock between the activated state and the deactivated state.

In some embodiments, when the cartridge is mounted within the cartridge receptacle, the cartridge housing is fluidically sealed from the external environment apart from the ambient air input port and the inhalation aperture.

In accordance with another aspect of this disclosure, which may be used alone or in combination with any other aspect, a cartridge encloses a fluid conduit and has a storage compartment for a vaporizable phyto material. The fluid conduit may extend throughout the length of the cartridge defining a substantially linear flow passage. This may facilitate the flow of air and vapor through the cartridge and make it easier for a user to inhale vapor from a vaporization device using the cartridge. The storage compartment may be arranged to surround the fluid conduit. This may also allow the cartridge to provide an increased storage volume for vaporizable material.

The heating element assembly may also be positioned concentrically with both the storage compartment and the fluid conduit, in between the storage compartment and fluid conduit. This may allow the heating element assembly to provide an increased surface area for vaporizing the material from the storage compartment. This may also allow the device to include additional apertures between the storage compartment and heating assembly.

In accordance with this broad aspect, there is provided a cartridge usable with a vaporizer device that includes a mouthpiece having an inhalation aperture, the cartridge comprising: a cartridge housing extending from a first end of the cartridge to a second end of the cartridge; an elongated storage compartment, the storage compartment being configured to store a vaporizable material, the storage compartment comprising an inner storage volume wherein the vaporizable material is storable in the inner storage volume, wherein the inner storage volume is enclosed by the cartridge housing; a heating assembly disposed at the first end of the storage compartment, the heating assembly comprising a heating element, a wicking element, and a storage interface member, wherein the heating element is in thermal contact with the wicking element, wherein the storage interface member surrounds the wicking element, and the storage interface member includes a plurality of circumferentially spaced fluid apertures fluidly connecting the wicking element to the inner storage volume; and a fluid conduit extending through the housing from a conduit inlet at the first end to a conduit outlet at the second end, wherein the fluid conduit is fluidly connected to the wicking element, the fluid conduit passes through the heating assembly; wherein the storage compartment, heating assembly and fluid conduit are concentrically disposed; wherein the storage compartment surrounds the heating assembly and the fluid conduit; and wherein the fluid conduit extends along the entire length of the elongated storage compartment.

In some embodiments, the elongated storage compartment has a first storage section and a second storage section, the second storage section surrounds the fluid conduit proximate the second end of the cartridge, and the first storage section surrounding the heating assembly and the fluid conduit; the inner storage volume in the first storage section has a first section inner radius; the inner storage volume in the second storage section has a second section inner radius; and the second section inner radius is less than the first section inner radius.

In some embodiments, the housing has a first housing section and a second housing section; the first housing section extends from the first end of the cartridge towards the second end, and the second housing section extends from the first housing section to the second end of the cartridge; a computer readable memory circuit and a plurality of electrical contacts are disposed within the first housing section; and the heating element and storage compartment are entirely contained within the second housing section.

In some embodiments, the cartridge includes a plurality of cartridge electrical contacts at the first end of the housing, the plurality of electrical contacts being engageable with corresponding base electrical contacts provided on the vaporizer device.

In some embodiments, the plurality of cartridge electrical contacts are flush with the housing at the first end of the cartridge.

In some embodiments, the housing has an elliptical cross section.

In some embodiments, the housing has planar side sections that extend perpendicular to the major axis of the elliptical cross-section.

In some embodiments, the housing is tapered from the first end to the second end, such that a first surface area of the elliptical cross-section proximate the first end is greater than a second surface area of the elliptical cross-section proximate the second end.

In some embodiments, the fluid conduit includes a first conduit section, a second conduit section, and a third conduit section, wherein the second conduit section is downstream from the first conduit section and upstream from the third conduit section; the first conduit section extends from the first end of the housing to an upstream end of the heating assembly; the second conduit section extends from the upstream end of the heating assembly to a downstream end of the heating assembly through the heating assembly, and the second conduit section is fluidly connected to the wicking element; the third conduit section extends from the downstream end of the heating assembly to the second end of the housing.

In some embodiments, the housing includes at least one mounting member that is engageable with corresponding mounting components of the vaporizer device; and the at least one mounting member is asymmetric whereby the housing is engageable with the corresponding mounting components in only one orientation.

In accordance with another aspect of this disclosure, which may be used alone or in combination with any other aspect, a cartridge encloses a fluid conduit and has a storage compartment for a vaporizable phyto material. The cartridge may include a viewing region formed in the cartridge housing that allows the interior of the storage compartment to be visible through the housing, even when the cartridge is installed for user. This may allow a user to easily assess the remaining quantity of vaporizable material in the storage compartment. The fluid conduit may also be visible from the exterior of the cartridge. A user may use the viewing region to assess the state of the fluid conduit while the cartridge is installed.

In accordance with this broad aspect, there is provided a cartridge usable with a vaporizer device that includes a mouthpiece having an inhalation aperture, the cartridge comprising: a housing extending from a first end of the cartridge to a second end of the cartridge; an elongated storage compartment, the storage compartment being configured to store a vaporizable material, the storage compartment comprising an inner storage volume wherein the vaporizable material is storable in the inner storage volume, wherein the inner storage volume is enclosed by the cartridge housing, wherein the cartridge housing includes a viewing region overlying at least a portion of the inner storage volume and the viewing region is at least partially transparent to enable the vaporizable material to be visible through the viewing region; a heating assembly disposed at the first end of the cartridge, the heating assembly comprising a heating element and a wicking element, wherein the heating element is in thermal contact with the wicking element, and wherein the wicking element is fluidly connected to the inner storage volume; and a fluid conduit extending through the housing from a conduit inlet at the first end to a conduit outlet at the second end, wherein the fluid conduit is fluidly connected to the wicking element; wherein the storage compartment surrounds the fluid conduit.

In some embodiments, the cartridge includes a plurality of cartridge electrical contacts at the first end of the housing, the plurality of electrical contacts being engageable with corresponding base electrical contacts provided on the vaporizer device; and a temperature sensor in thermal communication with the heating element; where the temperature sensor is electrically coupled with the plurality of cartridge electrical contacts, and the temperature sensor is configured to output a temperature signal indicative of a temperature of the heating element.

In some embodiments, the cartridge includes a plurality of cartridge electrical contacts at the first end of the housing, the plurality of electrical contacts being engageable with corresponding base electrical contacts provided on the vaporizer device; and a computer readable memory circuit having stored thereon a unique cartridge identifier for uniquely identifying the cartridge, where the memory is electrically coupled with the first plurality of electrical contacts.

In some embodiments, the cartridge housing has an elliptical cross section.

In some embodiments, the cartridge housing has planar side sections that extend perpendicular to the major axis of the elliptical cross-section.

In some embodiments, the cartridge housing is tapered from the first end to the section end, such that a first surface area of the elliptical cross-section proximate the first end is greater than a second surface area of the elliptical cross-section proximate the second end.

In some embodiments, the fluid conduit includes a first conduit section, a second conduit section, and a third conduit section, where the second conduit section is downstream from the first conduit section and upstream from the third conduit section; the first conduit section extends from the first end of the housing to an upstream end of the heating assembly; the second conduit section extends from the upstream end of the heating assembly to a downstream end of the heating assembly through the heating assembly, and the second conduit section is fluidly connected to the wicking element; and the third conduit section extends from the downstream end of the heating assembly to the second end of the housing.

In some embodiments, the cartridge includes a filling aperture that extends through the cartridge housing and into the inner storage volume, the filling aperture configured to allow the vaporizable material to be deposited into the inner storage volume; where the filling aperture is sealable by heating the filling aperture to a melting temperature to seal the inner storage volume with the vaporizable material deposited therein.

In some embodiments, the cartridge includes a plurality of cartridge electrical contacts at the first end of the housing, the plurality of electrical contacts being engageable with corresponding base electrical contacts provided on the vaporizer device; and a cartridge control unit electrically coupled with the plurality of cartridge electrical contacts.

In some embodiments, the heating assembly includes a storage volume interface member that engages an inner surface of the enclosed storage compartment; the storage volume interface member surrounds the wicking element; and the storage volume interface member includes a plurality of fluid apertures fluidly connecting the wicking element to the inner storage volume.

In some embodiments, the fluid apertures are circumferentially spaced around the storage volume interface member at regular intervals.

In some embodiments, the heating element has a ceramic outer layer having an annular cross-section with an inner heating element surface and an outer heating element surface; the heating element includes a resistive heating wire secured within the ceramic outer layer; the wicking element is wrapped around the outer heating element surface; and the inner heating element surface defines a portion of the fluid conduit.

In some embodiments, the viewing region is on a first outer surface of the storage compartment; and the storage compartment also includes an opaque region aligned with the viewing region.

In some embodiments, the fluid conduit is positioned between the viewing region and the opaque region, and the fluid conduit is at least partially visible through the viewing region.

In some embodiments, an interior surface of the opaque region includes a cartridge identification label.

In some embodiments, the opaque region is provided on an inner surface of the storage compartment.

In some embodiments, the cartridge housing includes at least one mounting member that is engageable with corresponding mounting components of the vaporizer device; and the at least one mounting member is asymmetric such that the housing is engageable with the corresponding mounting components in only one orientation.

In some embodiments, the fluid conduit protrudes beyond the second end of the housing, and the protruding section of the fluid conduit is configured to engage with the mouthpiece.

In accordance with another aspect of this disclosure, which may be used alone or in combination with any other aspect, a phyto material cartridge has a lid formed separately from the base. The lid and base may be sealed after being filled, which may simplify the process of filing the storage compartment. In some cases, the lid and base may using mating mechanical securing members to secure the lid to the base. This may allow the lid to be removed and the cartridge to be refilled.

In accordance with this broad aspect, there is provided a cartridge usable with a vaporizer device that includes a mouthpiece having an inhalation aperture, the cartridge comprising: a cartridge body extending from a first end of the cartridge to a second end of the cartridge, the cartridge body having a cartridge base and a cartridge cover; an elongated storage compartment that is configured to store a vaporizable material, the storage compartment including a compartment base and storage compartment sidewalls, the storage compartment sidewalls being defined by the cartridge base, the storage compartment sidewalls extending around the compartment base and the storage compartment sidewalls extending from the compartment base to an upper sidewall perimeter; a heating assembly disposed at the first end of the cartridge, the heating assembly comprising a heating element and a wicking element, wherein the heating element is in thermal contact with the wicking element, and wherein the wicking element is fluidly connected to the inner storage volume; and a fluid conduit extending through the housing from the first end to the second end, wherein the fluid conduit is fluidly connected to the wicking element; wherein the cartridge base and the cartridge cover are formed separately; and the cartridge cover is secured to the cartridge base with the cartridge cover engaging the storage compartment sidewalls throughout the upper sidewall perimeter to define an enclosed inner storage volume that is fluidly sealed along the upper sidewall perimeter, and the vaporizable material is storable in the inner storage volume;

In some embodiments, the cartridge cover is secured to the cartridge base at a plurality of securing locations around an outer periphery of the cartridge cover.

In some embodiments, the cartridge cover includes a plurality of cover engagement members and the cartridge base includes a corresponding plurality of base engagement members; and the cartridge cover is secured to the cartridge base, with the cartridge cover enclosing the inner storage volume, by engaging the cover engagement members with the corresponding base engagement members.

In some embodiments, the plurality of cover engagement members comprises snap fittings.

In some embodiments, the cartridge cover has a cover body that defines a top outer surface of the cartridge, the top surface facing in a first direction away from the inner storage volume; the plurality of cover engagement members project from the cover body in a second direction, the second direction being opposite to the first direction; and the plurality of base engagement members are provided on opposing lateral sides of the cartridge base.

In some embodiments, each cover engagement member comprises a first member section and a second member section, the first member section extending in the second direction from the cover body to a distal member end, and the second member section extends laterally inward of the first member section at the distal member end; and each base engagement member comprises a recess shaped to receive the second member section of the corresponding cover engagement member, and to retain the cover engagement member in the recess when the cartridge cover is mounted to the cartridge base.

In some embodiments, each cover engagement member is a resilient engagement member; and when the cartridge cover is lowered onto the cartridge base, the resilient engagement member automatically engages the corresponding base engagement member with the second member section inserted into the corresponding recess.

In some embodiments, the cartridge cover includes a viewing region overlying at least a portion of the inner storage volume and the viewing region is at least partially transparent to enable the vaporizable material to be visible through the viewing region.

In some embodiments, the cartridge includes a compressible seal member extending along the upper sidewall perimeter between the cartridge cover and the cartridge base, where when the cartridge cover is secured to the cartridge base, the seal member is compressed and defines the seal between the cartridge cover and the cartridge base.

In some embodiments, the compartment base is in thermal contact with the fluid conduit.

In some embodiments, the fluid conduit is in contact with the compartment base throughout the entire length of the elongated storage compartment.

In some embodiments, the fluid conduit defines a linear airflow passage throughout a majority of the cartridge housing.

In some embodiments, the wicking element extends into the inner storage volume.

In some embodiments, the cartridge includes a plurality of electrical contacts proximate the first end of the cartridge body, the plurality of electrical contacts being engageable with corresponding electrical contacts provided on the vaporizer device, the plurality of electrical contacts positioned on a bottom surface of the cartridge base.

In some embodiments, the cartridge body has a top surface defined by the cartridge cover and a bottom surface defined by the cartridge base that is opposite to the top surface; a central axis extends through the cartridge body from the first end to the second end, the central axis being equidistant from the top surface and the bottom surface; and the fluid conduit is positioned entirely on the bottom side of the central axis.

In accordance with another aspect of this disclosure, which may be used alone or in combination with any other aspect, the storage compartment of a phyto material cartridge may be filled prior to installing the lid of the cartridge. This may allow vaporizable liquids to be dispensed using wider dispensing nozzles, increasing the speed at which cartridges may be filled. This may also allow vaporizable material to be deposited in semi-fluid or even solid form and then enclosed within the storage compartment.

In accordance with this broad aspect, there is provided a method for filling a cartridge with a vaporizable material, the cartridge having a cartridge base and a cartridge lid, the cartridge base defining a bottom surface and a peripheral sidewall of a storage compartment that has an open top side, the method comprising: positioning the cartridge base within a filling tray with the bottom surface of the storage compartment facing upwardly; depositing vaporizable material into the open top side of the storage compartment; lowering the cartridge lid onto the cartridge base; and securing the cartridge lid to the cartridge base at a plurality of fastening locations around the perimeter of the cartridge lid.

In some embodiments, securing the cartridge lid to the cartridge base involves engaging corresponding frictional engagement members providing on the cartridge lid and on the cartridge base.

In some embodiments, the frictional engagement members engage automatically as the cartridge lid is lowered onto the cartridge base.

In some embodiments, the peripheral sidewall extends around the bottom surface and extends from the bottom surface to an upper sidewall perimeter, and the method includes: positioning a seal member around the upper sidewall perimeter; and compressing the seal member as the cartridge lid is lowered onto the cartridge base.

In some embodiments, depositing vaporizable material into the open top side of the storage compartment involves injecting liquid vaporizable material using an injection syringe.

In some embodiments, the vaporizable material is deposited into the open top side of the storage compartment in a solid or semi-solid state.

In accordance with another aspect of this disclosure, which may be used alone or in combination with any other aspect, the storage compartment of a phyto material cartridge is filled through a filling aperture formed in a cartridge housing manufactured of a thermoplastic material. The filling aperture may then be sealed by melting a section of housing adjacent to the aperture and using the melted section to form a wall sealing the filling aperture. This may allow a wider filling aperture to be used, while ensuring that the storage compartment is enclosed after being filled.

In accordance with this broad aspect, there is provided a method of filling a cartridge with a vaporizable material, the method comprising: providing a storage compartment having an outer wall defining an inner storage volume, the outer wall having a filling aperture formed thereon; inserting a filling nozzle into the filling aperture; injecting liquid vaporizable material through the filling aperture into the inner volume; and sealing the filling aperture after the liquid vaporizable material is injected to define an enclosed inner storage volume.

In some embodiments, the outer wall is formed from a thermoplastic material having a defined melting temperature, and method involves sealing the filling aperture by: heating an outer wall section adjacent the filling aperture to the defined melting temperature to provide a melted outer wall section; and forming the melted outer wall section over the filling aperture to seal the filling aperture.

In some embodiments, heating the outer wall section involves inserting a heated plunger into the filling aperture.

In accordance with another aspect of this disclosure, which may be used alone or in combination with any other aspect, a filling apparatus has a filling tray assembly and a robotic arm assembly. The arm assembly may automatically fill multiple cartridges positioned within the tray assembly. The arm assembly may also seal multiple cartridges after filling while they are positioned in the filling assembly. This may provide a more efficient method of filling multiple phyto material cartridges.

In accordance with this broad aspect, there is provided an apparatus for filling a cartridge with a vaporizable material, the cartridge having a cartridge base and a storage compartment, the apparatus comprising: an apparatus base; a tray secured to the apparatus base, the tray shaped to retain the cartridge base; a movable arm assembly secured to the apparatus base, the movable arm assembly including a dispensing nozzle; and a storage reservoir usable to house the vaporizable material, the storage reservoir fluidly coupled to the dispensing nozzle; wherein the movable arm assembly is operable to direct a nozzle outlet of the dispensing nozzle into the storage compartment; and the dispensing nozzle is operable to inject vaporizable material from the storage reservoir into the cartridge.

In some embodiments, the storage compartment has an outer wall defining an inner storage volume and a filling aperture formed in the outer wall; the dispensing nozzle is sized to be accommodated within the filling aperture; and the movable arm assembly is operable to insert the nozzle outlet into the filling aperture when the cartridge is positioned in the tray, and to inject the vaporizable material into the cartridge through the filling aperture.

In some embodiments, the outer wall is formed from a thermoplastic material having a defined melting temperature; the movable arm assembly includes an extensible plunger having a heatable distal end; the arm assembly is configured to heat the distal end of the plunger to a defined melting temperature, and to move the plunger to contact an outer wall section of the outer wall adjacent to the filling aperture to melt the outer wall section to seal the filling aperture.

In some embodiments, the movable arm assembly is configured to extend the heated plunger into the filing aperture to melt the outer wall section.

In some embodiments, the apparatus includes an array of trays secured to the base; each tray is shaped to retain the cartridge base of a corresponding cartridge; and the arm assembly is moveable direct the nozzle outlet of the dispensing nozzle into the storage compartment of the corresponding cartridge positioned in each tray.

In some embodiments, the arm assembly includes a lid support member operable to grasp a lid corresponding to each cartridge, and the arm assembly is configured to lower the lid onto the corresponding cartridge base positioned in each tray.

In some embodiments, the arm assembly is configured to compress the lid onto the corresponding cartridge base until the lid secures itself to the base.

In some embodiments, the arm assembly is configured to direct the nozzle outlet into an open top surface of the cartridge positioned in each tray.

In accordance with this broad aspect there is provided a vaporizer device comprising: a vaporizer body comprising: an elongated base extending from a first end to a second end, the elongated base including a pair of opposed sidewalls extending between the first end and the second end and a second end wall at the second end; a mouthpiece formed at the second end of the base, the mouthpiece comprising an inhalation aperture through the second end wall; an air intake manifold mounted to the base, the air intake manifold having a first manifold end and a second manifold end with a manifold fluid flow path defined therethrough, the air intake manifold comprising an ambient air input port disposed between the first manifold end and the second manifold end, the ambient air input port being exposed to an external environment; a fluid flow sensor assembly fluidly coupled between first manifold end and a second manifold with the manifold fluid flow path, the fluid flow sensor assembly for generating a fluid flow signal in dependence upon a flow of air through the manifold fluid flow exceeding a predetermined flow threshold; an elongated storage compartment, the storage compartment being configured to store a vaporizable material, the storage compartment comprising an inner storage volume wherein the vaporizable material is storable in the inner storage volume, the elongated storage compartment comprising a first end and a second end opposite the first end; a heating element assembly disposed at the elongated storage compartment first end, the heating assembly comprising a heating element, wherein the heating element is thermally coupled with the heating element assembly, and wherein heating element assembly is in fluid communication with the inner storage volume for wicking of the vaporizable material into the heating element assembly; and a fluid conduit extending parallel with the elongated storage compartment from the first end to the second end, the fluid conduit having a fluid conduit inlet proximate the elongated storage compartment first end and a fluid conduit outlet proximate the elongated storage compartment second end, wherein the fluid conduit is in fluid communication with the heating element assembly and the fluid conduit inlet is fluidly connected to the air intake manifold and the fluid conduit outlet is fluidly connected to the mouthpiece, and a fluid flow path is defined between the ambient air input port and the inhalation aperture, the fluid flow path passing proximate the heating element assembly; a control assembly substantially enclosed with the vaporizer body and electrically coupled with the fluid flow sensor assembly and the heating element, the control assembly for reading from a memory circuit which is for storing at least a pulse width modulation profile therein where upon the fluid flow signal being generated the at least a pulse width modulation profile stored within the memory circuit for controllably applying electrical power with respect to time to the heating element based upon the least a pulse width modulation profile, the heating element for heating of the heating element assembly and for creating an aerosol from the vaporizable material that is wicked into the heating element assembly and for the aerosol to flow into the fluid flow path and for the aerosol to mix together with the ambient air flow through the manifold fluid flow path for together to flow from the mouthpiece.

In some embodiments there is provided a wicking time where upon the creating an aerosol from the vaporizable material that is wicked into the heating element assembly, a subsequent application of the stored at least a pulse width modulation profile to the heating element is ceased for a predetermine amount of time to facilitate re-wicking of the vaporizable material into the heating element assembly proximate the heating element.

In some embodiments there is provided a pulse width modulation array comprises a plurality of pulse width modulation values stored in a pulse width modulation array, wherein generating a pulse width modulation value from within the array of pulse width modulations in a calibration phase comprises: applying a predetermined electrical power over time to the heating element as a first pulse width value and obtaining a first calibration temperature signal through a non-contact pyrometric observation of heating element assembly; comparing the first calibration temperature signal to a predetermined temperature signal; amending the first pulse width applied to the heating element to minimize a difference between the first calibration temperature signal and the predetermined temperature signal to create an amended first pulse width value; storing of the first pulse width value within the pulse width modulation array as a first entry.

In some embodiments there is provided applying a predetermined electrical power over time to the heating element as a second pulse width value and obtaining a second calibration temperature signal through a non-contact pyrometric observation of heating element assembly; comparing the second calibration temperature signal to the predetermined temperature signal; amending the second pulse width applied to the heating element to minimize a difference between the second calibration temperature signal and the predetermined temperature signal to create an amended second pulse width value; storing of the amended second pulse width value within the pulse width modulation array as a second entry.

In some embodiments there is provided populating of the pulse width modulation array through a plurality of applications of predetermined electrical power over time to the heating element and obtaining a plurality of temperature signal through a non-contact pyrometric observation of heating element assembly to generate a plurality of amended pulse width values to minimize a plurality of temperature differences between a plurality of temperature signals and the predetermined temperature signal; storing of the plurality of amended pulse width values as the at least a pulse width modulation profile within the memory circuit.

In some embodiments there is provided controllably applying electrical power with respect to time to the heating element based upon the least a pulse width modulation profile creates a substantially uniform temperature signal through the non-contact pyrometric observation of heating element assembly, wherein the substantially uniform temperature signal comprises a deviation from the predetermined temperature signal of about plus or minus 10 percent variation for less than 70% of time for which the pulse width modulation profile has been applied to the heating element.

In some embodiments there is provided a wicking time where upon the creating an aerosol from the vaporizable material that is wicked into the heating element assembly, a subsequent application of the stored at least a pulse width modulation profile to the heating element is ceased for a predetermine amount of time to facilitate re-wicking of the vaporizable material into the heating element assembly proximate the heating element wherein the predetermine amount of time is at least thirty seconds.

In some embodiments there is provided the heating element assembly comprises a 40-50% open porosity and a pore size ranging from 1 to 100 microns and where the heating element assembly comprises aluminum oxide.

In some embodiments there is provided the heating element assembly comprises a porous ceramic substrate inlaid with a heating element comprising a resistive wire attached to electrical couplings, wherein electrical couplings are extending from the heating element past an outside surface of the heating element assembly are spaced radially and extend axially from the heating element assembly wherein the electrical couplings are approximately parallel with the fluid flow passage.

In some embodiments there is provided the heating element assembly comprises a porous ceramic substrate inlaid with a heating element comprising a resistive wire, wherein electrical couplings extending from the heating element past an outside surface of the heating element assembly are spaced radially and extend axially from the heating element assembly wherein the electrical couplings are approximately perpendicular with the fluid flow passage.

In some embodiments there is provided thee heating element assembly comprises a 40-50% open porosity and comprising a tortuous pore structure with pore size ranging from 1 to 100 microns and where the heating element assembly comprises aluminum oxide and silicon carbide.

In some embodiments there is provided controllably applying electrical power with respect to time to the heating element based upon the least a pulse width modulation profile comprises: monitoring a flow of air through the manifold fluid flow exceeding the predetermined flow threshold and applying of the pulse width modulation profile to the heating element while the fluid flow is exceeding the predetermined flow threshold and ceasing to apply the pulse width modulation profile when the fluid flow is other than exceeding the predetermined flow threshold for a duration of the wicking time.

In some embodiments there is provided a cartridge receptacle formed within the elongated base, wherein the cartridge receptacle is defined between the sidewalls, second end of the air intake manifold and a cartridge is removably mountable in the cartridge receptacle, the cartridge comprising: a cartridge housing extending from a first cartridge end to a second cartridge end, wherein the elongated storage compartment is enclosed by the cartridge housing, wherein the heating assembly is disposed within the cartridge housing where the heating assembly disposed first end is proximate the cartridge housing first cartridge end wherein the memory circuit is disposed within the cartridge and the cartridge comprising a plurality of cartridge electrical contacts at the first cartridge, the plurality of electrical contacts being engageable with corresponding base electrical contacts provided on the vaporizer device wherein the control assembly is for reading from the memory circuit through the electrical engagement of the plurality of electrical contacts with corresponding base electrical contacts.

In some embodiments there is provided weighing of the vaporizer device to obtain a pre vaporization weight; generating of dosing data for the least a pulse width modulation profile within the memory circuit through coupling of the vaporizer device mouthpiece with a vapor sampling system; performing an inhalation using the vapor sampling system from the vaporizer device and triggering of the fluid flow sensor assembly to generate the fluid flow signal and for the at least a pulse width modulation profile to be applied to the heating element; weighing of the vaporizer device to obtain a post vaporization weight; subtracting of the pre vaporization weight to the post vaporization weight to obtain a vapor weight; storing of the vapor weight within the memory circuit corresponding with the least a pulse width modulation profile.

In some embodiments there is provided the stored vapor weight to a user after an inhalation by the user from the mouthpiece of the vaporize device.

In accordance with this broad aspect there is provided a vaporizer device comprising: a vaporizer body comprising: an elongated base extending from a first end to a second end, the elongated base including a pair of opposed sidewalls extending between the first end and the second end and a second end wall at the second end; a mouthpiece formed at the second end of the base, the mouthpiece comprising an inhalation aperture through the second end wall; an air intake manifold mounted to the base, the air intake manifold having a first manifold end and a second manifold end with a manifold fluid flow path defined therethrough, the air intake manifold comprising an ambient air input port disposed between the first manifold end and the second manifold end, the ambient air input port being exposed to an external environment; a fluid flow sensor assembly fluidly coupled between first manifold end and a second manifold with the manifold fluid flow path, the fluid flow sensor assembly for generating a fluid flow signal in dependence upon a flow of air through the manifold fluid flow exceeding a predetermined flow threshold; an elongated storage compartment, the storage compartment being configured to store a liquid vaporizable material, the storage compartment comprising an inner storage volume wherein the vaporizable material is storable in the inner storage volume, the elongated storage compartment comprising a first end and a second end opposite the first end; a heating assembly disposed at the elongated storage compartment first end, the heating assembly comprising a heating element thermally coupled with the heating element assembly comprising a porosity, and wherein the heating element assembly is in fluid communication with the inner storage volume for wicking of the vaporizable material into the heating element assembly; and a fluid conduit extending parallel with the elongated storage compartment from the first end to the second end, the fluid conduit having a fluid conduit inlet proximate the elongated storage compartment first end and a fluid conduit outlet proximate the elongated storage compartment second end, wherein the fluid conduit is in fluid communication with the heating element assembly and the fluid conduit inlet is fluidly connected to the air intake manifold and the fluid conduit outlet is fluidly connected to the mouthpiece, and a fluid flow passage is defined between the ambient air input port and the inhalation aperture, the fluid flow passage passing proximate the heating element assembly; a control assembly coupled with an energy storage member having a charge and substantially enclosed with the vaporizer body and electrically coupled with the fluid flow sensor assembly and the heating element, the control assembly for reading from a memory circuit which is for storing at plurality a pulse width modulation profile therein where upon the fluid flow signal being generated, one of the pulse width modulation profile stored within the memory circuit being selected for controllably applying electrical power with respect to time to the heating element based upon the selected pulse width modulation profile, the heating element for heating of the heating element assembly and for creating an aerosol from the vaporizable material that is wicked into the heating element assembly and for the aerosol to flow into the fluid flow passage and for the aerosol to mix together with the ambient air flow through the manifold fluid flow path for together to flow from the mouthpiece; wherein selecting of the selected pulse width modulation profile stored within the memory circuit is dependent upon at least one of a viscosity of the liquid vaporizable material and the porosity of the heating element assembly and the charge of the energy storage member.

In some embodiments there is provided a user input interface wherein the user input interface comprises at least a button for selecting of the selected pulse width modulation profile.

In accordance with this broad aspect there is provided a cartridge usable with the vaporizer device having a control circuit, the cartridge comprising a mouthpiece and having an inhalation aperture; a cartridge housing extending from a first end of the cartridge to a second end of the cartridge; an storage compartment, the storage compartment being configured to store a vaporizable material, the storage compartment comprising an inner storage volume wherein the vaporizable material is storable in the inner storage volume, wherein the inner storage volume is enclosed by the cartridge housing; a heating element assembly disposed at the first end of the storage compartment, the heating assembly comprising a heating element, a wicking element, wherein the heating element is in thermal contact with the wicking element, wherein the storage interface member surrounds the wicking element, and the storage interface member includes a plurality of circumferentially spaced fluid apertures fluidly connecting the wicking element to the inner storage volume; and a fluid conduit extending through the housing from a conduit inlet at the first end to a conduit outlet at the second end, wherein the fluid conduit is fluidly connected to the wicking element, the fluid conduit passes through the heating element assembly, wherein the storage compartment, heating element assembly and fluid conduit are concentrically disposed, wherein the storage compartment surrounds the heating element assembly and the fluid conduit, wherein the fluid conduit extends along the entire length of the elongated storage compartment; a memory circuit for storing at least a pulse width modulation profile therein for being read by the control circuit for providing of the at least a pulse width modulation profile to the heating element for heating at least a portion of the vaporizable material wicked into the heating element assembly for generating an aerosol therefrom into the fluid conduit.

In some embodiments there is provided a fluid flow sensor assembly fluidly coupled upstream of the heating assembly, the fluid flow sensor assembly for generating a fluid flow signal in dependence upon a flow of air through the fluid conduit exceeding a predetermined flow threshold for triggering of the at least a pulse width modulation profile being applied to the heating element.

In accordance with this broad aspect there is provided A vaporizer device and system comprising: providing a vaporizer device comprising a vaporizer body; providing a control assembly substantially enclosed with the vaporizer device, the control assembly comprising a first wireless communication module and the control assembly comprising a memory circuit for storing a first SSID and a first password and for executing steps of entering a wireless provisioning mode through creating a web server using the control assembly and the first wireless communication module by providing a first access point functioning as a web server having the first SSID and the first password and first IP address; provisioning the vaporizer device for wirelessly connect with a third wireless communication module as part of a router assembly having a third SSID and third password for and accessing a dosing data server having a dosing data server database through internet access by providing a second wireless communication module as part of a computing device having a display screen and a processing circuitry for executing a web browser and connecting of the second wireless communication module to the first wireless communication module first access point functioning as the web server in an administrator mode by using the first SSID and the first password and the first IP address through the web server displaying a vaporizer device HTML page wirelessly provided by the web server, wherein on the display screen the third SSID and third password is provided as input parameters to the displaying vaporizer device HTML page and the provided input is wirelessly provided to the control assembly, enabling storing of the third SSID and third password within the memory circuit of the control assembly for enabling of the first wireless communication module and the control assembly to directly connect with the dosing data server database through the internet access.

In some embodiments there is provided a keypad electrically coupled with the control assembly and entering a key sequence on the keypad for enabling of the control assembly for functioning as the web server.

In some embodiments there is provided s first IP address that comprises 192.168.X.X.

In some embodiments there is provided an elongated base extending from a first end to a second end, the elongated base including a pair of opposed sidewalls extending between the first end and the second end and a second end wall at the second end; a mouthpiece formed at the second end of the base, the mouthpiece comprising an inhalation aperture through the second end wall; an air intake manifold mounted to the base, the air intake manifold having a first manifold end and a second manifold end with a manifold fluid flow path defined therethrough, the air intake manifold comprising an ambient air input port disposed between the first manifold end and the second manifold end, the ambient air input port being exposed to an external environment; a fluid flow sensor assembly fluidly coupled between first manifold end and a second manifold with the manifold fluid flow path, the fluid flow sensor assembly for generating a fluid flow signal in dependence upon a flow of air through the manifold fluid flow exceeding a predetermined flow threshold; an elongated storage compartment, the storage compartment being configured to store a vaporizable material, the storage compartment comprising an inner storage volume wherein the vaporizable material is storable in the inner storage volume, the elongated storage compartment comprising a first end and a second end opposite the first end; a heating element assembly disposed at the elongated storage compartment first end, the heating assembly comprising a heating element, wherein the heating element is thermally coupled with the heating element assembly, and wherein heating element assembly is in fluid communication with the inner storage volume for wicking of the vaporizable material into the heating element assembly; and a fluid conduit extending parallel with the elongated storage compartment from the first end to the second end, the fluid conduit having a fluid conduit inlet proximate the elongated storage compartment first end and a fluid conduit outlet proximate the elongated storage compartment second end, wherein the fluid conduit is in fluid communication with the heating element assembly and the fluid conduit inlet is fluidly connected to the air intake manifold and the fluid conduit outlet is fluidly connected to the mouthpiece, and a fluid flow path is defined between the ambient air input port and the inhalation aperture, the fluid flow path passing proximate the heating element assembly; where the control assembly includes a vaporizer device memory circuit and is substantially enclosed with the vaporizer body and electrically coupled with the fluid flow sensor assembly and the heating element, the control assembly for reading from a memory circuit which is for storing at least a pulse width modulation profile therein where upon the fluid flow signal being generated the at least a pulse width modulation profile stored within the memory circuit for controllably applying electrical power with respect to time to the heating element based upon the least a pulse width modulation profile, the heating element for heating of the heating element assembly and for creating an aerosol from the vaporizable material that is wicked into the heating element assembly and for the aerosol to flow into the fluid flow path and for the aerosol to mix together with the ambient air flow through the manifold fluid flow path for together to flow from the mouthpiece, wherein upon inhalation from the mouthpiece an interaction is generated and also generating an interaction log by the control assembly, where for an interaction from a plurality of interaction with the vaporizer device a plurality of interaction parameters are stored are stored within the vaporizer device memory circuit.

In some embodiments there is provided a mouthpiece as part of the vaporization device and generating an interaction through inhalation through the mouthpiece and generating an interaction log by the control assembly for storing of the interaction log within the vaporization device memory circuit wherein for each interaction with the vaporizer device by the user, there may be a plurality of interaction parameters generated.

In some embodiments there is provided the interaction log that comprises data relating to at least two of the following interaction parameters comprising a start use time and an end use time and a duration of use time and a an ambient temperature surrounding vaporizer device and a battery level of the vaporizer device and an actual inhalation profile achieve through inhalation from the mouthpiece and a selected dose size and a pulse width modulation profile applied to the heating element and a corresponding calibrated measured dose value and an angle at which the vaporizer device is being held in relation to ground and a user reported dose effectiveness.

In some embodiments there is provided a keypad comprising a plurality of keys electrically coupled with the control assembly and post inhalation from the vaporization device for entering a key from the plurality of keys for receiving input from the user as the user reported dose effectiveness.

In some embodiments there is provided enabling of the first wireless communication module and the control assembly to directly connect with the dosing data server through the internet access comprises transferring of the interaction log stored within the vaporization device memory circuit to the dosing data server database as a stored interaction log.

In some embodiments there is provided an interaction log stored within the vaporization device memory circuit comprises a first interaction and a second interaction where data corresponding to the first interaction is different than data corresponding to the second interaction.

In accordance with this broad aspect there is provided a vaporizer device and system comprising: providing a vaporizer device comprising a vaporizer body; providing a control assembly substantially enclosed with the vaporizer device, the control assembly comprising a vaporization device memory circuit and a first wireless communication module and for storing a first SSID and a first password and a first IP address and for storing a third SSID and third password; providing a mouthpiece as part of the vaporization device and generating an interaction through inhalation through the mouthpiece and generating an interaction log by the control assembly for storing of the interaction log within the vaporization device memory circuit wherein for each interaction with the vaporizer device by the user, there may be a plurality of interaction parameters generated; wirelessly provisioning the vaporizer device using the first SSID and the first password for wirelessly connect with a third wireless communication module as part of a router assembly comprising the third SSID and the third password for and accessing a dosing data server having a dosing data server database through internet access; enabling of the first wireless communication module and the control assembly to directly connect with the dosing data server through the internet access comprises transferring of the interaction log stored within the vaporization device memory circuit to the dosing data server database as a stored interaction log.

In some embodiments there is provided a second wireless communication module as part of a computing device having a display screen and a processing circuitry for executing a web browser and connecting of the second wireless communication module to the first wireless communication module first access point functioning as the web server in an administrator mode by using the first SSID and the first password and the first IP address through the web server displaying a vaporizer device HTML page wirelessly provided by the web server, wherein on the display screen the third SSID and third password is provided as input parameters to the displaying vaporizer device HTML page and the provided input is wirelessly provided to the control assembly.

In some embodiments there is provided a removable cartridge assembly comprising a heating element assembly comprising a heating element a cartridge memory module for storing therein at least parameters relating to a PWM profile for being applied to the heating element; releasably electrically coupling of the cartridge memory module with the vaporization device memory circuit; applying of the at least parameters relating to a PWM profile to the heating element; storing of the of the at least parameters relating to a PWM profile applied to the heating element as at least an entry within the interaction log data stored within the vaporization device memory circuit.

In some embodiments there is provided a progressive web application for being executed within the web browser and visually represented on display screen; and receiving of stored interaction log from the dosing data server database; visually representing of the interaction log data stored on the dosing data server database server on the display screen.

In some embodiments there is provided disconnecting of the first wireless communication module from the third wireless communication module and connecting the first wireless communication module to the second wireless communication module; enabling of the vaporizer device control assembly to function as the web server; displaying the vaporizer device HTML page; production a visual indication of the interaction through inhalation from the mouthpiece through a progress indicator on the display screen.

In some embodiments there is provided the first, second and third wireless communication modules comprise an 802.11x protocol and operate between 2.4 GHz and 5 GHz.

In some embodiments there is provided an audio microphone and audio processing circuitry for having ability to record of audio post inhalation from the vaporization device from the user as the user reported dose effectiveness.

These and other aspects and features of various embodiments will be described in greater detail below.

### BRIEF DESCRIPTION OF THE DRAWINGS

For a better understanding of the described embodiments and to show more clearly how they may be carried into effect, reference will now be made, by way of example, to the accompanying drawings in which:

FIG. 1 is a top front perspective view of an example vaporization device with removable cartridge in an unlocked position in accordance with an embodiment;

FIG. 2 is a side perspective view of an example control circuit assembly removed from the base of the vaporization device of FIG. 1 in accordance with an embodiment;

FIG. 3 is a front perspective view of a base and cover of the body of the vaporization device of FIG. 1 in accordance with an embodiment;

FIG. 4 is an exploded perspective view of an example cartridge assembly in accordance with an embodiment;

FIG. 5 is a front perspective view of an example heating element assembly that may be used in the cartridge assembly of FIG. 4 in accordance with an embodiment;

FIG. 6 is a side cutaway view showing the example cartridge assembly of FIG. 4 in an unlocked position relative to a portion of the cartridge receptacle of the example vaporization device of FIG. 1;

FIG. 7 is an isolated perspective view of the example cartridge assembly of FIG. 4 and an example air intake manifold that may be used with the example vaporization device of FIG. 1;

FIG. 8 is a sectional view of the example air intake manifold of FIG. 7 attached to the example cartridge assembly of FIG. 4;

FIG. 9 is an enlarged view taken of a filling aperture of the example cartridge assembly of FIG. 4;

FIG. 10 is a top cutaway view of the example vaporization device of FIG. 1 showing the removable cartridge assembly in an installed position;

FIG. 11 is an example diagram of a cartridge identifier label that may be used with the cartridge assembly of FIG. 4 in accordance with an embodiment;

FIG. 12 is a top front perspective view of another example vaporization device and cartridge assembly in accordance with an embodiment;

FIG. 13 is a top front perspective view of the vaporization device base of FIG. 12 with the cartridge assembly removed in accordance with an embodiment;

FIG. 14 is a top front perspective view of an insert assembly of the vaporization device of FIG. 13 in accordance with an embodiment;

FIG. 15 is a bottom front perspective view of the cartridge assembly of FIG. 12 in accordance with an embodiment;

FIG. 16 is a side perspective view of the vaporization device of FIG. 12 with a vaporization body housing removed in accordance with an embodiment;

FIG. 17 is a side perspective view of a vaporization body housing that may be used with the vaporization device of FIG. 12 in accordance with an embodiment;

FIG. 18 is an isolation view of an example air intake manifold that may be used with the vaporization device of FIG. 12 in accordance with an embodiment;

FIG. 19 is an exploded view of the example air intake manifold of FIG. 18;

FIG. 20 is a top perspective view of the example air intake manifold of FIG. 18;

FIG. 21 is side section view of the example air intake manifold of FIG. 18 along line 21-21 shown in FIG. 20;

FIG. 22 is a side perspective view of the vaporization device of FIG. 12 with the cartridge assembly partially removed;

FIG. 23 is a rear side perspective view of the vaporization device of FIG. 22 with the cartridge assembly partially removed;

FIG. 24 is a front perspective view of the cartridge assembly of FIG. 12 with a cartridge cover removed in accordance with an embodiment;

FIG. 25 is a front perspective view of another example cartridge assembly that may be used with the vaporization device of FIG. 12 with a cartridge cover removed in accordance with an embodiment;

FIG. 26 is a cross-sectional side view of the cartridge assembly of FIG. 25 installed in the vaporization device of FIG. 12 in accordance with an embodiment;

FIG. 27 is a rear perspective exploded view of the cartridge assembly of FIG. 24 showing the cartridge body, cartridge cover and a sealing member in accordance with an embodiment;

FIG. 28 is a front perspective exploded view of the cartridge assembly of FIG. 27;

FIG. 29 is a front perspective isolation view of a storage compartment base and heating assembly that may be used with the cartridge assembly of FIG. 24 in accordance with an embodiment;

FIG. 30 is a rear perspective isolation view of the storage compartment base and heating assembly of FIG. 29;

FIG. 31 is a front perspective view of a heating assembly that may be used with the cartridge assembly of FIG. 24 in accordance with an embodiment;

FIG. 32 is a rear perspective view of the heating assembly of FIG. 31;

FIG. 33 is an exploded view of the heating assembly of FIG. 31;

FIG. 34 is a top perspective view of a heating element that may be used with the heating assembly of FIG. 31 in accordance with an embodiment;

FIG. 35 is a side view of the heating element of FIG. 34;

FIG. 36 is a top plan view of the heating element of FIG. 34;

FIG. 37 is a side view of another heating element that may be used with the heating assembly of FIG. 31 in accordance with an embodiment;

FIG. 38 is a top plan view of the heating element of FIG. 37;

FIG. 39 is a bottom plan view of the heating element of FIG. 37;

FIG. 40 is a top front perspective view of the cartridge cover of the cartridge assembly of FIG. 25 in accordance with an embodiment;

FIG. 41 is a top front perspective view of the cartridge base of the cartridge assembly of FIG. 25 in accordance with an embodiment;

FIG. 42 is a perspective cut-away view of the cartridge base of FIG. 41 with a portion of the base housing removed;

FIG. 43 is a perspective view of an example heating assembly that may be used with the cartridge assembly of FIG. 25 in accordance with an embodiment;

FIG. 44 is a perspective view of an example heating element and an example wick element that may be used in the heating assembly of FIG. 43;

FIG. 45 is a perspective view of the heating element of FIG. 44;

FIG. 46 is a top front perspective view of the cartridge cover of the cartridge assembly of FIG. 24 in accordance with an embodiment;

FIG. 47 is a top front perspective view of the cartridge base of the cartridge assembly of FIG. 24 in accordance with an embodiment;

FIG. 48 is a perspective cut-away view of the cartridge base of FIG. 47 with a portion of the base housing removed;

FIG. 49 is a perspective view of an example heating assembly that may be used with the cartridge assembly of FIG. 24;

FIG. 50 is a perspective view of an example heating element and an example wick element that may be used in the heating assembly of FIG. 49;

FIG. 51 is a perspective view of the heating element of FIG. 50;

FIG. 52 is a top front perspective view of the cartridge cover of another example cartridge assembly in accordance with an embodiment;

FIG. 53 is a top front perspective view of the cartridge base of the cartridge assembly of FIG. 52 in accordance with an embodiment;

FIG. 54 is a perspective cut-away view of the cartridge base of FIG. 53 with a portion of the base housing removed;

FIG. 55 is a perspective view of an example heating assembly that may be used with the cartridge assembly of FIG. 52;

FIG. 56 is a perspective view of the example heating assembly of FIG. 55 with a wick element removed;

FIG. 57 is a perspective view of the heating element of FIG. 56;

FIG. 58 is a perspective view of the heating element of FIG. 57 with a heating element cover removed;

FIG. 59 is a perspective view of another example vaporization device and cartridge assembly in accordance with an embodiment with the cartridge assembly removed;

FIG. 60 is a side perspective view of the vaporization device and cartridge assembly of FIG. 59 with the cartridge assembly removed;

FIG. 61 is a side perspective view of the vaporization device and cartridge assembly of FIG. 59 with the cartridge assembly installed in the vaporization device body;

FIG. 62 is a schematic sectional view of the cartridge assembly and cartridge receptacle of the vaporization device and cartridge assembly of FIG. 59 in accordance with an embodiment with the cartridge assembly removed;

FIG. 63 is a schematic illustration of a cartridge engagement member that may be used in the vaporization device of FIG. 59 in accordance with an embodiment;

FIG. 64 is a front perspective view of a cartridge filling apparatus in accordance with an embodiment;

FIG. 65 is a front perspective view of the cartridge filling apparatus of FIG. 64 with a cartridge base mounted to a cartridge engagement member in accordance with an embodiment;

FIG. 66 is a front perspective view of the cartridge filling apparatus of FIG. 64 with a cartridge cover mounted to a cartridge engagement member in accordance with an embodiment;

FIG. 67 is a top front perspective view of a cartridge testing assembly in accordance with an embodiment;

FIG. 68 is a top front perspective view of the cartridge testing assembly of FIG. 67 with a cartridge assembly being positioned within a cartridge receiving region;

FIG. 69 is a schematic circuit drawing of an example heating element sensing unit that may be used with a vaporization device in accordance with an embodiment;

FIG. 70 is an example plot illustrating heating element current and heating element temperature of an example vaporization device;

FIG. 71 is a top plan view an example vaporization device having a user input interface positioned on a device cover, in accordance with an embodiment;

FIG. 72 is a cutaway perspective view of the vaporization device of FIG. 71;

FIG. 73 is a side plan view of an example vaporization device having an activation sensor in accordance with an embodiment;

FIG. 74 is a bottom cut-away perspective view of a storage compartment base member that may be used in a cartridge assembly in accordance with an embodiment;

FIG. 75 is a top perspective view of the storage compartment base member installed within the storage compartment of a cartridge assembly in accordance with an embodiment;

FIG. 76 is a top perspective view of the storage compartment of the cartridge assembly of FIG. 75 with the storage compartment base member removed;

FIG. 77 is an example plot illustrating differential pressure measurements and inhalation volume measurements over a period of time;

FIG. 78 is another example plot illustrating differential pressure measurements and inhalation volume measurements over a period of time; and

FIG. 79 is a schematic drawing illustrating an example of a fluid manifold system that may be used with the cartridge assembly of FIG. 12 in accordance with an embodiment.

FIG. 80 is a side cutaway view of a vapor sampling system for airflow as well as phyto material extract calibration;

FIG. 81 is a portion of a side cutaway view of a vapor sampling system for airflow as well as phyto material extract calibration in an inhalation mode of operation;

FIG. 82 is a portion of a side cutaway view of a vapor sampling system for airflow as well as phyto material extract calibration in an exhalation mode of operation;

FIG. 83 is a rear perspective view of a vapor sampling system for airflow as well as phyto material extract calibration in a post exhalation mode of operation;

FIG. 84 is a front perspective view of a vapor sampling system for airflow as well as phyto material extract calibration in a post exhalation mode of operation;

FIG. 85 is a rear perspective view of a vapor sampling system for airflow as well as phyto material extract calibration in a post exhalation mode of operation;

FIG. 86 shows a graph is shown illustrating differences in VOCs and CO2 as detected by a first VOC sensor in three sequential tests where the first VOC sensor is exposed to vapor and then other than exposed to vapor;

FIG. 87, illustrates a graph of a first pulse-width modulation (PWM) profile as PWM200;

FIG. 88, illustrates a graph of a second pulse-width modulation (PWM) profile as PWM300;

FIG. 89, illustrates a graph of a third pulse-width modulation (PWM) profile as PWM350;

FIG. 90, illustrates a graph of a fourth pulse-width modulation (PWM) profile as PWM400;

FIG. 91 illustrates various PWM profiles applied to a heating element assembly and vapor captured from a vaporization device with resultant output signals for CO2eq signal and TVOC signal observed from a first VOC sensor;

FIG. 92 illustrates thermal imaging graphs obtained from using a FLIR measurement system at a sample rate of about 10 Hz when PWM400 PWM profile is applied to a heating element assembly;

FIG. 93 illustrates various inhalation profiles being applied to a vaporization device and a resulting differential pressure signals as reported by a mass airflow sensor;

FIG. 94a illustrates a first exemplary inhalation profile as IP1;

FIG. 94b illustrates a second exemplary inhalation profile as IP2;

FIG. 94c illustrates a third exemplary inhalation profile as IP3;

FIG. 94d illustrates a fourth exemplary inhalation profile as IP4;

FIG. 94e illustrates a fifth exemplary inhalation profile as IP5;

FIG. 95 illustrates a graph of different inhalation rates through a vaporization device and resulting CO2 and VOC signals as detected by a first VOC sensor;

FIG. 96 illustrates measurements of a dose and presented in a weight table created for a vaporization device for multiple tests;

FIG. 97 illustrates measurements of a dose and presented in a weight table created for a vaporization device for multiple tests for a varying PWM profile applied to a heating element assembly;

FIG. 98 illustrates a plurality PWM profiles and calibrated dose weight;

FIG. 99 illustrates a plurality of inhalation profiles and calibrated dose weight;

FIG. 100 illustrates an inhalation being performed by a user being compared to a lookup table stored inhalation profiles to provide a calibrated indicated dose weight to an end user;

FIG. 101 illustrates resulting CO2 and VOC signals as detected by a first VOC sensor in relation to battery power available to a vaporization device;

FIG. 102 illustrates a temperature profile of about 280 degrees Celsius for a heating element assembly;

FIG. 103 illustrates a corresponding PWM profile used to generate a temperature profile shown in FIG. 102;

FIG. 104 illustrate a plurality of vapor weight measurements using a vapor sampling system and a heating element having a second resistance;

FIG. 105 illustrate a plurality of vapor weight measurements using a vapor sampling system and heating element having a first resistance;

FIG. 106 illustrates a PWM profile applied to a first resistance and a second resistance of a heating element;

FIG. 107 illustrates an exemplary means of providing a dose progress indication to a user when using of a vaporization device;

FIG. 108 illustrates a portion of a cartridge assembly showing a heating element assembly in an exploded view;

FIG. 109 illustrates a cutaway view of a cartridge assembly;

FIG. 110 illustrates a provisioning process for a vaporizer device in accordance with an embodiment of the invention;

FIG. 111 illustrates a dosing data server having a dosing data server database stored therein for a plurality of users;

FIG. 112 illustrates an exemplary view of how a dosing data may be presented to the user on a display screen of a device executing a web browser;

FIG. 113 illustrates an exemplary view of how a dosing data may be stored within a vaporizer device memory circuit and how the data may correspond to data stored on a database server;

FIG. 114 illustrates an interaction of for a vaporizer device stored within the vaporization device memory circuit for a plurality of users and for a plurality of cartridge assemblies;

FIG. 115 illustrates a cartridge assembly as an embodiment of the invention is shown from a front cutaway render view;

FIG. 116 illustrates a cartridge assembly as an embodiment of the invention is shown from a rear cutaway render view;

FIG. 117 illustrates a cartridge assembly as an embodiment of the invention as a side cutaway render view;

FIG. 118 illustrates a heating element assembly from a front perspective view;

FIG. 119 illustrates a heating element assembly from a rear perspective view with a S-type heating element;

FIG. 120 illustrates a heating element assembly from a front perspective view with a W-type heating element;

FIG. 121 illustrates a cartridge assembly an embodiment of the invention from a front perspective exploded view;

FIG. 122 illustrates a cartridge assembly an embodiment of the invention from a rear perspective exploded view;

FIG. 123 illustrates a heating element assembly from a bottom view with a S-type heating element;

FIG. 124 illustrates a heating element assembly from a bottom view with a W-type heating element;

The drawings included herewith are for illustrating various examples of articles, methods, and apparatuses of the teaching of the present specification and are not intended to limit the scope of what is taught in any way.

### DETAILED DESCRIPTION

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising,” and variations thereof mean “including but not limited to,” unless expressly specified otherwise. A listing of items does not imply that any or all of the items are mutually exclusive, unless expressly specified otherwise. The terms “a,” “an,” and “the” mean “one or more,” unless expressly specified otherwise.

Embodiments described herein relate generally to vaporization of vaporizable material, such as phyto materials and phyto material products. Although embodiments are described herein in relation to vaporization of phyto material and phyto material products, it will be understood that other vaporizable materials, such as vaporizable nicotine products and/or synthesized vaporizable compounds, or combinations of vaporizable components may be used. For instance, various vaporizable products containing nicotine or plant derived extracts or oils, such as cannabis extract, CBD or terpine extracts and/or synthesized compounds may be used. Phyto material products may be derived from phyto materials such as the leaves or buds of cannabis plants.

Various methods of vaporizing phyto materials and phyto material products, such as cannabis products, are known. Phyto material is often vaporized by heating the phyto material to a predetermined vaporization temperature. The emitted phyto material vapor may then be inhaled by a user for therapeutic purposes.

Devices that vaporize phyto materials are generally known as vaporizers. In some cases, oils or extracts derived or extracted from the phyto materials may also be vaporized. For cannabis oils or extracts, temperatures in the range of about 500 to 700 degrees Fahrenheit may be applied to vaporize these phyto material products may generate phyto material vapor.

The phyto material vapor may be emitted at a temperature that is uncomfortable for a user to inhale. Accordingly, it may be desirable to cool the vapor prior to inhalation.

Phyto material products, such as oils and extracts, may be generated in batches. The batches may be mixed in a liquid or semi-liquid state. This may facilitate testing of the potency of the phyto material product and provide greater consistency of potency throughout a batch of phyto material product.

Phyto material products, such as oils and extracts may be provided in various liquid, semi-liquid/semi-solid, and solid forms. These liquid phyto material products may be stored in a cartridge or capsule that may be used with a vaporizer device.

In some cases, a vaporizable material may be added into a cartridge, and in turn, this cartridge is inserted into a vaporizer. However, it may be quite difficult to fill the cartridges with vaporizable material. Typically, a thin syringe is used to inject very dense oil through a very small applicator tip/orifice into the cartridge. This is a slow process that takes a significant amount of time and, as a result, is not very efficient. Some pressurized systems exist that allow for pressurized extracts to be injected into a cartridge. However, these systems tend to be very inefficient and require manual intervention.

Vaporization devices that provide for removable cartridges to be vaporized may allow users to adjust the type and/or potency of phyto material products being consumed. A user may insert a cartridge of a particular type into their vaporization device based on the desired therapeutic effect. If a different effect is desired, or the cartridge is spent, the old cartridge may be removed and a new or different cartridge may be inserted for subsequent vaporization.

Vaporization of material from a phyto material cartridge may involve airflow through the phyto material cartridge. However, it may be difficult to ensure consistent airflow through the cartridge as the space available within the vaporization devices limits the space available for a fluid conduit through the cartridge. Smaller fluid conduits through a phyto material cartridge may restrict airflow and cause user inconvenience or discomfort, since the user may be required to repeatedly puff or inhale short sharp intakes of air to encourage air flow through the cartridge.

Embodiments described herein related generally to methods and devices for vaporizing phyto material, in particular liquids containing phyto material such as medical cannabis. In embodiments discussed herein, examples of vaporization devices or vaporizer devices are described that may be used to vaporize cartridges containing vaporizable products such as liquid phyto material products. The example vaporizer devices may be associated with any suitable type of cartridge containing vaporizable liquid materials that is engageable with the vaporizer devices, such as the example cartridges described herein.

Similarly, in embodiments discussed herein, examples of cartridges usable to store liquid vaporizable materials that are vaporizable using vaporizer devices are described. The example cartridges may be associated with any suitable type of vaporizer device operable to receive the cartridges, such as the example vaporizer devices described herein.

Furthermore, in embodiments discussed herein, examples of apparatuses and methods for filling cartridges with liquid vaporizable material are described. The example filling apparatuses and methods may be associated with any suitable type of cartridge, such as the example cartridges described herein.

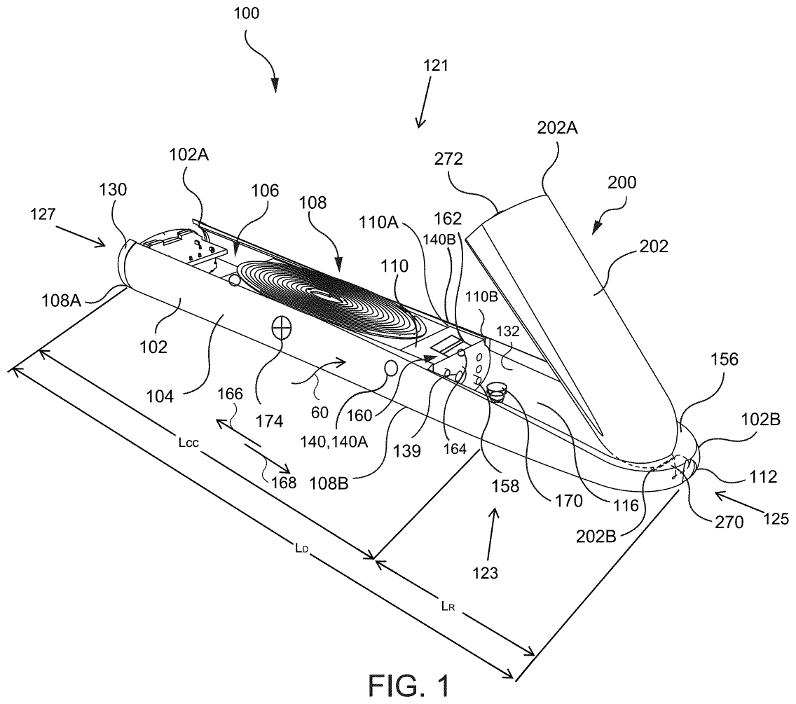

Referring now to FIGS. 1-11, shown therein is an example of a vaporization device 100. Vaporization device 100 is an example of a vaporization device that may be used to vaporize material that may be derived from or contain extracts from phyto materials such as cannabis. Vaporization device 100 may be used to vaporize phyto material products in a liquid or semi-liquid form, which may be referred to herein as vaporizable liquids or liquid vaporizable materials.

In the example shown, vaporization device 100 has a top side 121, a bottom side 123, a front side 125, a rear side 127, and opposed lateral sides. Vaporization device 100 generally includes a device body 102 that includes a base 104 and a cover 144. Base 104 defines a bottom surface and opposed lateral sides of vaporization device 100. The device body 102 may be used to house and retain various components of the vaporization device 100, such as a control assembly 108, air intake manifold 110, and a cartridge assembly 200.

Base 104 defines a cartridge receptacle 116 that is shaped to receive and engage a cartridge, such as cartridge assembly 200, used to store liquid vaporizable material. The cartridge assembly 200 may be removably mounted to the device body 102 in the cartridge receptacle 116. The vaporization device 100 may then be activated to vaporize the vaporizable liquid in the cartridge assembly 200 and generate phyto material vapor. A user may then inhale the emitted vapor through inhalation aperture 112 to achieve therapeutic effects.

Device body 102 extends from a first device end 102A to a second device end 102B. The terminology “first”, “second” and “third” and the like used herein is arbitrary and interchangeable. The inhalation aperture 112 may be provided at the second end 102B. A user may inhale through the inhalation aperture 112 to consume the phyto material vapor.

The device body 102 may have an elongated form that extends over a device length L_{D} from the first device end 102A to the second device end 102B. In the example shown, the device body 102 includes a base 104 that extends between the first device end 102A and the second device end 102B. The base 104 may define a housing or outer walls of the device body 102, such as a bottom wall and sidewalls for body 102. The base 104 may define an interior device cavity or recess 106 within the housing walls. Various components of the vaporizer device 100 may be positioned within the recess 106.

In the example shown, the base 104 defines a single combined bottom and sidewall extending between the first device end 102A and the second device end 1028. The base 104 has inwardly curved sidewalls, with a semi-annular shape along the length of device body 102. In alternative embodiments, the base 104 may be formed with various other configurations, such as triangular, rectangular, hexagonal, etc. In general, however, the base 104 may have at least one open or exposed (or at least partially exposed) side to allow components, such as a cartridge assembly 200, to be inserted into the device body 102.

The recess 106 defined by the base 104 may include a portion or section that defines a cartridge receptacle 116. In the example shown, the cartridge receptacle 116 is defined by the recess 106 proximate the second end 102b of device body 102. The cartridge receptacle 116 may be shaped to receive a phyto material cartridge such as cartridge assembly 200.

The recess 106 may include a plurality of sections or regions along the length of vaporizer device 100. For example, the recess 106 may include a first section 107 defining a control assembly receiving space and a second section 109 defining the cartridge receptacle 116. In the example shown, the second recess section 109 is defined proximate the second end 102B of vaporizer device 100 extending towards the first end 102A of the vaporizer device 100. The first recess section 107 is defined proximate the first end 102A of vaporizer device 100 extending towards the second end 102B of the vaporizer device 100.

In the example shown, the base 104 has an open first end 102A. That is, the recess 106 is not enclosed (i.e. the base 104 does not include a wall) at the first device end 102A. The base 104 may have a substantially closed second end 1028, apart from inhalation aperture 112. The recess 106 is thus mostly closed at the second device end 1028 by the base 104 other than inhalation aperture 112.

The inhalation aperture 112 may be defined in the sidewall of base 104. In the example shown, inhalation aperture 112 is provided in the end wall of base 104 at the second device end 1028. Inhalation aperture 112 may provide fluid communication between an external environment that surrounds the vaporization device 100 and the interior device cavity 106. As in the example shown, the inhalation aperture 112 may be formed in the portion of base 104 that defines cartridge receptacle 116. A fluid flow path through the vaporization device 100 to inhalation aperture 112 may then extend through a cartridge assembly 200 that is positioned in the cartridge receptacle 116.

In some cases, in the absence of a cartridge assembly 200, the vaporizer device 100 may not define an enclosed fluid flow path that extends to the inhalation aperture 112. For instance, the cartridge receptacle 116 has an open top side when the cartridge assembly 200 is removed. Thus, the cartridge assembly 200 may be required in order to complete a fluid flow path through vaporizer 100.

In some embodiments, the inhalation aperture 112 may be flush with the end wall of base 104, e.g. as shown. Alternatively, inhalation aperture 112 may be provided as part of a mouthpiece that extends outwardly from the outer surface of the end wall of base 104. The mouthpiece may include a removable mouthpiece cover that may be cleaned and/or replaced.

The vaporizer 100 may include a control assembly 108. The control assembly 108 may be positioned within the interior device space 106 (see e.g. FIG. 1). For instance, control assembly 108 may be positioned within the first recess section 107.

The control assembly 108 may be enclosed within the recess 106. For example, a cover 144 may be secured over the first section 107 of recess 106 within which the control assembly 108 is positioned. This may protect elements of control assembly 108 from exposure to dirt or debris from the external environment.

As shown, the control assembly 108 may be mounted to a support member 114. The support member 114 may extend from a first member end 108A to a second member end 1088. The support member 114 may define a control assembly length L_{CC} measured from the first member end 108A to the second member end 1088.

The support member 114 may be positioned within the device cavity 106 with the first member end 108A located at the first device end 102A. The support member 114 may include an end cover member 118 at the first member end 108A. The end cover member 118 may define a first end wall for the vaporizer device 100. The end cover member 118 may engage the first end 102A of the base 104 to enclose the first end 102A.

In some cases, the end cover member 118 may be wholly or partially rubberized. For example, an inner surface of the end cover member 118 (facing the second end 102B) may be rubberized. This rubberized end cover member 118 may engage the base 104 at the first end 102A of when the support member 114 is positioned within the device 102. This may assist with securing the support member 114 to device 102 and enclosing the first end 102A.

Control circuit assembly 108 may include a control circuit 120, one or more wireless communication modules (122, 124, 126) such as Bluetooth, near-field communication (NFC), and Wi-Fi module 126, and an energy storage module 128, such as one or more batteries. The control circuit 120, Bluetooth module 122, NFC module 124, Wi-Fi module 126, and energy storage module 128 may all be mounted on, or supported by, the assembly support base 114. In some embodiments, the assembly support base 114 may include a motherboard that permits electrical communication between all components mounted thereon.

Energy storage module 128 may be electrically coupled to the control circuit 120 and the one or more wireless modules. The control circuit 120 may be electrically coupled to the wireless modules and may be configured to control the operation of the Bluetooth module 122, the NFC module 124 and the Wi-Fi Module 126. The wireless modules may allow firmware installed on vaporizer device 100, such as the control circuit 120, to be updated remotely (e.g. from a central server or through a user application).

Control circuit 120 may be configured to monitor and control various components of vaporization device 100. For example, control circuit 120 may be used to monitor and control the flow of current from energy storage members 128. Control circuit 120 may also be used to provide user interface functionality and user feedback, such as audio or visual outputs. The control circuit 120 may also be used to control the operation of vaporization device 100, such as monitoring device activation and controlling operation of a heating assembly that is onboard vaporization device 100 (including heating assembly provided within removable phyto material cartridges). Control circuit 120 may also monitor the state of various components of vaporization device 100, such as battery discharge levels, air flow sensor activity, sensor signals, heating element temperature and so forth. Control circuit 120 may also monitor one or more device sensors and feedback indicators, examples of which are described in further detail below.

In some embodiments, energy storage module 128 may be a rechargeable energy storage module, such as a battery or super-capacitor. Vaporization device 100 may include a power supply port (e.g. a USB-port or magnetic charging port) that allows the energy storage module 128 to be recharged. The energy storage module 128 may optionally be removable to allow it to be replaced. For instance, energy storage module 128 may include non-rechargeable batteries in some alternative cases.

In some embodiments, the vaporization device 100 may include a plurality of device status indicators. The status indicators may include various types of status indicators, such as auditory indicators, visual indicators, haptic feedback (e.g. a vibrating motor). The device status indicators may provide a user with information or feedback on various aspects of the device operation, such as remaining battery capacity, on/off status, mode of operation (e.g., high heat, medium heat, or low heat), temperature of a heating assembly, fill status of a cartridge, presence or absence of a cartridge in cartridge receptacle 116, whether to initiate an inhalation, whether to inhale deeper, whether to stop inhalation and so on.

For example, one or more indicator lights (e.g. Light-emitting diodes) may be provided on the vaporization device 100. The indicator lights may be electrically coupled to the control circuit 120. Accordingly, the control circuit 120 may control the operation of the indicator lights.

The indicator lights may be positioned proximate the first member end 108A, e.g. at device end 102A. The indicator lights may be visible from the exterior of vaporizer device 100, to allow a user to easily identify the status of the vaporizer device 100.

In the example shown, the indicator lights may include a plurality light emitting diodes (LEDs) 130. The LEDs 130 may be positioned around the member base 118 at the first member end 108A.

The vaporizer device 100 may include a cover 144. The cover 144 may be secured to base 104 to enclose components of the vaporizer device 100.

As shown, the cover 144 may be secured to base 104 overlying the first recess section 107. The cover 144 may thus enclose the support member 114, and associated components mounted thereon, within the recess 106. FIG. 1 shows the vaporization device 100 with the cover 144 removed, illustrating the control assembly 108 that may be enclosed by cover 144.

Optionally, device cover 144 may be removably mounted to the body device 102. This may permit access to the control assembly 108 for repairs and/or replacement. In other cases, the device cover 144 may be fixed to base 104 with the control assembly 108 positioned within the recess 106. In some such cases, the control assembly 108 may still be accessible, e.g. by sliding the support member 114 out the first device end 102A. In some embodiments the device cover 144 may be formed with the base 104 as a unitary construction (i.e. a unitary cover and base).

In the example shown, the device cover 144 extends between a first cover end 144A and a second cover end 144B over a cover length L_{C}. The first cover end 144A may be secured to base 104 aligned with the first device end 102A.

In the example shown, device cover 144 may be attached to the device base 104 by sliding the device cover 144 in a forward direction 146 from the first device end 102A towards the second device end 102B until the first cover end 144A aligns with the first device end 102A. Similarly, to remove the device cover 144 from the device body 102, the device cover 144 may be slid in a rearward direction 148 towards the first device end 102A.

In some embodiments, the device cover 144 may have an indent or recess 150 formed thereon, e.g. as shown in FIG. 3. Indent 150 may provide a grip for a user to manipulate the device cover 144, e.g. by inserting a finger or fingernail in recess 150 to slide device cover in directions 146 and 148. In some embodiments, instead of sliding, the device cover 144 may be secured to the device body 102 by aligning the first cover end 144A with the first device end 102 and then applying pressure to the device cover 138 to secure it to the device body 102. For instance, the device cover 144 may be secured in an upper side of the base 104 by a friction fit.

In the example shown, the device cover 144 may have a first lateral edge 144C and a second later edge 144D. Base 104 may include a first lateral upper edge 104A and a second lateral upper edge 104B. Each upper edge 104A and 104B of the base 104 may have an inner lip for at least a portion of the recess 106. In the example shown, upper edges 104A and 104B include inner lips that are shaped to correspond to the lateral edges 114C and 144D, respectively. The inner lips may be defined as the curved upper edges of a semi-annular device base 104.

Preferably, the inners lips on upper edges 104A and 104B extend from the first device end 102A over the first recess section 107. In some cases, the inner lips of the upper edges 104A and 104B may also extend over a third section 111 of recess 106 that is between the first recess section 107 and the cartridge receptacle 116. The inner lips defined in upper edges 104A and 104B may assist in retaining components such as the control circuit assembly 108 and air intake manifold 110 secured within base 104.

The inner lips may be defined to extend for a length substantially equal to the cover length L_{C}. The outer edges 144C and 144D of device cover 144 may frictionally engage the lips of upper edges 104A and 104B. This frictional engagement between the outer edges 144C, 144D and the upper lips of edges 104A, 104B may maintain the device cover 144 in a fixed position when attached to the device base 104. Additionally, or alternatively, in other embodiments, the device cover 144 and base 104 may include other engagement members, e.g. mating engagement members such as snap fittings.

Device cover 144 may be manufactured of a non-conductive material. This may facilitate communication using the wireless modules disposed within the recess 106. In some embodiments, the device cover 144 may be from rubber or thermoplastic materials.

The device cover 144 may be manufactured using material with a higher coefficient of friction than device base 104. This may facilitate attaching and removing the device cover 144 from base 104. The cover 144 may also provide a different tactile sense for a user gripping vaporizer device 100.

The base 104 may include a lined inner surface. An inner surface 132 of recess 106 may be lined (wholly or in part) with a partially compressible, resilient material. This may allow components, such as a cartridge assembly 200 and/or support member 114 to be positioned in recess 106 and then secured by frictionally engaging the inner lining of surface 132. For instance, the inner surface 132 of the recess 106 may be lined with a rubberized material.

In the example shown, the support member 114 has a generally rectangular shape. The outer lateral edges of support member 114 may frictionally engage the inner surface 132 of the base 104 when the support member is positioned within recess 106. When support member 114 is inserted into base 104, the lateral edges of support member 114 may compress the lining on the inner surface 132. The inner lining may be formed using a resilient material inclined to return to its uncompressed state. The resilience of inner lining may then assist in retaining support member 114 within the recess 106.

In some embodiments, the support member 114 may include angled sections along its lateral edges. The angled sections may define undercuts along both lateral edges of support member 114. When the support member 114 (and control assembly 108) is positioned within the recess 106, the undercuts may frictionally engage the inner lining on inner surface 132 of base 104. This frictional engagement between the undercuts and the internal surface 132 may secure and retain the control assembly 108 in position within the recess 106.

In the example shown, the rectangular support base 114 includes a first outer edge 114A and a second outer edge 114B opposite the first outer edge 114A. Outer edges 114A and 114B include undercuts 134 that may engage the rubber lined inner surface 132 of the interior device cavity 106.

Base 104 may be manufactured using a metallic material. For example, the base 104 may be manufacturing using a machining process, such as a Computer Numerical Control (CNC) machining process. In other cases, the base may be manufacturing using a metal injection molding (MIM) process. In general, however, the base 104 may be formed as a unitary base (i.e. base 104 may have a unitary construction). In some cases, the inner surface 132 of base 104 may then be lined with a compressible, resilient material such as a rubber or thermoplastic material.

Alternative materials may also be used for the base 104. Ceramics, such as ceramics containing zirconium oxide, may be used to manufacture base 104. Alternatively, thermoplastic materials may be used to manufacture base 104.

Device body 102 may be tapered along its length. For example, FIG. 3 shows the device body 102 tapering from the first device end 102A to the second device end 102B (i.e. forward direction 146). In the example shown, a first device cross-section 152 taken proximate the first device end 102A may have a first sectional surface area 152A. Similarly, a second device cross-section 154 taken proximate the second device end 102B may have a second surface area 154A. As shown, the first surface area 152A is larger than second surface area 154A due to the taper of the device body 102. It will be appreciated that as the degree of the taper increases or decreases, the difference in size between first surface area 152A and second surface area 154A will correspondingly increase or decrease.

In the example shown, the device body 102 has a generally elliptical cross-section. The elliptical cross-section may prevent the vaporization device 100 from rolling when placed on a surface (e.g. for storage). In addition, the elliptical cross-section may provide a comfortable grip from the user's hand and improve structural integrity by minimizing sharp edges. In some embodiments, the device body 102 may have other cross-sectional configurations, such as circular, triangular, rectangular, hexagonal, etc.

The vaporizer 100 may also include an air intake manifold 110. The air intake manifold 110 may be positioned within the recess 106. In some, air intake manifold 110 may be provided on support assembly 114. For example, air intake manifold 110 may be provided along with the control assembly 108 on the support member 114. Alternatively, the air intake manifold 110 may be positioned within recess 106 adjacent to, and even contacting, the second end 108B of control assembly 114.

For example, recess 106 may include a third recess section 111 between the first recess section 107 and the second recess section 109. The third recess section 111 may receive the air intake manifold 110. In some cases, the third recess section 111 may not be enclosed by cover 144, but rather an upper surface of air intake manifold 110 may be externally accessible.

Alternatively, the cover 144 may overlie some or all of the air intake manifold 110. In such cases, the cover 144 may include a gap or access section allow a user to access a release actuator 162 usable to engage or disengage a cartridge assembly 200 within receptacle 116.

In some cases, the air intake manifold 110 may be fixed within the base 104. The air intake manifold 110 may then define a fixed first end of the receptacle 116.

The air intake manifold 110 may have a first manifold end 110A and a second manifold end 110B opposite the first manifold end 110B. In some embodiments, first manifold end 110A may be positioned to abut the second end 108B of the control assembly 108. In the example shown, the air intake manifold 110 may be mounted on the assembly support base 114. Mounting the air intake manifold 110 on the assembly support base 114 may permit the air intake manifold 110 to be held in position along with the control assembly 108. When mounted on the assembly support base 114, the second manifold end 110B may be substantially aligned with the second member end 108B. Thus, the support base 114 may be positioned in both the first and third sections of recess 106, with the control assembly 108 positioned in the first section 107 and the air intake manifold 110 positioned in the third section 111.

In some cases, the air intake manifold 110 may be secured within the base 104 while permitting a slight deflection or compression of air intake manifold 110. For instance, a gap or compressible coupling may be provided between air intake manifold 110 and the end 108B of control assembly 108. When a cartridge assembly 200 is inserted into receptacle 116, the air intake manifold 110 may be deflected towards the first end 102A of device body 102 to allow the cartridge assembly 200 to rotate into position within receptacle 116. The air intake manifold 110 may be biased or resiliently supported and encouraged to return to its base position, thus providing a further frictional engagement with the upstream end of a cartridge assembly 200 positioned within receptacle 116.

In some cases, the second manifold end 110B may include a compressible coupling member. The compressible coupling member may permit a slight deformation when cartridge assembly 200 is inserted in receptacle 116. This coupling member may then assist in securing cartridge within receptacle 116. For example, the coupling member may be in the form of a compressible seal member that extends around the perimeter of air intake manifold second end 110B.

Air intake manifold 110 may include a manifold fluid flow path 136 defined therethrough. The manifold 110 may include at least one air input aperture 138, which may be referred to as an ambient air inlet or ambient air aperture. The manifold 110 may also include a manifold outlet 139 at the second manifold end 110B. The manifold outlet 139 may be positioned facing the cartridge receptacle 116. The manifold fluid channel 136 may extend between the one or more ambient air inlets 138 and the manifold outlet 139, defining a fluid passage between the ambient air inlet and the cartridge receptacle 116.

In some embodiments one or more porous screens may be disposed within fluid channel 136, e.g. at inlets 138. The porous screens may be configured to encourage laminar air flow in the ambient air entering fluid channel 136. The screen or screens may have pores of about 0.1 mm or 0.2 mm or 0.3 mm. The screens may also filter the ambient air to prevent dirt or debris from entering fluid channel 136.

The ambient air inlet 138 may be aligned with a lateral side of the vaporizer base 104. The base 104 may also include at least one air input port 140 corresponding to the ambient air inlet 138. Each air input port 140 may be aligned with at least one of the air input apertures 138 of the air intake manifold 110 when the vaporization device 100 is assembled.

The ambient air inlet 138 may be positioned in the third recess section 111 (i.e. aligned with the location of air intake manifold 110 between the first end 102A and the second end 102B). As a result, the fluid flow path in vaporizer device 100 may not pass through any part of the first recess section 107 in which the control assembly 108 is positioned.

In some cases, the vaporizer device 100 may include a plurality of ambient air inlets. In the example shown, the at least one air input aperture 138 includes air input apertures 138A and 138B on opposite sides of the air intake manifold 110. The device base 104 includes corresponding input ports 140A and 140B corresponding to apertures 138A and 138B. The air input apertures 138A and 138B may be fluidly connected to the same manifold fluid channel, and may join together as they flow downstream towards the manifold outlet.

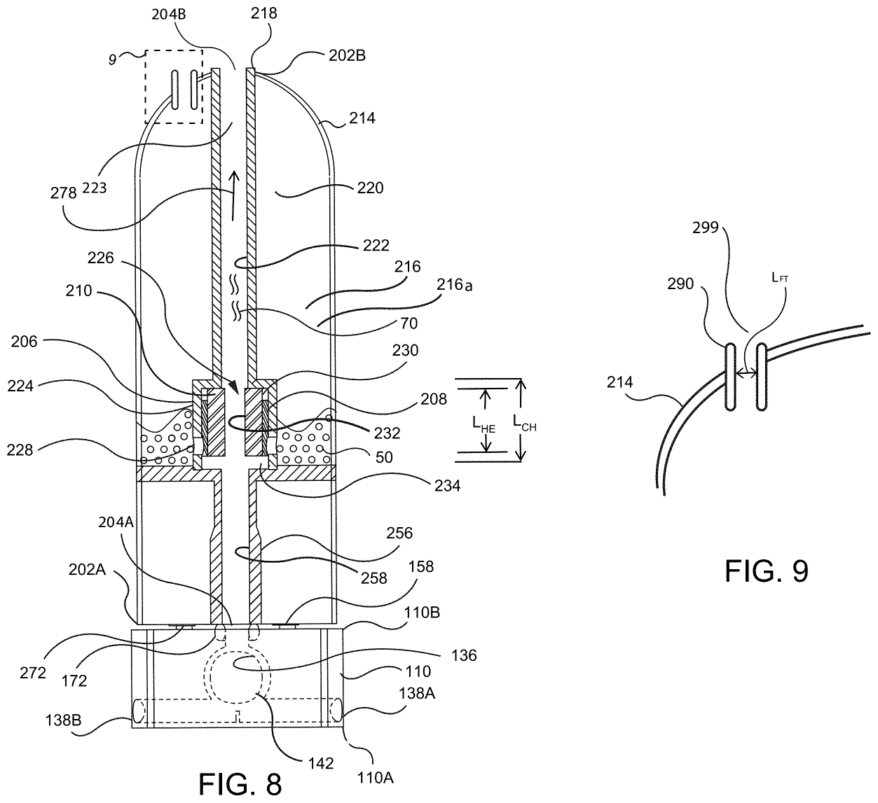

In some embodiments, the air intake manifold 110 may include a fluid flow sensor 142 (see e.g., FIG. 8). The fluid flow sensor 142 may be configured to determine a volume or mass of ambient air 60 being drawn into the manifold fluid flow path 136. Optionally, instead of, or in addition to, the fluid flow sensor 142, the air intake manifold 110 may include a puff sensor 9123 (FIG. 109) positioned within the manifold fluid flow path 136. The puff sensor 9123 and the fluid flow sensor 142 sensor may determine a volume of ambient air 60 passing through the air intake manifold 110. Optionally, an audio microphone may be positioned with the manifold fluid flow path 136 to determine a volume or mass of airflow passing through the air intake manifold 110. Optionally a single barometric pressure sensor is utilized to measure a start of an inhalation and an end of an inhalation and not in measuring a mass airflow. In some embodiments the puff sensor 9123 may be used to determine the start of an inhalation and an end of an inhalation and not in measuring a mass airflow through the manifold fluid flow path 136.

Air intake manifold 110 may be electrically coupled to the control circuit 120. In some embodiments, the air intake manifold 110 may be electrically coupled to the control circuit 120 through the assembly support base 114. The fluid flow sensor 142 may provide flow signals to control circuit 120. The control circuit 120 may use the flow signals to determine the air flow through the air intake manifold 110. Based on the detected airflow, the control circuit 120 may perform various operations, such as activating/deactivating the heating assembly and/or adjusting a temperature of the heating assembly.

In some embodiments, the vaporizer device 100 may include a lock unit usable to secure the cartridge assembly 200 within cartridge receptacle 116. For example, the air intake manifold 110 may have a lock unit 160 positioned proximate the second manifold end 110B (i.e. proximate the upstream end of receptacle 116).

Lock unit 160 may include a lock member 164 configured to engage the cartridge assembly 200 when cartridge assembly 200 is positioned within receptacle 116. For example, the lock member 164 may be in the form of a flange extending from the second manifold end 110B into the receptacle 116. The lock member 164 may be adjustable between an extended or locked position, in which lock member 164 extends into receptacle 116 and a retracted or unlocked position in which lock member 164 recedes from receptacle 116, e.g. into manifold 110.

Lock unit 160 may also include a release member or actuator 162. The actuator 162 may be usable by a user to adjust the lock member 164 between the locked and unlocked positions. For example, release actuator 162 may be in the form of a slider. A user may slide the actuator 162 to adjust the lock member 164 to the unlocked position to allow a cartridge assembly 200 to be removed. In some cases, the lock member 164 (and actuator 162) may be biased to the locked position. This may allow the cartridge to automatically lock into place in vaporizer 100 when lowered into the receptacle 116.

In some embodiments, the vaporizer device 100 may include a cartridge ejection actuator 170. The ejection actuator 170 may be mounted within the cartridge receptacle 116. The ejection actuator 170 may be operable to eject the cartridge assembly 200 from receptacle 116 when the lock member 164 is unlocked.

For example, the ejection actuator may be a spring attached to the base 104 of the vaporizer device 100 proximate the second manifold end 110B (within the receptacle 116). The spring may be movable between an extended position, in which the actuator extends into the receptacle 116, and a retracted position in which the actuator is receded to extend less (or retracted into the base 104).

When the removable cartridge assembly 200 is fully inserted within the cartridge receptacle 116 and held in place by the releasable locking unit 160, the spring 170 may be forced to a compressed state. When the lock unit is released, the spring's biasing to the extended position may encourage the cartridge assembly 200 to be ejected from receptacle 116.

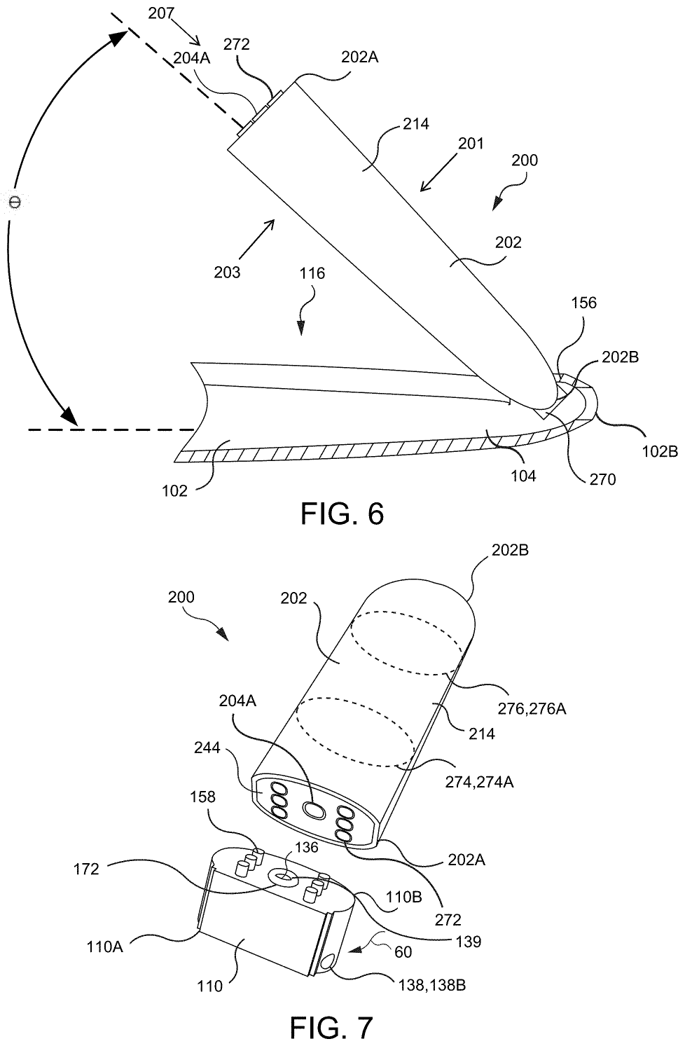

The base 104 may define a lip or overhang 156 in receptacle 116 proximate the second device end 102B. The lip 156 may extend from the second device end 102B towards the first device end 102A to cover a small portion of receptacle 116 inwardly adjacent to inhalation aperture 112. To insert a cartridge into the receptacle 116, an outlet end of the cartridge may be inserted under the lip 156, facing inhalation aperture 112. In some cases, the outlet end may extend into (and even through inhalation aperture 112). The cartridge may then be lowered from the position shown in FIG. 6, e.g. along angle θ, until the upstream end of the cartridge assembly 200 engages the air intake manifold 110 and the cartridge is secured by lock unit 160.

A cartridge receptacle length L_{R} may be measured between the second member end 108B and the second device end 1028. The cartridge receptacle length L_{R} combined with the support member length L_{CC} (including the air intake manifold 110) may define the device length L_{D}. A ratio of the cartridge receptacle length L_{R} to the device length L_{D} may be between 0.2 and 0.8. In the example shown, the ratio is approximately 0.25. That is, the control assembly length L_{CC} is about 75% of the device length L_{D} or the cartridge receptacle length L_{R} is 25% the device length L_{D}. It will be appreciated that the ratio between the cartridge receptacle length L_{R} and the device length L_{D} may vary.

The center of gravity 174 of the vaporization device 100 may be positioned closer to the first device end 102A than the second device end 1028. When a cartridge is removed from receptacle 116, or the vaporizable material 50 stored in the storage reservoir 216 decreases as it is vaporized, the center of gravity 174 may shift even closer to the first device end 102A. Having the center of gravity 174 positioned closer to the first device end 102A than the second device end 102B may make holding the vaporization device 100 to a user's mouth more comfortable, since the weight may be positioned near the first end 102A that is grasped by a user. When a user inserts the inhalation aperture 112 into their mouth, the device 100 will naturally tend to hang at an angle to the horizontal as this may provide a more comfortable use position for the user.

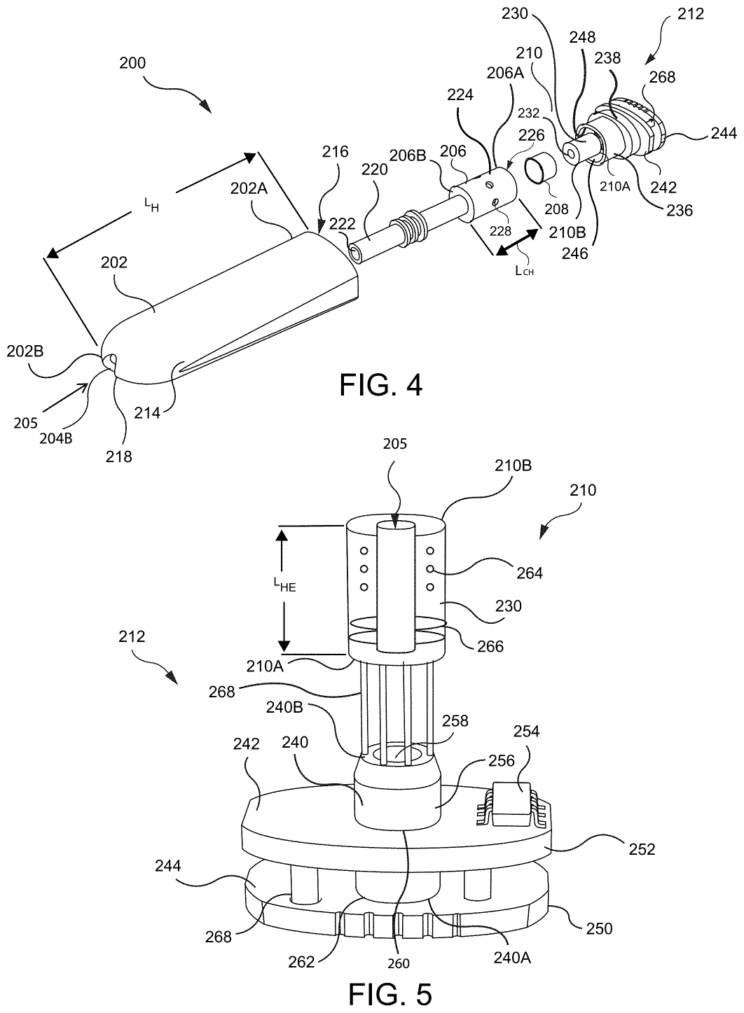

FIG. 4 shows an exploded perspective view of an example removable cartridge assembly 200. In the example shown, cartridge assembly 200 has a top side 201, a bottom side 203, a front side 205, a rear side 207, and opposed lateral sides. Removable cartridge assembly 200 may include a cartridge housing 202, a fluid conduit 204, a heating assembly that includes a heating chamber 206, a wicking element 208, and a heating element assembly 210, a housing end cover 212, and a storage compartment 216.

Cartridge housing 202 may extend between a first cartridge end 202A and a second cartridge end 202B opposite the first cartridge end 202A. A housing sidewall 214 may extend between the first cartridge end 202A and the second cartridge end 202B. A housing length L_{H} may be measured between the first housing end 202A and the second cartridge end 202B.

The fluid conduit 204 may extend through the cartridge housing 202 from the first cartridge end 202A to the second cartridge end 2028. The fluid conduit 204 may include a cartridge conduit inlet or upstream inlet 204A at the first cartridge end 202A. The fluid conduit 204 may include a cartridge conduit outlet or downstream inlet 204B at the second cartridge end 2028. The fluid conduit 204 may include a plurality of conduit sections, including a first or upstream section 258, a second or intermediate section 226, and a third or downstream section 223.

A cartridge aperture 218 may be defined in the cartridge housing 202 at the conduit outlet 204B. As will be described in more detail herein below, when the removable cartridge assembly 200 is positioned within the cartridge receptacle 116 of vaporization device 100, the cartridge aperture 218 may be aligned with, and engage, the inhalation aperture 112. The inhalation aperture 112 may thus be fluidly coupled to fluid conduit 204.

In some embodiments, the cartridge aperture 218 of the fluid conduit 204 may protrude from the housing 202 at the second cartridge end 202B, e.g. as shown in FIG. 8. In this configuration, the cartridge aperture 218 may thus provide an engagement member that may engage the inhalation aperture 112.

The storage compartment or reservoir 216 may be used to store vaporizable material for use with a vaporizer 100. The storage compartment 216 may be enclosed by the outer housing sidewall 214. In the example shown, the storage compartment 216 may surround the fluid conduit 204. That is, the fluid conduit 204 may define a passage that extends through the center of the storage compartment 216.

In the example shown, the storage compartment 216 has a substantially annular or toroidal shape. That is, the storage compartment 216 has an outer periphery or surface defined by cartridge housing 202 and an inner periphery or surface defined by wall 220. As shown, wall 220 may also define and enclose a downstream section 223 of the fluid conduit 204. The storage compartment 216 and the fluid conduit 204 may be concentrically disposed about a central axis of the conduit 204.

The storage compartment 216 may also be fluidly connected to a heating assembly. The heating assembly may be used to vaporize the material stored in the storage compartment 216 so that it may be inhaled by a user of the vaporizer device 100. As shown, inner wall 220 of the storage compartment 216 may also enclose a heating chamber section 226 of the fluid conduit 204.

The heating assembly may include a heating chamber 206 within the cartridge assembly 200. The heating chamber 206 may be surrounded by the storage compartment 216. The heating chamber 206 may be positioned proximate the end of the storage compartment 216. In cartridge assembly 200, the heating chamber 206, fluid conduit 204 and storage compartment 216 may be concentrically and coaxially positioned.

Heating chamber 206 may extend between a first chamber end 206A and a second chamber end 206B opposite the first chamber end 206A. The heating chamber 206 may be defined by an interface member or wall 224 that extends between the first chamber end 206A and the second chamber end 206B. A heating chamber length L_{CH} may be measured between the first chamber end 206A and the second chamber end 206B.

Interface member 224 may enclose a heating chamber cavity that defines a second section 226 of the fluid conduit 204. In the example shown, the heating chamber outer wall 224 extends cylindrically between the first and second chamber ends 206A and 206B, making the heating chamber 206 a cylindrical heating chamber. It will be appreciated that the heating chamber 206 may have many other configurations, such ovular, triangular, rectangular, hexagonal, etc.

The interface member 224 may also define a fluid coupling between the heating chamber 206 and the storage compartment 216. The interface member 224 may include a plurality of apertures 228 positioned facing the storage compartment 216. The apertures 228 may be circumferentially spaced around interface member 224. The inner wall 220 of storage compartment 216 may have one or more apertures aligned with the apertures 228 to allow vaporizable material to flow into the heating chamber 206. Alternatively, inner wall 220 may have a gap or void section that extends around its entire circumference aligned with the aperture 228.

In the example shown, heating chamber 206 is positioned surrounded by the storage reservoir 216. Fluid may flow into the heating chamber 206 from the surrounding storage reservoir 216 via apertures 228.

A heating element assembly 210 may be contained within the heating chamber 206. The heating element assembly 210 extends from a first assembly end 210A to a second assembly end 2108. A heating element length L_{HE} may be measured between the first assembly end 210A and the second assembly end 2108. The heating element assembly 210 may have an outer heating element surface 230 that extends between the first and second ends 210A and 210B. The fluid conduit 204 may pass through an inner surface of the heating element assembly 210.

In the example shown, the heating element assembly 210 is generally cylindrical in shape and may have radially spaced electrical couplings 268 that extend from the heating element assembly 210. The heating element assembly 210 may thus be positioned concentrically with the storage compartment 216. As shown, heating element assembly 210 is also concentric with fluid conduit 204.

The heating assembly may also include a wicking element 208. The wicking element 208 may at least partially surround the heating element assembly 210. The wicking element 208 may also be arranged concentrically and co-axially with the heating element assembly 210. The wicking element 208 may be thermally coupled to heating element assembly 210, e.g. by contacting the outer surface 230 of the heating element assembly 210.

The wicking element 208 may be positioned between the interface member 224 and the heating element assembly 210. Vaporizable material from the storage compartment 216 may be drawn to the heating element assembly 210 by wicking element 208. The vaporizable material in the wicking element 208 may then be heated by the heat emitted from the outer surface 230 of the heating element assembly 210.

Optionally, one or both of heating element assembly 210 and wicking element 208 may be manufactured using porous materials. For example, heating element assembly 210 may be manufactured using a porous ceramic. Further details on manufacturing of the ceramic substrate is outline below.

When assembled, the wick 208 and the heating element assembly 210 may be positioned with the heating chamber cavity 226 of heating chamber 106. The heating chamber cavity 226 may include a void or vapor aperture 234 fluidly connecting the wicking element 208 and the fluid conduit 204. Vapor emitted from heating the vaporizable material in wick 208 may then be drawn into fluid conduit 204 through vapor aperture 234.

Heating element assembly 210 may be positioned within the heating chamber cavity 226 with the wicking element 208 fluidly coupling the fluid conduit 204 to the storage compartment 216. As shown, wicking element 208 is in fluid communication with vaporizable material 50 held in the storage reservoir 216 via the plurality of vaporizable material receiving apertures 228 defined on the heating chamber outer wall 224. The vaporizable material 50 may thus be drawn towards the heating element assembly 210 by wicking element 208.

When energized, the heating element assembly 210 may emit heat to heat wick 208. The vaporizable material drawn into wick 208 may then be heated as well. By heating the vaporizable material 50 to a predetermined vaporization temperature, a phyto material vapor 70 may be emitted. The predetermined vaporization temperature may vary depending on user preference and/or the form of the vaporizable material.

The vapor may then pass through fluid flow gap 234 into the fluid conduit 204. The vapor may travel through the fluid conduit 204 towards the cartridge aperture 218. When the cartridge assembly 200 is positioned within the cartridge receptacle 116, with the cartridge aperture 218 engaged with inhalation aperture 112, the vapor may then be inhaled by a user of vaporizer device 100.

Preferably, heating chamber length L_{CH} is smaller than heating element length L_{HE}. Second element end 110B may abut the second chamber end 106B, e.g., as shown in FIG. 8. Since the heating chamber length L_{CH} is longer than the heating element length L_{HE}, a fluid flow gap 234 may be provided between the second element end 210B and the second chamber end 206B.

The heating element assembly 210 may include a resistive heating wire. Alternatively, a plurality of resistive heating wire bands 264 are positioned between the first and second element ends 210A and 210B, e.g. as shown. The resistive heating bands 264 may be energizable to emit heat by providing current through the bands 264. As shown in FIG. 5, the resistive bands 264 may be enclosed with an outer wall 230 of the heating element assembly. The outer wall may be manufactured of a material having limited thermal conductivity, such as a porous ceramic material. The porous ceramic material may initially provide a partial thermal and electrical insulator that allows the resistive heating element 264 to heat up relatively fast due to the low thermal inertia of wall 230. However, when the porous ceramic outer wall 230 is saturated with a vaporizable material, such as a phyto material extract, the thermal conductivity of outer wall 230 may increase. When energized, the heat emitted by the resistive heating wire flows outwardly through the heating element outer wall 230 to heat the wicking element 208. The plurality of resistive heating wire bands 264 may be in the form of a coiled wire embedded within the porous ceramic heating element assembly 230.

In some cases, the heating element assembly 210 may include a temperature sensor 266. Temperature sensor 266 may be able to measure a temperature of the heat emitted by the resistive heating wire. In some cases, the heating element assembly 210 may not utilize the temperature sensor 266 and the electrical couplings 268 may be used for provide power to the heating element assembly 210.

Heating element assembly 210 and, in particular, the resistive heating wire 264 and the temperature sensor 266 disposed therein, may be electrically coupled to a cartridge control circuit 242. For instance, electrical couplings 268 may extend between the heating element assembly 210 and control circuit 242.

In some embodiments, rather than, or in addition to the temperature sensor 266, cartridge control unit 242 may be configured to extrapolate the temperature of heating element assembly 210. For example, vaporization device may store a calibration lookup table usable to correlate the voltage and current through the resistive heating element 264 with the temperature of heating element assembly 210. The temperature of the resistive heating element 264 may be estimated by sensing a current applied to the heating element assembly 210.

The current applied may be measured by a current sensing integrated circuit, such as ACS722 (manufactured by Allegro MicroSystems) and an analog to digital converter (e.g. a 12, 14 or 16 Bit ADC) to measure battery rail voltage. With the combination of applied current and battery rail voltage, a temperature of the heating element assembly 210 or the resistive heating wire 264 may be extrapolated using a formula based on calibration data contained in a lookup table (LUT). In some embodiments the resistive heating wire 264 may use a temperature coefficient of resistance characteristic, in that its has a known resistance change with temperature and through determining the current flowing through the resistive heating wire 264 a temperature of the wire may be extrapolated.

The cartridge memory module 254 may also store temperature related calibration parameters for the heating element 164 or resistive wire. For example, a calibration relationship between a current through the resistive wire and an overall temperature of the heating element assembly 210 may be determined. The determined calibration values may be programmed into the cartridge memory module 254 during manufacturing production or at least some calibration values.

A cartridge may be installed in a testing apparatus, such as testing and calibration apparatus 1100 shown in FIGS. 67 and 68. A known current may be applied to the heating element assembly 210 and a temperature of the heating element assembly 210 may be measured. For example, a thermal sensing camera, such as one made by FLIR, or other remote temperature sensing apparatus may be used. The calibration apparatus 1100 may then determine a calibration relationship between the applied current and the measured temperature, and store the calibration relationships within the memory module 254.

This process may be repeated automatically for a plurality of currents and a plurality of resulting temperatures. The calibration apparatus 1100 may include low resistance current sensing resistor, for example, a current sensing resistor having a resistance of 50 μΩ or 100 μΩ or 1 milliΩ or a fraction of an Ohm may be used. The current sensing resistor is disposed in series with the heating element 264 or resistive wire resistive wire. An ADC may then be used to measure a voltage drop across this current sensing resistor to determine voltage across the heating element 264 (and thus the current).

Cartridge control circuit 242 may be used to control operation of the heating element assembly 210. Cartridge control circuit 242 may be used to activate/deactivate the heating element assembly 210, e.g. when the temperature measured by the temperature sensor 266 falls below a certain value. In some cases, the cartridge control circuit 242 may be used to selectively activate the heating element assembly 210 to heat only selected portions of the resistive heating wire. Cartridge control circuit 242 may also be used to adjust the settings of heating element assembly 210, such as adjusting the predetermined vaporization temperature. In some cases, the predetermined vaporization temperature may be adjusted based on the data stored in the cartridge memory module 254 indicating the type of vaporizable material stored in the storage compartment 216.

Cartridge control circuit 242 may monitor other operational characteristics of vaporization device 100, such as determining that the cartridge assembly 200 no longer contains, or has a low volume of vaporizable material. For example, control circuit 242 may determine that the heating element assembly 210 is increasing in temperature too rapidly (e.g. at a rate above a heating threshold). Control circuit 242 may then determine that heating element assembly 210 is no longer in contact with vaporizable material indicating that the cartridge assembly 200 is empty or nearly empty. Cartridge control circuit 242 may provide a feedback signal to control circuit 120, which in turn may provide an indication to the user that the cartridge assembly 200 is empty or nearly empty.

The cartridge assembly 200 may also include a base or end cap assembly 212. The base 212 may include a chamber sheath 236, a sheath support 238, an end cap conduit section 240, and a base closure member 244. The cartridge control circuit 242 may be mounted on base 212.

Chamber sheath 236 may enclose a portion 246 of the first conduit section 226. An outer dimension of the chamber sheath 236 may be substantially equal to, although slightly larger than, an outer dimension of the heating chamber 206. Accordingly, the heating chamber 206 may be, at least partially, inserted into the chamber sheath 236.

Chamber sheath 236 may be connected directly to the cartridge control circuit 242. Optionally, a sheath support 238 may be mounted to the chamber sheath 236 to provide added structural support. Sheath support 238 may connect the chamber sheath 236 to the cartridge control circuit 242, e.g. as shown. Frictional engagement between an interior surface 248 of chamber sheath 236 and the heating chamber outer wall 224 may secure the heating chamber 206 to the end cap assembly 210. For instance, the heating chamber 206 may be mounted in chamber sheath 236 in a friction fit.

FIG. 5 shows a front perspective view of the heating element assembly 210 attached to the end cap assembly 212. Both the chamber sheath 236 and the sheath support 238 of the end cap assembly 212 have been removed to illustrate internal components. As noted above, when the removable cartridge assembly 200 is assembled, storage reservoir 216 may be closed at the second cartridge end 202B by the end cap assembly 212. In the example shown, the base closure member 244 is configured to substantially match the configuration of the open first cartridge end 202A. The end cap assembly 212 may be inserted within the open first cartridge end 202A with the base closure member 244 acting as a plug to close the first cartridge end 202A. Frictional engagement between the housing sidewall 214 and an outer edge 250 of the base closure member 244 may secure the end cap assembly 210 within the outer housing 202. The outer edge 250 of base closure member 244 may include compressible material, such as a rubberized lining, that provides a snug engagement between base closure member 244 and the housing sidewall 214 when inserted in first end 202A.

Cartridge control circuit 242 may be electrically coupled to the base closure member 244. Optionally, the cartridge control circuit 242 may be configured to substantially correspond to the configuration of the base closure member 244, e.g. as shown. Frictional engagement between the housing sidewall 214 and an outer edge 252 of the cartridge control circuit 242 may further support engagement of the end cap assembly 210 and the cartridge housing 202. In alternative embodiments, the cartridge control circuit 242 may be mounted directly to the base closure member 244.

In the example shown, the cartridge control circuit 242 includes a memory module 254. Cartridge memory module 254 may store data associated with cartridge assembly 200, such as a unique identifier (e.g. an identification serial number) that may be used to identify the removable cartridge assembly 200. The memory 254 may store data (e.g., type, concentration, dose, etc.) regarding the vaporizable material 50 within the removable cartridge assembly 200. In some cases, the unique identifier may be used to retrieve data associated with cartridge assembly 200 and/or vaporizable material 50.

Closure member 244 may include an end cap conduit section 240 that forms an upstream portion of the first conduit section 258. The end cap conduit section may extend between a first end cap conduit end 240A and a second end cap conduit end 240B opposite the first end cap conduit end 240A. An end cap conduit outer wall 256 may extend between the first end cap conduit end 240A and the second end cap conduit end 240B. In the example shown, the end cap conduit outer wall 256 extends cylindrically between the first and second end cap conduit ends 240A and 240B, forming a cylindrical conduit section. Although a cylindrical end cap conduit section is shown, it will be appreciated that the end cap conduit 240 may have many other configurations, such ovular, triangular, rectangular, hexagonal, etc.

In the example shown, the end cap conduit section 240 extends through apertures 260 and 262 defined in the cartridge control circuit 242 and the base closure member 244, respectively. The apertures 260 and 262 may be sized to be substantially equal to, although be slightly larger than, an outer dimension of the end cap conduit section 240. Accordingly, when the end cap is being assembly, the outer wall 256 of end cap conduit section 240 may be inserted through the apertures 260 and 262. Preferably, outer wall 256 is inserted through apertures 260 and 262 until the first end conduit end 240A is flush with the base closure member 244, e.g. as shown in FIG. 7.

End cap conduit portion 240 may be fluidly connected with a sheath fluid conduit portion 246. Preferably, the second end cap conduit end 240B is axially aligned with the second heating element end 2108, e.g. as shown. When assembled, the end cap fluid portion 240 (defining first conduit section 258), the sheath fluid conduit portion 246 and heating chamber cavity (together defining second fluid conduit section 226), and the downstream section 223 together define the fluid conduit 204 extending throughout the length of cartridge assembly 200. That is, the fluid conduit 204 defines a cartridge fluid flow path 278 that extends the entire length of the cartridge housing 202 between the first cartridge end 202A and the second cartridge end 202B, e.g. as shown in FIG. 8. As shown in FIG. 8, the fluid flow path 278 may be a linear flow path throughout the length of cartridge assembly 200, which may facilitate air flow through the cartridge assembly 200 by reducing backpressure and airflow loss that might otherwise be caused by turns in the air flow passage.

FIG. 6 shows a side cutaway view showing the removable cartridge assembly 200 in an unlocked position relative to the vaporization device 100. Removable cartridge assembly 200 may be dimensioned to fit snugly within the cartridge receptacle 116 defined within the interior device cavity 106, e.g. shown in FIG. 1. The device body 102 and cartridge assembly 200 may include one or more registration features to ensure that cartridge assembly 200 is installed correctly within receptacle 116.

For example, housing sidewall 214 may define a registration feature that allows the removable cartridge assembly 200 to be inserted into the cartridge receptacle 116 is only one way. The registration feature may be referred to as a polarizing feature that restricts insertion of the removable cartridge assembly 200 to only one orientation. Accordingly, the user may be prevented from inserting the removable cartridge assembly 200 in the wrong way.

In the example shown, the registration feature may include a projection tab 270 that extends outwardly from the second cartridge end 202B. Projection tab 270 may have a projection aperture (not shown) defined therethrough. Projection aperture may substantially align with the cartridge aperture 218, thus enabling fluid communication between the projection aperture and the cartridge aperture 218. The projection tab 270 may extend outwardly from cartridge end 202B so that cartridge assembly 200 cannot be inserted in receptacle 116 unless the tab 270 is engaged with inhalation aperture 112.

Alternatively, the projection tab 270 may be integrally formed with the outer housing 202, e.g. formed by the housing sidewall 214. In embodiments where the projection tab 270 is integrally formed with the outer housing 202, the projection tab 270 may have the cartridge aperture 218 defined therethrough.

As shown in FIG. 6, to insert the cartridge assembly 200 into vaporization device 100, a user may insert the projection tab 270 into the inhalation aperture 112 through the cartridge receptacle 116 at the second device end 102A. Removable cartridge assembly 200 may be inserted at an insertion angle θ measured relative to the device body 102. Preferably, the insertion angle is approximately 45 degrees, e.g. as shown. However, insertion angles between 20 and 70 degrees are possible. Insertion angle θ may permit the projection tab 270 to enter the cartridge receptacle 116 (and inhalation aperture 112) beneath the overhang 156 formed by the housing sidewall 214, e.g. as shown.

A user may then fully insert the removable cartridge assembly 200 within the cartridge receptacle 116 by rotating cartridge assembly 200 relative to device body 100 to reducing the insertion angle θ to 0 degrees, i.e. lowering the first cartridge end 202A to be adjacent the second manifold end 210B. When the user is lowering the first cartridge end 202A into the cartridge receptacle 116, the overhang 156 (and inhalation aperture 112) may maintain the second cartridge end 202B in position within the cartridge receptacle 116. This may prevent dislodgement of the removable cartridge assembly 200 from the cartridge receptacle 116 during the insertion process. The overhang 156 may also prevent side to side rotation of the cartridge assembly 200 when being inserted into receptacle 116, or after insertion, by engaging the top surface of cartridge assembly 200.

As shown in FIG. 6, a plurality of cartridge electrical contacts 272 may protrude from the first cartridge end 202A. The plurality of cartridge electrical contacts 272 may extend from the base closure member 244, e.g. as shown in FIG. 7. The plurality of cartridge electrical contacts 272 may be in electrical communication with cartridge control circuit 242. The electrical contacts 272 may also be electrically connected to heating element assembly 210 to allow current from energy storage module 128 to be directed through the heating element 264.

Referring again to FIG. 1, a plurality of device electrical contacts 158 may be contained within device body 102. The device electrical contacts 158 may extend outwardly from the second manifold end 110B. The device electrical contacts 158 may be electrically connected to control circuit 120 and energy storage module 128.

As noted above, the air intake manifold 110 may be electrically coupled to the control circuit 120. In some embodiments, the air intake manifold 110 may be electrically coupled to the control circuit 120 through the assembly support base 114. Alternatively, the air intake manifold 110 may be directly electrically coupled to the control circuit 120. Accordingly, the plurality of manifold electrical contacts 158 may be in electrical communication with control circuit 120 through manifold 110.

The registration feature of the removable cartridge discussed above (e.g., projection tab 270) may ensure that the plurality of manifold electrical contacts 158 substantially align and engage with the plurality of cartridge electrical contacts 272 when the removable cartridge assembly 200 is fully inserted within the cartridge receptacle 116. As a result, when fully inserted, the cartridge control circuit 242 and heating element assembly 210 of the removable cartridge assembly 200 may be in electrical communication with the control circuit 120. Energy storage module 128 may be used to energize the cartridge control circuit 242 and the heating element assembly 210. Control circuit 120 may also be used to control the operation of the cartridge control circuit 242.

As mentioned above, the device body 102 may further include a releasable lock unit 160 defined proximate the second manifold end 110B. The lock unit 160 may include a lock member 164 that may project into the receptacle 116. As the first cartridge end 202A of the removable cartridge assembly 200 is lowered into the cartridge receptacle 116 during insertion, the lock member 164 may be forced, from contact with the first cartridge end 202A, to move in an unlocking direction 166 toward the first manifold end 110A.

When the removable cartridge assembly 200 is completely inserted into the cartridge receptacle 116, the lock member 164 may automatically move back in a locking direction 168 to protrude from the second manifold end 110B. The lock member 164 may thus automatically secure the removable cartridge assembly 200 within the cartridge receptacle 116. When the removable cartridge assembly 200 is positioned within the cartridge receptacle 116 and held in place by the releasable locking unit 160, the removable cartridge assembly 200 may be considered to be in a secured position.

In the secured position, the cartridge aperture 218 may be substantially aligned with the inhalation aperture 112. Accordingly, the cartridge aperture 218 and the inhalation aperture 112 may be in fluid communication. Thus, when the removable cartridge assembly 200 is in the locked position, the cartridge fluid flow path 278 may be in fluid communication with the external environment surrounding the vaporization device 100 through inhalation aperture 112 and ambient air inlet ports 138. The cartridge fluid flow path 278 may otherwise be fluidically sealed from the external environment.

To release the removable cartridge assembly 200 from the cartridge receptacle 116 (e.g. after vaporization), the release member 162 may be moved in the unlocking direction 166. For example, a user may grip the slider 162 with their fingers and slide it in the unlocking direction 166. Moving the release member 162 in the unlocking direction 166, may retract the lock member 164 such that it no longer protrudes outwardly from the second manifold end 110B to engage cartridge assembly 200. As a result, the lock member 164 may no longer retain the removable cartridge assembly 200 within the cartridge receptacle 116. The ejection actuator 170 may then promote ejection of the cartridge assembly 200 from receptacle 116.

Additionally, or alternatively, a fingernail groove (not shown) may be formed between the cartridge housing 202 and base 104 to facilitate removal of the removable cartridge assembly 200 from the cartridge receptacle 116. The fingernail groove may extend in a direction substantially orthogonal to the housing length L_{H}, and preferably be formed proximate the first cartridge end 202A. The fingernail groove may have a width suitable for a user to insert one of their fingernails or a tool such as a pin or knife into, for e.g. preferably between 0.5 and 2 mm. For example, as the lateral slider 162 is moved in the unlocking direction 166 to release the removable cartridge assembly 200 from being retained by the lock flange 164, the fingernail groove may be accessed by the user's fingernail to pull the removable cartridge assembly 200 out of the cartridge receptacle 116.

FIG. 7 shows a rear perspective view of the air intake manifold 110 separated from the removable cartridge assembly 200. FIG. 7 illustrates the corresponding plurality of cartridge electrical contacts 272 of the removable cartridge assembly 200 and manifold electrical contacts 158 of the air intake manifold 110.

In order to fit snuggly within the cartridge receptacle 116, the cartridge housing 202 may be dimensioned to correspond to the taper of the device body 102. In the example shown in FIG. 7, the cartridge housing 202 tapers from the first cartridge end 202A to the second cartridge end 202B. A first housing cross-section 274 taken proximate the first cartridge end 202A may have a first surface area 274A. Similarly, a second housing cross-section 276 taken proximate the second cartridge end 202B may have a second surface area 276A. Due to the taper of the outer housing 202, the first surface area 274A may be larger than second surface area 276A. It will be appreciated that as the degree of the taper increases or decreases, the difference in size between first surface area 274A and second surface area 276A will correspondingly increase or decrease.

In the example shown, the outer housing 202 has an elliptical cross-section. The elliptical cross-section of cartridge housing 202 may correspond substantially to the elliptical cross-section of the device body 102 at the cartridge receptacle 116 (although cartridge housing 202 may be slightly narrower).

The elliptical cross-section may prevent the removable cartridge assembly 200 from rolling when placed on a surface (e.g. for storage). In addition, the elliptical cross-section may improve structural integrity of the removable cartridge assembly 200 by minimizing sharp edges. In some embodiments, the outer housing 202 may have other configurations, such as circular, triangular, rectangular, hexagonal, etc. to substantially match the configuration of the device body 102.

FIGS. 7 and 8 illustrate the manifold fluid flow path 136 defined within the air intake manifold 110. In the example shown, the manifold fluid flow path 136 extends inwardly from the second manifold end 110B towards the first manifold end 110A. However, in the example shown the manifold fluid channel 136 does not extend to the second manifold end 110B, but rather to lateral input apertures 138. In the example shown in FIG. 7, an air input aperture 138B is positioned proximate the first manifold end 110A. Air input aperture 138A is similarly positioned on the opposite side of the air intake manifold 110 (see e.g. FIG. 8). Air input apertures 138A and 138B may be fluidly connected with the manifold fluid flow path 136 and define upstream ends of fluid flow channel 136.

Ambient air 60 may enter the manifold fluid flow path 136 via air input apertures 138A and 138B. The air input ports 140A and 140B defined on opposite sides of the device body 102 may be aligned with the air input apertures 138A and 138B of the air intake manifold 110, respectively, when the vaporization device 100 is assembled. Accordingly, ambient air 60 from the external environment surrounding the vaporization device 100 may be drawn into the manifold fluid flow path 136 through the air input ports 140A and 140B and the air input apertures 138A and 138B, respectively.

When removable cartridge assembly 200 is positioned in receptacle 116, the manifold fluid flow path 136 may be aligned with the first conduit section 258. The manifold outlet 139 may fluidly engage the cartridge conduit inlet shown as end cap conduit end 240A. Accordingly, the manifold fluid flow path 136 may be in fluid communication with the cartridge fluid flow path 278 defined within the removable cartridge assembly 200. A continuous flow may be defined between, the air input apertures 138 and the inhalation aperture 122 extending through the manifold fluid flow path 136 and the cartridge fluid flow path 278.

FIG. 8 show a sectional view of the removable cartridge assembly and the air intake manifold 110 taken along their lengths with the removable cartridge assembly 200 installed and engaging the manifold 110. As shown in FIG. 8, the fluid conduit 204 defines a linear fluid flow passage throughout the length of cartridge assembly 200.

The plurality of cartridge electrical contacts 272 of removable cartridge assembly 200 are shown electrically connected with the plurality of manifold electrical contacts 158 of air intake manifold 110. Manifold fluid flow path 136 is shown in fluid communication with first cartridge conduit section 258. Optionally, a sealing element 172 may be provided at the second manifold end 110A, e.g. as shown. Sealing element 172 may surround the cartridge conduit inlet 240A when the removable cartridge assembly 200 is in the locked position. Sealing element 172 may prevent air and/or vapor from escaping the continuous fluid flow path between the second manifold end 110B and the fluid conduit 240. The sealing element 172 may be a compressible seal member that is defines a gasket seal between manifold 110 and cartridge assembly 200 when the cartridge assembly 200 is installed in receptacle 116.

When a user inhales from the inhalation aperture 112, ambient air 60 may be drawn from the external environment into the manifold fluid flow path 232 via the at least one air input port 240 and the at least one air input aperture 238. Ambient air 60 flows through the manifold fluid flow path 232 before entering the cartridge fluid flow path 278 at the junction of the second manifold end 210B and the cartridge conduit inlet 240A. While being drawn by the user's inhalation through the cartridge fluid flow path 178, the ambient air 60 may mix with the vapor 70 emitted within the heating chamber conduit section 226 prior to exiting the inhalation aperture 112.

Preferably, user inhalation and the vaporization of the vaporizable material 50 may be synchronized. In some cases, the control assembly 108 may activate the heating element assembly 210 (or provide a signal to cartridge control circuit to activate the heating element assembly 210) in response to the fluid flow sensor 142 detecting ambient air passing through the air intake manifold 110. Additionally, or alternatively, the plurality of LEDs 130 may indicate that the heating element assembly 210 is heated to the predetermined vaporization temperature. This may indicate that the vaporization device 100 is ready for a user inhalation. In other cases, alternative status indicators may be used. For instance, a vibration notification may be used to notify the user to initiate inhalation, to stop inhalation and/or to increase a depth of inhalation.

It may be desirable for mixture of ambient air and emitted vapor flowing out of the heating chamber cavity 226 may enter the downstream conduit section 223 at a first temperature T1 and exit through cartridge aperture 218 at a second temperature T2 that is lower than the first temperature T1. That is, the mixture may cool as it flows within the housing downstream conduit section 223 toward the cartridge aperture 218. This may provide the user with a more comfortable, and safer, temperature of vapor for inhalation.

By enclosing the downstream portion 223 of the fluid conduit 204 within the storage compartment 216, cooling of the emitted vapor may be encouraged. The inner walls 222 of the storage compartment 216 may permit heat transfer between the inner volume of the storage compartment 216 and the fluid conduit 204. As the vaporizable material stored in the storage compartment 216 is maintained at a temperature (typically near room temperature) lower than the vaporization temperature, the heat transfer may serve to cool the vapor before it reaches the inhalation aperture 112. Similarly, the vapor may warm the vaporizable material to reduce viscosity and facilitate fluid flow from the storage compartment 216 to wicking element 208.

FIG. 9 shows an enlarged view taken of a filling aperture 290 of cartridge assembly 200. The enlarged view of FIG. 9 corresponds to region 9 shown in FIG. 8. When cartridge assembly 200 is initially manufactured, a filling tube or aperture may be defined in the housing sidewall 214. Filling tube 290 may fluidly connect the storage reservoir 216 to the external environment. In the example shown, the filling tube 290 is defined proximate the second cartridge end 202B. Filling tube 290 may be used to fill the storage reservoir 216 with the vaporizable material 50. For example, a predetermined amount of vaporizable material 50 may be added to the storage reservoir 216. In this way, the filling tube 290 may provide for predetermined amounts of vaporizable material 50 to be filled into the storage reservoir 216.

Once the predetermined amount of vaporizable material 50 has been added to the storage reservoir 216, the filling tube 290 may be sealed, for e.g. by heat sealing. In some embodiments an elastomeric plug may be used to seal the filling tube 290. In some embodiments an elastomeric plug may be first inserted in to the filling tube 290 and a needle may be used to pierce the elastomeric plug and fill the cartridge in an inverted manner whereby air contained within the cartridge escapes from the storage reservoir 216 during the filling operation through the wicking element and through the plurality of apertures 228 formed in the interface member 224.

An internal dimension L_{ET} of the filling tube may be between 2 to 5 mm. The internal dimension L_{ET} may permit the filling of viscous liquid vaporizable material 50 into the storage reservoir 216 using a wider filling nozzle. It will be appreciated that the preferred internal dimension L_{ET} of the filling tube 280 may depend on the type and viscosity of the liquid vaporizable material 50 to be added to the storage reservoir 216.

The cartridge may also comprise a mouthpiece and having an inhalation aperture formed proximate its cartridge aperture 218. The cartridge housing 202 extending from a first end 202A of the cartridge to a second end 202B of the cartridge. An elongated storage compartment 216 configured to store a vaporizable material 50. The storage compartment comprising an inner storage volume 216a wherein the vaporizable material is storable in the inner storage volume 216a and where the inner storage volume 216a is enclosed by the cartridge housing 202.

A heating element assembly 210 disposed at the first end of the storage compartment 202A, the heating element assembly 210 comprising a heating element, a wicking element, wherein the heating element is in thermal contact with the wicking element, wherein the storage interface member surrounds the wicking element, and the storage interface member includes a plurality of circumferentially spaced fluid apertures fluidly connecting the wicking element 208 to the inner storage volume 216a. A fluid conduit 204 extending through the housing 202 from a conduit inlet 204A at the first end to a conduit outlet 204B at the second end, wherein the fluid conduit is fluidly connected to the wicking element 208, the fluid conduit passes through the heating element assembly 210. In this embodiment the storage compartment, heating assembly and fluid conduit are concentrically disposed and the storage compartment surrounds the heating assembly and the fluid conduit and the fluid conduit extends along the entire length of the elongated storage compartment. A memory circuit or cartridge memory module 254 may be provided for storing at least a pulse width modulation profile therein for being read by in some embodiments by the cartridge control circuit 242 and in some embodiments by a control assembly 408 that include control circuit 420 (such as that for vaporization device 400) for providing of the at least a pulse width modulation profile to the heating element 264 for heating at least a portion of the vaporizable material wicked into heating element assembly 230 for generating an aerosol therefrom into the fluid conduit. The vaporization device 400, 100, 200 in accordance with embodiments of the invention may include a memory circuit 420m within the control assembly 420 of the vaporization device 400. The memory circuit 420m may be in the form of a FLASH memory or an EEPROM or other storage medium that upon electrical power being other than applied to the memory circuit 420m, data stored within the within memory circuit 420m remains until an erasing operation is executed.

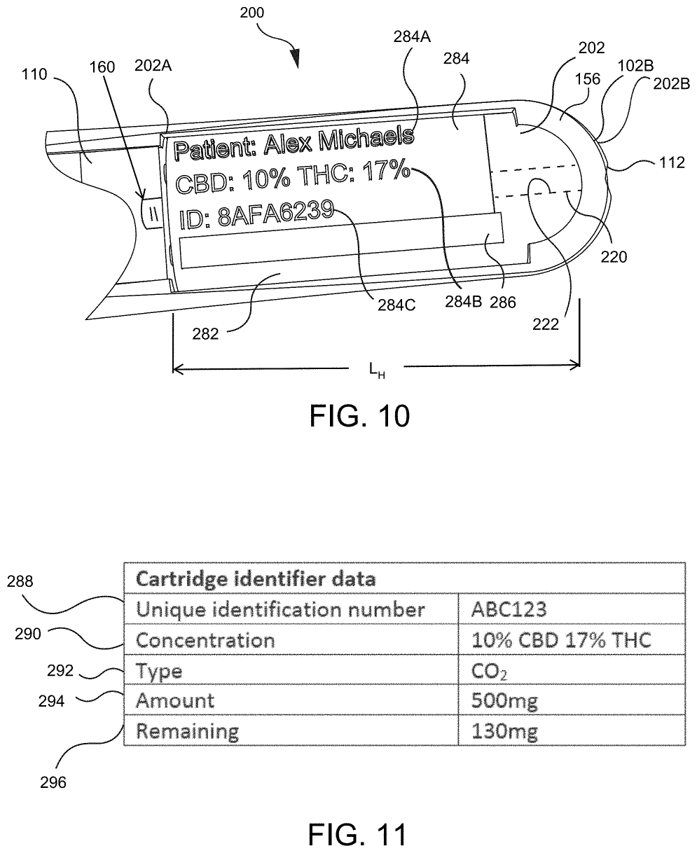

FIG. 10 shows a top cutaway view of the vaporization device 100 with the removable cartridge assembly 200 in the locked position. As shown, a portion of the outer housing 202 of the removable cartridge assembly 200 may be made from a non-transparent material 282 (e.g. opaque material). Accordingly, vaporizable material 50 within the storage reservoir 216 may not be visible through the non-transparent material 282. Non-transparent material 282 may include a label 284 printed thereupon. Label 284 may be visible to a user of the vaporization device 100 and/or a user handling the removable cartridge assembly before inserting it into the vaporization device 100. Label 284 may include a patient name 284A, a vaporizable material type 284B, and/or a unique identification number 284C, e.g. as shown.

Outer housing 202 and/or the label 284 may also include a marking or markings (not shown) (e.g. with a characteristic UV, IR or other wavelength-specific ink) that may be detected by the vaporizer device 100. For example, the marking(s) may include an infrared-scannable barcode located on the outer housing 202 and/or label 284. In some embodiments, the marking(s) may be a pattern, such as a QR code, bar code, etc., that indicate information about the removable cartridge assembly 200 and/or the contents (e.g. vaporizable material 50) within the cartridge removable cartridge assembly 200. In some cases, the marking(s) may be a symbol and/or alphanumeric.

The marking(s) may be “read” or detected directly by the vaporizer device 100, which may include a camera, scanner or other optical detector (not shown), or it may be indirectly detected via communication with a second device (e.g., a user's smartphone, tablet, etc.) having a camera or an optical detector. For example, the marking(s) on the outer housing 202 and/or label 284 may be detected by the user's smartphone using an application (e.g., software) on the user's smartphone usable to identify characteristics of the cartridge assembly 200. For instance, the application may be configured determine one or more cartridge properties from a look-up table (LUT), or it may directly communicate the marking to the vaporization device 100 that may look up the properties, and/or it may communicate with an external server (not shown) that may look up the properties and communicate them to the vaporizer device 100 directly or through the user's smartphone or Wi-Fi connection. In some embodiments to conserve battery power, the vaporizer device 100 may communicate using a wireless module (e.g. Bluetooth or Wi-Fi radio) when the device 100 is being recharged. In some embodiments, device firmware may be updated while the device 100 is being recharged. The device 100 (i.e. control circuit 120) may be configured to update only while recharging, to prevent unnecessary battery drain.

In some cases, the outer housing 202 may have a viewing region that includes a transparent window 286 defined in the housing sidewall 214. Transparent window 286 may extend partially along the housing length L_{H}, e.g. as shown. Storage reservoir 216 may be visible through the transparent window 286. Thus, a user may be able to see the vaporizable material 50 contained in the storage reservoir 216 when the removable cartridge assembly is in the locked position. That is, the user may be able to assess the quantity and type of the vaporizable material 50 through the transparent window 286 when the removable cartridge assembly 200 is inserted within the cartridge receptacle 116. Preferably, the transparent window 286 is made from a material that is BPA free and is of medical and food grade.

In some cases, the fluid conduit 204 may also be visible through the window 286. For instance, a portion of the inner wall 222 may be transparent allowing a user to view fluid conduit 204. This may allow a user to assess the state of conduit 204 and identify any clogging or blockage.

FIG. 11 shows an example diagram of cartridge identifier data that may be encoded within the cartridge memory module 254 of the removable cartridge assembly 200. The cartridge identifier data shown in FIG. 11 may also be provided on the cartridge assembly 200 and/or as feedback on a digital display of the vaporizer device 100. In some cases, the cartridge identifier label may be indicated on an inner surface of storage compartment 216 visible through the window 286.

The cartridge identifier data 1773 (FIG. 114) may include a unique identification number 288, e.g. “ABCD123” as shown. The cartridge identifier data may also include a concentration 290, such as 10% CBD and 17% THC, or other data related to concentration. The cartridge identifier data may also include a vaporizable material type 292, such as such as cannabis or nicotine. The cartridge identifier data may also include a fill amount 294, such as a quantity of vaporizable material 50 that was filled into the storage reservoir 216, e.g. “500 mg” as shown. The cartridge identifier data may also include a remaining amount 296, such as a quantity of vaporizable material 50 that remains in the storage reservoir 216.

Other cartridge identifier data that may be stored in the cartridge memory module 254 may include configuration of the removable cartridge assembly 200 (e.g. electrical properties of heating element assembly 210), a lot number of the removable cartridge assembly 200, a date of manufacture of the removable cartridge assembly 200, an expiration date of the vaporizable material 50, information of the apparatus used to fill the removable cartridge assembly 200, viscosity properties of the vaporizable material 50, etc. This cartridge identifier data may be directly encoded in the cartridge memory module 254 or a reference indicator (e.g. unique identification number 288) may be provided that the control circuit 120 may use as an index to look up some or all of this information, or a combination of the reference number and the directly encoded cartridge identifier data may be provided.

A filling apparatus (described in more detail herein below) used to fill the vaporizable material 50 into the removable cartridge assembly 200 may retrieve the cartridge identifier data stored in the cartridge memory module 254 and fill the storage reservoir 216 according to the retrieved cartridge identifier data. Alternatively, the filling apparatus may program or encode the cartridge identifier data into the cartridge memory module 254 after filling the storage reservoir 216 of removable cartridge assembly 200.

In some cases, the filling apparatus may be used in conjunction with a calibration apparatus 1100 usable to enable operation of the heating element and probe the heating element temperature. The calibration apparatus 1100 may store calibration values in memory module 254, such as a lookup table correlating temperature with the current applied to the heating element.

A predetermined amount of vaporizable material 50 may be filled into the storage reservoir 216 of removable cartridge assembly 200 (e.g. using filling tube 280). The predetermined amount of vaporizable material 50 may be added using either a “volume-based”or “weight-based” method. After filing the storage reservoir 216 of removable cartridge assembly 200 with the predetermined amount of vaporizable material 50, the cartridge memory module 254 (FIG. 5) may be encoded or programmed with cartridge identifier data. As discussed above, the cartridge memory module 254 may be in electrical communication with the plurality of cartridge electrical contracts 272. As a result, when the removable cartridge assembly 200 is in the locked position, by virtue of the electrical coupling of the plurality of cartridge electrical contracts 272 with the plurality of manifold electrical contacts 158, the cartridge memory module 254 may be in electrical communication with control circuit 120 of control circuit assembly 108.

Control circuit 120 may be wirelessly coupled with the external server through at least one of the Bluetooth module 122, the NFC module 124 and the Wi-Fi module 126. Accordingly, operating parameters of the control circuit 120 may be adjusted based on the cartridge identifier data stored on the circuit module 254 as well as the information/data received from the external server.

When the removable cartridge assembly 200 is in the locked position, the cartridge identifier data stored in the cartridge memory module 254 may be accessed and read by the control circuit 120. The control circuit 120 may adjust the operation of the heating element assembly 210 based on the cartridge identifier data, e.g. adjust the temperature, increase/decrease the power supply from energy storage module 128, etc. Control circuit 120 may also perform calculations based on the mass of air flow entering the vaporization device 100 (e.g. measured by the fluid flow sensor 142) and the cartridge identifier data to achieve a predetermined dose. The control circuit 110 may also perform calculations based on the mass of air flow entering the vaporization device 100 in conjunction with cartridge identifier data.

In some embodiment, cartridge memory module 254 may generally be implemented using any memory circuit, such as RAM, ROM, Flash, and an electrically erasable programmable read-only memory (EEPROM). The removable cartridge assembly 200 may be recognized and/or identified by communication between the cartridge memory module 254 within the removable cartridge assembly 200 and the control circuit 120 within the vaporizer device 100. It may be advantageous to use one or more of the electrical connections on the cartridge (e.g., plurality of manifold electrical contacts 158) that are also used to energize and/or control the heater element assembly 210 to communicate with the memory module 254.

Generally, communication between the removable cartridge assembly 200 and the vaporizer device 100 may be one way (e.g., reading information about the removable cartridge assembly 200 and/or the vaporizable material 50 contained in the removable cartridge assembly 200 stored in the cartridge memory module 254 by the vaporizer device 100) or it may be two-way (e.g., reading information about the removable cartridge assembly 200 and/or the vaporizable material 50 contained in the removable cartridge assembly 200 and writing information about the operation of the vaporization device 100 into the memory module 254, e.g., number of uses, duration of use, temperature settings, etc.). That is, information may be written in the cartridge memory module 254 of removable cartridge assembly 200, and this information may be used to derive other information about the removable cartridge assembly 200, including the amount of material left in the cartridge, etc. The information written in the cartridge memory module 254 of removable cartridge assembly 200 may also include air flow data of the mass and/or volume of ambient air 60 passing through the air intake manifold 110 (e.g. collected by fluid flow sensor 142).

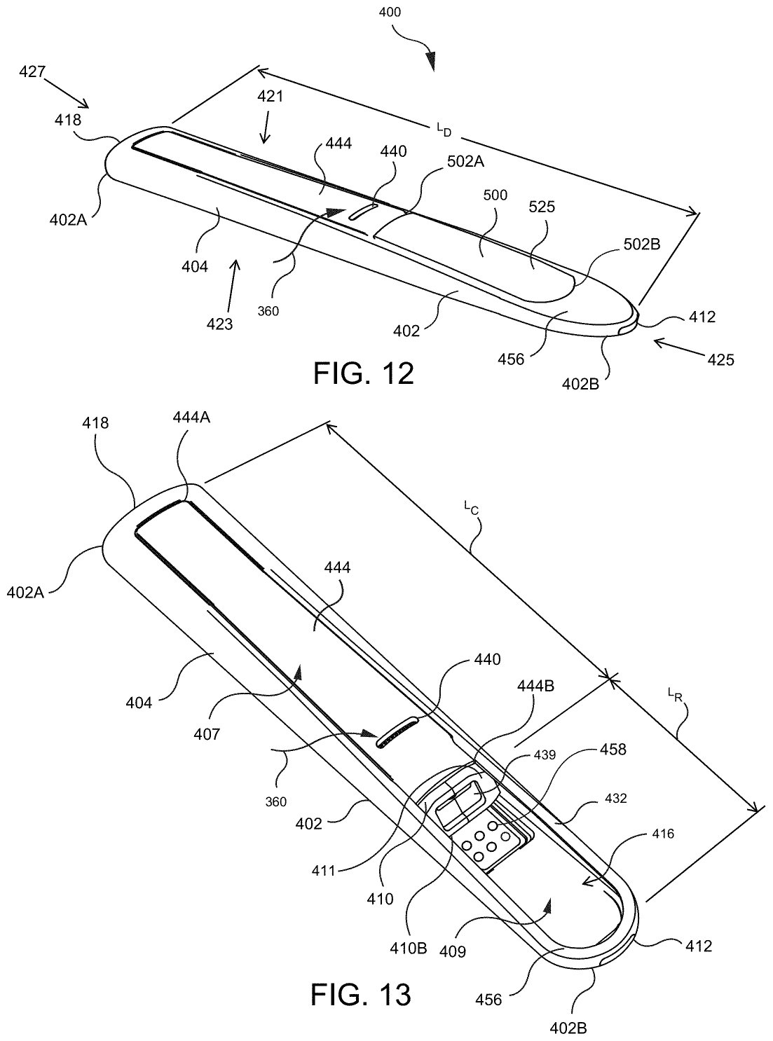

Referring now to FIGS. 12-24, shown therein is an example of a vaporization device 400. Vaporization device 400 is another example of a vaporization device usable to vaporize vaporizable material. Vaporization device 400 may be used to vaporize vaporizable material that is provided in a semi-liquid and/or liquid form. In some cases, vaporization device 400 may allow vaporizable materials to be inserted and/or stored in a solid or semi-solid form and subsequently vaporized in a semi-liquid or liquid form. Elements in vaporization device 400 having similar structure and/or performing similar function as those in the example vaporizer device 100 of FIGS. 1-11 are numbered similarly, with the reference numerals incremented by 300.

Vaporization device 400 will be described in combination with another example of a cartridge assembly 500. Cartridge assembly 500 is another example of a cartridge assembly that may be used to store vaporizable material for use with vaporization device 400. Elements in cartridge assembly 500 having similar structure and/or performing similar function as those in the example cartridge assembly 200 of FIGS. 1-11 are numbered similarly, with the reference numerals incremented by 300.

The vaporizer device 400 has a top side 421, a bottom side 423, a front side 425, a rear side 427 and a pair of opposed lateral sides. As shown, vaporization device 400 includes a device body 402 and a removable cartridge assembly 500. In FIG. 1, the removable cartridge assembly 500 is shown in a locked position with respect to the vaporization device 400. Removable cartridge assembly 500 may contain vaporizable material therein for vaporization.

The device body 402 may include a base 404 and a cover 333. The device base 404 may include a plurality of device sections. A first device section 407, proximate the first end 402A, may contain various components of the vaporization device such as a control assembly and/or energy storage member and/or a battery. A second device section 409, proximate the second end 402B may define a receptacle 416 for the cartridge assembly 500.

The base 404 of vaporizer 400 may define a recess 406 similar to recess 106. In vaporizer 400, the recess 406 extends generally from the first end 402A of body 402 to the second end 402B of body 402. In some cases, as with base 104, the base 404 may be open at the first end 402A. A control assembly 408 may be inserted into the first section 407 of base 404. The control assembly 408 may include a first end closure member 418 that encloses the first end 402A. The closure member 418 may also have an outer rim or lip that may help secure the cover 444 to base 404, similar to closure member 118.

The control assembly 408 may be secured within the base 404, e.g. by frictional engagement with an inner surface 432 of base 404. As with base 102, the inner surface 432 of base 404 may be lined to provide a compressible material that allows the control assembly 408 to be inserted therein with a frictional fit. For instance, the control assembly 408 may be slid into the base 404 initially from the first end 402A. The control assembly 408 may also be further secured to base 404 using fasteners such as screws, bolts, and/or adhesives for example. In some embodiments the control assembly 408 may be secured in place by the cover 444. The cover 444 may be secured to control assembly 408 and/or base 404 using a specialized mechanical fastening. A specialized tool corresponding to the fastening may be used to couple and uncoupled the cover 444 from control assembly 408 and/or base 404.

The base 402 may also have a tapered structure, similar to base 102. The base 402 may have a larger cross-sectional area 452 proximate the first end 402A than the cross-sectional area 454 proximate the second end 402B. The first section of the vaporizer 400, with a larger cross-sectional area, may provide recess 406 with an enlarged space within which to store components of the vaporizer such as the control assembly 408 and energy storage members 428. The reduced cross-sectional area of vaporizer 400 proximate the second end 402B, may allow device 400 to provide an inhalation aperture 412 with a size that is more approachable for a user to partially insert into their lips for inhalation.

The control assembly 408 may include a control circuit 420 and one or more energy storage members 428. The control assembly 408 may also include various components generally similar to the first recess section of vaporization device 100, such as the control circuit 420, wireless communication modules 422, 424, 426, energy storage members 428, feedback indicators 430 and so forth.

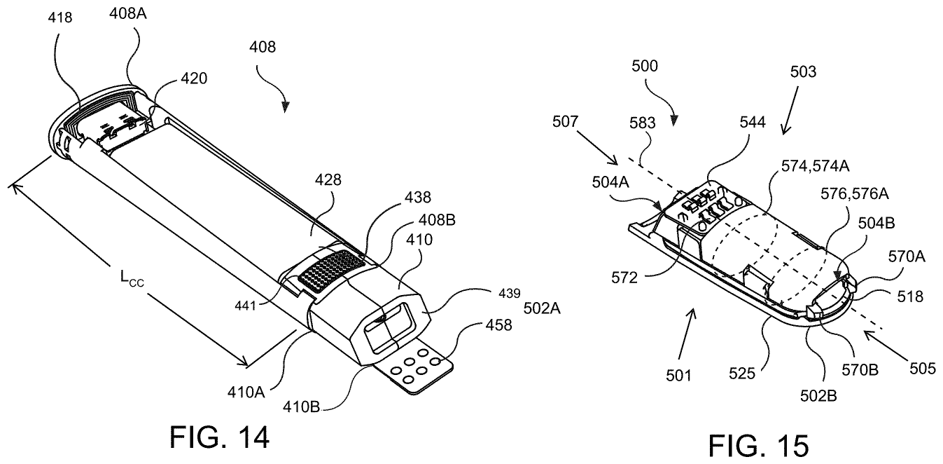

As shown in FIG. 14 and FIG. 33, the air intake manifold 410 in vaporizer 400 may be provided with the control assembly 408. The control assembly 408 may also include a plurality of electrical contacts 458 that are positioned at the second end 410B of air intake manifold 410. In the example shown, the device electrical contacts 458 extend beyond the second manifold end 458B towards the second end 402B of vaporizer 400. As shown, the device electrical contacts 458 are positioned on a bottom surface of receptacle 416 facing upwards into receptacle 416.

The contacts 458 may be positioned to engage corresponding electrical contacts 544 on the cartridge assembly 500 when inserted into receptacle 416. The electrical contacts 458 may allow for various signals to be transferred between the vaporizer control assembly 408 and the cartridge assembly 500, such as power signals, sensor signals, control signals and the like. The corresponding electrical contacts 544 on the cartridge assembly 500 may be attached to a cartridge circuit board 542 which may also include an onboard memory storage module 554 attached thereto. The heating element assembly 510 may be electrically coupled with the corresponding electrical contacts 544 on the cartridge assembly 500 with electrical couplings 568 (FIG. 33) that extend from the heating element assembly 510. The electrical contacts 458 may electrically couple with the corresponding electrical contacts 544 on the cartridge assembly 500 and with the with electrical couplings 568 when the cartridge assembly 500 is inserted into the vaporization device 400.

The vaporizer device 400 may also include a cover 444 that may be used to enclose the first section of the vaporizer base 404. FIGS. 12 and 13 show the vaporization device 400 with the cover 444 connected to base 404.

The cover 444 may protect the components of the control assembly 408 from concussive damage and exposure to dirt or debris. As with cover 144, the cover 444 may be manufactured using a non-conductive material to facilitate wireless communication by the control assembly 408. In some cases, the main body of cover 444 may be manufactured using metallic materials that may interfere with signal transmission. In such cases, the end closure member 418 of control assembly 408 may be formed using a non-conductive material, such as plastic, to facilitate signal transmission therethrough.

In some embodiments, the cover 444 may be manufactured using materials having a higher coefficient of friction from base 404. This may provide a user with a different hand feel when grasping device 400. In some cases, the cover 444 may be electrically insulated from the base 404 when secured to base 404. This may facilitate conductive sensing by the control assembly 408, as a user's hand grasping the vaporizer 400 may be detected via capacitive sensing (as the user's hand may couple the base 402 to the cover 444). The control assembly 408 may use these capacitive sensing signals (the base 402 being electrically insulated from the cover 444) to activate the control circuit 420 from a low-power mode to a more active mode in anticipation of user inhalation.

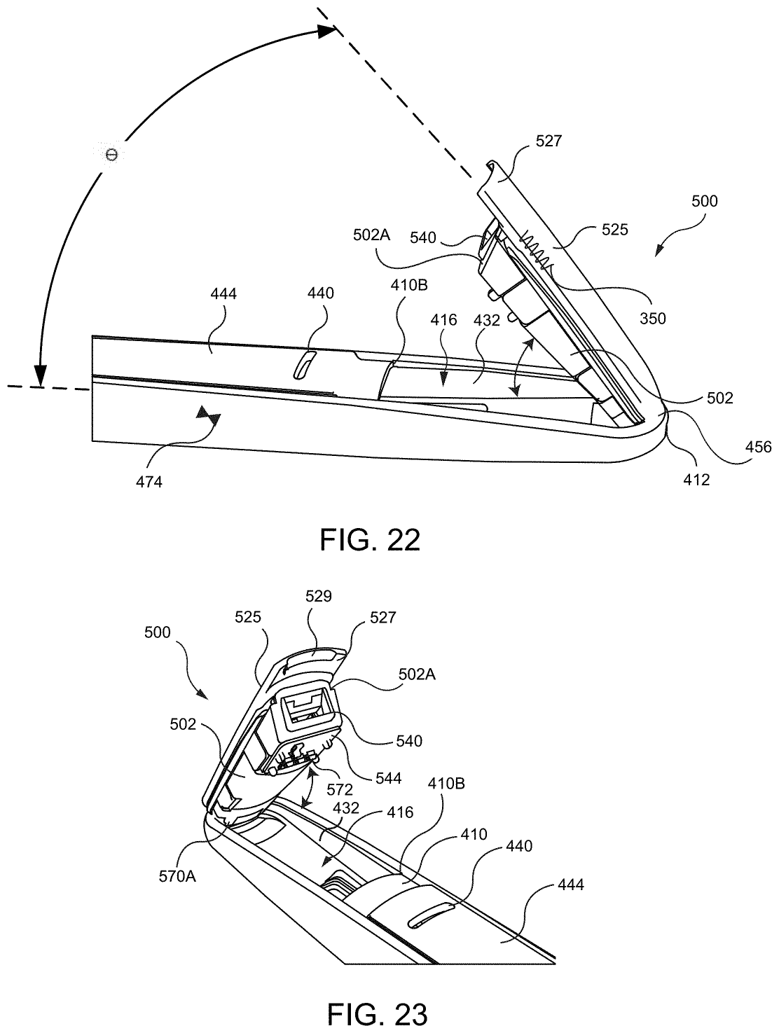

As with vaporizer 100, the center of gravity 474 of vaporizer device 400 may be positioned closer to the first end 402A than to the second end 402B of the device 400 (see e.g. FIG. 22). The heavier components of vaporizer 400, such as the energy storage members 428, may be positioned within the first device section 407. By providing the majority of the weight of vaporizer device 400 nearer to the first end 402A, the vaporizer device 400 will provide a user with a balanced weight when grasped near the first end 402A. As the inhalation aperture 412 is positioned proximate the second end 402B, a user may be inclined to grasp the vaporizer device 400 around the first section 407 so that the second end 402B may be raised to contact the user's lips and mouth for inhalation.

The base 404 of the vaporizer body may be manufactured in a manner similar to base 102. For instance, the base 404 may be formed as a unitary construction. The base 404 may be manufactured using metal, thermoplastic or ceramic materials such as zirconium oxide or other ceramics. When the base 404 is manufactured using metal, machining processes or metal injection molding processes may be used.

The vaporizer 400 may include a mouthpiece having an inhalation aperture 412 at the second end 402B. The inhalation aperture 412 may be formed as a void section in the second end 402B. Optionally, a removable mouthpiece cover may also be provided with aperture 412.

The base 404 may also define a receptacle 416 configured to receive the cartridge assembly 500. The receptacle 416 may be defined in the second portion 409 of the device base 402 proximate the second end 402B. The receptacle 416 may be formed as a recess within the base 402 into which the cartridge assembly 500 may be inserted.

The inhalation aperture 412 may be fluidly connected to the cartridge receptacle 416. When the cartridge assembly 500 is inserted into the receptacle 416, the inhalation aperture 412 may be fluidly connected to a fluid conduit 504 that extends through cartridge assembly 500 from a cartridge conduit inlet 504A to a cartridge conduit outlet 504B. In some cases, a downstream end 518 of the fluid conduit 504 may extend outward through the mouthpiece to define a protruding inhalation aperture 412. In other cases, the inhalation aperture 412 may be flush with the second end 402B of the device body 402, e.g. as shown.

As with vaporizer 100, the vaporizer 400 may also include an air intake manifold 410. The air intake manifold 410 may be configured to allow ambient air to be drawn into vaporizer device 400 and directed into a cartridge assembly 500 positioned within the cartridge receptacle 416. The air intake manifold 410 may be positioned within a third, central section 411 of the device body 402. In vaporizer device 400, unlike vaporizer 100, the cover 444 extends over the air intake manifold 410 as well as the control assembly 408. As shown, the cover 444 may include an ambient air aperture 440 that may be fluidly coupled to an ambient air inlet 438 of air intake manifold 410. A screen or filter 441 may optionally be positioned at the ambient air inlet 438 to filter ambient air entering the air intake manifold 410 (see e.g. FIG. 14).

Air intake manifold 410 may extend from a first manifold end 410A to a second manifold end 410B. The first manifold end 410A may be positioned within the recess 406 adjacent to, or contacting, the second end 408B of the control assembly 408. As with air intake manifold 110, the air intake manifold 410 may be mounted to support member 414 and/or positioned adjacent a front end of the support member 414. The second manifold end 410B may face into the cartridge receptacle 416. A manifold outlet 439 may be positioned at the second manifold end 410B. A manifold fluid flow path 436 may extend between the ambient air inlet 438 and the manifold outlet 439.

The air intake manifold 410 may include a fluid flow sensor 442. The fluid flow sensor assembly 442 may be used to identify ambient air 360 being drawn into the vaporizer 400 via ambient air inlet 438. In some cases, the fluid flow sensor assembly 442 may be configured to identify the volume of air being drawn into the vaporizer 400. The fluid flow sensor assembly 442 may provide flow signals to control circuit 420, to allow control circuit 420 to activate/deactivate the cartridge heating element assembly 510 and/or adjust the temperature of the heating element 564.

In some embodiments electrical contacts extending from the heating element past an outside surface of the heating element assembly may be spaced radially and extend axially from the heating element assembly wherein the electrical contacts may be approximately perpendicular with the fluid flow passage.

In the example shown, a fluid flow sensor assembly 442 in the form of a mass airflow sensor is used. The mass airflow sensor has an upstream input port 442a and a downstream input port 442b. The mass airflow sensor may include a pressure sensing element disposed between the upstream port 442a and downstream port 442b. The pressure sensing element may determine the mass of air being drawn past the upstream port 442a and downstream port 442b by determining the difference in pressure between upstream port 442a and downstream port 442b. In some cases, a thermal hot wire anemometer, or solid state hot wire mass airflow sensor may be used for mass airflow sensor 442. In other cases, individual barometric pressure sensors may be provided at each of the upstream port 442a and downstream port 442b. A difference between the barometric pressure sensors (resulting from the pressure drop element within the fluid channel) may be used to determine the mass airflow.

The output signal from the fluid flow sensor assembly 442 may be used by control circuit 420 to determine the volume or mass of air being drawn into vaporization device 400, e.g. using a lookup table with values providing a correlation between a measured pressure difference and mass air flow.

In some cases, the correlation between the mass air flow sensed and the volume of air entering the air intake manifold 410 may vary based on the temperature of the ambient air. The air intake manifold 410 may include an air temperature sensor (that may be embedded into fluid flow sensor assembly 442 or separate). The air temperature sensor may be configured to measure a temperature of air propagating in a bypass configuration between the between the upstream port 442a and downstream port 442b. The control circuit 420 may then use the measured temperature and air flow mass to determine the volume of air entering air intake manifold 410 (and in turn fluid conduit 504).

In some embodiments, the air intake manifold 410 may include an auditory sensor 443 disposed proximate the air inlet 438. The auditory sensor 443 may be a microphone disposed facing the manifold fluid flow path 436 proximate ambient air inlet 438. The auditory sensor 443 may be used to detect air flow into the ambient air inlet 438. The auditory sensor 443 may output a volume signal to the control circuit 420 that may be used to determine whether ambient air 360 is being drawn into the air intake manifold 410. In some cases, the auditory sensor 443 may be configured with a volume threshold. When the volume threshold is reached, the auditory sensor 443 may transmit an air flow detection signal. This signal may be used (as an alternative to, or in combination with signals from mass airflow sensor 442) to wake the control circuit 420 from a low power or sleep mode. In some cases, the auditory sensor 443 may be mounted within the air intake manifold by an insulating material, such as rubber, to reduce false triggers.

Additionally, or alternatively, other airflow sensors, such as puff sensors (443p FIG. 109) may be used to detect airflow through the air intake manifold 410. For example, signals from the puff sensor may be used to enable/disable operation of a portion of control circuit 420 and/or mass airflow sensor 442. This may ensure that the control circuit 420 and/or fluid flow sensor 442, such as a mass airflow sensor, are not unnecessarily active and draining power from energy storage members 428 in the absence of airflow. In some embodiments, an accelerometer may be used to power down significant portions of the control assembly or control circuit 420 such that upon a lifting action of the vaporization device 400, the accelerometer is activated and wakes up the control assembly.

Using signals from the airflow sensor 442 and/or auditory sensor 443 to activate the control circuit 420 may allow the vaporization device 400 to conserve energy when the device 400 is not being used. In some cases the airflow sensor 442 in the form of a mass airflow sensor may be configured to operate semi-continuously (e.g. at 0.5 Hz, 1 Hz, 2 Hz) in a low power mode to measure a pressure differential between upstream port 442a and downstream port 442b. The lower power mode of mass airflow sensor may be configured to trigger an activation signal to enable/disable operation of a portion of control circuit 420.

Optionally, vaporizer 400 may include a cartridge detection circuit. For example, the electrical contacts 458 may include a pair of cartridge detection contacts that may be connected when the cartridge assembly 500 is inserted into the receptacle 416. The vaporizer 400 may use the cartridge detection circuit as an initial enabling signal that allows the control circuit 420 to be activated. For instance, the cartridge detection circuit may be required to be completed prior to signals from the airflow sensors, described herein above, are able to activate the control circuit 420.

The vaporizer device 400 and cartridge assembly 500 may also include one or more registration features. The registration features may be configured to ensure that cartridge assembly 500 is installed in receptacle 416 in the proper orientation.

For example, the base 404 may define an inwardly projecting lip or overhang 456 in receptacle 416 proximate the second end 402B, e.g. as shown in FIG. 13. The lip 456 may extend from the second end 402B towards the first end 402A to cover a small portion of receptacle 416 adjacent to inhalation aperture 112.

The cartridge assembly 500 may include a corresponding registration feature configured to engage the lip 456. For instance, cartridge assembly 500 may include registration projections 570A and 570B that may be inserted into the receptacle 416 under the lip 456. The projections 570A and 570B may prevent cartridge assembly 500 from being installed within receptacle 416 in an incorrect orientation.

To install cartridge assembly 500 in the receptacle 416, the second end 502B of cartridge assembly 500 may be initially inserted into the second end 402B of device body 402 (i.e. with cartridge aperture 518 engaging inhalation aperture 412). The cartridge assembly 500 may then be lowered into receptacle 416 with the projections 570A and 570B engaging the inner surface 432 of base 402 under lip 456. The electrical contacts 572 on the base of cartridge assembly 500 may also engage corresponding electrical contacts 458 extending from air intake manifold 410. Accordingly, electrical contacts 572 may also define an additional registration feature that may prevent cartridge assembly 500 from being installed within receptacle 416 in an incorrect orientation.

A plurality of LEDs 430 may be provided on the control assembly 408. The LEDs 430 may correspond to apertures 430A formed in the base 402 of vaporizer 400. The LEDs may be used to indicate various operational characteristics of vaporizer 400. For example, the LEDs 430 may vary in color and/or intensity to indicate different states or functions of the vaporizer 400.

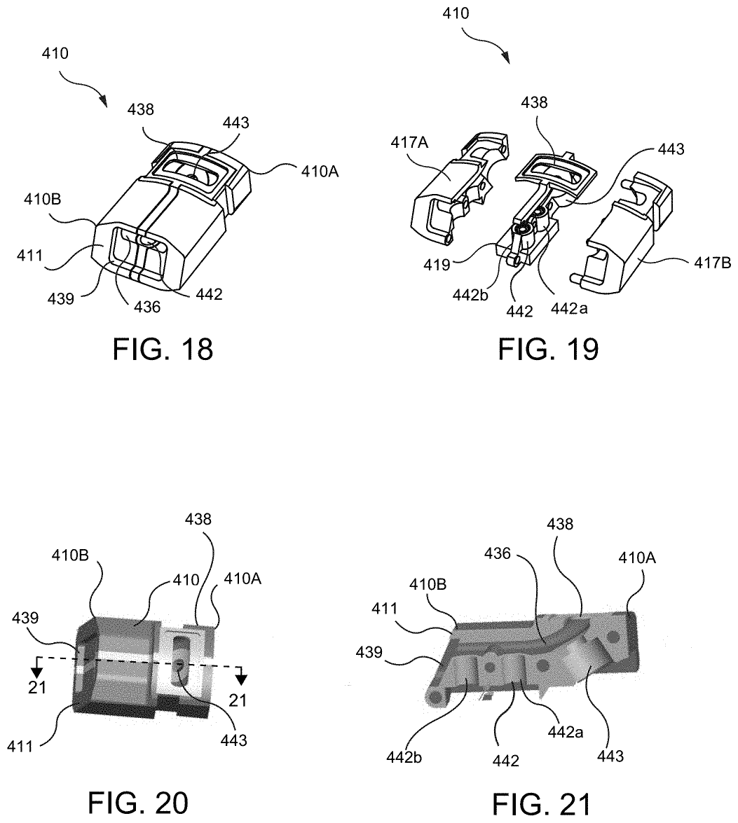

In some embodiments, the air intake manifold 410 may be constructed from a pair of manifold housing shells. For example, FIGS. 18 and 19 illustrate an example of how the air intake manifold 410 may be formed using two outer shell sections 417A and 417B. The air intake manifold may be manufactured using a dual injection molding process. Each shell section 417A and 417B may be manufacturing of thermoplastic materials and joined using a thermoplastic elastomer such as polycarbonate and TPU.

The outer shell sections 417A and 417B may be joined together around a central manifold member 419. The central manifold member 419 may define a manifold air input aperture 438 that is externally exposed in vaporization device 400. The airflow sensor 442 and/or auditory sensor 443 may be mounted to the central manifold member 419. Together, the outer shell sections 417A and 417B may substantially enclose the central manifold member 419 defining the manifold air flow passage 438 therebetween. The air input aperture 438 on central manifold member may be positioned overlying, and sealed to, both shell sections 417A and 417B when assembled.

The cartridge receptacle 416 may be defined in the base 404 of vaporizer 400 extending between the second manifold end 410B and the second end 402B of the vaporizer body 402. The cartridge receptacle 416 may be shaped to frictionally engage the cartridge assembly 500 when cartridge assembly 500 is lowered into receptacle 416. As with receptacle 116, the cartridge receptacle 416 may include a lined, or partially line, inner surface 432 that is formed of a compressible material such as rubber. The cartridge assembly 500 may compress the inner surface of receptacle 416, and the resilience of the inner lining may frictionally engage and secure the cartridge assembly 500 within receptacle 416.

When cartridge assembly 500 is positioned in receptacle 416, the upstream end of cartridge assembly 500 may be fluidly connected to the manifold outlet 439. A vaporizer flow path may then be defined from the ambient air inlet 438/air aperture 440 to inhalation aperture 412 through the cartridge assembly 500.

As shown in FIGS. 13 and 21, the second end 410B of the air intake manifold 410 may be arranged at an angle. That is, when air intake manifold 410 is positioned in vaporizer 400, the second manifold end 410B may have a second end surface 411 that is sloped at an angle to the horizontal plane of vaporizer 400. The upstream end of cartridge assembly 500 may be formed with a corresponding angled or sloped surface. Thus, when cartridge assembly 500 is inserted into the receptacle 416, the interface between cartridge assembly 500 and the air intake manifold 410 may be angled/sloped. This may promote an enhanced seal between cartridge assembly 500 and air intake manifold 410 to reduce or prevent air flow losses at the interface between the intake manifold 410 and cartridge assembly 500.

The cartridge assembly 500 has a top side 501, a bottom side 503, a front side 505, a rear side 507, and opposed lateral sides. As with cartridge assembly 200, the cartridge assembly 500 includes a fluid conduit 504, a heating assembly having a wicking element 508 and a heating element assembly 510, and an elongated storage compartment 516. The storage compartment 516 may be configured to store vaporizable material in a liquid or semi-liquid form (e.g. having a wax-like consistency), similar to storage compartment 216. Cartridge assembly 500 may facilitate the insertion of vaporizable material into a storage compartment 516 in a semi-liquid or even solid form. Nonetheless, during operation of vaporizer device 400, the vaporizable material may flow from compartment 516 into the heating assembly in a liquid or semi-liquid form.

When cartridge assembly 500 is positioned within the receptacle 516, the upstream end 504A of fluid conduit 504 may be fluidly connected to the manifold outlet 439. The fluid conduit 504 may then define a cartridge flow passage that extends from manifold outlet 439 through the cartridge assembly 500 (and also through receptacle 416) to the inhalation aperture 412 formed at the second end 402B of vaporizer 400. The cartridge flow passage, in combination with the manifold fluid flow path 436 may define an enclosed vaporizer fluid flow passage that extends from the ambient air aperture 440 to inhalation aperture 412.

The cartridge assembly 500 may enclose a fluid conduit 504 having a wider cross-sectional area to facilitate airflow. This may allow a user to inhale from vaporization device 400 more easily, without requiring multiple subsequent puffs. Instead, a user may inhale through inhalation aperture 412 more naturally, e.g. using some of the lung tidal volume to reduce the effort required to inhale the vapor emitted within vaporization device 400.

Enabling a user to perform a deep inhalation (e.g. an inhalation that approaches a lung tidal volume such as 0.3 L, 0.4 L, or 0.5 L), rather than merely a puff (e.g. 0.1 L or less), increases the likelihood of the aerosolized vaporizable material in the emitted vapor penetrating more deeply into the user's lungs. This may allow for improved absorption by the user's alveoli.

For example, the fluid conduit 504 may have a cross-sectional area of about 4 mm² or greater. In some cases, the cross-sectional area of the fluid conduit 504 may be about 5 mm² (e.g. a width of about 5 mm and a height of about 1 mm). In some cases, the cross-sectional area of fluid conduit 504 may be about 6 mm² (e.g. a width of about 6 mm and a height of about 1 mm).

With cartridge assembly 500 installed in receptacle 416, the vaporizable material 350 in storage compartment may be vaporized by activating the heating element assembly 510. The vaporizable material 350 may be drawn from storage compartment 516 and into wicking element 508 that is thermally connected to the heating element assembly 510. Current from the energy storage members 428 within the recess 406 of vaporizer 400 may be directed through a resistive heating element 564. The heat emitted by resistive heating element 564 may heat the vaporizable material in wicking element 508 to a predetermined vaporization temperature. When a user inhales from inhalation aperture 412, the vapor emitted by heating the vaporizable material may be drawn into the fluid conduit 504 and entrained with the ambient air that has been drawn into the ambient air inlet 440. This mixture of ambient air and vapor may be inhaled by a user through inhalation aperture 412.

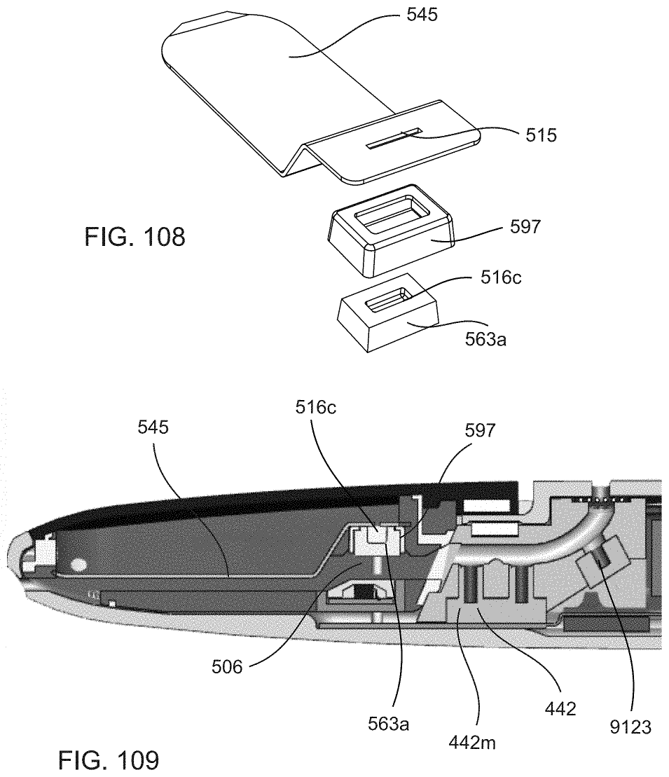

In some cases, the wicking element 508 may be formed integrally with the heating element assembly 510. For example, the heating element assembly 510 may be manufactured from a porous material (e.g. porous ceramics) with pores sized to receive the vaporizable material. The pores may also allow the emitted vapor to pass therethrough when heating element 564 is energized, where in some embodiments a 40-50% open porosity with a tortuous pore structure with a pore size ranging from 20 to 90 microns. In the case when the heating element assembly 510 is used without the wicking element 508, such as shown in FIGS. 108 and 109, then a heating element assembly seal member 597 may be used for creating a frictional seal between the heating element assembly 510 and the interface member 524 and the heating element assembly 510 and the fluid apertures 515.

The fluid conduit 504 and the heating element assembly may be oriented in such a manner that the heating element assembly may be in a direct airstream fluid coupling when air flows within the fluid conduit 504 and further downstream to the mouthpiece (when the cartridge assembly is inserted into the receptacle 416 for the cartridge assembly 500. As air propagates within the fluid conduit 504 it skims vapor from the heating element assembly 510. Having the heating element proximate to the fluid conduit 504 allows for aerosol or vapor emitted from the heating element assembly 510 to be directed into the airstream. This may reduce a condensation build up within the cartridge assembly.

When the cartridge assembly 500 is removed from receptacle 416, the receptacle 416 may be open or exposed to ambient air. Thus, when the cartridge assembly 500 is absent, the vaporizer 400 may not have an enclosed fluid passage that extends to inhalation aperture 412. In vaporizer 400, only the manifold fluid flow path 436 is defined by the device body 402. The majority of the fluid flow passage through vaporizer 400 is instead defined within the cartridge assembly 500.

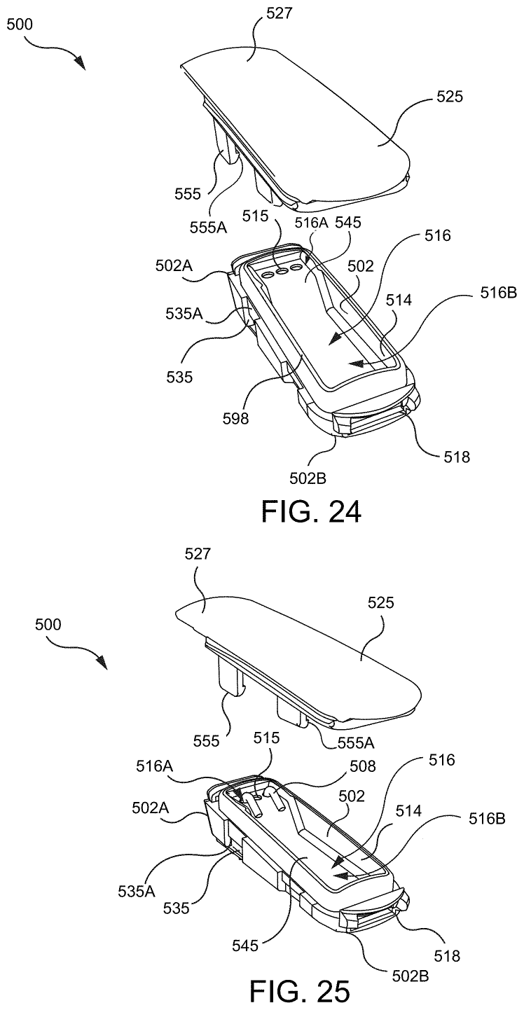

As shown, for example in FIG. 24, the cartridge assembly 500 may have a cartridge base unit 502 and a cover 525. The base unit 502 includes an inner storage volume 516v configured to contain the vaporizable material. The cartridge cover 525 and base 502 may enclose the inner storage volume 516v.

The cartridge base 502 and cartridge cover 525 may be formed separately and then secured to one another. Once the storage volume 516 is filled with vaporizable material, the cover 525 may be secured to the base unit 502 to enclose the storage volume 516. The base unit 502 and cover 525 may be configured to frictionally engage one another to provide the enclosed cartridge.

In some embodiments a wicking gap or space may be provided between the cover 525 and rear end of tongue 545 in a rear portion 516A of the storage compartment 516. For instance, spacer 561 may provide a wicking gap within the storage compartment 516 (see e.g. FIGS. 42,47 and 53).

In the storage compartment shown in FIGS. 74-76, the wicking gap may be positioned proximate the apertures 515b. The wicking gap may hold a portion of the liquid vaporizable material proximate the apertures 515b due to the viscosity of the liquid vaporizable material. This may ensure that vaporizable material remains proximate apertures 515b regardless of the orientation of the vaporization device 500. The size of the wicking gap may vary depending on the viscosity of the liquid vaporizable material. For example, the wicking gap may be in a range of about 0.2 mm-0.3 mm to facilitate maintain some liquid vaporizable material therein.

The inner surface of the cover 525 may define an upper wall (or upper inside surface) of the storage compartment 516. The inner surface of cover 525 may be positioned facing the bottom of storage compartment 516, and may be generally parallel with the bottom of storage compartment 516. The space between the cover 525, the bottom surface of storage compartment 516 and the sidewalls 514 of storage compartment 516 defined by base 502 define the inner storage volume 516v for vaporizable material.

The cartridge assembly 500 may include mechanical engagement members that are used to secure the cover 525 and base 502. The mechanical engagement members may facilitate mounting the cover 525 to base 502 after the storage compartment 516 has been filled with vaporizable material. The mechanical engagement members may also allow the cover 525 to be removed, so that storage compartment 516 may be re-filled and cartridge assembly 500 may be re-used.

The cover 525 may include a plurality of cover engagement members 555. The base unit 502 may include a corresponding plurality of base engagement members 535. The base engagement members 535 and cover engagement members 555 may be aligned around the perimeter of the cartridge assembly 500. When the cover 525 is lowered onto the base unit 502, the engagement members 555 and 535 may engage one another in a frictional engagement, securing the cover 525 to the base 502.

The cover engagement members 555 may be in the form of snap clips. The engagement members 555 may extend or project downward from the main body of the cover 525. At the distal ends of the projection, the engagement members 555 may include an inwardly extending section 555A. The inwardly extending sections 555A may have a substantially flat upper inner surface. In some cases, the inwardly extending sections 555A may even be angled slightly with an acute angle relative to the downwardly extending sections.

The base engagement members 535 may be defined as recesses in the lateral sides of the cartridge base 502. The recesses may be shaped to accommodate the inwardly extending sections 555A of the cover engagement members 555. When the cover 535 is lowered onto base unit 502, the inwardly extending sections 555A may be received within the corresponding recesses in base unit 502. An upper inner surface 535A of the recesses may engage the upper surfaces of the inwardly extending sections 555A and prevent the cover 525 from separating from base unit 502.

The cover engagement members 555 may be resilient engagement members. When the cover 525 is lowered on to base 502, the engagement members 555 may be pushed outwardly by the sides of base 502. When the engagement members 555 meet engagement members 535, the cover engagement members 555 may resiliently return to a substantially vertical alignment with the inwardly extending sections 555A of engagement members 555 secured in the recesses of base engagement members 535.

Additionally, or alternatively, the cover 525 and base 502 may be secured to one another using other fastening means, such as ultrasonic welds and/or adhesives. The cover 525 and base 502 may include a plurality of fastening locations around the circumference/perimeter of cover 525. In some cases, the fastening locations may be formed as a continuous weld or adhesive extending along the circumference of cover 525. In some embodiments the cover 525 may have grooves and the base 502 may have rails and the cover 525 is slid onto the base 502 for the grooves to engage the rails.

The cartridge assembly 500 may also include a storage compartment seal member 598. The storage compartment storage compartment seal member 598 may extend around the upper periphery of the storage compartment 516. The storage compartment seal member 598 may be secured between the cover 525 and base 502. The storage compartment sidewalls 514 defined by base 502 may extend to upper edges defining an upper perimeter or upper peripheral edge of the storage compartment 516. The storage compartment seal member 598 may extend around the entire upper perimeter of storage compartment 516.

The storage compartment seal member 598 may define a fluid seal between the cover 525 and base 502, enclosing the inner volume of storage compartment 516. The storage compartment seal member 598 may prevent leakage at the interface between the cover 525 and base 502. The storage compartment seal member 598 may provide a gasket seal between cover 525 and base 502.

The storage compartment seal member 598 may be formed of a compressible material. The storage compartment seal member 598 may be provided initially on one of the cover 525 and base 502. The storage compartment seal member 598 may be secured temporarily or permanently to the one of base 502 and cover 525 (e.g. using an adhesive or formed integrally with the periphery of base 502 or cover 525). When the cover 525 is secured to base 502, the seal 598 may be compressed to provide a gasket seal surrounding the upper perimeter of the storage compartment 516.

Providing a cover 525 that may be secured to the base 502 using mechanical engagement members 535 and 555 (while sealing storage compartment 516) may facilitate filling the vaporizable material into storage compartment 516. As is described in further detail below, vaporizable material may be deposited initially into the storage compartment 516 of base 502 prior to cover 525 being secured thereto. This may allow more viscous fluid or waxy vaporizable materials to be easily deposited into storage compartment 516. For example, viscous cannabis extracts, such as shatter or crystals may be used within the elongated storage compartment 516. In some cases, the vaporizable material may be deposited into storage compartment in a semi-solid or solid form. For instances, sections of vaporizable material may be cut or formed into the shape of storage compartment 516 and then deposited therein. Following deposition of the vaporizable material into the storage compartment 516, the cover 525 may be secured to base 502 enclosing the vaporizable material within storage compartment 516.

The cover 525 may extend along the entire length of the base unit 502 on the upper side of cartridge assembly 500. In some cases, the cover 525 may extend beyond the base unit 502, e.g. beyond the first end 502A of base 502 as shown in FIG. 22).

The cover 525 may include a tail portion 527 that extends rearward of the first end 502A of base 502. The tail portion 527 may provide a grip or groove 529 for a user to insert the cartridge assembly 500 into receptacle 416 or remove cartridge assembly 500 therefrom, e.g. as shown in FIG. 23. When cartridge assembly 500 is installed in receptacle 416, the tail portion 527 may extend at least partially over the air intake manifold 410. A gap may be provided between the tail portion 527 and the cover 444 of the vaporizer 400. The gap may allow a user to grasp the tail portion 527 and remove the cartridge assembly 500 from receptacle 416. The gap may be sized to allow a user to insert a fingernail or tool and access the rear end of tail portion 527.

In some embodiments, cover 525 may be impermeable to prevent any air or fluid flow therethrough. This may prevent leakage from storage compartment 516.

In other embodiments, the cover 525 may include one or more vent apertures. The vent apertures may be shaped to allow airflow communication between the storage compartment 516 and the external environment, while substantially reducing or preventing an amount of vaporizable material from exiting storage compartment 516. This may facilitate pressure equalization for the storage compartment 516 to facilitate flow of the vaporizable material out of the storage compartment 516 and onto wicking element 508. In some cases, the vent aperture is about 0.1 mm in diameter. In some cases, wicking elements or pads may be disposed proximate the vent apertures to further prevent any loss of vaporizable material. For instance, a porous material may be positioned proximate the vent aperture of (e.g. having a pore diameter of about 100 micrometers) to further prevent leakage of vaporizable material.

In some cases, the cover 525 may include a series of channels connecting the vent apertures to the storage compartment 516. Additionally, a screen or filter may be provided between vent apertures and storage compartment 516. In some cases, a gas permeable liquid impermeable membrane may be provided with the vent apertures to prevent leakage. This may facilitate ambient air flow while reducing or preventing the flow of vaporizable material out through the vent apertures.

The storage compartment 516 in cartridge assembly 500 may be provided separately from the fluid conduit 504. Unlike with cartridge assembly 200, the storage compartment 516 is not annular in shape and does not surround the fluid conduit 504. Rather, the storage compartment 516 occupies a majority of the upper portion 584A of the cartridge assembly 500 while the fluid conduit 504 is positioned almost entirely in a lower portion 584B of the cartridge assembly 500. The storage compartment 516 may also occupy some of the lower portion 584B of the cartridge assembly 500.

For instance, the cartridge assembly 500 may define a central axis 583 extending from a first end 502A to a second end 502B of the cartridge assembly 500. A horizontal plane along central axis 583 may bisects the cartridge assembly 500 into an upper portion 584A and a lower portion 584B. The fluid conduit 504 may be contained almost entirely within the lower portion 584B, while the majority of the storage compartment 516 is positioned in the upper portion 584A of cartridge assembly 500. As shown, the sections of the fluid conduit 504 that are aligned with, and downstream from, the heating assembly are entirely contained in the lower portion 584B.

As shown, the storage compartment 516 overlies the fluid conduit 504 for the entire length of the storage compartment 516. By providing the fluid conduit 504 in the lower section 584B of the cartridge assembly 500, without any lateral portion of the storage compartment 516 occupying the lateral width of the cartridge assembly 500 where the fluid conduit 504 is positioned, a wider fluid conduit 504 may be provided. As shown, the fluid conduit 504 may extend across substantially all of the internal width of the lower portion 584B. This may provide an increased cross-sectional area throughout fluid conduit 504, resulting in easier air flow and inhalations from vaporizer 400.

In cartridge assembly 500, the fluid conduit 504 extends from the first end 502A of base 502 to the second end 502B of base 502. The fluid conduit 504 may extend generally in parallel with storage compartment 516. The fluid conduit 504 extends from a first conduit end 504A at cartridge inlet aperture 540 to a second end 504B at cartridge outlet aperture 518. The fluid conduit 504 may define a fluid flow passage through the cartridge assembly 500 that is linear throughout the majority of the length of cartridge assembly 500. When installed in receptacle 416, the cartridge inlet aperture 540 may engage manifold outlet 539 and cartridge outlet aperture 518 may engage inhalation aperture 412.

In alternative embodiments, the fluid conduit may be formed between the base 402 and the cartridge inserted into receptacle 516. The cartridge may define an enclosed fluid passageway there beneath when inserted in receptacle 5176.

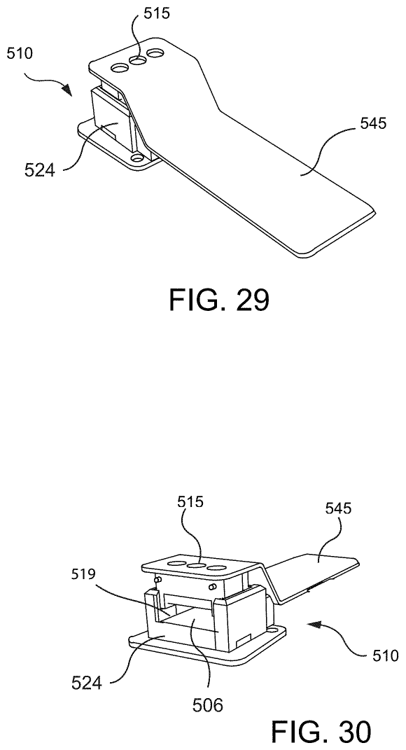

The base or bottom surface of the storage compartment 516 may contact the fluid flow path that extends through cartridge assembly 500. The base may be defined by a tongue 545 (see e.g. FIG. 29) that extends the majority of the length of storage compartment 516. The tongue 545 may define an upper wall of the section of fluid conduit 504 downstream from the heating assembly.

The tongue may facilitate heat transfer between the fluid conduit 504 and storage compartment 516. For example, the tongue 545 may be manufactured of a thermally conductive material, such as a metal (e.g. steel, copper, or gold plated copper) or thermally conductive ceramic.

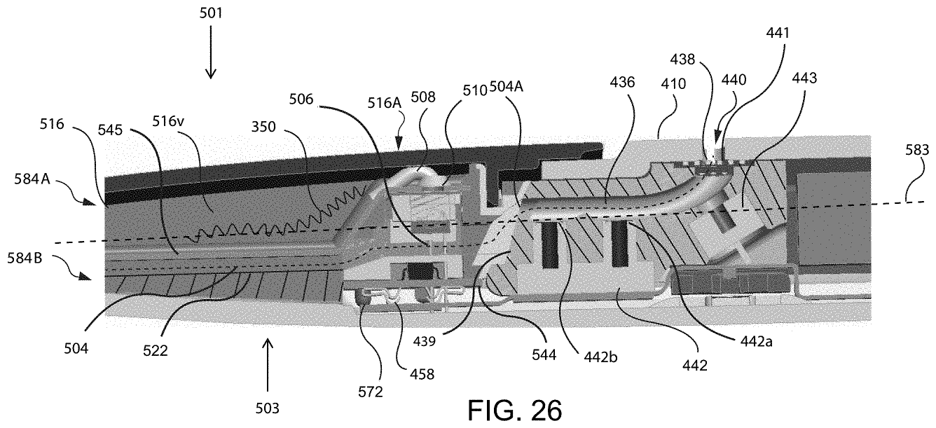

As shown in FIG. 26, the fluid conduit 504 may extend along the length of the storage compartment 516. The fluid conduit 504 may be thermally coupled to the bottom of the storage compartment 516 by tongue 545. This may encourage thermal transfer between fluid conduit 504 and storage compartment 516, which may promote cooling of the vapor through fluid conduit 504 as well as heating of the liquid vaporizable material 350 in storage compartment 516. This may provide a user with a more comfortable temperature of vapor for inhalation. This may also reduce the viscosity of the liquid vaporizable material 350 in storage compartment 516, which may facilitate uptake into the heating assembly (e.g. into wicking element 508 or through apertures 515b formed in proximity of a resistive heating element 564d as shown in FIGS. 74-76).

In certain examples, the vaporizable material 350 may have a viscosity between about 100 and 25,000 Centipoise. In other embodiments, the vaporizable material may exhibit a viscosity between about 1000 and 5000 Centipoise. As the tongue 545 is heated by heating element assembly 510 and vapor is flowing through conduit 504, the tongue 545 may transfer this heat to the vaporizable material 350 and reduce the viscosity of the vaporizable material proximate a first end 516A of the elongated storage compartment 516. This may facilitate the flow of vaporizable material into wicking element 508 and/or through fluid apertures 515.

The cartridge assembly 500 may include a heating chamber 506 disposed at the first end 516A of the storage compartment. The heating chamber 506 may include a wicking element 508 and a heating element assembly 510.

The wicking element 508 may be arranged in fluid contact with the interior of the storage compartment 516. The wicking element 508 may draw vaporizable material from storage compartment 516 into the heating chamber 506. As shown in the example of FIGS. 26 and 41, the wicking element 508 may extend into the inner volume of the storage compartment 516. Alternatively, the wicking element 508 may be positioned at the end of the storage compartment 516, or adjacent thereto, and coupled via apertures 515. In some cases, the wicking element 508 may be integrated into the heating element assembly (see e.g. FIGS. 74-76). The wicking element may be in fluidic contact with the fluid apertures 515 and the heating element assembly 510 may be in fluidic contact with the wicking element 508. In some embodiments the heating element assembly 510 may be in direct contact with the interior of the storage compartment 516. In some embodiments the wicking element 508 may be planar shaped and in some embodiments, rod shaped. In some embodiments the heating element assembly may be rod shaped or may be cylindrical shaped.

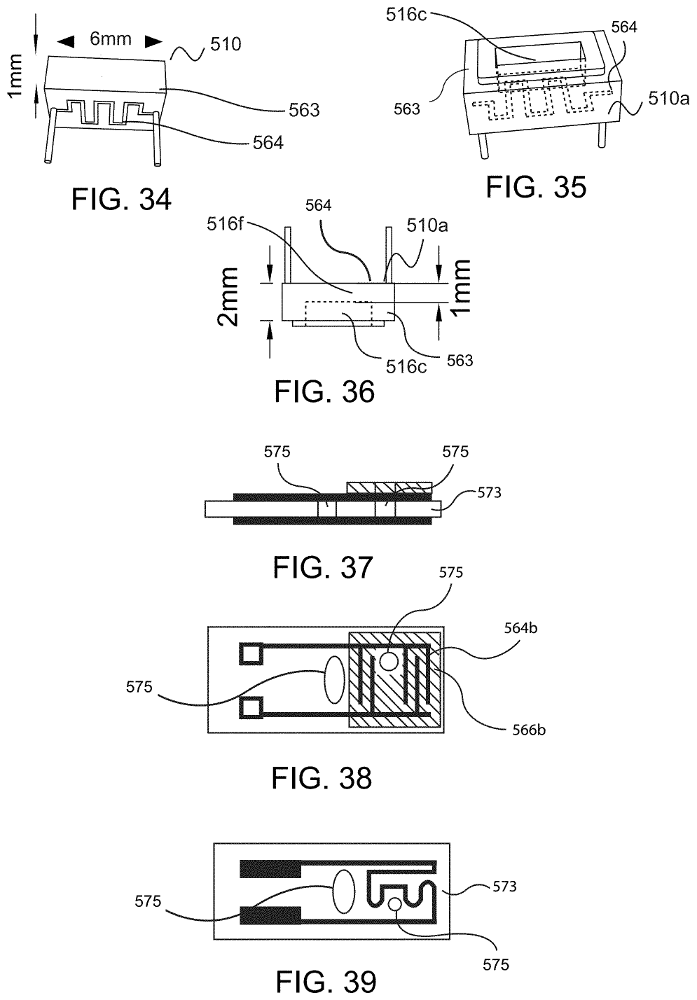

Referring to FIG. 34, FIG. 35 and FIG. 36, the heating element assembly 510 may include a resistive heating element 564. The resistive heating element 564 may be activated to emit heat by directing current from energy storage members 428 therethrough. The heating element assembly 510 may be positioned in thermal contact with the interior of the storage compartment 516. The heat emitted by heating element 564 may heat the vaporizable material that was drawn into heating element assembly 510 to a predetermined vaporization temperature to generate phyto material vapor.

Referring to FIG. 34, FIG. 35 and FIG. 36 as shown, a heating element assembly 510 and 510a that is generally rectangular in shape. FIG. 34 illustrates the heating element enclosure 563 that is without a cavity formed therein, whereas FIG. 35 and FIG. 36 show a cavity 516c formed within the heating element enclosure 563a or a secondary reservoir 516a for being formed in conjunction with the storage reservoir 516 and fluidly coupled therewith through ports 515 or in some embodiments directly with the storage reservoir 516. The heating element assembly 510 may be approximately 6 mm×1 mm×4 mm in dimensions and heating element assembly 510a may be approximately 6 mm×2 mm×4 mm. A heating element assembly cavity floor 516f having a thickness may be formed between a lowest point of the cavity 516c and a sidewall of the heating element assembly proximate the heating element. The cavity floor 516f may in some embodiments be 1 mm in thickness and in other embodiments to be 0.8 mm or 0.6 mm or 0.5 mm in thickness. The cavity 516c allows for vaporable material to flow into the cavity and to be substantially retained within the cavity 516c. A thinner cavity floor 516f allows for a faster rate of flow of vaporizable material from the cavity 516c to the heating element. A thinner floor, for example 0.6 mm vs 0.7 mm provide for a less tortuous path through a porous structure of the heating element assembly and for a lower flow resistance. In some cases, for higher viscosity oils, for example 10,000 Centipoise a 0.6 mm thickness of the floor 516f is preferable and in some cases with a lower viscosity oil, for example 5,000 Centipoise a 0.8 mm thickness of the floor 516f may be used.

The heating element assembly 510 or 510a may be held in place by an interface member 524 (FIG. 50) where the wicking element 508 is exposed to the vaporizable material from the storage compartment 216 may be drawn to the heating element assembly 510 by wicking element 508. The vaporizable material in the wicking element 508 may then be heated by the heat emitted by the heating element 564 or resistive wire.

Referring to FIG. 35 and to FIG. 108, in some embodiments, when a wicking element 508 is not utilized, a heating element assembly seal member 597 may be utilized that is manufactured from an elastomeric and deformable material that may form a frictional seal between the heating element assembly 510 and the interface member 524 and the heating element assembly 510 and the fluid apertures 515, where the heating element assembly seal member 597 is compressed between the interface member 524 and the tongue 545 or the cartridge body 502. The heating element assembly 510 may be manufactured with a partially embedded heating element 564, in the form of a resistive wire, embedded in a porous material forming the heating element enclosure 563a or 563.

For example, the heating element enclosure 563a or 563 may be manufactured using a porous ceramic and the porous ceramic acts as the wicking element. The heating element enclosure 563a or 563 may be manufactured using a porous ceramic substrate inlaid with a heating mesh or a planar curved heating wire that forms the heating element 564 or resistive wire, where the heating element 564 or resistive wire may be partially embedded within the ceramic substrate or the heating element enclosure 563a or 563. The resistive heating may be at least partially embedded with the ceramic substrate.

In manufacturing the resistive heating wire together with the heating element enclosure 563a or 563 is manufacturing using a process of hardening molding in-cavity. The resistive heating wire is first prepared (for example stamped or laser cut or coiled). The resistive heating wire may be uniformly manufactured for providing of rapid and uniform heating throughout its length. The resistive heating wire may include materials such as nickel-chromium alloy, iron-chromium-aluminum alloy, stainless steel, pure nickel, titanium or nickel-iron material. The resistive heating wire may be manufactured using laser cutting technology, stamping technology or etching technology and a thickness of about 0.03 mm to 0.4 mm.

In preparation of the heating body of the porous ceramic, a ceramic slurry is prepared with paraffin wax and a ceramic powder. A weight ratio of paraffin is about 30%-50% and a weight of the ceramic powder is 70%-50%. For manufacturing of the ceramic slurry, the paraffin wax is first made into a molten state molten state and then stirred with the ceramic powder for about 3 hours until the paraffin wax and the ceramic powder are completely mixed uniformly. The ceramic powder includes one or more of silica flour, clay, emery powder, silicon carbide, medical stone powder, mullite powder and cordierite powder. Furthermore, the paraffin wax and ceramic powder slurry may include includes one or more of alumina, potassium oxide, magnesium oxide, ferric oxide, silicon dioxide and calcium peroxide.

The resistive heating is placed into the mold and then the molten and stirred ceramic slurry is poured into the mold cavity containing the resistive heating wire. The ceramic slurry is then injected in the mold cavity that contains the resistive heating wire and hardened. This hardened molded ceramic slurry forms a green body of the ceramic matrix that includes the resistive heating wire that is embedded in the green body of the ceramic matrix. The resistive heating wire and green body of the ceramic matrix form a ceramic heating body blank.

For the sintering process, the ceramic heating body blank is taken out from the mold cavity and sintered in an aerobic environment with temperature of between about 200° C.-600° C., which makes the paraffin wax turn into a gas and separate from the ceramic green body at this temperature. This ceramic heating body blank is then further heated under vacuum at about 1100° C.-1400° C. in order to obtain a dry and structurally stable ceramic heating body as the heating element enclosure 563a or 563. For example, a porous ceramic material used with heating element assembly 510 or 510a may have a 40-50% open porosity and with a tortuous pore structure and use pore sizes ranging from 1 to 100 microns, where more specifically it may have pore sizes of 10, 15, 30, 50, 60 and 100 microns. In some embodiments a higher porosity heating element enclosure 563a or 563 is used with a higher viscosity material for vaporization.

In some embodiments the heating element assembly 510a may be manufactured from a porous ceramic material containing aluminum oxide and silicon carbide, then sintered and with a uniform porous structure having an 40-50% open porosity with a tortuous pore structure with a pore size ranging from 20 to 90 microns. The heating element may be silk screened onto the heating element assembly using a silver (Ag) conductive ink for creating of the resistive heating element having a resistance of about 1.1 ohms to 1.3 ohms and in some cases 1.15 ohms.

The heating element assembly 510 or 510a may have the heating element extend at least partially or be facing the heating chamber 506. The secondary reservoir 516 may be about 4 mm long×1 mm in depth by about 2 mm in width. The secondary reservoir 516a may be formed as the cavity of the heating element enclosure 563a as seen in FIG. 35 heating element assembly 510a.

In an alternative embodiment, the heating element may be provided by an ultrasonic or vibrational heating element. A high-frequency vibrational heating element may be used in combination with a resistive heating element in some cases. The vibrational heating element may operate to heat, as well as atomize, the liquid vaporizable material simultaneously.

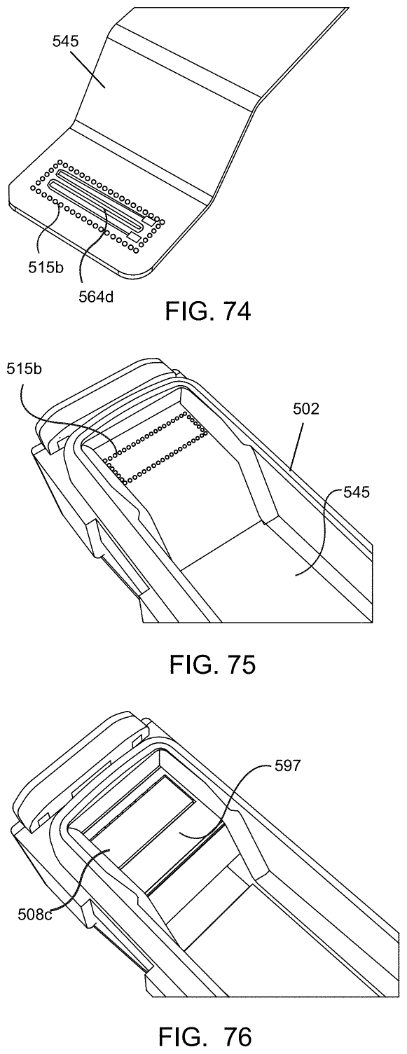

In some embodiments, the heating element assembly 510 may be thermally insulated from the cartridge body 502. For instance, an air gap may be provided between heating element assembly 510 and body 502. In some cases, a seal member may be positioned between heating element assembly 510 and body 502. For example, a silicone rubber seal member or other elastomeric seal may be used (see e.g. heating element assembly seal member 597 shown in FIG. 76). The seal member may prevent leakage of the vaporizable material into other portions of cartridge assembly 500, such as fluid conduit 504, prior to vaporization. The heating element assembly seal member 597 forming a frictional seal between the heating element assembly 510 and the interface member 524.

The heating element assembly 510 may also be thermally insulated from the tongue 545 by wicking element 508. An enclosure or heating element assembly 510 (e.g. ceramic housing which may include an elastomeric seal 597) may also provide further separation between heating element 564 and tongue 545.

In some cases, the heating element assembly 510 may also be thermally insulated from the tongue 545 using a thermoplastic elastomeric seal 597 (FIG. 108). In the example shown in FIGS. 74 and 76, the seal 597 may be positioned about the heating element assembly 510 to enclose apertures 515b about their periphery by the thermoplastic elastomeric (e.g. TPU, silicone) seal.

Optionally, a temperature sensor 566 may be in thermal communication with the heating element 564. The temperature sensor 566 may generate a temperature signal indicative of the temperature of heating element 564 and/or heating chamber 506. The temperature sensor 566 may be electrically connected to the first plurality of electrical contacts 572 on cartridge assembly 500. When cartridge assembly 500 is installed in receptacle 416, the calibration temperature signals from temperature sensor 566 may be provided to control circuit 520 via electrical contacts 572.

The heating chamber 506 may be positioned generally at the first end 516A of storage compartment 516 (proximate first end 502A). The heating chamber 506 may include a heating element assembly 510 that is in thermal communication with a wicking element 508. The heating element assembly 510 is also in fluid communication with a fluid conduit 504 extending through the cartridge assembly 500.

The heating chamber 506 may include a heating element fluid port 519 coupling the heating chamber 506 to fluid conduit 504. Air entering the fluid port 519 may be heated by heating element 564 in thermal communication with the heating chamber 506. Vaporizable material may be drawn through fluid apertures 515 (e.g. using wicking element 508) and then heated by heating element 564 to generate vapor. The vapor may mix with the air drawn in through fluid port 519 and then pass out the downstream heating element outlet along fluid conduit 504 to inhalation aperture 412.

In some embodiments, the apertures 515 may be formed overlying the heating chamber 506. A wicking element 508 may be provided extending through apertures 515, or underlying the apertures 515. The fluid may then flow into wicking element 508 which, in turn may be heated by heating element assembly 510.

In some embodiments, as shown in FIGS. 74-76, fluid apertures 515b may be formed in tongue 545 surrounding the perimeter of a heating element assembly that includes a heating element 564d and wicking element 508c. In the example shown, the heating element assembly and heating element 564d may be arranged in thermal contact with a portion of tongue 545. The apertures 515b surrounding the heating element assembly may at least partially isolate the remainder of tongue 545 from the heat emitted by heating element 654d.

The apertures 515b may also allow the vaporizable material from the storage compartment 516 to flow through to heating element 564d and/or a wicking element 508c (that may be provided using a porous ceramic in some instances). For example, the apertures 515b may be formed through tongue 545 using a laser drilling process. The diameter of the apertures 515b may be selected to permit flow of liquid vaporizable material therethrough. For instance, apertures 515b may have aperture diameters in the range of about 0.06 mm to 0.08 mm.

As shown, the heating element 564d is formed as a film heating element on the underside of tongue 545. The heating element 564d may be a thick film heater that is deposited onto a substrate (e.g. ceramic or stainless steel) through a thick-film screen printing process. Insulating materials, heating resistors, conductors and a protective glaze may also be provided in the deposition process. The apertures 515b may be formed around the perimeter of the deposited heating element 564d to provide thermal insulation as well as increasing the available flow passages for vaporizable material. As with the various heating element assemblies described herein above, a temperature sensor may also be provided in proximity to the heating element 564d.

In general, the heating chamber 506 may include one or more fluid apertures 515 that allow vaporizable material from storage compartment 516 to pass through to be heated by the heating element assembly 510. Wicking elements 508 may be provided either extending into storage compartment 516 through apertures 515 (see e.g. FIG. 24) or outside the storage compartment in fluid communication with apertures 515 (e.g. a wicking sheet or pad). The wicking element 508 may be thermally coupled to (e.g. in contact with) heating element assembly 510 to allow the collected vaporizable material to be heated and then entrained into fluid conduit 504. For example, the wicking element 508 may be secured to one or more outer surfaces of a heating element assembly 510.

In some embodiments, a resistive heating element 564b may be patterned and sintered into a substrate 573 (see e.g. FIGS. 37-39). For example, the substrate 573 may be a ceramic or stainless steel substrate. The heating element 564b may be formed on substrate 573 using a thick film process.

Vapor apertures 575 may be formed within the substrate 573 to facilitate the flow of vapor from a wicking element, such as wicking element 508′ disposed on the surface of the substrate 573 to the fluid conduit 504. The substrate may include a resistive film 566b usable to sense a temperature of the heating element 571.

Optionally, one or more micro-heaters may be formed on a silicon substrate using a conducting MEMS process. Through the MEMS process, silicon under the bridge micro heater is etched away to release a thin resistive wafer having a serpentine resistive conductor. The heating element assembly thus formed may provide micro-heaters suspended as a bridge from a silicon substrate. Because the micro heater is etched out and has a low thermal mass, the heater may be rapidly heated (e.g. up to approximately 280 Celsius within less than a second) using low current levels. The micro-heaters may operate similar to a miniature hot plate when current is applied thereto from the control circuit 420.

In some cases, the micro-heaters may also include a thermally coupled resistor. The thermally coupled resistor may be configured to operate as a temperature sensor providing for real-time thermal monitoring and control.

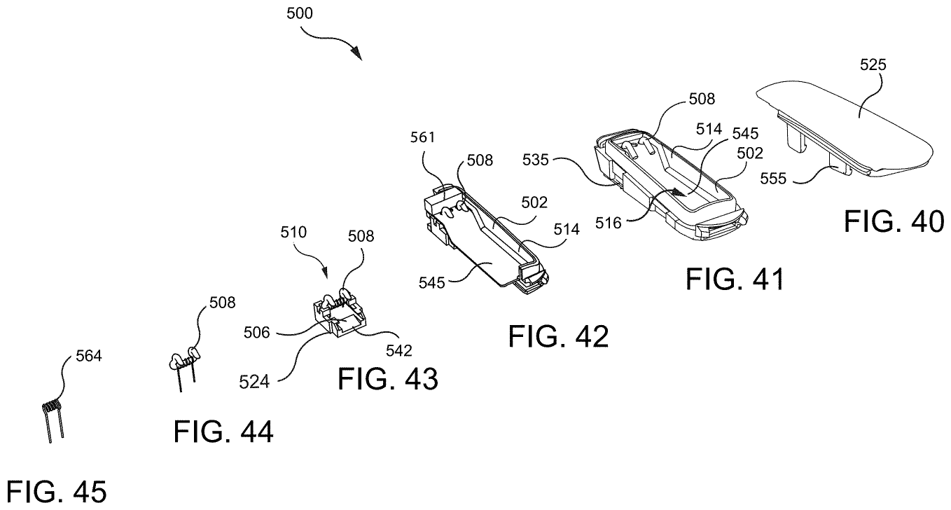

FIGS. 40-45 illustrate an example of cartridge assembly 500 with a first example heating assembly where the heating element assembly may be in the form of a resistive heating coil wrapped around a wicking element.

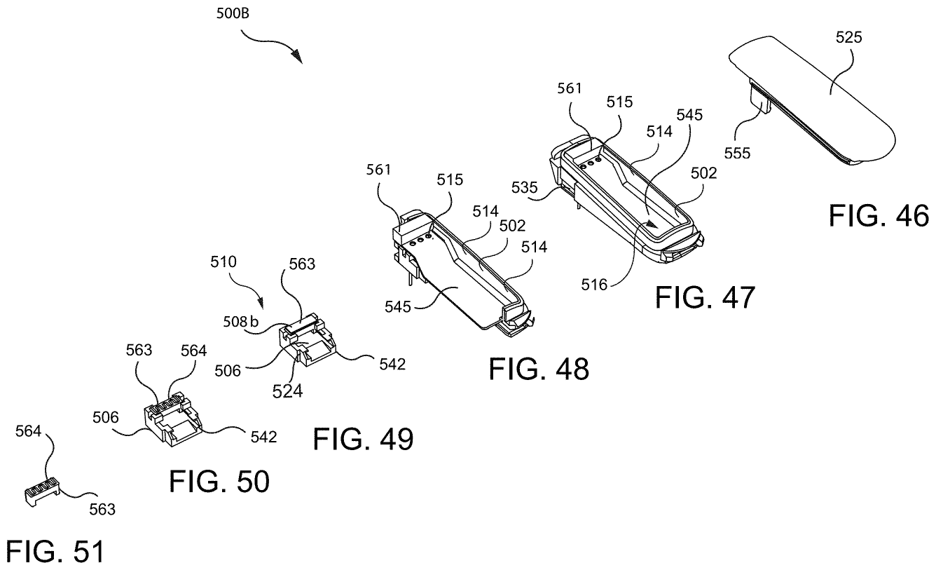

FIGS. 46-51 illustrate an example of a variant cartridge assembly 500B with a second example heating element assembly where the heating element assembly may include a planar heating element stamped metal resistive coil embedded within the heating element assembly or printed on a surface of the heating element assembly.

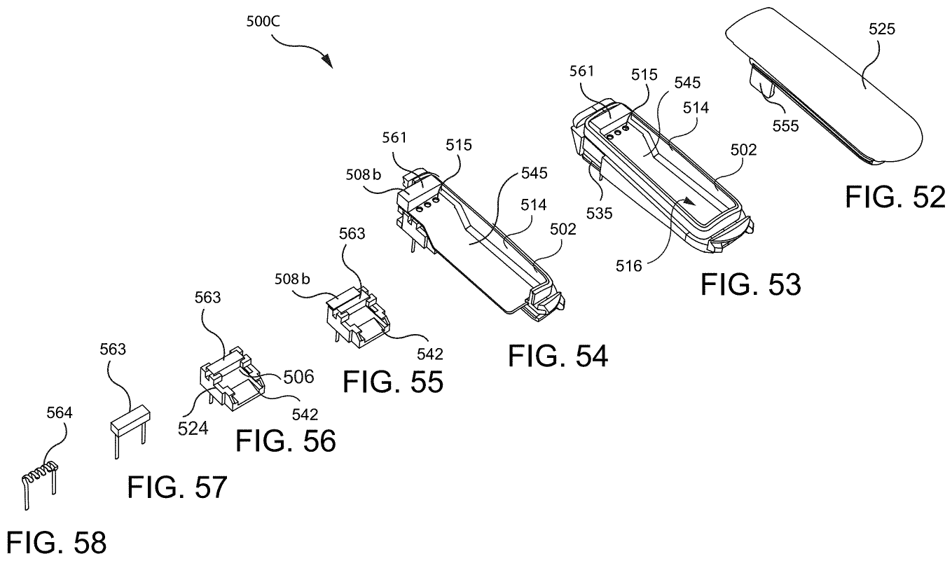

FIGS. 52-57 illustrate an example of a variant cartridge assembly 500C with a third example heating assembly where a cylindrical resistive heating coil may be at least partially enclosed within the heating element assembly.

In general, the body 502, storage compartment 516 and cover 525 are the same for cartridges 500, 500B and 500C. However, a slightly modified heating assembly is used in each cartridge.

Cartridge assembly 500 includes a heating element assembly 510 in which a resistive coil wire 564 is enclosed within an outer heating element enclosure or heating element assembly 510. The heating element enclosure 563 may be manufactured from a porous ceramic material and may enclose the resistive coil 564 therein. Heat may then be transferred to the vaporizable material in wick 508 through the outer surface of enclosure 563. In some embodiments the heating element assembly 510 may include a plurality of resistive wire coils as the heating element 564. Each coil may be separately coupled to the control circuit and individually operable. Each coil may be individually triggered in response to control signals from the control circuit, e.g. based on mass airflow data from the vaporization device 400.

Wicking element 508 extends into the storage compartment 516 through apertures 515. The wicking element 508 may include first, or proximal ends that are secured to the heating element 564. The second, or distal ends of wicking element 508 may extend into the storage compartment 516. This may facilitate capillary action of wicking element 508 in drawing the vaporizable material from compartment 516 to the proximity of heating element assembly 510.

In cartridge assembly 500B, the heating element assembly 510 may include the heating element 564 or resistive wire disposed on the surface of a heating element enclosure 563. The enclosure 563 may be formed using a porous ceramic material and may provide a substantially flat contact surface for vaporization. The heating element 564 or resistive wire may be exposed on the contact surface of the heating element assembly 510.

A substantially planar wicking element 508b may be positioned on the surface of the enclosure 563. This may provide an extended contact surface area between wicking element 508b and the heating element assembly 510. For instance, a cotton sheet or pad with a thickness of about 0.1-0.3 mm may be used for wicking element 508b. The wicking element 508b may be positioned in the heating chamber 506, external to the storage compartment 516.

Referring to FIGS. 52 to 58, in cartridge assembly 500c, the heating element assembly 510 may include the heating element 564 or resistive wire embedded within the heating element enclosure 563. The enclosure 563 may be formed using a porous ceramic material and may provide a substantially flat contact surface for vaporization. A wicking element 508b may then be provided on the surface of enclosure 563.

When the cover 525 is secured to base 502, the storage compartment 516 may be entirely enclosed (by cover 525, tongue 545 and sidewalls 514) with the exception of one or more fluid apertures 515 fluidly coupling storage compartment 516 to heating chamber 506. The fluid apertures 515 may be formed in a first end of the tongue 545. In the example shown, the fluid apertures 515 are shown as circular apertures. However, alternative shapes of fluid apertures, such as slots, square, and oval apertures may also be used. The typically size of the fluid apertures 515 may range between about 0.1 mm to about 2 mm in diameters. Examples of suitable aperture diameter may include range of approximately 0.1 mm to 1 mm in diameter, and about 1.1 mm to 1.5 mm. In some cases, fluid apertures 515 having diameters between about 0.05 mm and 0.08 mm provided, e.g. using a laser drilling process where a plurality of these apertures is used and vaporizable material is facilitated to be wicked into these apertures.

The diameter of the fluid apertures 515 may vary based on the viscosity of the material stored in storage compartment 516. In general, where the vaporizable material has a high viscosity, the size of fluid apertures 515 may be increased.

The cartridge assembly 500 may be manufactured using a dual injection and insert molding process. Initially, tongue 545 may be inserted and held within an injection mold. A thermoplastic polymer, such as a polycarbonate, may then be injecting around tongue 545 to form body 502. Subsequently, a soft thermoplastic elastomer may be injected about the upper periphery of 502 (e.g. the upper edges of sidewalls 514) to define the seal member 594. In some cases, the elastomeric material may also be provided about the periphery of cartridge inlet 540 and cartridge outlet 518 to define seals for the ends of fluid conduit 504. Providing compressible or elastomeric seal members about the periphery of inlet 540 and outlet 518 may facilitate the creation of an enclosed fluid flow path through the vaporizer 400 when cartridge assembly 500 is installed therein.

The cartridge assembly 500 may have a semi-elliptical cross-section (e.g. the cover 525 may define a semi-elliptical upper section of compartment 516). As with cartridge assembly 200, cartridge assembly 500 (as well as storage compartment 516 to a lesser extent) may be tapered having a larger cross-sectional area proximate the first end 516A of storage compartment 516 and a smaller cross-sectional area proximate the second end 5168 of the storage compartment 516.

As with cartridge assembly 200, the cartridge assembly 500 may have a viewing region that includes a transparent window defined in the base 502 and/or cover 525. The transparent window may extend partially along the length of the storage compartment 516. Storage reservoir 516 may be visible through the transparent window.

Preferably, the window may be formed in cover 525. Thus, a user may be able to see the vaporizable material contained in the storage reservoir 516 when the removable cartridge assembly is installed in receptacle 416. That is, the user may be able to assess the remaining quantity of vaporizable material when the removable cartridge assembly 500 is inserted within the cartridge receptacle 416.

An opaque area may also be formed on a portion of the base 502 and/or cover 525. The opaque area may be used to print or mark identifying data, such as a cartridge identifier and/or patient identifier associated with cartridge assembly 500.

The cartridge assembly 500 may also include an onboard memory storage module 554 (e.g. RAM, flash, or EEPROM memory). The memory may be usable to store cartridge identifying data, such as a unique cartridge identifier. The memory may also be used to store data indicative of the vaporizable material in storage compartment 516. When the cartridge assembly 500 is filled, data regarding the vaporizable material deposited in storage compartment 516 may be stored in the memory. The memory module 554 may be coupled to cartridge coupling circuit 544 to allow vaporizer 400 to access the stored cartridge data. This may allow control circuit 420 to use the stored data to adjust configuration settings of the vaporizer, such as the predetermined vaporization temperature, based on the vaporizable material in the cartridge. This may also allow the control circuit 420 to provide a user with feedback regarding the cartridge assembly 500 and/or the material in storage compartment 516.

Optionally, the vaporizer 100/400 or cartridge assembly 200/500 may include an air quality sensor, such as a volatile organic compound sensor (e.g. a SGP30 or CCS811 sensor). The air quality sensor may be disposed proximate the inhalation aperture 112/412. The air quality sensor may be coupled with the control circuit and operable to evaluate the mixture of air and vapor prior to it being inhaled from the mouthpiece.

The cover 525 and base 502 of the cartridge assembly 500 need not be made of the same material, in particular where snap fit engagement members are used. For example, the base unit 502 that includes the heating assembly may be made from ceramic with an optionally integrated vapor tube. The cover 525, in turn, may be manufactured using a polycarbonate that may be partially or completely transparent.

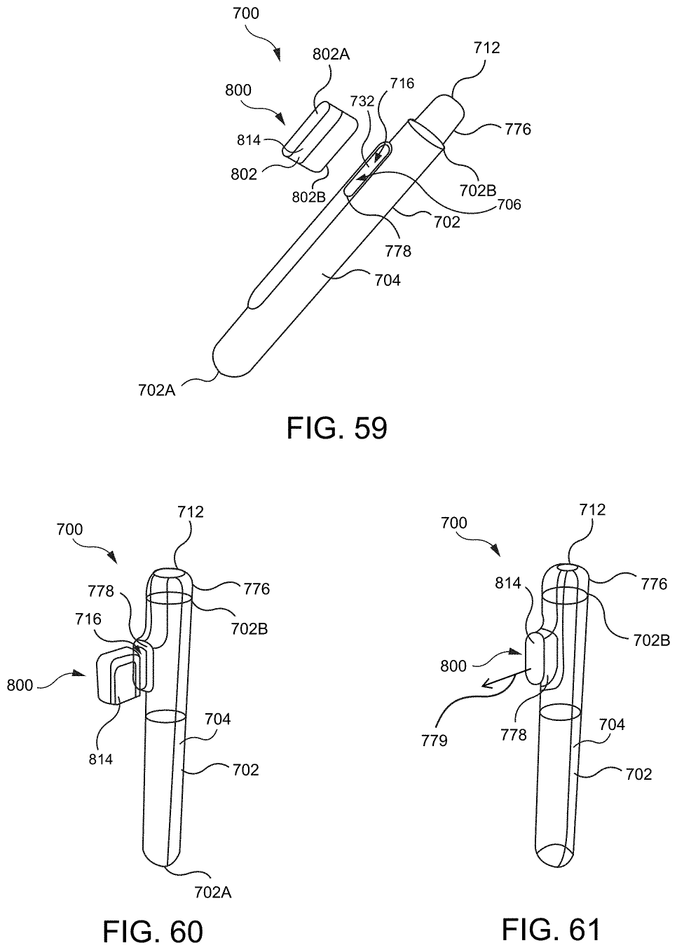

Referring now to FIGS. 59-63, shown therein is another example of a vaporization device 700. Vaporization device 700 is another example of a vaporization device usable to vaporize liquid vaporizable material, such as vaporizable material derived from various materials, such as nicotine, synthetic compositions and phyto materials such as cannabis. Vaporization device 700 may be used to vaporize vaporizable material in liquid or semi-liquid (e.g. waxy) forms. Elements having similar structure and/or performing similar function as those in the example vaporization device 100 of FIGS. 1-11 are numbered similarly, with the reference numerals incremented by 600.

FIG. 59 shows a side perspective view of the vaporization device 700. Vaporization device 700 includes a device body 702 and a removable cartridge assembly 800. FIG. 1 shows the removable cartridge assembly 800 removed from the vaporization device 700. Removable cartridge assembly 800 may contain vaporizable material therein for vaporization.

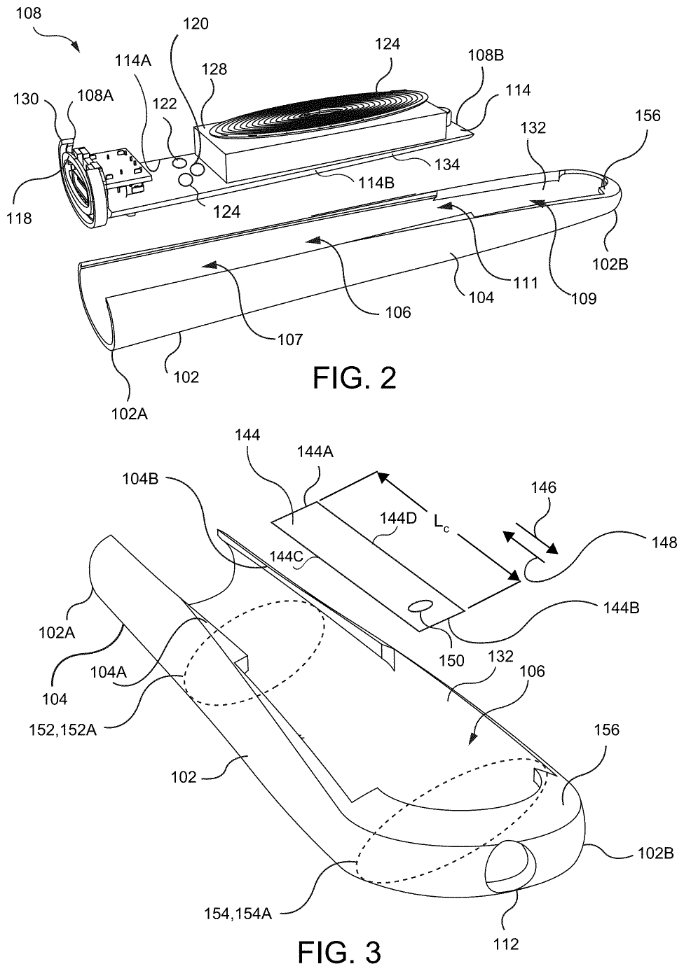

Device body 702 may have a first device end 702A and a second device end 702B opposite the first device end 702A. A device base or sidewall extends between the first device end 702A and the second device end 702B. In the example shown, a sidewall of base 704 extends between the first device end 702A and the second device end 702B to define an interior device cavity or recess 706. Interior device space 706 may contain a control assembly similar to control assembly 108 of FIG. 2.

In the example shown, the interior device cavity 706 is closed at both the first device end 702A and the second device end 702B by base 704. An inhalation aperture 712 may be defined in the base 704, for instance at the closed second device end 702B as shown. Inhalation aperture 712 may permit fluid communication between an external environment that surrounds the vaporization device 700 and the interior device cavity 706.

In some embodiments, the inhalation aperture 712 may be flush with the sidewall of base 704. Alternatively, the inhalation aperture 712 may be defined within a mouthpiece 776 that extends outwardly from the sidewall of base 704, e.g. as shown. In the example shown, the inhalation aperture 712 is mounted to a mouthpiece 776. Mouthpiece 776 is removably mounted to the device body 702 at the second device end 702B. Mouthpiece 776 may be removable to allow the mouthpiece 776 to be cleaned and/or replaced.

A cartridge receptacle 716 may be defined by the device base 704. Preferably, the cartridge receptacle 716 is defined closer to the second device end 702B than the first device end 702A, e.g. as shown. In this position, a control assembly (e.g. control circuitry, energy storage members, output indicators, communication modules etc.) may be positioned within the interior device space 706 between the first device end 702A and the cartridge receptacle 716.

Cartridge receptacle 716 may be defined by an outer edge 778 and an internal surface 732 extending from the outer edge 778 within the interior device cavity 706, e.g. as shown. In some embodiments, the internal surface 732 may be lined with a rubber material. In the example shown, the outer edge 778 is an elliptical outer edge. However, it will be appreciated that the outer edge 778 be any number of possible configurations, such as square, rectangular, triangular, etc.

Removable cartridge assembly 800 may include an outer cartridge housing 802. Cartridge housing 802 may have a first housing end 802A and a second housing end 802B opposite the first housing end 802A. A housing sidewall 814 may extend between the first housing end 802A and the second housing end 802B. Housing sidewall 814 may define and enclose a storage compartment or reservoir 816.

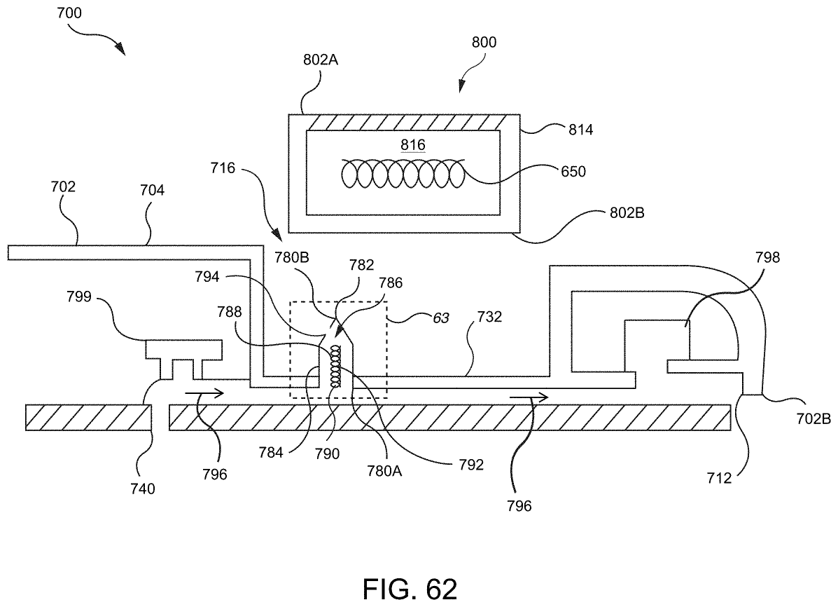

In the example shown, storage reservoir 816 is closed at both the first housing end 802A and the second housing end 802B by the housing sidewall 814. That is, the housing sidewall 814 may fully enclose the storage reservoir 816. Storage reservoir 816 may hold a vaporizable material 650 (e.g. FIG. 62) for vaporization. Preferably, the vaporizable material 650 is a liquid vaporizable material similar to the liquid vaporizable material 50 of FIG. 8.

Housing sidewall 814 may be configured to correspond to the outer edge 778 of the cartridge receptacle 716. The removable cartridge assembly 800 may be sized to fit snuggly into the cartridge receptacle 716. In some cases, the cartridge receptacle 716 may have a resilient inner lining to allow the cartridge assembly 800 to be positioned in receptacle 716 and then held therein by frictional engagement with the sides of receptacle 716.

FIGS. 60 and 61 illustrate an example of how removable cartridge assembly 800 may be loaded into vaporization device 700. FIG. 60 shows the removable cartridge assembly 600 in an unloaded position with respect to the vaporization device 700, while FIG. 61 shows the removable cartridge assembly 600 in a loaded position.

In FIG. 60, the removable cartridge assembly 800 is shown oriented to fit within the outer edge 778 of the cartridge receptacle 716. A user may then insert the removable cartridge assembly 800 into the cartridge receptacle 716, e.g. by sliding the cartridge assembly 800 downward into receptacle 716.

Frictional engagement between the housing sidewall 814 of removable cartridge assembly 800 and the internal surface 732 of the cartridge receptacle 716 may retain the removable cartridge assembly 800 in the loaded position. In some embodiments, the internal surface 732 may be lined with a rubber material to increase the frictional engagement between the housing sidewall 814 and the rubber lined internal surface 732 of the cartridge receptacle 716.

As shown in FIG. 61, when in the loaded position, a portion of the removable cartridge assembly may protrude out of the cartridge receptacle 716. To remove the removable cartridge assembly 800 from the cartridge receptacle 716, a user may apply force in a direction 779, away from the vaporization device 700, to the protruding portion of removable cartridge 800 strong enough to overcome the frictional engagement between the between the housing sidewall 814 and the internal surface 732.

FIG. 62 is a cutaway view of the vaporization device 700 showing the insertion of the removable cartridge assembly 800 into the cartridge receptacle 716. Cartridge receptacle 716 may include a heating element assembly 780 positioned therein. Heating element assembly 780 may have a first element end 780A and a second element end 780B opposite the first element end. First element end 780A may connect to the internal surface 732 of the cartridge receptacle 716. Second element end 780B may define a cartridge engagement member 782.

Heating element assembly 780 may extend from the internal surface 732 into the cartridge receptacle 716. The heating element assembly 780 may include a projecting engagement member 782 that is configured to pierce the cartridge 800 when the cartridge is positioned in receptacle 716. The projection 782 may include a sharpened or pointed end facing outward from the base of receptacle 716.

A heating element assembly outer wall 784 extends from the first element end 780A to the projection 782 at the second element end 780B. Heating element assembly outer wall 784 may define a heating chamber 786. A heating element 788 may be positioned within the heating chamber 786.

Heating element 788 may have an outer surface or layer manufactured from a porous ceramic material. Heating element 788 may include a resistive heating wire 790 disposed within the outer enclosure. Resistive heating wire 790 may be a resisting heating wire coil, e.g. as shown, that extends along the length of the heating element 788. As explained above, the heating element may also include a high frequency atomizer (e.g. an ultrasonic atomizer). The high-frequency atomizer may be used to heat as well as agitate the vaporizable material to generate vapor.

In some embodiments, the heating element 788 may be integrated with projecting engagement member 782. For example, a resistive heating element may be formed on, or enclosed within, the projecting engagement member 782.

For example, projection 782 may be manufactured from stainless steel. A thick film tubular heating element may be formed on projection 782 using a thick-film screen printing process as discussed above.

Heating element assembly 780 may also include a wicking element 792. In the example shown, the wick 792 at least partially surrounds the heating element 788. When energized, the heat emitted by the resistive heating wire 790 flows outwardly into the wick 792 surrounding the heating element 788. In embodiments where the heating element 788 is made from the porous ceramic material, heat emitted from the resistive heating wire 790 may flow outwardly through pores defined in the porous ceramic material to heat the wick 792.

As the user inserts the removable cartridge assembly 800 into the cartridge receptacle 716, the projection 782 of the heating element assembly 780 may penetrate the housing sidewall 814 at the second housing end 802B, e.g. as shown. The heating element assembly 780 may then extend at least partially into the storage reservoir 816.

In some embodiments, the cartridge housing sidewall 814 may be manufactured from a penetrable material. That is, the housing sidewall may be manufactured from a material that is easily punctured by the tip of projection 782. Alternatively, only a portion of the housing sidewall 814 is made of penetrable material. This may help maintain the structural integrity of the removable cartridge assembly 800 and avoid inadvertently puncturing the cartridge 800 prior to installation.

The penetrable portion of the housing sidewall 814 may include an identifier or marking. For instance, the penetrable portion may include a bullseye marking or have a different surface color from the rest of the housing sidewall 814. The marked portion may provide an indication to a user of the orientation in which to insert the removable cartridge assembly 800 in receptacle 716.

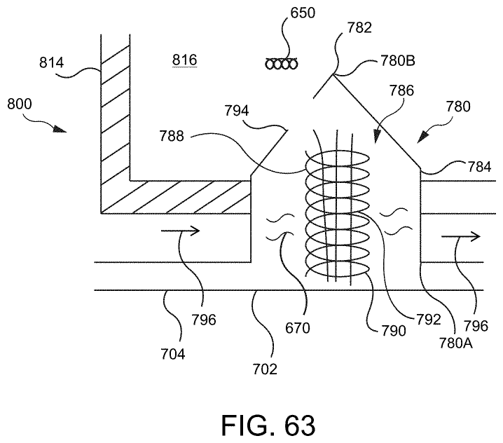

FIG. 63 is an enlarged view taken of portion 63 in FIG. 62. Heating element assembly outer wall 784 may have at least one vaporizable material receiving aperture 794 defined therethrough. When the removable cartridge is in the loaded position, the at least one vaporizable material receiving aperture 794 may permit the heating chamber 786 to be in fluid communication with the storage reservoir 816 of removable cartridge assembly 800. Accordingly, in the loaded position, the vaporizable material 650 contained in the storage reservoir 816 may enter the heating chamber 786 via the at least one vaporizable material receiving aperture 794. In the example shown, the vaporizable material receiving aperture 794 is positioned near the puncturing tip 782, proximate the second element end 780B.

Wick 792 may be in fluid communication with the vaporizable material 650 as it enters the heating chamber 786 via the at least one vaporizable material receiving aperture 794. When energized, the heating element 788 may heat wick 792 positioned around it. As wick 792 is in fluid communication with the vaporizable material 650, the heated wick 792 may heat the vaporizable material entering the heating chamber 786 via the at least one vaporizable material receiving aperture 794. Vaporizable material 650 may be vaporized when heated to a vaporization temperature. An emitted vapor 670 may then be inhaled by a user for therapeutic purposes.

Referring again to FIG. 62, the device body 702 may include an air input port 740 defined therein along its length. A fluid flow path 796 may extend within the interior device cavity 706 between the air input port 740 and the inhalation aperture 712, e.g. as shown. Accordingly, ambient air 660 from an external environment surrounding the vaporization device 700 may be drawn into the fluid flow path 796 through the air input port 740.

As shown, heating element assembly 780 is open at the first element end 780A. Heating element assembly 780 may be connected to the internal surface 732 such that the heating chamber 786 is in fluid communication with the fluid flow path 786 via the open first element end 780A.

When a user inhales from the inhalation aperture 712, ambient air 860 may be drawn from the external environment into the fluid flow path 796 via the air input port 740. While being drawn by the user's inhalation through the fluid flow path 796, the ambient air 660 may mix with the emitted vapor 670 within the heating chamber 786 prior to exiting the inhalation aperture 612.

The mixture of ambient air and vapor flowing out of the heating chamber 786 may enter the fluid flow channel 796 at a first temperature T₁ and exit through inhalation aperture 712 at a second temperature T₂ that is lower than the first temperature T₁. That is, the mixture may cool as it flows within the fluid flow path 796 between the heating chamber 786 and the inhalation aperture 712. This may provide the user with a more comfortable, and safer, temperature of vapor for inhalation.

Optionally, a seal (not shown) may be provided around the outer edge 778 of the cartridge receptacle 716. For example, the seal may be a rubberized or other elastomeric seal. In the inserted position, the seal may provide additional fiction between the outer edge 778 and the housing sidewall 814. The seal may also prevent the escape of vaporizable material 650 and/or emitted vapor 670 from the cartridge receptacle 716.

In some embodiments, the heating element assembly 780 may be removably connected to the internal surface 732 of the cartridge receptacle 716. Accordingly, the heating element assembly 780 may be removed from the vaporization device 700 for cleaning and/or maintenance. Alternatively, the heating element assembly 780 may be replaced with a replacement heating element assembly, that may be the same or different.

In the example shown, vaporization device 700 includes the heating element assembly 780. Accordingly, the removable cartridge assembly 800, e.g. as shown, may not include a heating element assembly. In comparison to removable cartridges 200 and 500, removable cartridge 800 may provide for a simpler and less expensive construction with fewer parts.

In some embodiments, the vaporization device 700 may include a fluid quality sensor 798. Fluid quality sensor 798 may be contained within the interior device cavity 706. Preferably, the fluid quality sensor 798 is in fluid communication with the fluid flow path 796 downstream of the heating element assembly 780, e.g. as shown. Accordingly, the mixture of ambient air and emitted vapor may pass through the fluid quality sensor 798 as it drawn down the fluid flow path 796 toward the inhalation aperture 712. Fluid quality sensor 798 may be electrically coupled to the control assembly. Fluid quality sensor 798 may be used to measure an amount of volatile organic compounds (VOCs) in the mixture to determine the quality or density of the vapor being inhaled.

In some embodiments, the vaporization device 700 may also include a fluid flow sensor 799. Fluid flow sensor 799 may operate in a similar manner as the fluid flow sensor 142 of vaporization device 100. Fluid flow sensor 799 may be contained within the interior device cavity 706. Preferably, the fluid flow sensor 799 is in fluid communication with the fluid flow path 796 upstream of the heating element assembly 780, e.g. as shown. Accordingly, the fluid flow sensor 799 may measure the mass or volume of ambient air 660 drawn into the fluid flow path 796. Fluid flow sensor 799 may be electrically coupled to the control assembly. Fluid flow sensor 799 may also be used to assist in dose control, as discussed herein.

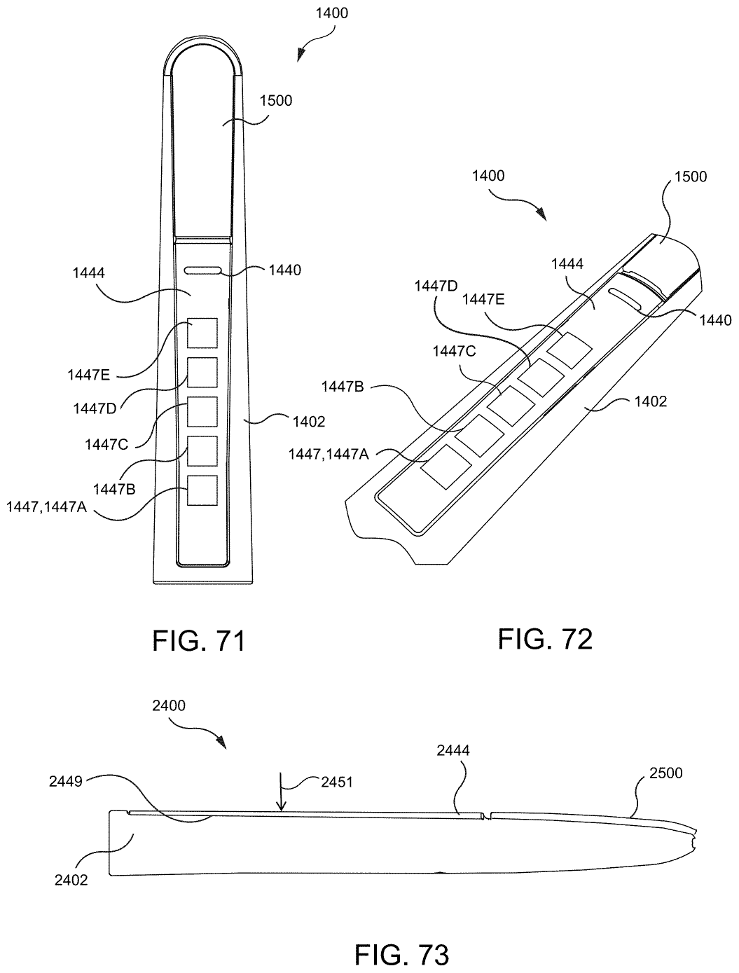

Referring now to FIGS. 71-72, shown therein is another example of a vaporization device 1400. Vaporization device 1400 provides a schematic illustration of vaporization activation security features that may be used to prevent unwanted or unauthorized use of the vaporizer. For instance, the security features may be used to prevent children from activating the vaporization device 1440. The features described in reference to vaporization device 1400 may be incorporated into the various other embodiments of vaporization devices (100, 400, 700) described herein.

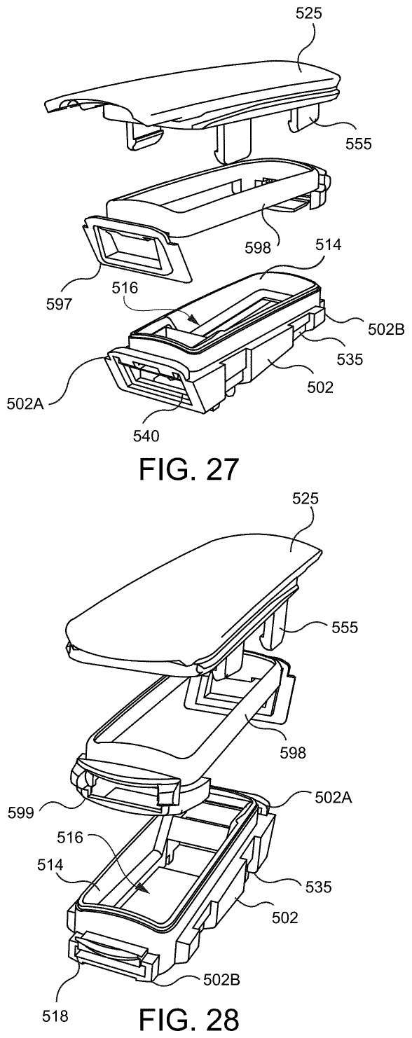

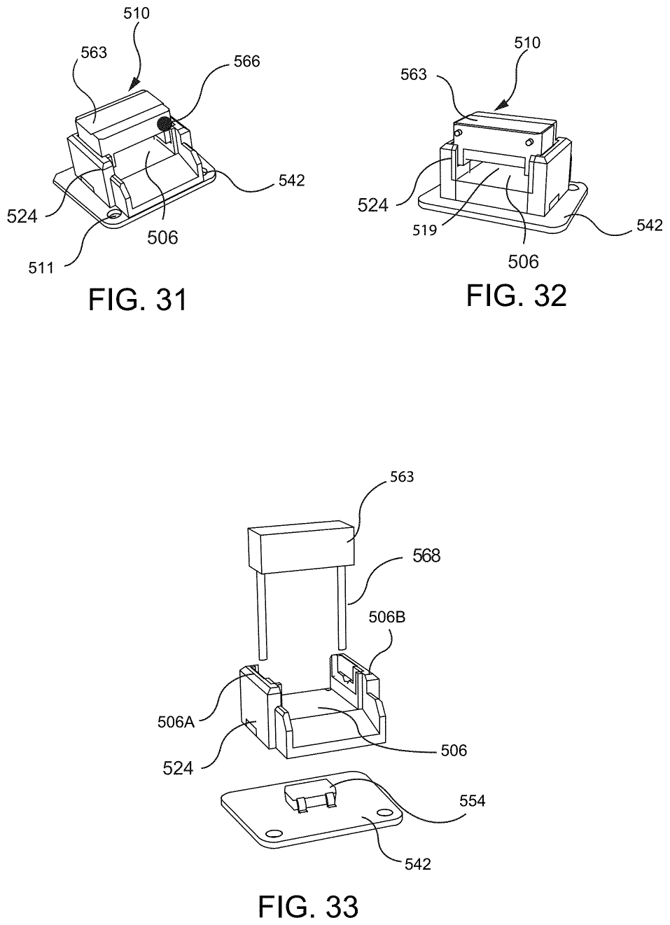

The vaporization device 1400 may be generally similar to the vaporization device 400 shown in FIG. 12-58, except that the vaporization device 1400 includes an activation interface on the outer surface of the device body. The activation interface may be usable to control a device activation lock of the vaporization device 1400. Elements having similar structure and/or performing similar function as those in the example vaporization device 400 in FIGS. 12-58 are numbered similarly, with the reference numerals incremented by 1000.

The vaporization device 1400 may include an activation lock that may be configured to control whether vaporization device is enabled to vaporize vaporizable material inserted therein. The activation lock may be adjustable between an activated state and a deactivated state. In the activated state, the activation lock enables the vaporization device heating assembly to be energized to heat vaporizable material. In the deactivated state, the activation lock prevents the heating assembly from being heated to a vaporization temperature. The activation lock may be provided in various forms, such as an electronic lock managed by the control circuit, or a switch (mechanical or otherwise) usable to connect/disconnect the heating assembly and an energy storage member.

In the example shown, the activation interface includes a keypad 1445 positioned on the device cover 1444. Keypad 1445 may be used to prevent unauthorized use of the vaporization device 1400. The vaporization device 400 shown in FIG. 12-58 may of course be used with the activation interface including the keypad 1445 positioned on the device cover 1444.

Keypad 1445 may be usable to enable activation of the vaporization device 1400 by controlling the activation lock. For example, prior to using the vaporization device 1400, a user may be required to unlock the activation lock using the keypad 1445. Similarly, after use, the user may use the keypad 1445 to lock the device 1400 (i.e. adjust activation lock to the deactivated state), thereby preventing unauthorized use.

In some cases, vaporization device 1400 may be automatically secured after a specific time has elapsed since last use or since being unlocked. Locking the vaporization device 1400, in general, may mean that the vaporization device 1400 is unable to vaporize the vaporizable material contained therein. For example, locking the vaporization device 1400 may be accomplished by preventing the energization of the heating element assembly. In contrast, when the vaporization device 1400 is unlocked, the heating element assembly may be energized to vaporize the vaporizable material.

Keypad 1445 may include at least one user input 1447. The user input 1447 may be provided in various forms, such as a button on the cover 1444 or as an input to a touch screen in device cover 1444.

For example, the user input 1447 may be operable using a capacitive sensing circuit. Device cover 1444 may be manufactured from a non-metallic material while the device body 1402 is made from a metallic material. A sensing circuit may be positioned beneath the at least one button 1447 within the device body 1402. The circuit may be able to detect a touch applied by the user the at least one button 1447. The circuit may be electrically coupled to a processor (e.g. control circuits 120, 420) positioned within the device body 1402. The processor may be configured to receive and process signals received from the circuit. The processor may be configured to control operation of the activation lock.

The at least one button 1447 and the circuit may be a capacitive touchscreen and a capacitive circuit, respectively. Alternatively, the at least one button 1447 and the circuit may be a resistive touchscreen and a resistive circuit, respectively. In the example shown, the keypad 1445 includes five capacitive touch segments 1447A to 1447E positioned in sequence along the length of the device cover 1444. Accordingly, a capacitive circuit (not shown) may be positioned beneath the capacitive buttons 1447A to 1447E within the device body 1402. The capacitive buttons may be labelled, as for example “A”, “B” “C” “D” “E”.

In some cases, vaporization device 1400 may be manufactured with a preset code stored in the memory module as part of the control circuit 420 that is uniquely associated to that vaporization device. The user may enter the preset code, for example “ABDE” using the keypad 1445 to lock or unlock their vaporization device 1400. When the user is entering the preset code, the capacitive circuit may detect each of the user's touches on capacitive touchscreens 1447A to 1447E. The capacitive circuit may send a signal to the processer after each touch. The processor may determine the entered code, based on the received signal and compare this entered code to the preset code in the memory module. If the codes match, the vaporization device may be unlocked. If the codes do not match, the vaporization device may not be unlocked. In some cases, after a predetermined number of incorrect codes have been entered, the vaporization device 1400 may be locked for a preset period of time. For example, after five successive incorrect attempts to enter the code, the vaporization device may be locked for a lockout period (e.g. 30 minutes) and unable to be unlocked for that time period.

A user may, in some embodiments, use their device (e.g. smartphone or tablet) to connect to the vaporization device to allow the device to be unlocked in a time less than the lockout period. In some embodiments a notification may be provided to the user's device that the device has had attempted unlocking operations without success, in such a case it may be with the use of a smartphone companion application in order to do so.

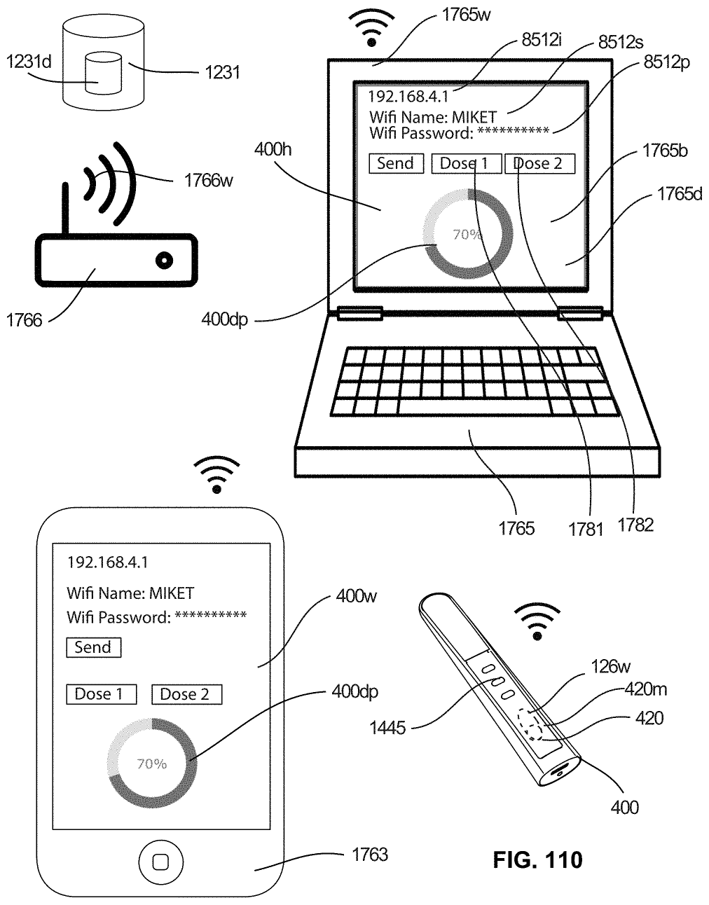

In some cases, the code may be used to personalize the device to a unique user. In other cases, a single device may be used by multiple users and each user may have a corresponding user-specific code. Each user may also have a user profile associated with the device that may be stored and monitored using an application on their device (e.g. a smartphone or tablet app) or on a remote server (see FIG. 110 to FIG. 114).

In some embodiments, the at least one button 1447 may be a single capacitive touchscreen capable of detecting a directional swipe or pattern entered by the user. For example, the user may enter a two-dimensional pattern on the capacitive touchscreen. The capacitive circuit may detect the user's touch and send a signal to the processor. The processor may determine the entered pattern, based on the received signal and compare this entered pattern to a preset pattern in the memory module. If the patterns match, the vaporization device may be unlocked. If the patterns do not match, the vaporization device may not be unlocked.

In some embodiments, the user may apply a plurality of touches, each touch having a touch duration, to the capacitive touchscreen 1447 (e.g. similar to Morse code). The capacitive circuit may detect each touch and the touch direction of each touch and send a signal to the processor. The processor may determine an entered code, based on the received signal and compare this entered code to a preset code in the memory module. If the codes match, the vaporization device may be unlocked. If the codes do not match, the vaporization device may not be unlocked.

The vaporization device 1400 may allow a user to define create a new activation code or pattern. The new activation may replace any previous code or pattern in the memory module. In some cases, the user may create a new code or pattern with a user device (e.g. a smartphone or tablet) that is wirelessly coupled to the memory module. In some cases, the user may operate a corresponding application on the smartphone or tablet to control activation/deactivation of the activation lock.

In some embodiments, instead of keypad 1445 positioned on the device cover 1444, the vaporization device 1400 may have a dial or combination lock for locking and unlocking the device. The dial or combination lock may be positioned on the device cover 1444. Alternatively, it may be positioned on the device body 1402. In some embodiment, a membrane switch may be positioned on the device cover 1444. The membrane switch may be used to lock and/or unlock the vaporization device, in a similar manner as the keypad 1445. In some embodiments using a keypad or buttons may consume less electrical power than using capacitive buttons and may be preferable.

Referring now to FIG. 73, shown therein is another example of a vaporization device 2400. The vaporization device 2400 is similar to the vaporization device 400 shown in FIG. 12-58, except that the vaporization device 2400 includes a pressure sensor 2449 positioned beneath device cover 2444 within the device body 2402. Elements having similar structure and/or performing similar function as those in the example vaporization device 400 in FIGS. 12-58 are numbered similarly, with the reference numerals incremented by 2000.

FIG. 73 shows a side plan view of the vaporization device 2400. Pressure sensor 2449 may detect a force 2451 applied by a user to the vaporization device 2400 through the device cover 2444. When the force 2451 applied by the user is a force greater than a preset force, the vaporization device 2400 may be unlocked. Similarly, if the force 2451 is less than or equal to the preset force, the vaporization device 2400 may not be unlocked. The preset force may be defined at a force threshold that is difficult for a child to achieve, thereby preventing them from unlocking the vaporizer device 2400.

Pressure sensor 2449 may be electrically coupled to a processor (e.g. control circuits 120, 420) positioned within the device body 2402. The processor may be configured to receive and process signals received from the pressure sensor 2449.

Vaporization device 2400 may be shipped with the preset force defined in the memory module. The vaporization device 2400 may give the user an option to create a new preset force. The new preset force may replace the previous preset force in the memory module. In some cases, the user may create a new preset force with the user device (e.g. the user's smartphone) that is wirelessly coupled to the memory module. In some cases, however, the preset force may be fixed for the vaporization device 2400 (e.g. unable to be lowered). This may ensure that the vaporization device 2400 cannot be activated by a child.

Alternatively, a user device may be used to lock and/or unlock a vaporization device associated to that user device. For example, the user device may be a smartphone, tablet, notebook computer, desktop computer, etc. The user device may be associated to a vaporization device through a registration process. The user device may be wirelessly coupled to a control circuit or processor of the vaporization device via a wireless communication module positioned within the device body 2402.

In some cases, a user device proximity threshold may be used to lock and/or unlock an associated vaporization device. That is, when the user device is within a proximity threshold, the vaporization device may be unlocked. In contrast, when the user device is outside the proximity threshold, the vaporization device may be locked.

For example, the vaporization device may employ a relative received signal indicator (RSSI) electrically coupled to a processor. The RRSI may be used to measure the power present in a received signal from the user device. The processor may convert the measured power into a measured proximity. If the measured proximity is greater than the proximity threshold, the vaporization device may be unlocked. In contrast, if the measured proximity is less than or equal to the proximity threshold, the vaporization device may be locked. For example, the proximity threshold may be set at 2 meters. In some cases, the proximity threshold may be adjusted by the user with their user device.

In some embodiments, a fingerprint scanner may be used to lock and/or unlock the vaporization device. The fingerprint scanner may be positioned on the device cover or elsewhere on the vaporization device. The fingerprint scanner may be electrically coupled to a process within the vaporization device. The fingerprint scanner may also be wirelessly coupled to the memory module. Memory module may store a plurality of fingerprint records, each fingerprint record being associated with a vaporization device. To lock or unlock a vaporization device, a user may scan their finger using the fingerprint scanner on the vaporization device. The processor may compare the scanned fingerprint to the fingerprint records stored in the memory module. If the scanned fingerprint matches the fingerprint associated with that vaporization device, the vaporization device may be lock or unlocked. In some cases, a user may unlock the vaporization device by inputting a fingerprint to their smartphone or tablet while interacting with an application configured to control the vaporization device. In some other cases the user may be required to inhale a predetermined pattern of inhalations to unlock the device. For example, three quick puffs or a single puff and then a longer inhalation and then a puff.

In some embodiments, a preset air flow velocity is required to energize the heating element assembly. The air flow velocity of each inhalation may be detected by an air flow sensor positioned within the vaporization device (e.g. fluid flow sensors 142, 442). If the measured air flow is greater than the preset air flow velocity, the heating element assembly may be energized. If the air measured air flow velocity is less than or equal to the preset air flow velocity, the heating element may not be energized. The preset airflow velocity may be set such that is difficult for a child to achieve, thereby preventing them from energizing the heating element assembly.

Embodiments described herein may also facilitate filling cartridges with liquid vaporizable materials. In many existing processes, cartridges may be filled through extremely small apertures in the cartridge surface, which may require long filing times or pressurized filling systems. This process may be inefficient and reduce the number of cartridges that may be produced by a manufacturer. Embodiments described herein may facilitate rapidly filling one or more cartridges.

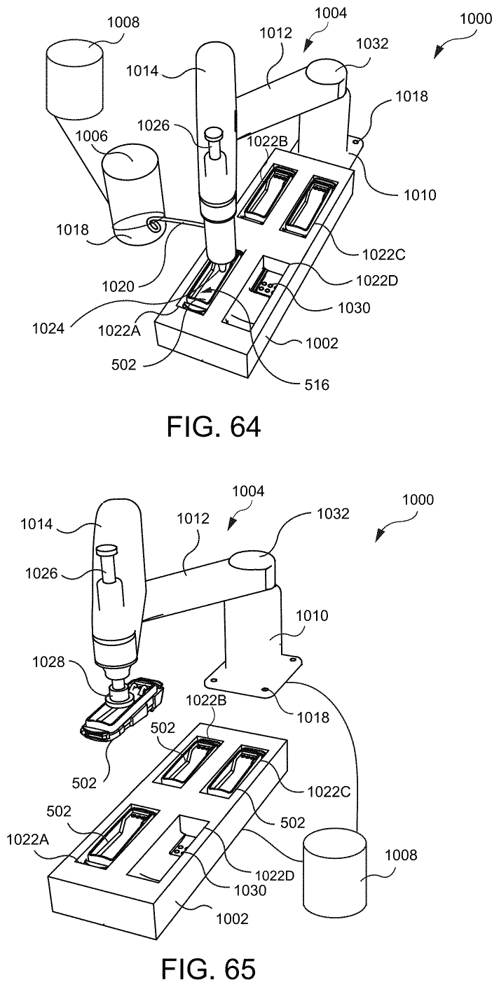

FIG. 64 shows a side perspective view of an example apparatus 1000 that may be used to fill cartridges, such as the cartridge assemblies 200, 500 and 800 described herein. As shown, cartridge filling apparatus 1000 may include a cartridge base or tray assembly 1002, an arm assembly 1004, a phyto material reservoir 1006, and a data server 1008.

The tray assembly 1002 may include a plurality of trays within which cartridges may be positioned. The cartridge trays may be configured into an array usable to hold a plurality of cartridges.

Arm assembly 1004 may be referred to as a robotic arm assembly. The arm assembly 1004 may be configured to automatically fill cartridges positioned within the trays in cartridge base 1002.

The arm assembly 1004 may include a support base 1010 and a multi-axis robotic arm 1012 movably connected to the support base 1010. The arm 1012 may include one or more operative attachments usable to engage cartridges to be filled. For example, a fluid dispenser 1014 may be removably connected to the multi-axis robotic arm 1012.

Support base 1010 may be positioned on a support surface (not shown), and may be secured to the support surface using fasteners such as bolts, screws or rivets for example. In the illustrated example, the support base 1010 includes four mounting apertures 1016. For example, support base 1010 may be mounted to the support surface with four fasteners (not shown) that respectively pass through the four mounting apertures 1016.

Phyto material reservoir 1006 may store a vaporizable material that is to be dispensed into the cartridges. Vaporizable material may be a liquid vaporizable material 1018, e.g. as shown.

Vaporizable material reservoir 1006 may be fluidly connected to the fluid dispenser 1014. In the example shown, the vaporizable material reservoir 1006 is fluidly connected to the fluid dispenser 1014 via a linking tube 1020. Accordingly, the liquid vaporizable material 1018 may pass from the vaporizable material reservoir 1006 to the fluid dispenser 1014 via the linking tube 1020.

Cartridge base 1002 may include a plurality of molds or trays configured to hold cartridge assemblies. Each mold may be configured to accommodate a specific configuration of the cartridge assembly being filled. That is, each mold may be dimensioned such that the specific cartridge assembly fits inside. In the illustrated example, the cartridge tray 1002 includes four molds 1022A, 10228, 1022C and 1022D. It will be appreciated that the cartridge tray 1002 may be configured with differing numbers of molds and mold configurations defined therein.

Fluid dispenser 1014 may include a filling nozzle 1024 extending from the fluid dispenser 1014, e.g. as shown. Filling nozzle 1024 may direct the vaporizable material 1018 from the fluid dispenser 1014 into the cartridge assemblies that are being held in the plurality of molds.

Multi-axis robot arm 1012 may be movably connected to the support base 1010 by a universal joint 1032, e.g. as shown. Universal joint 1032 may allow the multi-axis robot arm 1012 to move in three-dimensions with respect to the support base 1010.

Cartridge base 1002 may be connected to the support base 1010, e.g. as shown in FIG. 64. The cartridge base 1002 may be aligned with support base 1010 to provide a defined arrangement of trays relative to base 1002. This may provide a pre-defined sequence of movements for the arm assembly 1012 to engage the cartridges to be filled and then closed. Movement of the multi-axis robotic arm 1012 may be automated according to the arrangement of the base 1002.

Accordingly, the multi-axis robotic arm 1012 may position the filling nozzle 1024 above a mold prior to dispensing the vaporizable material 1018 into the cartridge assembly held in that mold. For example, FIG. 64 shows filling nozzle 1024 positioned by the multi-axis robot arm 1012 over mold 1022A. The filling nozzle 1024 may include a filling nozzle valve that is operable to enable and disable fluid flow through nozzle 1024. The valve may be automatically operated by a control application, e.g. provided on server 1008.

For example, if cartridge filling apparatus 1000 is used to fill the removable cartridge assembly 200, the filling nozzle 1024 may be positioned within the filling tube 280 prior to dispensing the liquid vaporizable material 1018. In this way, the vaporizable material 1018 may flow from the vaporizable material reservoir 1006 through the linking tube 1020 into the fluid dispenser 1016 and then be dispensed from the filling nozzle 1024 directly into the storage reservoir 216 via filling tube 280. In some embodiments the liquid vaporizable material may be heated to facilitate its flow through the linking tube 1020 into the fluid dispenser 1016. After being heated, the liquid vaporizable material may be dispensed from the filling nozzle 1024.

In some embodiments, fluid dispenser 1014 may include a heated plunger 1026, e.g. as shown. This may be particularly useful where cartridge assembly 200 is being filled. Heated plunger 1026 may be heatable to a temperature greater than the melting temperature of the filling tube 280. After filling, heated plunger 1026 may extend (i.e. plunge) to contact filling tube 280. The plunger 1026 may contact filling tube 280 and cause the filling tube 280 to melt and thus seal the filling tube 280. The liquid vaporizable material 1018 (e.g. vaporizable material 50 of FIG. 8) may then be enclosed within the storage reservoir 216.

Filling nozzle 1024 may dispense a predetermined amount of liquid vaporizable material 1018 from the fluid dispenser 1014 into the storage reservoir of each cartridge assembly. A “volume-based” or weight-based” method may be used to dispense the predetermined amount.

Apparatus 1000 may include a liquid flow sensor in fluid communication with the filling nozzle 1024. The liquid flow sensor may monitor the volume of vaporizable material dispensed from filling nozzle 1024. The filling apparatus 1024 may be configured to automatically operate the filling nozzle valve to disable fluid nozzle 1024 after a predetermined volume of vaporizable material has been dispensed.

In some embodiments, tray 1002 may include a weight sensor or scale beneath molds 1022. The weight sensor may monitor the weight of cartridges positioned within the molds 1022A-1022D. For example, the weight sensor may measure an initial weight when the cartridges are installed. The weight sensor may continuously monitor the weight of the cartridges as vaporizable material is being dispensed. When weight sensor determines that a predetermined weight of vaporizable material has been dispensed, filling nozzle valve may be automatically operated to deactivate filling nozzle 1024.

After filling the cartridge assembly to the predetermined amount (weight or volume), a memory module (e.g., memory module 254) may be programmed with a unique identification number (e.g. unique identification number 288) and/or additional cartridge identification data. Cartridge filling apparatus 1000 may program or encode the unique cartridge identification number and/or the cartridge identification data into the memory module.

FIG. 64 shows three cartridge assemblies, one being held in each of molds 1022A, 1022B and 1022C, respectively (e.g. each cartridge assembly may be the removable cartridge assembly 500 of FIG. 25). Each cartridge assembly may have its lid removed, e.g. as shown, exposing its internal storage reservoir (e.g. storage reservoir 516). With the cartridge assembly's lid removed, the storage reservoir may be open to filling nozzle 1024 during filling. That is, removing the lid of the storage compartment 516 may allow vaporizable material to be easily filled in storage compartment 516.

For example, wide bore filling tubes or syringes may be used to insert vaporizable material that may have a high viscosity. For instance, a wider tube may be heated to allow a semi-liquid or waxy vaporizable material to flow more easily into the storage compartment 516.

In some cases, the vaporizable material may be provided in a semi-solid form. For instance, vaporizable material may be die cut from a sheet of vaporizable material into shapes corresponding to the storage compartment. Vaporizable material may rolled into a sheet having a predetermined thickness. A die having a predetermined shape may be used to stamp out predetermined weights or volumes of the vaporizable materiel in the semi-solid form. This may facilitate inserting a harder, more solid, extract or derived phyto material product within the storage compartment, which may not otherwise be insertable through a small filling tube due to its viscosity.

Alternatively, filling nozzle 1024, e.g. in the form of a vacuum chuck, may be used to dispense solid vaporizable material, e.g. cooled tablets or segments of vaporizable material. For example, where the filling apparatus 1000 is used to fill cartridge assembly 500, solid vaporizable material may be deposited into the storage compartment 516 from the top side prior to the cover 525 being attached. The cover 525 may then enclose the vaporizable material within storage compartment 516. In some cases, the cover 525 may compact the deposited vaporizable material and/or force the vaporizable material to spread throughout the storage compartment 516.

In some cases, the vaporizable material may be provided as semi-solid or solid tablets or formed segments. The formed segments may be formed into the desired size for storage compartment 516. In some cases, the segments may be formed with a defined weight or size of vaporizable material that cartridge assembly 500 is intended to deliver. The formed segments may be maintained below their melting point (in some cases cooled and hardened) prior to insertion into storage compartment 516. Once cover 525 is secured to base 502, the deposited material may be allowed to increase in temperature (e.g. to room temperature) and melt to spread throughout storage compartment 516.

In some cases, the filling apparatus may include a vacuum chuck operable to load the formed segments into the vaporizable material reservoir 1006. In such cases, the segments may be heated to melt prior to deposition into a storage compartment via the filling nozzle.

Filling apparatus 1000 may also be configured to load cartridges into the cartridge tray 1002 prior to filing. The filling apparatus 1000 may include a cartridge adapter 1028 at the end of arm 1012. In some cases, the cartridge adapter 1028 may be provided in combination with the filling nozzle 1024 (e.g. an electromagnetic adapter surrounding the filling nozzle). In other cases, the nozzle 1024 may be removed and replaced with cartridge adapter 1028 when cartridges, and/cartridge covers are being positioned.

In some cases, the filling apparatus 1000 may provide a combined loading and filling apparatus that loads the cartridge tray 1002 with cartridge assemblies and then fill the cartridge assemblies in successive loading and filling processes. In other cases, a sequence of filling apparatus may be provided, a first using a cartridge adapter 1028 and a second using a filling nozzle 1024. After the cartridge tray 1002 has been loaded with cartridge assemblies, the tray 1002 may be moved (e.g. on a conveyor belt) downstream to the cartridge filling apparatus 1000.

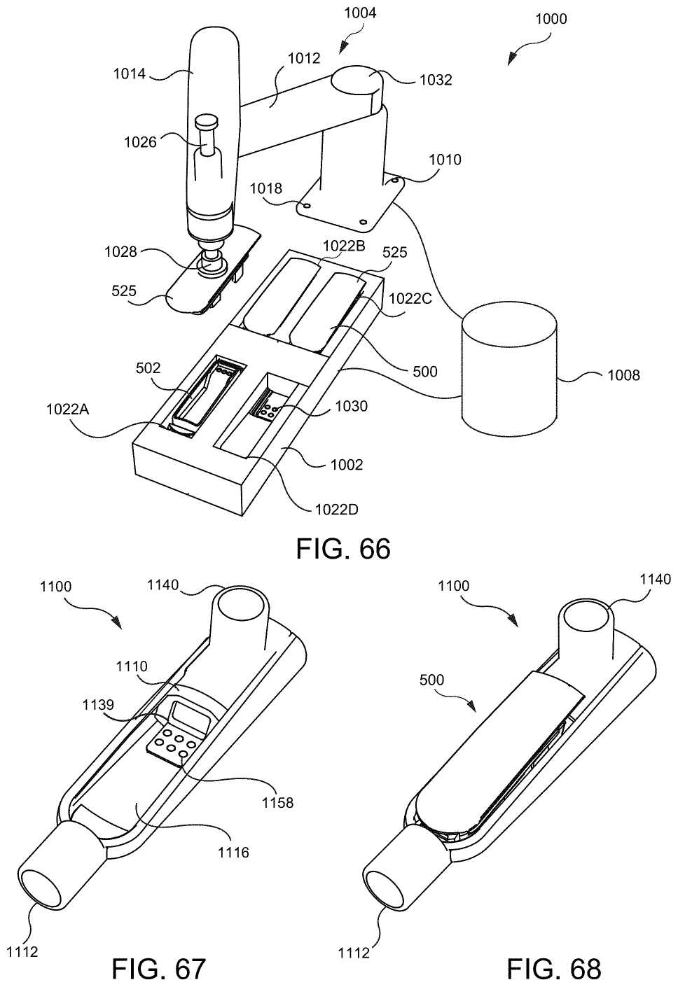

FIG. 66 shows an example of filling apparatus 1000 being used as a cartridge sealing apparatus. Cartridge sealing apparatus 1000 may be used to seal the cartridge assemblies, filled previously with vaporizable material 1018, with a lid or cover 525, e.g. as shown. In some cases, the filling apparatus may provide a combined loading, filling and sealing apparatus that loads the cartridge tray 1002 with cartridge assemblies, then fill the cartridge assemblies with liquid vaporizable material 1018, the seal the cartridge assemblies with the lid, in successive loading, filling and sealing processes. In other cases, after the cartridge assemblies have been filled with liquid vaporizable material 1018, the cartridge tray 1002 may be moved (e.g. on a conveyor belt) downstream to another filling apparatus 1000 configured as a cartridge sealing apparatus. The sealed cartridges may subsequently be inserted into a blister packaging machine and blister packed for transport.

In some embodiments, a data server 1008 may be communicatively coupled to the vaporizable material reservoir 1006, e.g. as shown in FIG. 64. In some embodiments, the data server 1008 may be communicatively coupled to the arm assembly 1004 and the cartridge tray 1002, e.g. as shown in FIG. 65. In some embodiments, the data server 1008 may be communicatively coupled to the vaporizable material reservoir 1006, the robotic arm assembly 1004 and the cartridge tray 1002.

Empty mold 1022D shows electrical contacts 1030. Data server 1008 may be communicatively coupled to the cartridge filling device 1000, the cartridge loading device 1000′ and the cartridge sealing device 1000′. Because the cartridges held within the cartridge tray 1022 have the PCB on an opposite side of the filling side, electrical contact may be made between the electrical contacts 1030 of the filling system and the plurality of electrical contacts 272, 572 of the cartridge assemblies 200 and 500. Cartridge identification data may then be programmed into the memory storage module of each cartridge assembly. The cartridge identification data may also be stored within the data sever 1008. The vaporizer devices may then access the cartridge identification data from the memory storage module (or from the data server) when the cartridges are installed into the cartridge receptacles. This allows the vaporizer device to determine the volume, weight and type of vaporizable material provided, and may adjust various operational settings (e.g. vaporization temperature) using this information.

Once a cartridge is filled with vaporizable material, or during filling by the filling apparatus 1000, a memory circuit disposed within the cartridge may be programmed with a unique identification number. This unique identification number may be stored on server 1008 to allow that cartridge to be uniquely identified and tracked. In some embodiments the unique identification number may used to determine whether the cartridge has been legitimately produced (e.g. filled by an authorized filling station such as an authorized licensed producer or authorized agent).

Filling apparatus 1000 may also be configured to apply a label to the lid or cover (e.g. label 284 of FIG. 10). In some cases, the label may be applied to an inner surface of the storage compartment 516. In such cases, the storage compartment may include a viewing region to allow the label to be visible from outside the storage compartment 516. For instance, cover 525 may injection molded from a transparent plastic material. An outer surface of cover 525 may be painted or obscured with a dark color. A laser removal process may be used in order to expose the viewing region. This process may provide a cleaner finish than using a spray mask. In some cases a laser removal process may also be used to create a machine readable optical pattern (e.g. a barcode or QR code).

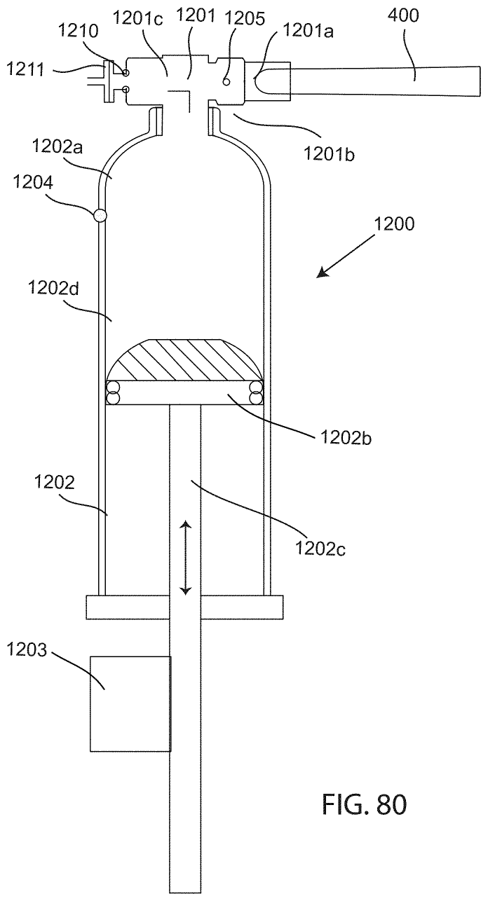

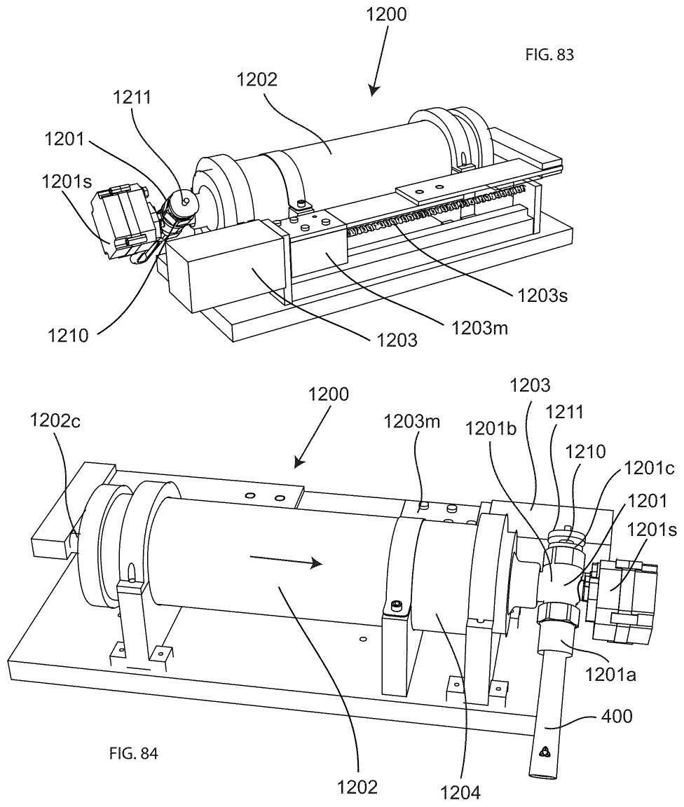

FIGS. 67 and 68, in conjunction in FIGS. 80, 81, 82 and 83, show an example of a cartridge testing apparatus 1100. Cartridge testing apparatus 1100 may be used to test and calibrate a cartridge inserted therein. The cartridge testing apparatus 1100 may test various aspects of cartridge testing apparatus, such as its function, heating chamber, airflow, etc.

The testing apparatus 1100 may define a testing receptacle 1116 shaped to receive a cartridge assembly 500. The receptacle 1116 may include contacts 1158 positioned to engage the cartridge contacts 572 of an inserted cartridge. The testing apparatus 1100 may use this coupling to update the memory module of the cartridge assembly 500, e.g. with calibration data or identifier data.

The testing apparatus 1100 may include a fluid inlet 1140 that may be coupled to the cartridge by a fluid flow manifold 1110. Manifold 1110 may be shaped to correspond to manifold 410, so that the manifold outlet 1139 may engage the fluid conduit 504 of an inserted cartridge.

In some cases, testing apparatus 1100 may measure volatile organic compounds (VOCs) emitted from an inhalation aperture 1112 upon heating up of the heating element assembly and it emitting phyto material extract vapor. The testing apparatus 1100 may also include sensors to detect small amounts of THC or CBD or nicotine being atomized when the heating assembly is energized. For example, vaporization device may be used to vaporize small volumes of ingredients of interest (e.g. THC, CBD or nicotine) when inserted in testing apparatus 1100. The emitted vapor may be directed into a sampling container at the outlet of testing apparatus 1100. The contents of sampling container may be analyzed using various analysis systems, such as Raman Spectroscopy, mass spectrometers or HPLC, FAIMS, or combinations thereof. This may allow quantitative measurement of the vaporizable material of interest. This may also permit dose calibration of the liquid vaporizable material after it has been atomized.

As mentioned above, testing apparatus 1100 may also perform a calibration process with the mass airflow sensor and other sensors to determine a correlation between a quantity of vaporized material that is emitted per volume or mass of air that is propagated through the fluid flow path. The emitted quantities of ingredients of interest (e.g. THC, CBD or nicotine) and airflow through the cartridge may be monitored. The calibration results may be stored in the memory module in the cartridge in relation to at least some of the mass airflow rate, heating chamber temperature, current, voltage, current, PWM profile applied to the resistive heating element, viscosity of the material for vaporization and so forth.

FIGS. 80, 81, 82, 83, 84 and 85 illustrate an embodiment of a vapor sampling system for airflow as well as phyto material extract calibration. An embodiment of the vapor sampling system 1200 involves the building of an “artificial lung” or a dosing calibration system whereby vapor is drawn through the vaporization device 400 (for example device 400 is shown in this embodiment, however other vaporization devices may be used as described herein such as vaporization device 100, 200, 500) through a 3 port L valve 1201 using a large syringe 1202 for vapor capturing. Furthermore, the vapor sampling system may also be used for calibration of the fluid flow sensor.

A first port 1201a of the L valve 1201 is fluidly coupled with the vaporization device 400. A second port 1201b (common port) of the L valve is coupled with the large syringe 1202. A large syringe 1202 may be a syringe that has a total volume of about 1 liter, or about 1.5 liters or about 400 to 600 ml. Preferably a volume of the large syringe 1202 is similar to that or larger than an approximate tidal lung volume of a user that would be using the vaporization device 400, where an average tidal volume may be about 500 ml.

As is shown in FIG. 80, the large syringe 1202 may have an inhalation and expelling volume 1202a and a plunger 1202b for moving within the large syringe 1202 coupled with a plunger shaft 1202c. The plunger shaft 1202c may be coupled with the syringe actuator 1203, which may use a lead screw mechanism 1203s or rack and pinion or linear motor mechanism, that is coupled with a slider mechanism 1203m that slides on a track and the slider mechanism 1203m is coupled with plunger shaft 1202c where a draw rate is controllable to create a draw profile or artificial inhalation profile.

The syringe actuator 1203 is for having a controllable source of power applied and is programmatically controlled using a software algorithm in order to controllably move slider mechanism 1203m in time to create a desired and predetermined inhalation profile through suction through the L valve 1201. Where the syringe actuator 1203 may have its rate of motion such that inhalations of about 0 L to 0.5 L per second are controllably achievable in 1 ml increments through the inhalation aperture of the vaporization device 400. This process may be used to calibrate the fluid flow sensor by recording differential pressure or barometric pressure or pressure sensor or puff sensor or an audio level of air propagating through the manifold to calibrate the fluid flow sensor.

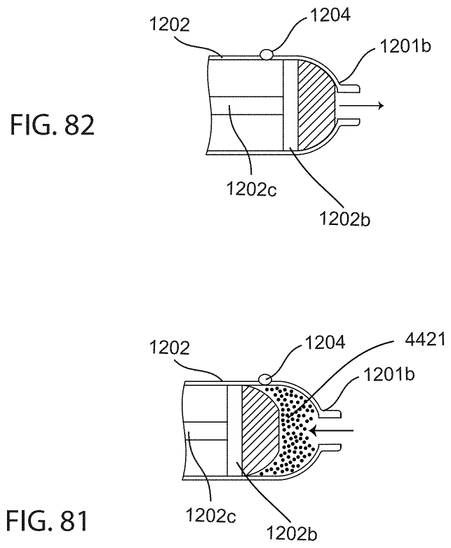

Referring to FIG. 81, in use, in a process of inhalation from the vaporization device 400, the valve of the 3 port L valve 1201 is fluidly coupled with its first port 1201a to the vaporization device 400 and with its second port to the syringe cavity 1202d forming an inhalation fluid pathway and the third port 1201c of the L valve 1201 is other than fluidly coupled with this inhalation fluid pathway. As the syringe actuator 1203 moves the plunger shaft 1202c, air and vapor are drawn through the vaporization device 400 into the inhalation and expelling volume 1202a at the controllable rate until a predetermined inhalation volume is achieved, for example 500 ml.

Referring to FIG. 82, in use, in the process of exhalation from the vaporization device 400, the valve of the 3 port L valve 1201 is fluidly coupled with its third port 1201c and with its second port to the syringe cavity 1202d forming an exhalation fluid pathway and the first port 1201a of the L valve 1201 is other than fluidly coupled with vaporization device 400 inhalation aperture. As the syringe actuator 1203 moves the plunger shaft 1202c, air and vapor are expelled from the third port 1201c at the controllable rate until the predetermined inhalation volume is approximately zero.

In the inhalation mode, the plunger of the large syringe is pulled (creating suction) so that vapor flows into a syringe cavity 1202d in a controlled rate or in a variable rate (simulating the inhalation of a user—where in some cases the user may inhale faster and, in some cases, slower and in some cases faster and then slower) through the use of the controllable actuator 1203.

FIG. 85 illustrate the testing system 1200 from a perspective view in a fully extended inhalation mode, where for example the inhalation and expelling volume 1202a is about 400 ml with vapor 4421 (FIG. 81) within this cavity post inhalation from the vaporization device 400.

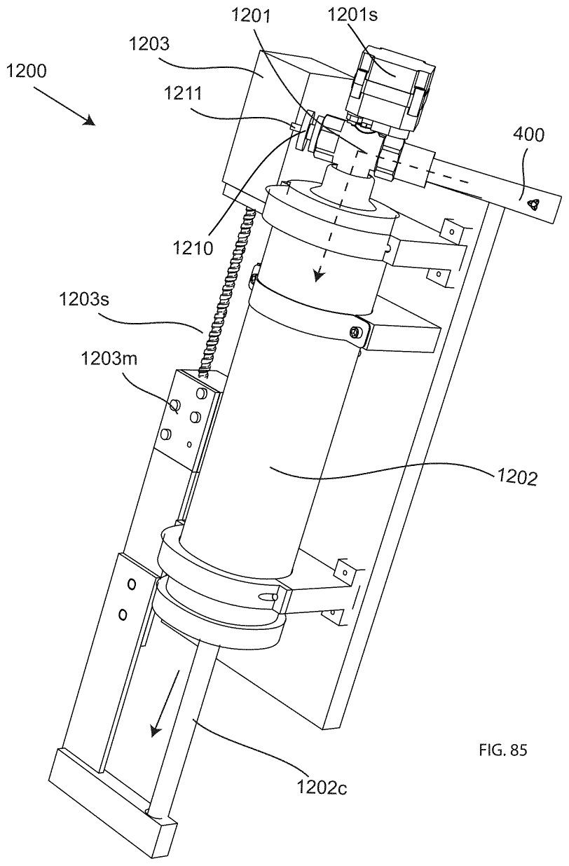

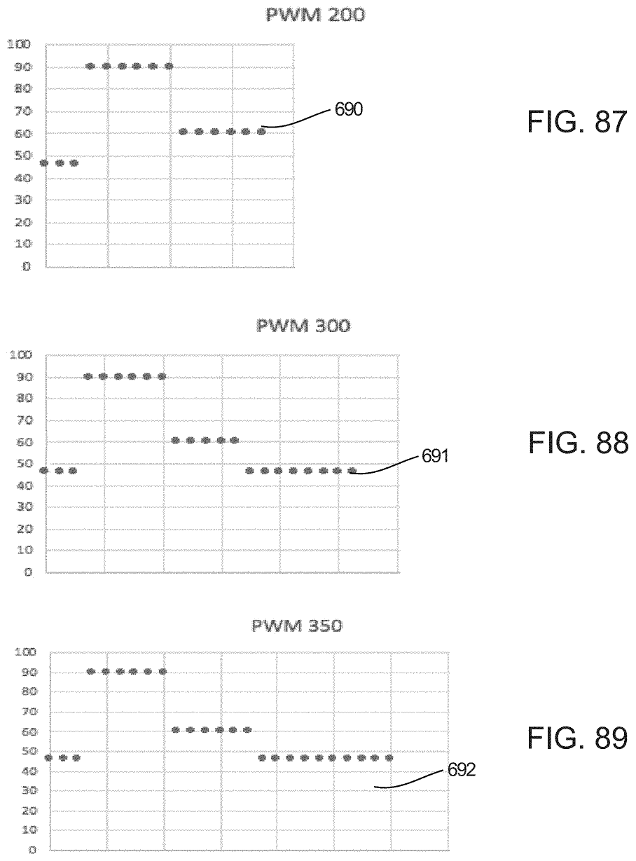

FIG. 84 illustrates the syringe plunger 1202b having expelled almost all of the vapor and air mixture (FIG. 82) and it is ready for inhalation again. In some embodiments the L valve 1201 is movable by hand and in other embodiments it is movable using a solenoid or pneumatic or motorized actuator 1201s.

In some embodiments of the testing system 1200, VOC (volatile organic compound) sensors as a first VOC sensor 1205 and a second VOC sensor 1204 may be utilized in the testing system 1200. Where VOC sensor may be a multi-pixel gas sensor, such as SGP30 by Sensirion, for the measurement of indoor air quality which may have multiple metal-oxide sensing elements and may measure volatile organic compounds in the air as well as carbon dioxide.

The SGP30 by Sensirion may provide a reading of TVOC signal 0 ppb to 60000 ppb and may provide a CO2eq signal of 400 ppm to 60000 ppm or in some cases a SGPC3 may also be used that provides a TVOC signal 0 ppb to 60000 ppb.

In some embodiments the first VOC sensor 1204 may be coupled with an internal volume of the large syringe 1202 exposed where it is fluidly coupled with its sensing port with the syringe cavity 1202d. Prior to the inhalation mode, the first VOC sensor 1204 is substantially exposed to ambient air plunger as the plunger of the large syringe is proximate the second port 1201b of the L valve 1201. In an inhalation mode as the plunger of the large syringe is moved away from the second port 1201b of the L valve 1201 by about, for example 2 cm, the first VOC sensor 1204 is substantially exposed to the vapor 4421 as inhalation takes place through the vaporization device 400.

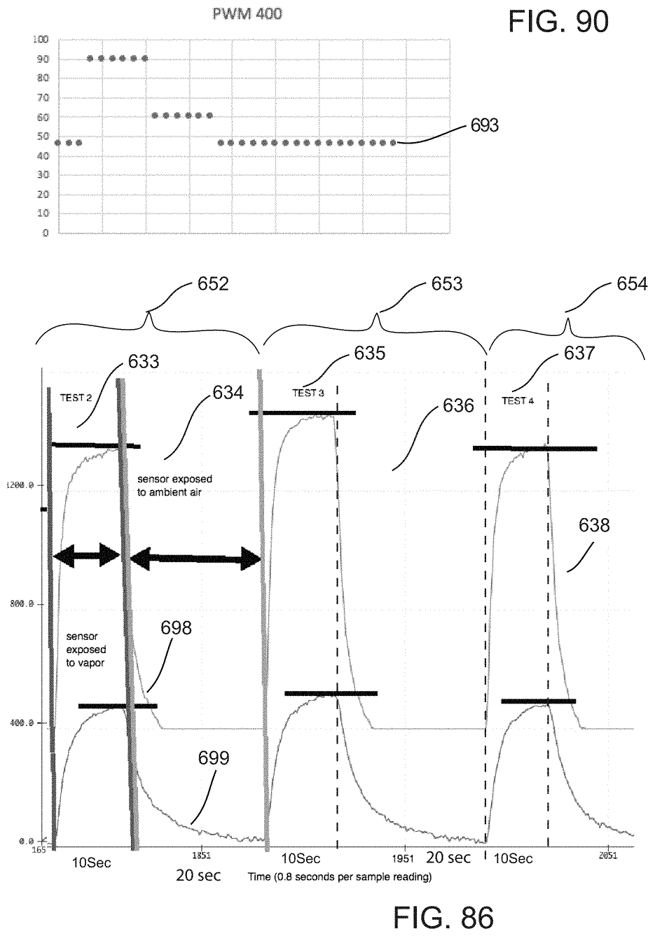

Referring to FIG. 86, a graph is shown illustrating differences in VOCs and CO2 as detected by the first VOC sensor in three sequential tests where the first VOC sensor is exposed to vapor and then other than exposed to vapor. The X axis is time where and the Y axis shows the CO2eq signal 698 in ppm and the TVOC signal 699 in ppb has been scaled down and is not in absolute units but is represented in relative units between tests. As shown on the graph, there is a second test 652, a third test 653 and a fourth test 654. In three sections of the chart as indicated by numbers 634, 636, 638, the first VOC sensor 1204 is exposed to ambient air. As vapor 4421 is drawn through the vaporization device 400 into the syringe cavity 1202d in an inhalation mode, as the plunger moves away from the second port 1201b, the first VOC sensor 1204 is exposed to vapor 4441 as is shown into the cavity 1202d (and FIG. 81), as indicated on the chart by sections 633, 635 and 637. In this testing embodiment the vapor 4421 and air mix is held for reading by the first VOC sensor 1204 for about 10 seconds post the actuator stopping and then it is expelled through the third port 1201c of the L valve. In some embodiments the second VOC sensor 1205 may be coupled proximate the inhalation aperture of the vaporization device 400 between the first and second ports 1201a and 1201b of the L valve 1201 to measure the vapor directly being inhaled from the vaporization device. In some embodiments through using the second VOC sensor it may offer for a means to measure vapor density that may be used to approximate dosing and may be used to provide a quantitative analysis on an amount of vapor that is produced as a result of inhalation using the testing system and through varying inhalation profiles and varying PWM profiles applied to the heating element assembly.

Referring back to FIGS. 80, 81, 82, 83, 84 and 85, some further elements that may be used in the vapor sampling system 1200 are as follows. Coupled with the third port 1201c there may be a male luer lock syringe fitting 1210 to pipe NPT ¼″ coupling where the third port may be a NPT ¼″ female and the male luer lock syringe fitting to pipe NPT ¼″ may be a male coupling. Other types of couplings may be used and this one is just exemplary. Fluidly coupled with the male luer lock syringe fitting may be a Syringe Filter 1211 Nylon 25 mm Diameter with a 0.45 um pore size with an optional downstream syringe filter nylon 25 mm diameter with a 0.22 um pore size.

Using syringe filters, air and vapor expelled from the expelled volume from the large syringe 1202 is then approximately trapped by the at least one syringe filter having pores and post capturing this filter is then weighed on a 0.1 mg Digital Analytical Balance Weighing Precision Lab Scale or a 0.01 mg Analytical Balance Weighing Precision Lab Scale where a mass of the syringe filter pre vaporization is subtracted from a mass of the syringe post vaporization and this difference may be a quantitative amount of vapor that may be inhaled by the system as the dose. Optionally the syringe filters are then eluted and the liquid therefrom is analyzed using a HPLC. Optionally a Field Asymmetric Waveform Ion Mobility Spectrometry is used for analysis of vapor or a RAMAN spectrometer is used to measure the vapor for determining a vapor density and quantity of a measurable ingredient that is vaporized as the dose that is captured by the testing system. In some embodiments the vaporization device 400 may be weighted on the Analytical Balance Weighing Precision Lab Scale and then post vaporization weighed again and a difference in the weight being a measure of the dose or an amount of vapor generated by the vaporization device 400. In some embodiments the third port 1201c of the L valve may be coupled directly with a Lonestar VOC Analyzer from www.owlstonemedical.com that uses FAIMS technology. Furthermore, preferably there is a minimum of dead air space in the L valve 1201 of the sampling system in the order of less than 5 ml.

Using a controllable actuator coupled with a syringe plunger, the inhalation profiles are preferably adjustable to be able to simulate various user inhalations. For the current figures, these differential pressures are based on a predetermine inhalation volumes, for example 0.4 L. In some embodiments an inhalation volume may be 400 ml or 550 ml or 650 ml or 0.42 L or 0.8 L. In use, when the user inhales from the vaporization device 400, in some embodiments an inhalation velocity of the user must cross a predetermined differential pressure threshold to trigger the heater to receive a corresponding PWM profile from the control circuit.

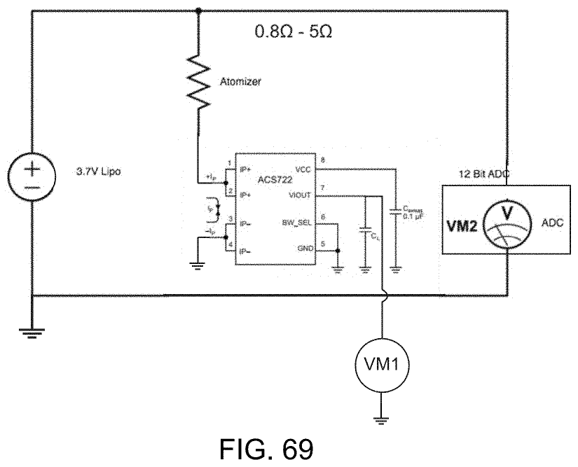

Referring now to FIG. 69, shown therein is an example of a temperature estimation circuit that may be used in embodiments of the vaporization devices or cartridges described herein. In some embodiments, the temperature of a resistive heating element such as a wire or of the heating element assembly, may be estimated by sensing a current being applied to the heating element assembly (atomizer) and for a predetermined voltage being applied to the heating element assembly. Through a temperature coefficient of resistance (TCR) of the heating element assembly, a temperature at which the heating element assembly is operating may be determinable.

A current sensing integrated circuit may be used to measure a first voltage VM1 and a 12-bit ADC, or 14 bit ADC, may be used to measure battery rail voltage being applied to the heating element assembly and for providing a second voltage VM2. The temperature of the heating element assembly or atomizer heating element may then be determined, e.g. using calibration values stored in a look-up table on the memory module of the device or cartridge that correlates applied voltage and current with the TCR of the heating element assembly, as is well known in the art. For example, the look-up table may include calibration values correlating the heating element temperature with the current through, and voltage across, heating element assembly.

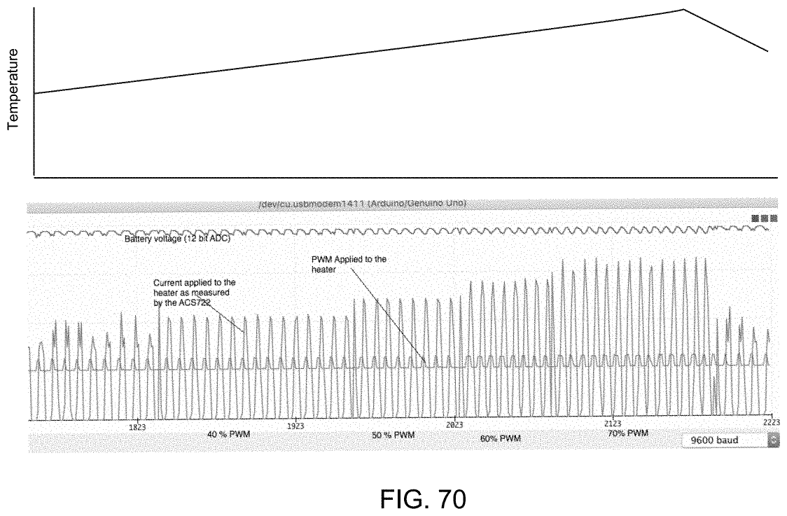

FIG. 70 illustrates different pulse width modulations (PWM) applied to the heating element as part of the atomizer. FIG. 70 illustrates atomizer temperature. As shown, when the PWM is increased a current is increased as well as a voltage drop is increased. Through calibration with a known atomizer wire resistance, or a resistance of the heating element assembly, an approximate temperature of the atomizer may be extrapolated

In many cases when PWM from the control circuit is being applied to the heating element assembly is over a very short timeframe, such as less than 4 seconds or around 2 second or in some cases around 1.8 seconds, it may be difficult to provide for a control circuit that in a cost-effective manner may operate to measure current and voltage and to vary the PWM applied to the heating element assembly such that effective temperature control of the heating element assembly is achieved. Using a temperature probe to measure the temperature of the heating element assembly in most cases will remove thermal energy from the heating element assembly during its heating. Furthermore, using the temperature probe may also result in the heating element assembly to provide thermal energy to the temperature probe and thus take away heat from the heating element assembly and as such probed heating element assembly temperature measurements may not be accurate and also delayed in their readings.

In the case of when a FLIR thermal imaging camera may be used to observe the heating element assembly in operation through empirical observation. The FLIR thermal imaging camera may be used to view the heating element assembly in operation and to provide temperature data as an output signal where this temperature data may be correlated with the current, voltage drop and known atomizer wire resistance to approximate the temperature of the heating element assembly and possibly of the heating chamber. A mass of air that is entering the ambient air input port may be measured using the calibration configuration shown in FIGS. 67-68 as well as FIGS. 80, 83, 84, 85 where air flowing past the heating element assembly may act to provide cooling to the heating element assembly in use.

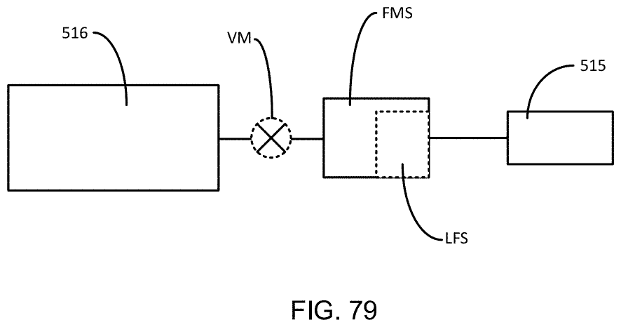

Referring now to FIG. 79, shown therein is a schematic drawing illustrating a fluidic manifold system (FMS) that may be used with cartridge assembly 500 in accordance with an embodiment. As shown in FIG. 79, a fluidic manifold system (FMS) may be positioned between the storage compartment 516 and fluid apertures 515. The FMS may be housed within the compartment 516 along with the vaporizable material. The fluidic manifold system (FMS) may be used to couple fluid apertures 515 to the vaporizable material within storage compartment 516. The FMS may be used to monitor and/or control the flow of vaporizable material through apertures 515. In some cases, the FMS may include a liquid flow sensor (LFS). The LFS may be configured to measure the flow of liquid vaporizable material from the storage compartment 516 to the fluid apertures 515. The LFS may be configured to provide flow rates in the low microliter/minute range, and upwards of 1 ml/min. For example, a planar microfluidic glass substrate with down-mount fluidic ports may be used for the LFS. The LFS may be manufactured so that glass is the only wetted material. The LFS may include a combination of microfluidic chips and digital micro-sensor chips. This may facilitate the measurement of liquid flow within the planar glass substrate.

The digital micro-sensor chip used in LFS may be configured to process the received flow measurements and generate a linearized digital output that may be provided to the control circuit. The micro-sensor chip may be calibrated with cartridge assembly 500, and may provide temperature compensation for the fluid measurements. The LFS may be implemented with a low thermal mass, enabling response times below 30 ms to be reached.

Optionally, a valve mechanism (VM) may be positioned downstream or upstream of the LFS. The valve VM may be operable to enable or prevent the flow of vaporizable material through apertures 515. For instance, where a predetermined volume of vaporizable material has passed through LFS (e.g. a defined volume per period time), the valve VM may be activated to prevent further vaporizable material from passing therethrough. This may prevent excess vaporizable material from existing the storage compartment 516 prior to preceding vaporizable material having been vaporized.

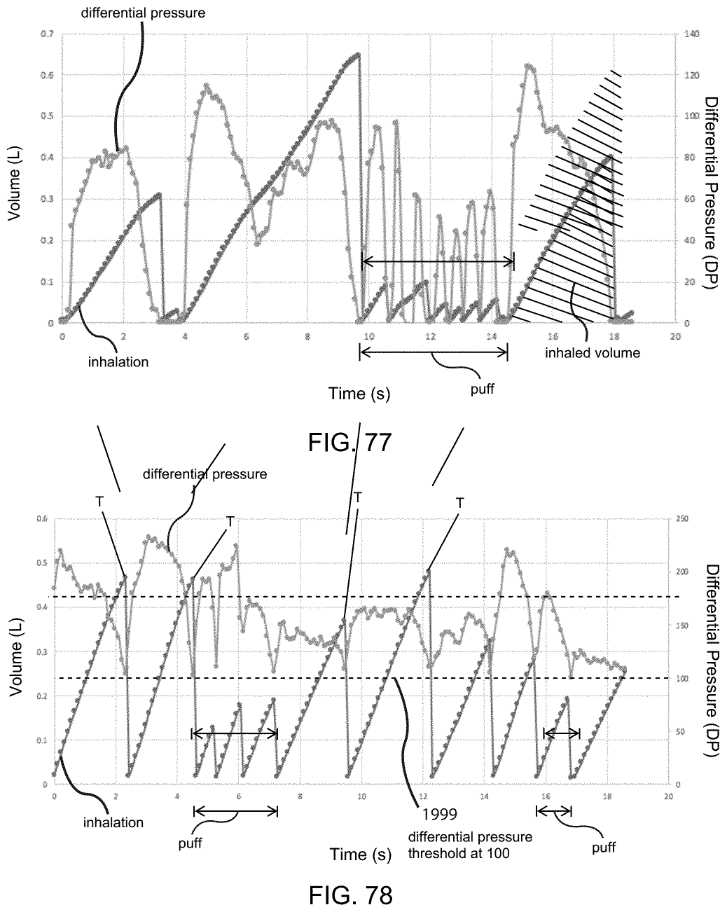

Referring now to FIGS. 77 and 78, shown therein are plots of inhalation volume, and differential pressure measured by, an example air intake manifold 410 that includes a differential pressure sensor 442. The plot of FIG. 77, a cumulative inhalation volume as air is drawn in a breath is shown on the left axis and a differential pressure measured by the airflow sensor 442 as the mass airflow sensor is shown on the right axis. The differential pressure is shown in kPA without a calibration factor applied (i.e. a raw reading).

The area under the curve is the total volume inhaled in a single inhalation. In the plot shown in FIG. 77, the graph includes three inhalations that use an approximately tidal volume (i.e. approximately 0.5 L) of inhalation (shown by the major peaks of the inhalation plot line) and then there are a plurality of puffing inhalations where the user puffs on the vaporizer and these result in much smaller volumes (shown by the minor peaks of the inhalation plot line between the second and third major peaks). The tidal inhalation volumes illustrated represent about 0.3 L, 0.65 L and 0.4 L inhaled respectively. The puffing inhalations each have about less than 0.1 L in volume. As explained above, inhalation volumes above puffing inhalation volumes may facilitate or improve vapor absorption in a user's lungs.

The plot shown in FIG. 78, illustrates tidal type inhalations that are labeled with ‘T’ that are that have a total inhaled volume of about 0.35 L per inhalation. A number of puffing inhalations are also shown. Puffing inhalations may occur in many pen-style vaporizer devices having small inhalation apertures and vapor conduits. Due to the small size of the flow passage, it may be difficult to achieve a tidal style inhalation because of flow restrictions in the diameter of the fluid conduit.

FIG. 78 shows an example of a plot in which a differential pressure threshold 1999 of 100 pascals has been defined. As a result, the mass airflow is not measured unless the differential pressure is equal to or greater than 100 pascals for this embodiment. If the differential pressure is less than the differential pressure threshold 100, a mass airflow measurement is not performed and electrical power is other than applied to the heating element. This may ensure that the vaporization device monitors inhalations of greater volumes (closer to tidal inhalations) and does not monitor shorter inhalations (i.e. puffs). These mass airflow measurements may then be converted to volumetric air flow using known techniques. The fluid flow sensor assembly 442 may be used to gauge air flowing through the manifold fluid flow path 436.

FIGS. 87, 88, 89, 90 illustrate graphical representations of PWM (pulse-width modulation) profiles applied over time to a heating element assembly, such as for example heating element assembly 510 shown in FIG. 34. The PWM profile represents a duty cycle that is applied from the control circuit to the heating element assembly. For example, for a PWM value of 100, the duty cycle is 100% and for a PWM value of 50 the duty cycle is about 50%. Each value from the PWM profile is held for about 120 ms when applied to the heating element assembly. A first pulse width modulation (PWM) profile “PWM200” 690 is graphically represented in FIG. 87, which corresponds to approximately 15 data points at 120m spacing, meaning this profile is applied for about 1.8 seconds. FIG. 88 illustrates a second pulse width modulation (PWM) profile “PWM300” 691 that is approximately being applied for 22×120 ms=2.6 seconds. FIG. 89 illustrates a third pulse width modulation (PWM) profile “PWM350” 692 that is approximately 25×120 ms=3 seconds in duration. FIG. 90 illustrates a fourth pulse width modulation (PWM) set “PWM400” 693 that is approximately 32×120 ms=3.8 seconds in duration. The PWM profile I applied for 120 ms, in that a value from within the PWM profile LUT, for example 90 means that the resulting PWM that is applied to the heating element assembly is 90% ON and 10% off at a frequency of about 500 Hz during the 120 ms window.

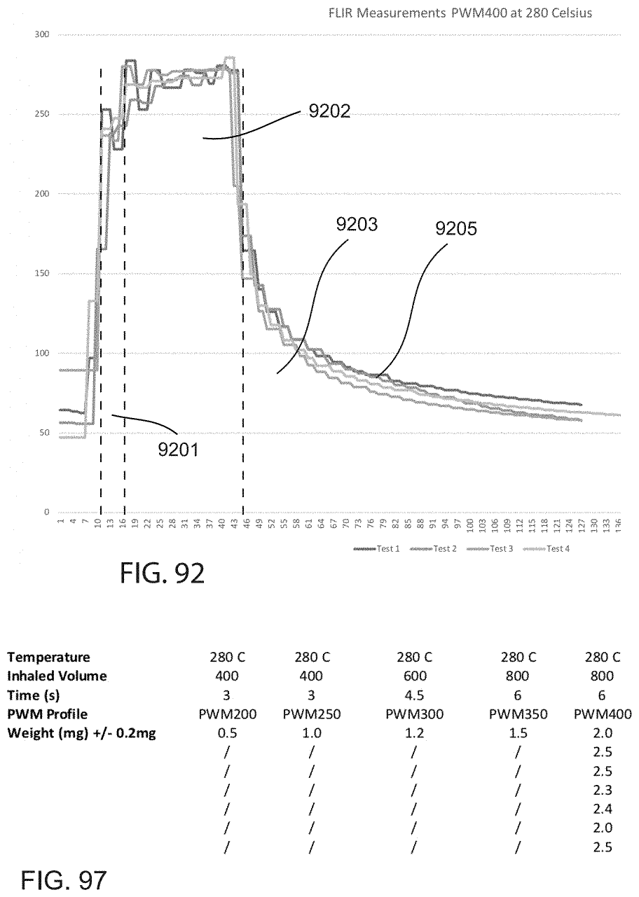

FIG. 92 illustrates thermal imaging graphs obtained from using the FLIR measurement system at a sample rate of about 10 Hz. The fourth PWM profile, PWM400 693 is applied to the heating element assembly 510 (FIG. 34) and through non-contact pyrometry a plurality of tests are executed for populating an array that is used for generating the PWM profile.

The heating element assembly may have an active heating area of about 4 mm×2 mm or about 5 mm×3 mm or about 4 mm×3 mm. A thermal imaging temperature data is generated and a shown profile is obtained using a thermal imaging camera whereby through non-contact pyrometry the heating element assembly is observer using the thermal imaging camera, such as a A310 thermal imaging camera by FLIR Systems.

In this embodiment the FLIR systems camera is using a close-up lens and imaging points of interest are selected on a surface of the heating element assembly for obtaining of the thermal imaging temperature data. The heating element assembly is wicked with phyto material extract and a PWM profile, such as PWM400 is applied to the heating element assembly using the PWM400 profile and the resultant temperature is observed from the thermal imaging camera as indicated. For generating this graphic, four tests were executed, Test1, Test2, Test3 and Test4, where temperature in degrees Celsius is shown on the Y axis and time is shown on the X axis, where the time shown is time in tens of seconds, to obtain seconds, divide the value shown by ten. From this figure, it may be observed that the PWM400 693 PWM profile provides for an approximately flat temperature profile over time when the PWM400 heating profile is provided to the heating element assembly 510.

For creating of the PWM profile, the PWM profile consists of a plurality of PWM values stored in a PWM array, wherein generating a pulse width modulation value from within the array of pulse width modulations in a calibration phase may be accomplished with the below steps. Initially for obtaining a first value a predetermined electrical power is applied over time to the heating element as a first pulse width value, for example for 100 ms, and obtaining a first calibration temperature signal is obtained from the FLIR camera through the non-contact pyrometric observation of heating element assembly. This obtained first calibration temperature signal is compared with a predetermined temperature signal, which is a desired temperature for the heating element assembly. Thereafter, the first pulse width applied to the heating element is amended in order to minimize a difference between the first calibration temperature signal and the predetermined temperature signal to create an amended first pulse width value. If the first pulse width applied yields first calibration temperature signal that is too low in value then the pulse width is increased. If the pulse width is at a maximum value, say 100%, and the and the predetermined temperature signal is not attained in this time duration, then this value may be stored as the first pulse width value within the pulse width modulation array as a first entry. If the predetermined temperature signal is exceeded, then the then the pulse width is decreased.

For determining a subsequent entry into the array, a second pulse width value is applied to the heating element and obtaining a second calibration temperature signal through a non-contact pyrometric observation of heating element assembly. A process the is taken in comparing the second calibration temperature signal to the predetermined temperature signal. Thereafter amending the second pulse width applied to the heating element to minimize a difference between the second calibration temperature signal and the predetermined temperature signal to create an amended second pulse width value, similar to what was aforementioned and then storing of the amended second pulse width value within the pulse width modulation array as a second entry.

The PWM profile array for a provided predetermined temperature signal is then populated with trough a plurality of applications of predetermined electrical power over time to the heating element and obtaining a plurality of temperature signal through a non-contact pyrometric observation of heating element assembly to generate a plurality of amended pulse width values to minimize a plurality of temperature differences between a plurality of temperature signals and the predetermined temperature signal and then storing of the plurality of amended pulse width values as the at least a pulse width modulation profile within the memory circuit within the control circuit 420.

When observing of the thermal imaging temperature data 9205 and in reading of a plurality of calibration temperature signals from the thermal imaging camera, resultant heating of the heating element assembly through an applied PWM profile may be observed as having a rising portion 9201, a plateau portion 9202, and a ramp down portion 9203 when analyzing of the thermal imaging temperature data. Based on the graph, the PWM400 profile is applied at time=0.7 seconds, where the rising portion 9201 is observed at 0.7 s to about 1.6 seconds, with a time of about 0.8 seconds, then the plateau portion 9202 is observed about 1.6 seconds to about 4.3 seconds and the ramp down portion 9203 is observed from about 4.3 seconds onwards. The PWM profile, ex. PWM400, is applied to the heating element during the rising portion 9201 and the plateau portion 9202.

Not shown in the graph is an additional time of the ramp down portion 9203, which may occur for about 2 minutes in order for the heating element assembly 510 to reach around 30 degrees Celsius or close to an ambient temperature of about room temperature or to about 40 degrees Celsius or to about 35 degrees Celsius. Post the ramp down portion 9203 and elapsed time of about 2 minutes when the fourth PWM profile, PWM400 693 is applied to the heating element assembly 510 subsequently, then an approximately same thermal imaging temperature data is observed. For the purposes of generating this graph, for Test 1, PWM400 was applied, the heating element assembly and then the heating element assembly may be allowed to cool for 2 minutes during the ramp down phase or ramp down portion 9203 and then PWM400 was applied again and this generated Test 2, and so forth until Test 4.

As the PWM400 heating profile is applied, the heating element assembly 510 increases in temperature and in dependence upon a thermal inertia of the heating element assembly 510, there may be a ramp up time 9201 where in order for the heating element assembly to fully reach a desired temperature, the plateau portion 9202, for example 280 degrees Celsius in this case, it may not be instantaneous. The ramp down portion 9203 facilities cooling of the heating element may be allowed to cool for about 30 seconds to about 120 seconds, which may facilitate a re-wicking time for the heating element assembly, which is further described hereinbelow. A subsequent application of the stored at least a pulse width modulation profile to the heating element is ceased for a predetermine amount of time to facilitate re-wicking of the vaporizable material into the heating element assembly proximate the heating element.

For the ramp down portion 9203 the observed temperature of the heating element may approximate an exponential decay function, for the plateau portion 9202 the observed temperature of the heating element may follow a plateau or having a slope varying by about plus or minus 20 degrees from horizontal and the ramp up time 9201 may be dependent upon a thermal inertia of the heating element assembly. In some embodiments the ramp up time 9201 may be about 1 second or 0.9 seconds, or 0.8 seconds or 0.7 seconds or 0.6 seconds.

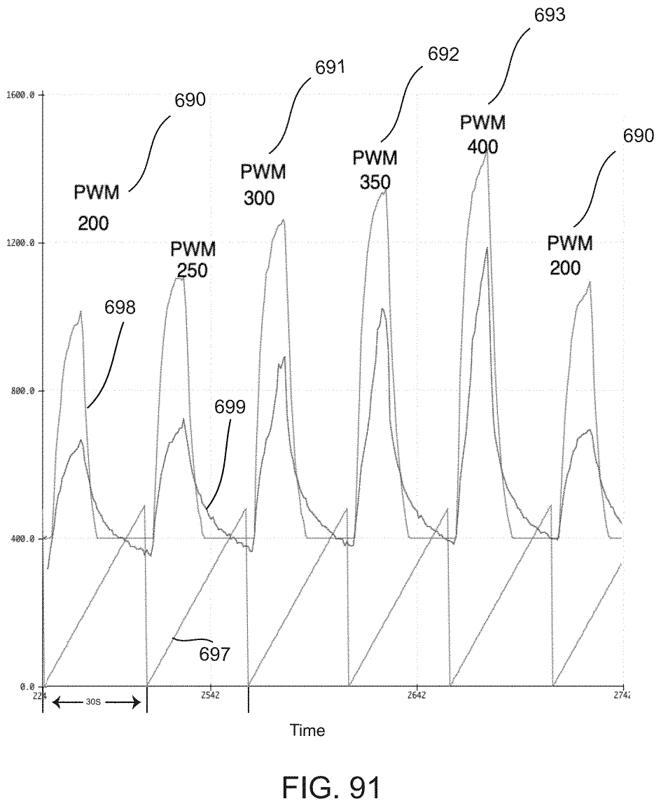

FIG. 91 illustrates various PWM profiles applied to the heating element assembly 510 and vapor captured from the vaporization device 400 using the vapor capture system 1200 with resultant output signals for CO2eq signal 698 in ppm and the TVOC signal 699 in ppb from the first VOC sensor 1205. The PWM profiles that are applied are indicated on the graph. A timing signal 697 is also shown that has a frequency of about 30 seconds, meaning that the inhalation and exhalation through the vapor testing system is taking place about every 30 seconds, where the vapor is contacting the first VOC sensor 1205 for about 10 seconds. From the graph it is observed as the PWM profile is increased from PWM200, to PWM250 (not show in FIGS. 87 to 90) to PWM 300 to PWM350 to PWM400 and back down to PWM200, a relative amount of vapor that is generate by the vaporization device as detected by the first VOC sensor 1205 is increasing from PWM200 to PWM400. A PWM LUT (PWM lookup table) is utilized to store therein data to achieve the corresponding PWM200 690, PWM300 691, PWM350 692, PWM400 693 as graphically illustrated. In some embodiments the PWM LUT may be stored within the control circuit and in some embodiments, it may be stored in the memory circuit disposed within the cartridge.

An inhalation volume using the vapor sampling system as show in FIGS. 80 to 85 that is utilized for each test is about 800 ml, where this is inhaled into the system in about 4 seconds. A suction process may be preferred through a vaporization device as this is its primary means of operation. Of course other volumes may also be drawn through the vaporization device, such as 600 ml. Form the graph for the same vapor volume being drawn into the system it is observed that as PWM profile that is applied to the heating element assembly the various PWM profiles, there is an additional relative vapor density observed through the use of the first VOC sensor 1204 where the first VOC sensor 1204 is either exposed to the vapor during the inhalation process or is exposed to ambient air and this is where the CO2 reading drops down to about 400 ppm post exhalation from the system.

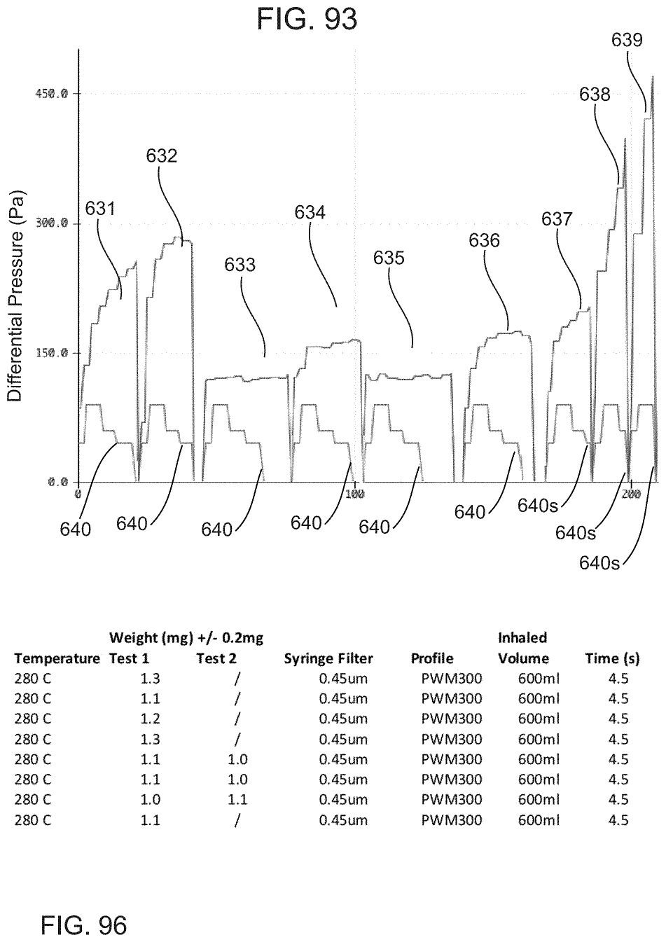

FIG. 93 illustrates various inhalation profiles being applied to the vaporization device and resulting differential pressure signals as reported by the fluid flow sensor assembly 442 in the form of a mass airflow sensor. For these graphs the vapor sampling system 1200 is used and the plunger 1202b of the large syringe 1202 is either actuated using the actuator 1203 or it is disengaged and a user performs an inhalation from the vaporization device 400. For generating this graph, the PWM300 691 PWM profile is applied to the heating element assembly 510 and shown in conjunction with the mass airflows sensor data. A volume of air that is drawn through the sampling system that is about 400 ml to 625 ml or about 600 ml. Of course, a larger or smaller volume of air may be drawn through the system, however the PWM profile is applied for a predetermine duration during upon the inhalation threshold 100 being surpassed when inhaling from the vaporization device 400.

The scale on the x axis is time and the vertical axis is differential pressure as measured by the differential pressure sensor 442 in Pascals and for this various inhalation profiles are shown. For tests 631 and 632 an inhalation is provided from the user and in item test 635 an actuator is used with a controlled draw for test 634 the actuator is used with manual assistance (i.e. pulled by additional manual force to increase an inhalation or draw rate from the vaporization device). For test 631, 632 and 636 a controlled user inhalation is provided and 637, 638 to 639 controlled breath is used whereby the inhalation velocity is increased throughout the inhalation. What is observed from this chart is that in using the actuator 1203, for tests 633, 634 and 635, the inhalation is more consistent as well as the differential pressure that is provide as an output signal from the differential pressure sensor or the mass airflow sensor is steadier with a flatter profile. In this embodiment the differential pressure sensor cut off differential pressure readings when a predetermined volume of airflow has occurred, which is about 600 ml in this case.

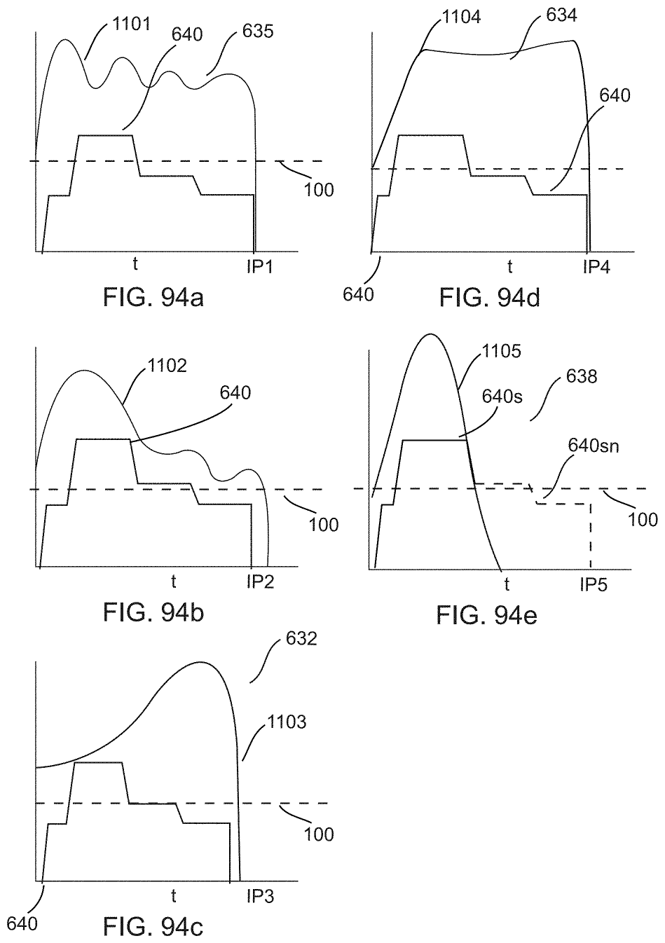

In the cases of inhalation profiles 637, 638, 639 these are inhalation profiles that resemble a partial dose, such as that shown in FIG. 94e 1105, whereby the inhalation from the vaporization device 400 was terminated prior to the end of the PWM300 being applied to the heating element assembly, meaning that an amount of vapor being inhaled by the end user is not that representative of a complete dose and is closer to a partial dose where the PWM300 profile was cut short as a shortened PWM profile 640s having a non-heating portion 640sn, where the PWM300 profile was cut short because the inhalation profile IP5 1105 dropped below the inhalation threshold 100 prior to the PWM300 profile finishing being applied to the heating element. It is preferable for a user of the vaporization device to inhale continuously and for their inhalation velocity to be over that of the inhalation threshold 100 in order for the end user to have completed an effective dose of phyto material extract. For example, an inhalation profile that resembles IP4 shown in FIG. 94d, is more optimal for a proper dose.

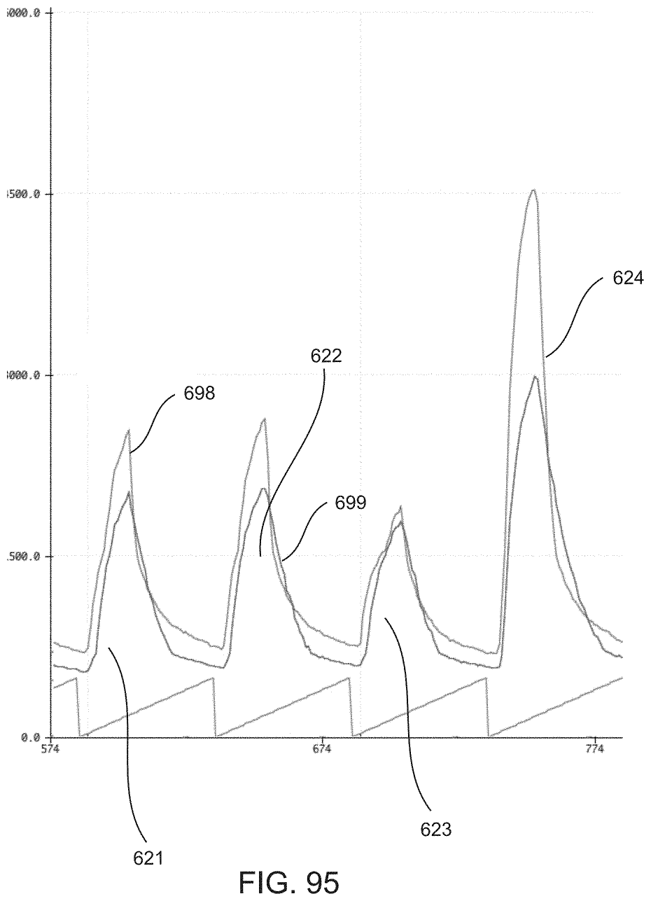

FIGS. 94a through 95d illustrate exemplary inhalation profiles, labelled to as IP1, IP2, IP3, IP4 and IP5, respectively. An inhalation profile is adjustable in the vapor sampling system through controllably controlling a rate and duration and volume being inhaled by the vapor sampling system, where for example various inhalation profiles are shown in FIGS. 94a though 94d, different inhalation profiles are shown that measure differential pressure over time, where the inhalation profiles are meant to resemble those that are potentially observed when being inhaled by the end user inhaling from the vaporization device. In conjunction with the inhalation profile shown the PWM profile of PWM200 is also illustrated.

Referring to FIG. 94a for example IP1 1101 has a fairly constant draw rate or inhalation rate, such as that also show in FIG. 93. For example, IP2 1102 of FIG. 94b has a sharp draw start and the draw rate tapers over time, for example IP3 1103 FIG. 94c has a slow draw rate and then the draw increases in rate over time, for example IP4 1104 FIG. 94d has a draw rate that is quite consistent over time, for example IP5 1105 FIG. 94e has a very quick inhalation rate, such as 638 shown in FIG. 93. This may prove to be problematic as there is not enough time for the PWM profile to apply heat to the heating element assembly and as such this case would be a partial dose. Referring to FIG. 94d, IP4 1104 may have a consistent draw rate that may be similar to that shown in FIG. 93 item 643.

Referring to FIG. 93, for a fixed PWM applied to the heating element and using an exemplary PWM300 691 and a fixed temperature, such as around 280 degrees Celsius. There is approximately a same air volume being inhaled (within about 10% to 20% of each other) through the vaporization device 400 when used with the sampling system is approximately 0.6 L. For the user, in some embodiments, a dose tracking process is initiated when they inhale form the vaporization device 400 and their inhalation rate surpasses the differential pressure threshold 100 (FIG. 94a). This is preferable as it prevents the user from puffing on the vaporization device. Publications relating to this are “Fundamentals of aerosol therapy in critical care” https://www.ncbi.nlm.nih.gov/pmc/articles/PMC5054555/ and “Pulmonary drug delivery. Part I: Physiological factors affecting therapeutic effectiveness of aerosolized medications”, by N R Labiris and M B Dolovich Department of Medicine at McMaster University, Hamilton, Ontario, Canada.

Having the user inhale so that a predetermined differential pressure threshold 100 is exceeded and maintained throughout the dose (inhalation from the vaporization device 400) this may allow for a deeper pulmonary inhalation (as opposed to sucking the vapor into their mouth first and then inhaling into the lungs as is typical of many puff based devices) and thus resulting in a larger amount of alveoli to be in contact with the inhaled vapor and this may increase the effectiveness of the treatment. For a given dose the user reports increased effectiveness for a lesser amount of active ingredient that is vaporized from the phyto material extract when performing an inhalation directly into the lungs vs puffing on the vaporization device. This may then save the user money on their phyto material extracts as less doses are required to achieve a similar effectiveness. In some embodiments the user may be educated on how to vary their inhalation in order to obtain improved dosing wen using of the vaporization device, for example the user inhales along a inhalation profile that is IP1 with PWM200 and they obtain higher dose, a larger amount of vapor, than when the user inhales at inhalation profile IP5 with PWM200 (FIG. 94e). Deeper inhalation into the lungs leads to a better efficacy than puffing the vaporization device. Potentially through education on vaporization device use, the user is instructed to maintain their inhalation past the differential pressure threshold 100 (FIG. 94a) where a tutorial may be utilized in order to educate the user on how to properly inhale from the vaporization device.

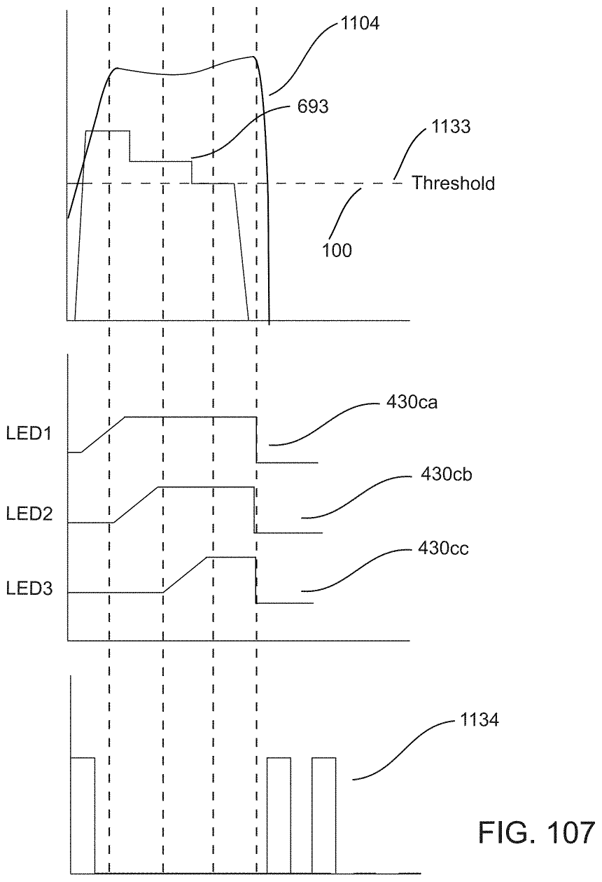

FIG. 95 illustrates a graph of different inhalation rates through the vaporization device 400 and resulting CO2 698 and VOC 699 as detected by the first VOC sensor. Referring to FIG. 95 and FIG. 93, a same volume of air in inhaled from the vaporization device 400, with a same PWM profile, PWM200, and the heating element is provided with a same PWM profile that should result in a same heating element assembly temperature, of about 280 degrees Celsius. When a standard inhalation is performed, such as that shown as inhalation profile 636 in FIG. 93, a resulting CO2 698 and VOC 699 signals as detected by the first VOC sensor as part of the sampling system, where the inhalation rate is about 2.5 seconds and the inhaled volume is about 400 ml, then a certain relative vapor density is observed by the first VOC sensor. When the inhalation profile is slower, such as that of FIG. 93 inhalation profile 633, for test 624 an inhalation rate of about 3 seconds for test 624, then a higher vapor density is observed. When the inhalation rate is faster, such as about 2 seconds for 400 ml, such as for inhalation profile 638 FIG. 93, then the relatively lower vapor density is observed in test 623. The inhalation rate through the vaporization device has a bearing on a vapor density produced. A faster inhalation rate cools the heating element assembly more than a slower inhalation rate. As the ambient air flows into the vaporization device it flows as the heating element assembly and as a result will provide cooling to the heating element assembly. Through a dose training process (see FIG. 107) the user may be presented with an indication of an optimal rate at which to inhale from the vaporization device to be provided with optimal dosing.

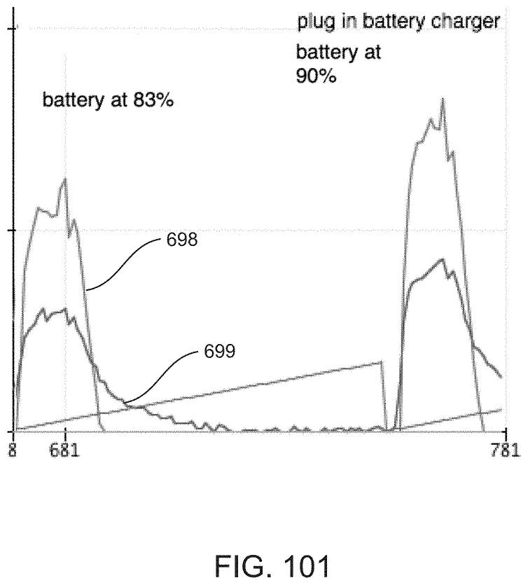

FIG. 101 illustrates resulting CO2 and VOC signals as detected by the first VOC sensor in relation to battery power available to the vaporization device. The CO2eq signal 698 in ppm and the TVOC signal 699 in ppb from the first VOC sensor 1205 where with lower power being available in the battery a relative vapor density is reduced as compared with a higher battery power available. For example, where a battery level is at 83% availability for the control circuit to provide power to the heating element assembly then there is relatively less vapor produced than when the control circuit is provided with a higher power from the battery, where for example the vaporization device is plugged into a charging supply to increase a voltage of the battery to about 90%. From this it is observed that battery power has an impact on the PWM profile being applied to the heating element assembly and relative vapor density measurement.

Generally, for the creation of the tables shown in FIGS. 96 and 97, the below process was followed. For calibration purposes, a phyto extract (distillate or resin) is first tested for its critical ingredients profile, for example, THC, CBD percentages as well as a room temperature viscosity. This extract is then filled into the cartridge where in some embodiments it may use the filling system that is described above. The vaporization device 400 is then coupled with the sampling system. In the case of the vaporization device 400 is then coupled with the sampling system and a controlled draw using inhalation profiles, for example IP1 1101 at first takes place and vapor is captured within the large syringe. This vapor is then may be exposed within the large syringe to a VOC sensor over a period of for example 10 seconds (such as that shown in FIG. 91). The vapor is then expelled from the large syringe into a syringe filter (0.22 um and or 0.44 um pore size) is then approximately trapped by the at least one syringe filter having pores. The syringe filter is weighed pre-vapor capturing and post vapor capturing. Post vapor capturing the filter or filters are then weighed on a 0.1 mg Digital Analytical Balance Weighing Precision Lab Scale or a 0.01 mg Analytical Balance Weighing Precision Lab Scale where a mass of the syringe filter pre-vaporization is subtracted from a mass of the syringe post vaporization and this difference may be a quantitative amount of vapor (by weight) that was inhaled by the system as the dose. Optionally the syringe filters are then eluted and the liquid therefrom is analyzed using a HPLC to further determine amounts of active ingredients released into the vapor and captured by the syringe filter. Optionally, if syringe filters are not being used then the vaporization device is removed from the vapor sampling system and then weighed pre and post vaporization to obtain a vapor weight that is created by the heating element assembly.

FIG. 96 illustrates measurements of a dose and presented in a weight table created for the vaporization device 400 using a phyto material extract having a viscosity of about 3000 Centipoise to about 5000 Centipoise with a heating element assembly having a porosity of about 50%, such as heating element assembly 510, and a wire heating element having a resistance of about 1.2 Ohms and embedded within the porous ceramic as the heating element assembly and a battery voltage of about 3.7V. The aforementioned PWM profiles are applied to a heating element assembly that includes a resistive heating element 264, in the form of a planar stamped heating element that is embedded at least partially within a porous ceramic heating element assembly. With a phyto material extract utilized at about room temperature, an inhaled volume of about 600 ml during about 4.5 seconds using a flat inhalation profile, such as 633, and with the application of the PWM300 PWM profile to the heating element assembly, vapor being captured using a 0.45 um syringe filter, with the syringe filter then being weighed on an analytical scale having a measurement error of about plus or minus 0.2 mg, the table shown in FIG. 96 is a result of the experiment. Two tests were conducted, Test 1 and Test 2 as shown in this FIG. 96, where an average dose size of about 1.1 mg was obtained.

FIG. 97 illustrates a table created for the vaporization deice 400 using a phyto material extract having a viscosity of about 3000 Centipoise to about 5000 with a heating element assembly having a porosity of about 50% and a wire heating element having a resistance of about 1.2 Ohms and embedded within the porous ceramic and a battery voltage of about 3.7V. The aforementioned PWM profiles are applied to a heating element assembly that includes a resistive heating element 264, in the form of a planar stamped heating element that is embedded within a porous ceramic as aforementioned as the heating element assembly. With a phyto material extract utilized at about room temperature, an inhaled volume of about 400 ml, 600 ml and 800 ml, in about 3 s, 4.5 s and 6 s, respectively, using a flat inhalation profile, such as profile 633 of FIG. 93) and with the application of the PWM profile as shown on the table to the heating element assembly. Vapor being captured using a 0.45 um syringe filter, with the syringe filter then being weighed on an analytical scale having a measurement error of about plus or minus 0.2 mg. The table shows vapor weight for varying dose sizes where for a PWM profile of PWM200 and an inhaled volume of about 400 ml there was a vapor weight of about 0.5 mg and for PWM profile of PWM250 and an inhaled volume of about 400 ml there was a vapor weight of about 1 mg and for a PWM profile of PWM300 and an inhaled volume of about 600 ml there was a vapor weight of about 1.2 mg and for a PWM profile of PWM350 and an inhaled volume of about 800 ml there was a vapor weight of about 1.5 mg and for a PWM profile of PWM400 and an inhaled volume of about 800 ml there was a vapor weight on average of about 2.3 mg.

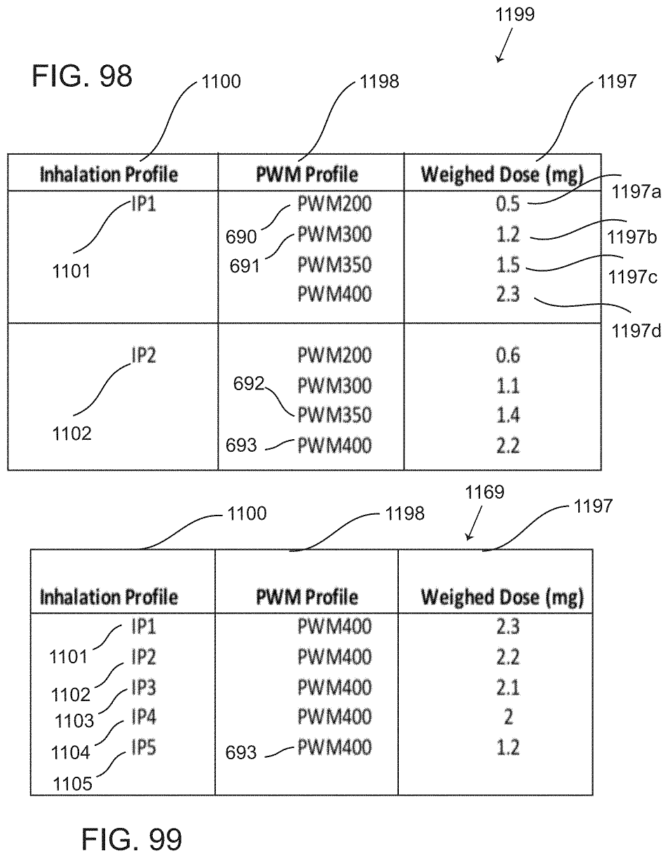

FIG. 98 illustrates a plurality of PWM profiles applied for the first and second inhalation profiles and corresponding vapor weights obtained post sampling of the vapor using the sampling system. In the calibration phase for a plurality of inhalation profiles 1100 (IP1 1101, IP2 1102) and having a plurality of PWM profiles 1198 (PWM200 690, PWM300 691, PWM 350 692, PWM400 694) a first dosing data lookup table 1199 (DDLUT) may generated where based on quantitative analysis using the sampling system to generate a measured dose value 1197a through 1197d from a plurality of measured dose values 1197 through weight.

These entries then may be stored into the first DDLUT 1199 to create a plurality of dose values that are dependent upon at least an inhalation profile, the inhalation profile being above the inhalation threshold, the PWM profile applied to the heating element assembly, the viscosity of the phyto material extract, and parameters associated with the heating element assembly, such as porosity of the wicking element, the resistance of the heating element, a type of heating element assembly, ambient temperature. The DDLUT 1199 is optionally expanded to include a plurality of varying inhalation profiles. In some embodiments it may be preferable to have the inhalation volume being fixed or manageable for an end user and something that is comfortable for being inhaled by the user, for example from 400 ml to 800 ml, that is close to a tidal volume.

Referring to FIG. 99 in some embodiments the inhalation profile is stored in relation to a single PWM profile within a second DDLUT 1169, thus creating the second DDLUT where an inhalation profile (IP1, IP2, IP3, IP4, IP5) and a single PWM profile (PWM400) for example is correlated with vapor weight value as determined through the calibration process as aforementioned. In many cases a single PWM profile may be utilized for ease of use for controllably providing of power to the heating element assembly. In using such a single PWM profile, the heating element assembly is heater using the single PWM profile.

In some embodiments, a single PWM profile a substantially uniform temperature that is provided to the heating element assembly, where for example a target predetermined temperature signal as determined through the non-contact pyrometric observation of heating element assembly may include a deviation from the predetermined temperature signal of about plus or minus 10 percent variation for less than 70% of time for which the pulse width modulation profile has been applied to the heating element assembly.

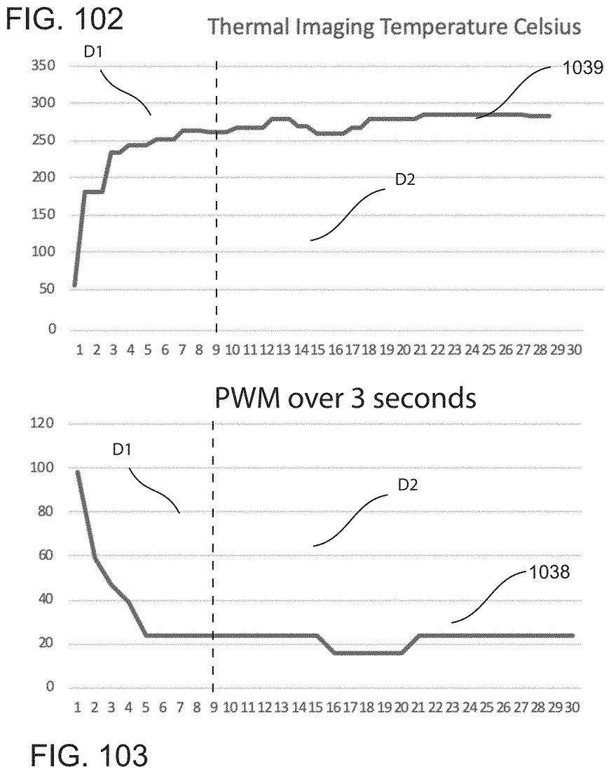

Referring to FIGS. 102 and 103, where FIG. 102 illustrates an example of a non-contact pyrometric observation of heating element assembly, such as heating element assembly shown in FIG. 36, however instead of the heating element assembly having embedded therein a heating element, the heating element may be silk screened onto the heating element assembly on a same surface from where the heating element contacts protrude. A silk screened or printed heating element having a lower thermal inertia than an embedded heating element that is stamped from resistive metal or coiled from resistive metal. Silk screening has an advantage over embedding the heating element as part of the heating element assembly in that the heating element assembly with the embedded wire is sintered at a lower temperature than the heating element assembly that is first sintered and then having the heating element silk screened thereon, typically porous ceramics that make up the heating element assembly that is used on conjunction with silk screening are formed from aluminum oxide and silicon carbide with a more uniform porosity than the heating element assembly that is used on conjunction with the embedded heating element that is formed mainly from aluminum oxide.

FIG. 103 illustrates a corresponding PWM profile, PWM280C 1038 used to generate a temperature profile (TP208C 1039) shown in FIG. 102 using the heating element assembly with the silk screened or printed heating element. As an example, the PWM280C 1038 illustrated is broken down into sections D1 and D2, where for a user if they inhale for a certain volume, for example 400 ml from the vaporization device, they may trigger the heating element assembly to be enabled with the PWM280C profile for generating a vapor weight corresponding to D1, which in this case may be for around 1.4 seconds or so. The user if they inhale for example 600 ml to 700 ml from the vaporization device, they may trigger the heating element assembly to be enabled with the PWM280C profile for generating a vapor weight corresponding to D2, which in this case may be for around 2.8 seconds or so. The PWM280C profile applied to the heating element may have resemble an exponential decay (from about 100% PWM to about 20% PWM) during the rising portion 9201 and may resemble an approximately flat PWM of about 20% being applied during the plateau portion 9202. In some cases, the PWM may have a slight negative slope during the plateau portion 9202.

The PWM profile that is applied to the heating element assembly for generating the varying heating profiles as shown is with the use of a PWMLUT that stores the PWM profile that provide for a temperature that is approximately 280 degrees Celsius or 270 degrees Celsius or range that is 290 degrees Celsius plus or minus about 10 degrees Celsius or so as extrapolated from the FLIR measurement system at a sample rate of about 10 Hz. In some embodiments the PWM profile varies in 100 ms regions, for example for a first region a PWM of 100% is applied (meaning full power for 100 ms) and then for a subsequent region 95% PWM may be applied for 100 ms and so forth).

A thermal inertia of the heating element and the heating element assembly affects the PWM profile and resulting temperature that is attained by the heating element assembly as well as the ramp up time 9201 of the heating element assembly to the plateau portion 9202 and less so for the ramp down portion 9203. Factors affecting the thermal inertia for the ramp up time 9201 are porosity, a type of porous ceramic, and heating element construction. For example, for a resistive wire coil embedded into the heating element assembly (for example the coil shown in FIG. 5, where the plurality of resistive heating wire bands 264 may be in the form of a coiled wire embedded within the porous ceramic heating element assembly 230 may have the PWM profile as follows:
PWM_320Ccoil[48]={100,100,100,100,100,100,100,100,100,100, 80,80,80,70,70,70,70,70,70,70, 60,60,60,60,60,60,57,55,52,50, 60,60,50,50,60,60,50,50,60,60, 60,50,60,60,60,50,60,50};

For example, for a silk-screened heating element that is printed onto the porous heating element assembly, such as that shown similar to FIG. 34 (however without the heating wire being embedded and silk screened onto the heating element assembly surface having a porosity of about 50%), the heating element assembly may have the PWM profile as follows:
PWM_320C_S[48]={100,100,90,90,90,94,80,70,70,70, 80,80,80,70,70,70,70,70,70,70, 60,60,60,60,60,60,57,55,52,50, 60,60,50,50,60,60,50,50,60,60, 60,50,60,60,60,50,60,50};

From the above PWM profiles shown, for the PWM_320Ccoil profile there is significantly more power being applied to the heating element than for the PWM_320C_S PWM profile. The resistive metal wire has a higher thermal inertia than the silk-screened heating element and as such requires additional full power PWM to bring its temperature up to the plateau portion during the ramp up time. The silk-screened heating element may therefore have a steeper temperature slope when observed using the non-contact pyrometry as compared with the embedded heating wire solution.

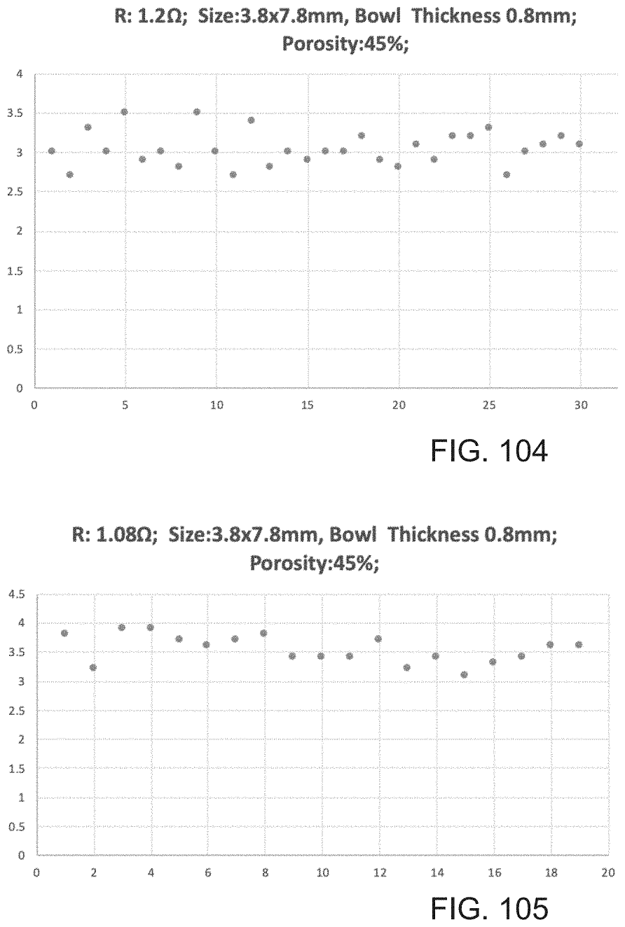

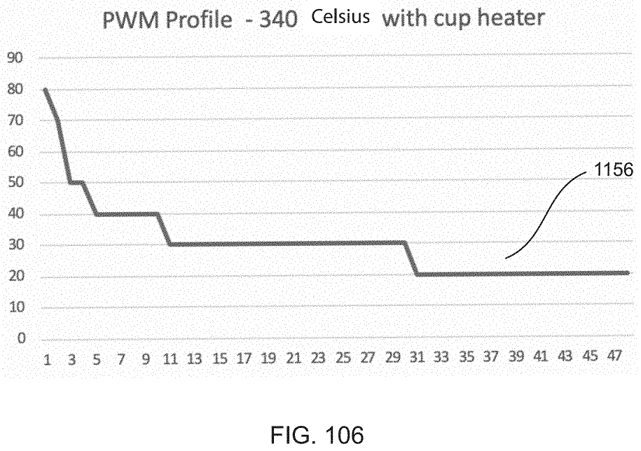

FIG. 104 and FIG. 105 illustrate vapor weight as measured using the vapor sampling system 1200 and corresponding methodology of use as described hereinabove to measure a vapor weight emitted from the vaporization device using a PWM profile shown in FIG. 106, PWM340C 1156 applied to the first resistance and the second resistance of the heating element for generating the FIG. 104 and FIG. 105 graphs. The temperature signal generated by the non-contact pyrometry when using of this PWM340C 1156 PWM profile results in an average temperature of about 340 degrees Celsius. Generally, the PWM profile is at 100% to get the heating element to reach the desired predetermined temperature and during the plateau portion the PWM profile may be reduced.

For the vapor weights measured in FIG. 104, a second resistance of 1.2 Ohm resistance heating element is used and the heating element is about 3.8 mm×7.8 mm, similar to the heating element assembly shown FIG. 36 and in this case with a silk-screened heating element. A plurality of samples are taken. For the vapor weights measured in FIG. 104, a first resistance of 1.08 Ohm resistance heating element is used and the heating element is about 3.8 mm×7.8 mm, similar to the heating element assembly shown FIG. 36 and in this case with a silk-screened heating element. Both heating elements have a floor thickness (FIG. 37) of around 0.8 mm. In comparing these two graphs what is illustrated is that for a heating element assembly having a lower resistance heating element (FIG. 105) there is an average higher vapor weight being produced, around 3.5 mg per inhalation than for a higher resistance heating element (FIG. 104) there is a lower vapor weight being produced, around 3 mg per inhalation.

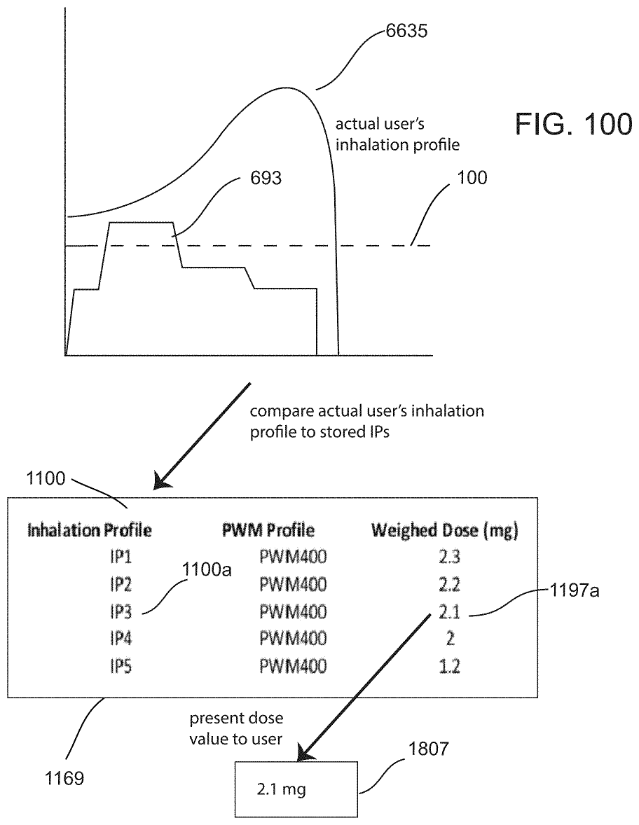

FIG. 100 illustrates an exemplary flowchart of how the second DDLUT 1169 may be practically utilized with the vaporization device and for meaningful information to be presented to the user. illustrates an inhalation being performed by a user being compared to a lookup table of stored inhalation profiles to provide a calibrated indicated dose weight to an end user.

In some embodiments the user performs an inhalation according to an actual inhalation profile 6635 from the vaporization device, for exemplary purpose vaporization device 400, surpassing the inhalation threshold 100. For this case the PWM400 693 profile is applied to the heating element assembly during the user's inhalation while they are surpassing the inhalation threshold 100.

During the user's inhalation for creating of the actual inhalation profile 6635 is in realtime compared to the stored inhalation profiles 1100 stored within the second DDLUT 1169. For example, each inhalation profile has 20 values stored in an array at 100 ms intervals sample rate and the actual inhalation profile 6635 is sampled at 100 ms sample rate. For obtaining an inhalation profile then the fluid flow sensor 442 is in the form of the mass airflow sensor 442m (FIG. 109). A least square fit algorithm is executed by the processor to compare the actual inhalation profile 6635 as detected by the mass airflow sensor 442m with stored inhalation profiles 1100 stored within the second DDLUT 1169.

Based on a resulting least square fit calculation, a closest IP 1100a from within the stored IPs 1100, for example in this case IP3 1103 may act as an index within the second DDLUT 1169 and a corresponding calibrated measured dose value is then presented to the user as a presented dose value post inhalation from the vaporization device 400. This presented dose value 1807 may be presented using a display screen, such as an OLED display screen as part of the vaporization device or sent to a user's smartphone, tablet, etc. This value may also may also be stored within the remote server 1008 and corresponding to the cartridge identifier data may include a unique identification number 288, which is also stored in correlation with the measured dose value 1197a.

The second DDLUT 1169 or the first DDLUT 1199 may be stored in a memory circuit within the control assembly of the vaporization device or stored within the memory circuit of the cartridge, such as the onboard memory storage module 554, which may be an EEPROM or FLASH type of memory. In some embodiments the second DDLUT 1169 or the first DDLUT 1199 may also be stored on the data server 1008 and indexed by the cartridge identifier data. In some embodiments the memory circuit of the cartridge may contain a first portion at least one of the second DDLUT 1169 or the first DDLUT 1199 stored therein and through the cartridge identifier data it may access the data server 1008 and wirelessly received a second portion at least one of the second DDLUT 1169 or the first DDLUT 1199 to be stored within the memory circuit of the cartridge.

In some embodiments the cartridge identifier data that may be encoded within the cartridge memory module 254 of the removable cartridge assembly may contain only partial information that is not related to the dosing determination and dose weights because a viscosity of the vaporizable material is not known and upon filling of the cartridge with the vaporizable material and then performing a calibration test using the calibration system, the data for the at least one of the second DDLUT 1169 or the first DDLUT 1199 may be generated and stored onto the data server 1008. Whereby upon the cartridge electrical contracts being in communication with the with the control circuit of control circuit assembly, the cartridge memory module may then have missing data from the at least one of the second DDLUT 1169 or the first DDLUT 1199 programmed by the control circuit of control circuit assembly.

In some embodiments the user is presented with a dose they consumed by looking up the corresponding vapor weight as determined through the calibration process from one of the first and second DDLUT 1169, 1199 post inhalation from the vaporization device. In some embodiments as the user is inhaling from the vaporization device, a real-time calculation is performed on a presented dose value that is presented to the user and during the inhalation process the presented dose value increments changes from an initial zero value to approximately a measured vapor weight value stored in the second DDLUT 1169, or in some cases an approximation of the measured vapor weight value stored in the second DDLUT 1169. This incrementing of the presented dose value may occur during differential pressure threshold 100 or in the case when a puff sensor or an audio microphone is used, an inhalation threshold whereby the puff sensor or an audio microphone or barometric pressure sensor detects airflow through the manifold fluid flow path.

FIG. 108 and FIG. 109 illustrate a portion of a vaporizer device in accordance with embodiments of the invention where the vaporization device includes a vaporizer body 402 formed from an elongated base 404 extending from a first end 402A to a second end 402B, the elongated base 404 including a pair of opposed sidewalls extending between the first end and the second end and a second end wall at the second end. A mouthpiece formed proximate the inhalation aperture 412 at the second end 402B of the base 404, the mouthpiece comprising an inhalation aperture 412 through the second end wall. FIG. 108 illustrates a portion of a cartridge assembly showing the heating element assembly and FIG. 109 illustrates a cutaway view of the cartridge assembly and where both figures show the cavity 516c formed within the heating element enclosure 563a

An air intake manifold 410 is mounted to the base 404, the air intake manifold having a first manifold end 458A and a second manifold end 458B with a manifold fluid flow path 436 defined therethrough, the air intake manifold comprising an ambient air input port 440 disposed between the first manifold end and the second manifold end, the ambient air input port 440 being exposed to an external environment.

The fluid flow sensor assembly 442 fluidly coupled between first manifold end and a second manifold with the manifold fluid flow path 436, the fluid flow sensor assembly for generating a fluid flow signal in dependence upon a flow of air through the manifold fluid flow path 436 exceeding a predetermined flow threshold 1999. The elongated storage compartment 516, the storage compartment being configured to store a vaporizable material 350, the elongated storage compartment 516 comprising an inner storage volume 516v wherein the vaporizable material is storable in the inner storage volume 516v, the elongated storage compartment comprising a first end and a second end opposite the first end. The heating element assembly 510 disposed at the elongated storage compartment first end, the heating assembly 510 comprising a heating element, wherein the heating element is thermally coupled with the heating element assembly 510, and wherein heating element assembly is in fluid communication with the inner storage volume 516v for wicking of the vaporizable material 350 into the heating element assembly 510. The fluid conduit 504 extending parallel with the elongated storage compartment 516 from the first end to the second end, the fluid conduit having a fluid conduit inlet proximate the elongated storage compartment first end and a fluid conduit outlet proximate the elongated storage compartment second end. The fluid conduit 504 is in fluid communication with the heating element assembly 510 and the fluid conduit inlet is fluidly connected to the air intake manifold and the fluid conduit outlet is fluidly connected to the mouthpiece, and a fluid flow passage is defined between the ambient air input port and the inhalation aperture, the fluid flow passage passing proximate the heating element assembly 510.

A control assembly (not shown in this figure) is substantially enclosed with the vaporizer body and electrically coupled with the fluid flow sensor assembly and the heating element, the control assembly for reading from a memory circuit which is for storing at least a pulse width modulation profile therein where upon the fluid flow signal being generated the at least a pulse width modulation profile stored within the memory circuit for controllably applying electrical power with respect to time to the heating element based upon the least a pulse width modulation profile, the heating element for heating of the heating element assembly and for creating an aerosol from the vaporizable material that is wicked into the heating element assembly and for the aerosol to flow into the fluid flow passage and for the aerosol to mix together with the ambient air flow through the manifold fluid flow path for together to flow from the mouthpiece. In some embodiments a length of the storage compartment may be approximately same as its width or some cases longer than its width. For example, the storage compartment may have a length of about 10 mm and a width of about 15 mm.

In some embodiments the current sensing integrated circuit maybe used to determine a current flowing through the heating element (such as shown in FIG. 69). Through measuring of the first voltage VM1 and the second voltage VM2 upon an application of a PWM profile to the heating element, in dependence upon a rate of climb of the first voltage VM1, the control assembly may be used to determine whether the heating element assembly is wicked with vaporizable material or whether it is approximately dry. In the case when the heating element assembly is dry and the PWM profile is applied, the heating element will generate heat at a faster rate and will have a sharper slope than when the heating element assembly is saturated and the heating element will generate heat at a slower rate and will have a shallower slope. The control assembly may detect this slope and cease providing of the PWM profile to the heating element assembly in the case when through a detection operation it is determined that the heating element assembly is dry. This may prevent overheating of the heating element and may prevent burning out of the heating element. In some embodiments, the storage compartment may be oriented in such an orientation that the vaporizable material may not be contacting of the heating element assembly and the heating element assembly has a predetermined amount of vaporizable material contained therein and upon a first heating operation with the application of a PWM profile, the predetermined amount of vaporizable material is utilized and the heating element assembly should undergo a wicking time in order to re-wick additional vaporizable material from the storage compartment into the heating element assembly. In the case when this time is not sufficient, for example the user takes another inhalation from the vaporization device, then the control circuit may prevent this operation thr9ugh detecting that the heating element assembly is approximately dry. Thereafter the user may position the vaporization device in such an orientation for vaporizable material to to flow from the storage compartment to the heating element assembly for a re-wicking operation.

Referring to FIGS. 80, 81, 82, 83, 84 and 85 it is envisaged that such a calibration system is semi-automated or automated whereby the various inhalation profiles (IPs) are in sequence executed and vapor is captured using the sampling system and then the syringe filters are weighed or the vaporization device is weighted pre and post vaporization. In some embodiments a calibrated FAIMS (High-Field Asymmetric Waveform Ion Mobility Spectrometry) may be used to quantify the vapor emitted from the sampling system in order to perform calibration of at least one of the first and second DDLUT, 1199 and 1169.

It is envisaged that during a filling process of the cartridges that there may be a periodic calibration verification of each cartridge. In some embodiments when the vaporizable material or phyto extract that is filled into the storage compartment of the cartridges is altered in formulation then a new calibration will take place whereby different measured dose values will be stored in the at least one of the first and second DDLUT, 1199 and 1169.

In some embodiments when populating of the at least one of the first and second DDLUT, 1199 and 1169, ambient air temperature and may also include with stored calibration parameters. Where a temperature of air being inhaled into the air input port prior to entering into the manifold fluid flow path 436.

In some embodiments the operation of the heating element assembly may be disabled for a period of time after taking a dose or draw from the vaporization device 400. There may be the re-wicking time during the ramp down portion 9203, this may be for the heating element to return back to ambient temperature through an approximately exponential decay and also for the user not to continuously draw on the vaporization device 400 but to make a conscious decision as to whether they desire to take another dose. Absent a temperature sensor for actively monitoring the temperature of the heating element assembly through a PID control loop, allowing the heating element assembly to operate through a ramp down portion 9203 or ramp down time, may allow for the heating element to other than overheat and potentially damage itself or provide too much of a dose on subsequent time due to the heating element exceeding a temperature that is created by applied PWM profile. As the heating element may become damaged, its resistance may increase and a damaged heating element may produce less vapor than an undamaged heating element.

The temperature of the heating element assembly during the plateau portion 9202 may affect a flavor the vapor generated from the material for vaporization. For example, the higher the temperature of the heating element, an adverse effect may be experienced by the user when vaporizing of the material for vaporization, however it may provide for an increase in vapor density. For a lower temperature of the heating element, there may be a more optimal flavor experienced when the user vaporizes a material for vaporization and less vapor density. Determining a PWM profile that is applied to the heating element assembly may correlate both a flavor profile as well as providing for sufficient vapor density to be inhaled by the user. There may be a balance between amount of vapor generated as well as a flavor of the vapor when inhaled by the user. In addition, an inhalation rate at which the user inhaled from vaporization device, or inhalation profile, may also have a bearing on a vapor density produced. A faster inhalation rate may cool the heating element assembly more than a slower inhalation rate. As the ambient air flows into the vaporization device it flows as the heating element assembly and as a result will provide cooling to the heating element assembly. Through a dose training process (see FIG. 107) the user may be presented with an indication of an optimal rate at which to inhale from the vaporization device to be provided with optimal dosing.

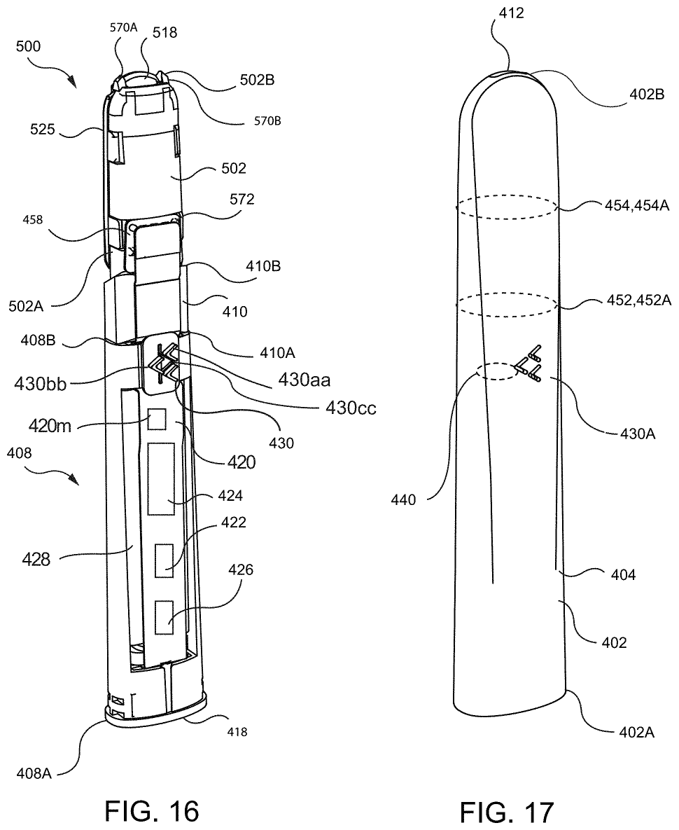

FIG. 107 illustrates an exemplary means of providing a dose progress indication to the user when using of the vaporization device when for example they inhale using an inhalation profile IP4 1104 FIG. 94d. Referring to FIG. 16, for example, LEDs may vary colors and or intensities to indicate different states or functions of the vaporizer 400. For example, the plurality of LEDs 430 includes a first LED 430aa, a second LED 430bb and a third LED 430cc, which may be oriented in a circle or a line. As a dose is inhaled from the vaporization device, each of the plurality of LEDs are illuminated to indicate a dose progress so that the user is informed of the dose as it is progressing when they are inhaling from the vaporization device. The first LED 430aa may have its light intensity ramped up upon the inhalation threshold being surpassed and during the rising portion 9201 and the second LED may 430bb may have its light intensity ramped up during second LED may 430bb and the third LED 430cc may have its light intensity ramped up post the second LED may 430bb and also during the plateau portion. In some embodiments the first LED may remain illuminated while the second LED is ramping up and the second LED may remain luminated as the third LED is ramping up. In some embodiments the LEDs ramping up may be associated with a time or they may be associated with a predetermined flow or air flowing through the manifold channel that a used is supposed to inhale for a predetermined dose. For example, if the user is to inhale 600 ml or air as part of the dose, then the second LED may have its light intensity ramped up at about 200 ml to 300 ml and remain illuminated thereafter and the third LED may have its light intensity ramped at about 300 ml and remain illuminated thereafter.

Another representation of the dose progressing may be presented on a graphical display (such as that of an OLED or a smartphone 1763 display screen (FIG. 110). There may be a circular graph such as a dose progress wheel 400dp (FIG. 110) or dose progress linear bar and the user is expected to complete the circle or linear bar with their inhalation as they inhale from the vaporization device 400.

Upon starting the dose, a mass airflow may exceed a mass airflow threshold and the user starts their inhalation and as the user maintains the mass airflow above this threshold then the circle keeps filling in. The end point of the circle (i.e. a 359-degree mark) is where the dose ends. For example, a dose having a volume of 400 ml in mass airflow conversion, needs to be achieved, so the user inhales and when their inhalation is above the mass airflow threshold then as the mass increases the circle fills in until they have inhaled about 400 ml.

In some embodiments a vibration notification 1134 is provided to the user as they are inhaling from the vaporization device, where a first vibration when the user starts to inhale and a set of two vibrations when the user is done inhaling to indicate the end of a dose., where a predetermined volume or mass of air has been inhaled from the vaporization device. In some embodiments if the user inhalation is below the inhalation threshold, then the LEDs are turned off and user is informed that their dose has been stopped and this may facilitate the re-wicking time where the device provides an indication for the user that the device is not able to be used for a subsequent dose until this waiting time has elapsed.

Depending on a rate at which the user inhales from the vaporization device, there may be created a different type of dose. If the user inhale slower, the heating element may be at a higher temperature and it may emit more vapor than when they inhale faster. This has a benefit to the user as they may select a desired dose for the vaporization device and they may vary their rate of inhalation, as long as it is above the inhalation threshold 100, to achieve this desired dose through activation of the heating element.

The user may select a dose size they wish to inhale from the vaporization device. Upon the user inhalation, when a predetermined inhalation volume is achieved, then the dose is completed (see FIG. 95 and FIGS. 94c and 94e). Operation of the heating element is based upon operating during the inhalation where the inhalation threshold is surpassed. The volume required is stated so that the dose may be completed in that amount of time if the user inhales slower. If they inhale faster the may receive a partial dose as the predetermined inhalation volume may be completed before the heating of the heating element is completed.

Referring back to FIGS. 71-72, the keypad 1445, which may be used to prevent unwanted access to the vaporization device, may also be used to provide additional functionality, such as dose efficacy or effectiveness reporting and dose size selection or PWM profile selection. For example, the buttons 1447A to 1447E may be used to select a PWM profile, such as button 1447A may be used to select PWM400, and button 1447B may be used to select PWM350, and button 1447C may be used to select PWM300, and button 1447D may be used to select PWM250, and button 1447E may be used to select PWM200. The user then may use the vaporization device in accordance with their selected PWM profile. Post usage of the VD 400, or VD 1400, the user may then optionally during a re-wicking time for example to rate how effective was their dose in terms of how it made them feel, so for example within a window of 2 minutes after the user has completed their inhalation from the VD 200, the user may then use the buttons 1447A to 1447E to rate dose effectiveness on a scale of five (button 1447A) down to one (button 1447E), where a five may indicate it was excellent and a one it wasn't so effective for them. Having buttons for the user to interact with pose completing a dose may obviate a need to utilize a smartphone or other external device to provide for effectiveness data for their dose. The effectiveness data may then be used for the user to enable them to log their experience with using of the VD or it may also serve to facilitate using of the VD in for example clinical trial settings. In clinical trial settings the VD may store many parameters about its operation automatically and may rely on the user to input effectiveness or efficacy of their treatment.

In some embodiments an audio microphone and associated audio processing circuitry may be provided as part of the control circuit and the user may speak to the device post use for establishing how effective was their dose post vaporization device use and the microphone may record the audio received and this audio may be compressed and stored within the memory circuit 420m.

Control circuit 120 may be configured to monitor and control various components of vaporization device 100. For example, control circuit 120 may be used to monitor and control the flow of current from energy storage members 128. Control circuit 120 may also be used to provide user interface functionality and user feedback, such as audio or visual outputs

A dry heater may result in heating up too fast and may later adversely affect a taste of the vapor. The control circuit may be used for current sensing for power being applied to the heater so that based on sensed current over time, it may enable or disable power applied to the heater in order for the heater not to become excessively hot.

In some embodiments when the material for vaporization is viscous, the heating element may be pre heated with a PWM of about 20% or 10% or 15% for about 1 second or 0.5 seconds to reduce a viscosity of the material for vaporization that is wicked within the heating element assembly that is proximate the heating element. This may facilitate the heating element to produce additional vapor density from the material for vaporization with the pre-heating operation performed vs without the pre-heating operation. In some embodiments if the variation device is used in a colder environment, for example one that is less than room temperature, say 15 Celsius, then the pre-heating operation may prove advantageous. In some embodiments the pre-heating PWM duration and time for the pre-heating operation may vary with ambient temperature. In some embodiments with a higher viscosity material for vaporization it may be preferable to use a higher porosity heating element assembly than with a lower viscosity material, where a lower porosity heating element assembly may be advantageous.

FIG. 110 illustrates a provisioning process for the vaporizer device 400 in accordance with an embodiment of the invention that may use a wireless connection over Wi-Fi. The control assembly 408 which may include the control circuit 420 and what is generally referred to as a Wi-Fi module 126, which may be in the form of an 802.11 technology that provides for an over-the-air interface between a wireless client and a base station or between two wireless clients. 802.11 and 802.11x may refer to a family of specifications developed by the IEEE for a wireless LAN or WLAN technology. A frequency that may be used for the 802.11 may be 2.4 GHz and 5 GHz or around 2.4 GHz or around between 2.401 GHz and 2.495 GHz.

Initiating of a provisioning mode may be setup through a key sequence entered onto the keypad 1445. In the provisioning process a portion of the control circuit as well as the first wireless communication module 126w as a Wi-Fi module may act as a standalone web server and provide a local network having a Wi-Fi station wireless name and a station password as a first SSID and a first password and a first IP address 8512i.

A purpose of the provisioning mode is mainly for enabling a headless device, such as the vaporizer device 400, for being able to ultimately connect to a dosing data server (DDS) 1231 for sending data from the vaporizer device 400 to the DDS 1231. A router assembly 1766 may include a third wireless communication module 1766w as part of a router assembly having a third SSID and third password and for accessing the DDS 1231 through internet access.

This provisioning process may be achieved by providing the control assembly for substantially being enclosed with the vaporizer device 400, and where the control assembly comprises a first wireless communication module 126w as a Wi-Fi module coupled with the control assembly 420 having the vaporizer device vaporizer device memory circuit 420m where the vaporizer device memory circuit 420m is for storing a first SSID and a first password and for executing steps of entering a wireless provisioning mode through creating a web server using the control assembly 420 and the first wireless communication module 126w by providing a first access point functioning as a web server having the first SSID 8512s and the first password 8512p and first IP address 8512i.

A direct wireless connection may then be established using a device with Wi-Fi capabilities, such as a smartphone 1763, or tablet or a laptop computer 1765 having a second wireless communication module. The device with the with Wi-Fi capabilities may then connect using the second wireless communication module 1765w to the first wireless communication module 126w and to the control circuit 420. The device with Wi-Fi capabilities, such as computing device 1765 having a display screen 1765d and may include processing circuitry for executing a web browser 1765b and may include the second wireless communication module 1765w. The computing device 1765 may connect with the second wireless communication module 1765w to the first wireless communication module 126w where the control assembly 420 may provide a first access point functioning as the web server in an administrator mode by using the first SSID and the first password 8512p and the first IP address 8512i through the web server displaying a vaporizer device HTML page 420h wirelessly provided by the web server.

Upon connection of the computing device 1765 the web browser being executed on the computing device 1765 is then able to direct login as an administrator with the first IP address 8512i, for example the first IP address may be 192.168.X.X. and more specifically may be 192.168.4.1, to wirelessly access an admin panel of the control circuit 420 of the vaporizer device 400.

Using the web browser being executed on the computing device 1765 the third SSID 8512s and third password 8512p may be provided as input data to the displaying HTML page 420h, where these input parameters may then wirelessly be provided to the control assembly. This provisioning processes may enable storing of the third SSID 8512s and third password 8512p within the vaporizer device memory circuit 420m of the control assembly 420 for enabling of the first wireless communication module 126w and the control assembly 420 to directly connect with the DDS 1231 through the internet access via the router assembly 1766.

In some cases, this provisioning of the vaporizer device 400 may be accomplished using a Bluetooth protocol using a smartphone device and a smartphone application, where Bluetooth may operate on a 2.4 GHz frequency and more specially 2.402 and 2.480 GHz, or 2.400 and 2.4835 GHz. In some cases, an application called WebBluetooth (as is known in the art) may be used with a desktop computer however this may require executing of a smartphone application on the smartphone and also may not be supported by all web browsers that are executed on the laptop computer 1765. Therefore, it may be preferable to provision using the wireless connection over Wi-Fi and any device with Wi-Fi capabilities that supports web browser capabilities.

Referring back to the provisioning portion, once the device with Wi-Fi capabilities is connected to the vaporizer device 400 acting as the first access point mode of operation, control parameters, such as setting a dose, for example Dose 11 781 or Dose 21 782 through sending values back to the vaporization device. In some embodiments amending of the preset code (i.e. this may be the code used to child proof the vaporization device) stored in the memory module that is uniquely associated to that vaporization device may also be amended through selecting of such on the vaporizer device HTML page 420h of the connected device with Wi-Fi capabilities and then sending the amended code back to the vaporization device.

Furthermore, the control assembly 408 may also display other parameters on the vaporizer device HTML page 420h, such as the dose progress wheel 400dp where there is a direct Wi-Fi wireless connection between the two devices in the admin mode. While the connected device with Wi-Fi capabilities is wirelessly coupled with the vaporizer device 400, data is exchanged bi-directionality populate the 420h being displayed on the display screen.

Upon provisioning of the vaporizer device 400 to wirelessly couple with the router assembly 1766 or hotspot or wireless access point, the device with Wi-Fi capabilities may then be wirelessly disconnected from the control circuit 420 and Wi-Fi module 126. The control circuit 420 and Wi-Fi module 126 may then directly connect with the router assembly 1766 for wireless data exchange therebetween.

In some embodiments the wireless data exchange between the control circuit 420 and Wi-Fi module 126 and the router assembly 1766 is encrypted. In some embodiments the vaporizer device 400 may include an optical data transmitting and, in some cases, a receiving port and an intermediate optical transceiver may be in optical communication with the optical data transmitting and, in some cases, a receiving port and the intermediate optical transceiver may be wirelessly coupled with the router assembly 1766 to couple data being transmitted from the vaporizer device memory circuit 420m within the control assembly 420 of the vaporization device 400 with the home router 1766. For example, the optical data transmitting, the optical receiving port and optical transceiver may operate at infrared frequencies modulated around 38 kHz. In some embodiments an infrared transmitter for use with the control circuit 400 of the vaporizer device 400 may be more cost effective than a wireless module.

The dosing data server (DDS) 1231 may also be provided for wirelessly connecting to a plurality of VDs 400 through a plurality of router assemblies 1766. The DDS 1231 may include a processor and a memory unit and may also include a DDS database 1231d stored on the DDS 1231 for storing information related to usage of the plurality of VDs 400. In some embodiments the VDs the DDS 1231 may be in the form of an Amazon Web Services Internet of Things (AWS IOT) server and it may connect with the control circuit 420 using a MQTT protocol.

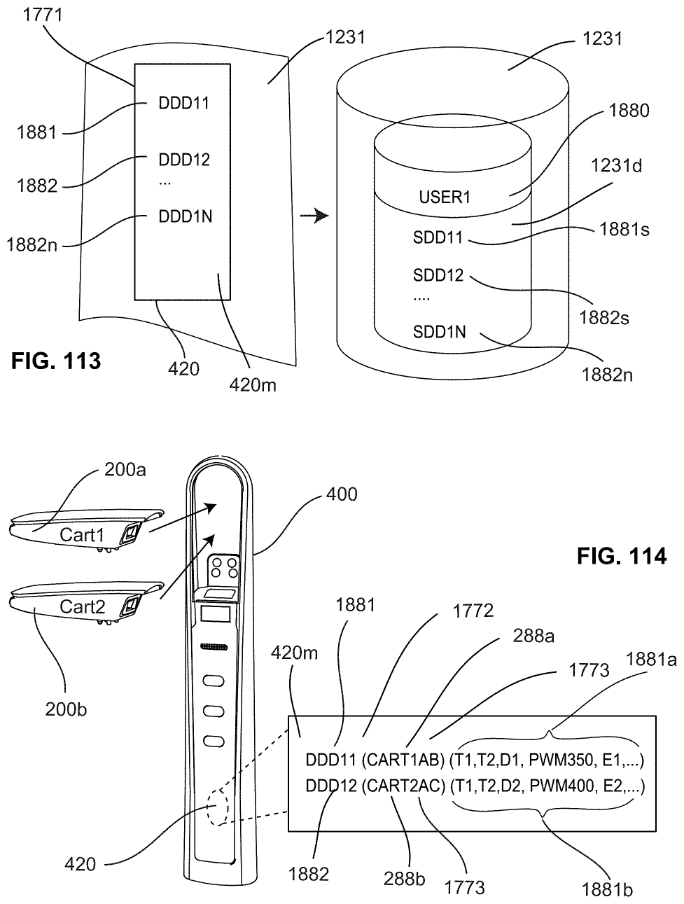

Referring to FIG. 113 and FIG. 114, upon the user interacting with vaporizer device 400, there may be an interaction log 1771 generated by the control assembly 408, where for each interaction with the vaporizer device 400 by the user, there may be a plurality of interaction parameters 1772 stored within the vaporizer device memory circuit 420m and these may be referred to as dosage, usage frequency and effectiveness data or generally as DUFE DATA and these may be related to the cartridge identifier data 1773.

Of course, other interaction parameters may also be stored and not just limited to those aforementioned and more or less of these parameters may be stored as the DUFEDATA, generally. The control assembly 408 may include a real-time clock and therefore may include interaction parameters related to a time at which the vaporizer device 400 is used as a start use time, for example T1 and it may store a time at which the usage is stopped and an end use time, for example T2, it may also store: a duration of use, an ambient temperature surrounding the vaporizer device 400, a battery level of the vaporizer device 400, an actual inhalation profile 6635 that is performed by the user (for example sampled at 100 ms intervals or 250 ms intervals to save on data packet size), a selected dose size by the user (for example D1 as Dose 1 or D2 as Dose 2), a PWM profile that is applied to the heating element, a corresponding calibrated measured dose value 1197a, an angle at which the vaporizer device 400 is being held in relation to ground (with the use of a gravity sensor or accelerometer), a potential dose effectiveness (as reported by the user post usage of the vaporizer device 400 and other usage parameters that may not have been mentioned.

For example, as shown in FIG. 114, with the use of the cartridge assembly 200, for example in the form of a first cartridge assembly CART1 200a. With CART1 200a being inserted into the vaporizer device 400, the control assembly 420 may create a first entry within the vaporizer device memory circuit 420m as a first device DUFE DATA (DDD11) 1881 and store the unique identification number 288, such as a first unique identification number 288a as CART1AB. Upon use of the VD, the control assembly 420 may create first additional entries 1881a within the vaporizer device memory circuit 420m, where these may be for T1=dose start time, T2=dose end time, D1=dose size, and PWM350=PWM profile used for the consumed dose, E2=dose effectiveness).

In some embodiments with the use of the cartridge assembly 200, for example in the form of a second cartridge assembly CART2 200b. With CART2 200b being inserted into the vaporizer device 400, the control assembly 420 may create a subsequent and second entry within the vaporizer device memory circuit 420m as a second device DUFE DATA (DDD12) 1882 and store the unique identification number 288, such as a second unique identification number 288b as CART2AC. Upon use of the VD400, the control assembly 420 may create second additional entries 1881b within the vaporizer device memory circuit 420m, where these may be for T1=dose start time, T2=dose end time, D1=dose size, and PWM400=PWM profile used for the consumed dose, E1=dose effectiveness). Of course, additional device DUFE DATA entries may be created within the vaporizer device memory circuit 420m as the user, in this case USER1, interacts with the VD. There may be a plurality of DUFE DATA stored as DDD11 1881, DDD12 1882, to DDD1n 1882n, for a plurality of interactions with the vaporizer device 400 by the first user, USER1.

The DUFE DATA stored within the vaporizer device memory circuit 420m, the interaction log stored within the vaporization device memory circuit, may then be transferred to the DDS database 1231d stored on the DDS 1231 (when within wireless range of the vaporization device and the router assembly), as the stored interaction log, pertaining to information related to usage of the vaporizer device 400, for example for USER1 where the DDD11 1881, DDD12 1882, to DD1n 1882n for the plurality of interactions with the vaporizer device 400 by the first user, USER1, may be stored within the DDS database 1231d where DDD11 1881 may be stored within the DDS database 1231d as a first stored DUFE DATA 11 (SDD11) 1881s, or a first stored interaction data, and DDD12 1882s may be stored within the DDS database 1231d as a second stored DUFE DATA 12 (SDD12) 1882s or a second stored interaction data, and up to DDD1n 1882n for the plurality of interactions as an Nth stored DUFE DATA 1N (SDD1N) 1882n. In some embodiments the DUFE DATA stored within the vaporizer device memory circuit 420m, the interaction log stored within the vaporization device memory circuit, may then be transferred to the DDS database 1231d when the vaporization device is plugged in for recharging of the battery.

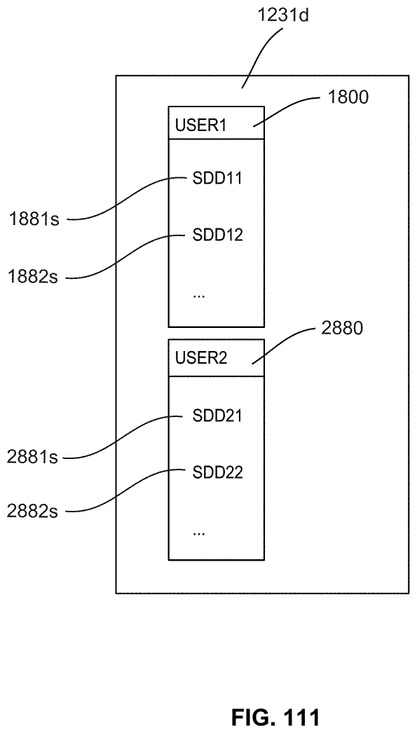

Referring to FIG. 111, for example, the DDS database 1231d stored on the DDS 1231 may include entries for a plurality of users, such as USER 1 and USER 2. Each of the users may have associated with their entries a user profile, such as USER1 and USER2. Furthermore, each of the plurality of users may also have associated with them, for example the first stored DUFE DATA 11 (SDD11) 1881s and DDD12 1882s may be stored within the DDS database 1231d and associated with a first user profile USER1 1880 and for example a stored DUFE DATA 21 (SDD21) 2881s and DDD22 2882s may be stored within the DDS database 1231d and associated with a second user profile USER2 2880.

For example, USER1 interacts with a first vaporizer device 400 and USER2 interacts with a second VD. Through using of the vaporizer device 400 one by USER 1 there may be a plurality of DUFEDATA11 and DUFEDATA12 generated and through using of the vaporizer device 400 two by USER 2 there may be a plurality of DUFEDATA21 and DUFEDATA22 stored within the DDS database 1231d upon the vaporizer device 400 synching its vaporizer device memory circuit 420m contents with the DDS database 1231d.

In some embodiments instead of using the keypad to rate dose effectiveness the user may use the recorded audio as aforementioned and this audio may be compressed and stored within the vaporizer device memory circuit 420m within the corresponding DUFE DATA, which they may further be sent to the DDS database 1231d stored on the DDS 1231. Audio processing and interpretation of the effectiveness recorded audio by the user may then be processed by the server and a digitized form of this may be stored in cofunctions with the stored DUFE DATA.

The DUFEDATA may be stored within the memory circuit within the control assembly of the vaporization device and then upon the VAPORIZER DEVICE 400 being within range of the router assembly 1766 the DUFEDATA stored within the memory circuit within the control assembly of the vaporization device is wirelessly transmitted to the DDS database 1231d stored on the DDS 1231 may include entries for a plurality of users, such as user profiles USER1 1880 and USER2 2880. The vaporizer device memory circuit 420m within the control assembly 420 of the vaporization device 400 (FIG. 16) may be a FLASH memory or an EEPROM or other storage medium for storing of the DUFEDATA therein, until the DUFEDATA has been wirelessly transmitted to the DDS database 1231d and then the DUFEDATA may be erased from the memory circuit within the control assembly of the vaporization device 400.

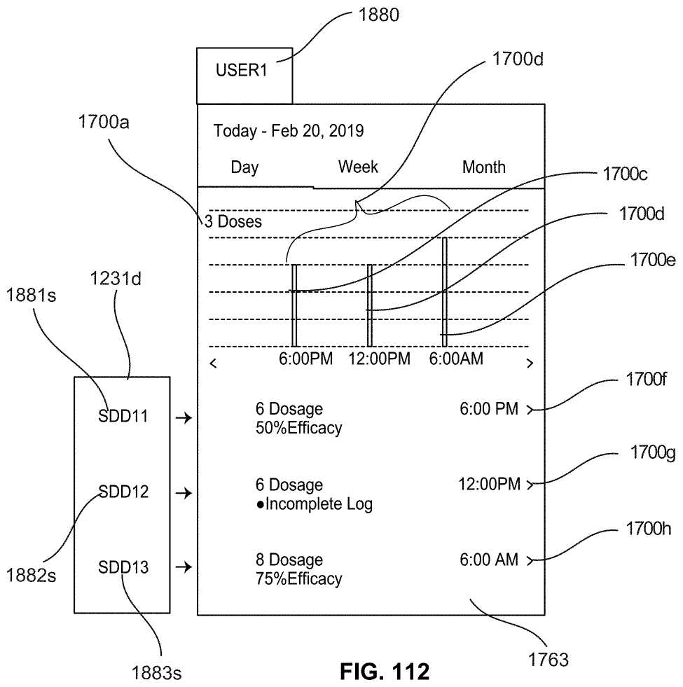

FIG. 112 illustrates an exemplary view of how DUFEDATA may be presented to the user on a display screen of a platform that uses a standards-compliant browser, such as a smartphone 1763, or tablet or a laptop computer 1765. The user may be able to view their treatment protocol while using of the VD. The platform that uses a standards-compliant browser, such as a smartphone 1763 may wirelessly connect with the DDS database 1231d stored on the DDS 1231 for receiving of corresponding data from the DDS database 1231d.

A progressive web application (PWA) may be used to view the DUFEDATA that is stored the DDS database 1231d stored on the DDS 1231. It may be preferable to use a PWA since this type of application is intended to work on any platform that uses a standards-compliant browser, which means that this obviates a need to create a device specific application, such as that which is required for iOS or Android devices.

The DUFEDATA, for example as shown for SDD11 1881s, SDD12 1882s and SDD13 1883s for USER1 profile 1880, this may be represented as a number of doses taken 1700a, a frequency of doses 1700b over a period of time and individual doses 1700c, 1700d, 1700e, where each dose is then may be listed as having its dose magnitude (i.e. amount of vapor and or mass airflow or combination thereof that is inhaled as a plurality of data points with respect to time) and the efficiency of that does (i.e. as being self-reported by the user through the buttons 1447A to 1447E to rate dose effectiveness on a scale of five (button 1447A) down to one (button 1447E). These may be displayed in a graphical form that allows for easy scrolling by the end user to view their dosing schedule and past activity and for the user to monitor/track their performance.

The DUFEDATA represented by the line items 1700f, 1700g, 1700h correspond do the individual doses 1700c, 1700d, 1700e and also display an effectiveness of the dose as self-reported by the user, as shown in FIG. 112. The usage frequency may be how often the USER1 is using the vaporizer device 400 and at what times, so for example the USER1 may use the vaporizing device in the morning at say T1=6 AM with an E1=50% effectiveness and at T1=12:00 PM with an incomplete dose and at T1=6:00 AM with a E3=75% effectiveness. Through connectivity by the platform that uses a standards-compliant browser, such as a smartphone 1763, or tablet or a laptop computer 1765, the DUFEDATA1 for USER 1 or DUFEDATA2 for USER 2 may be viewed on the display of such a device and as the USER1 or USER2 interacts with the vaporizer device 400, the DUFEDATA1 or DUFEDATA2 is increasingly populated.

In some embodiments when the user is setting up their profile, for example USER1 1880, the user may be able to select from a drop-down list of symptoms, which they wish to treat or for which they want to be dosing. In some embodiments the user may be able to rank a severity of their symptom before vaporization. Upon completing of the dose by the user, the user may then select the dose effectiveness as aforementioned using the keypad 1445. In some embodiments the user may connect their device with Wi-Fi capabilities to wirelessly couple with the vaporizer device 400 and to directly report on their wireless device dose feedback to vaporizer device HTML page 420h.

In some embodiments the DUFEDATA may be stored on one DDS 1231 or other DDS in dependence upon HIPAA/PIPEDA compliance or other medical data storage standards. In some embodiments the remote server 1008 may be a different server than the DDS 1231.

Other vaporization devices may also be used that are compatible with storing data as the DUFE DATA. In some embodiment's vaporizations device may be used to vaporize loose and/or ground phyto material having heating chambers for heating of the ground phyto material as well as a control circuit and airflow sensing. In some embodiments an air-cooling assembly may be used with the vaporizer device 400 in order to cool vapor emitted from the heating chamber prior to entering the mouthpiece. In some embodiments there may be a doctor and a licensed producer portal to view the DUDE DATA. Other devices that are aerosol generators, such as conventional medical inhaler devices, may also be used with advantages of some of the aforementioned embodiments of the invention. The inhaling device may be controlled by an electrical solenoid mechanism to control atomization from a pressurized canister where DUFE DATA may also be generated when the user is using of a conventional inhaler.

In some embodiments the user is rewarded user points for providing volumetric/mass air flow data to the system. In some cases the vaporization device registers the inhalation profile of the user as a code to understand whether a same user is using the device or whether another user has been using the vaporization device.

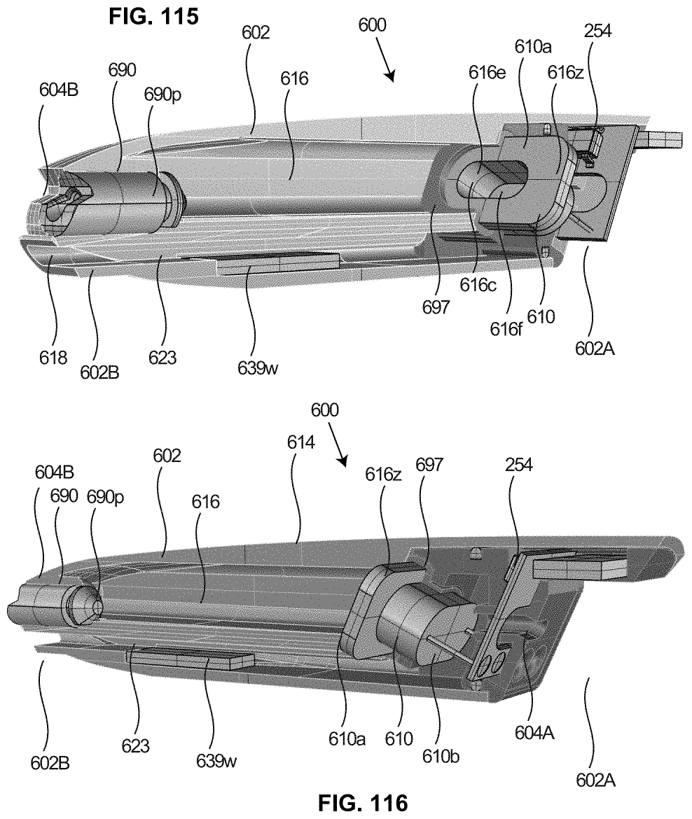

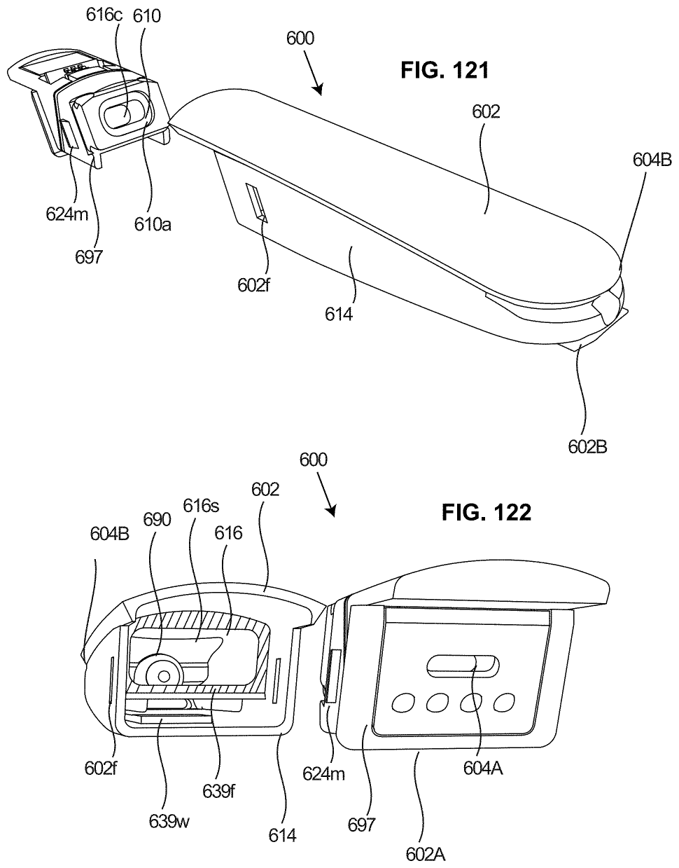

FIGS. 115, 116, 117, 121 and 122 illustrates a cartridge assembly 600 as another embodiment of the invention from various views. Referring to FIG. 115, cartridge assembly 600 as another embodiment of the invention is shown from a front cutaway render view and may be formed from a cartridge housing 602 may extend between a first cartridge end 602A and a second cartridge end 602B opposite the first cartridge end 602A. A housing sidewall 614 may extend between the first cartridge end 602A and the second cartridge end 602B. A housing length L_{H} may be measured between the first housing end 602A and the second cartridge end 602B. FIG. 116 illustrates the cartridge assembly 600 from a rear cutaway render view.

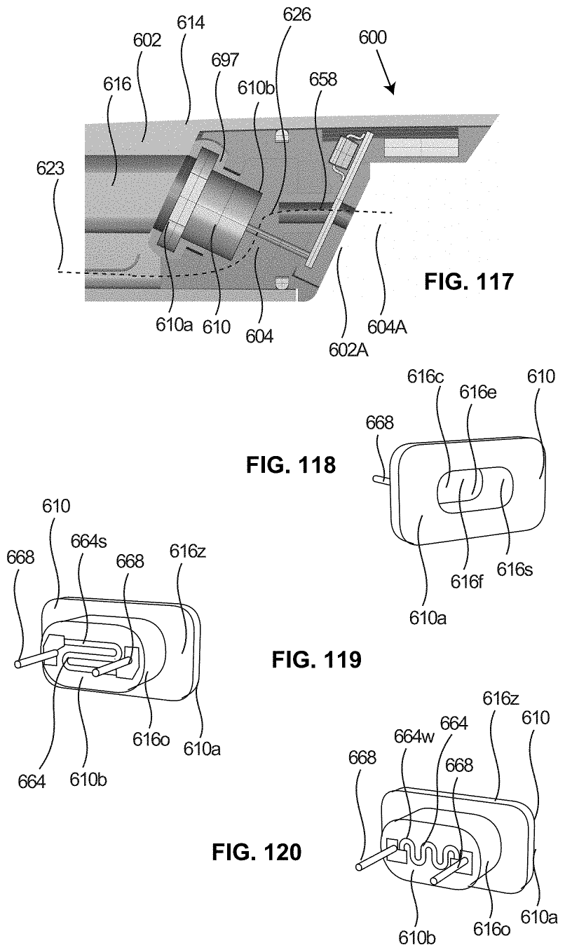

A fluid conduit 604 (FIG. 117) may extend through the cartridge housing 602 from the first cartridge end 602A to the second cartridge end 602B. The fluid conduit 604 may include a cartridge conduit inlet or upstream inlet 604A at the first cartridge end 602A. The fluid conduit 604 may include a cartridge conduit outlet or downstream inlet 604B at the second cartridge end 602B. The fluid conduit 604 may include a plurality of conduit sections, including a first or upstream section 658, a second or intermediate section 626, and a third or downstream section 623. The first or upstream section 658 may fluidly couple with the air intake manifold 410 (not shown in this FIG.). The air intake manifold 410 may be configured to allow ambient air to be drawn into vaporizer device and directed into a cartridge assembly 600 positioned within the cartridge receptacle 416 (not shown in this FIG.).

A cartridge aperture 618 may be defined in the cartridge housing 602 at the conduit outlet 604B. When the removable cartridge assembly 600 is positioned within the cartridge receptacle of vaporization device, the cartridge aperture 618 may be aligned with, and engage, the inhalation aperture. The inhalation aperture may thus be fluidly coupled to fluid conduit 604.

In some embodiments, the cartridge aperture 618 of the fluid conduit 604 may protrude from the housing 602 at the second cartridge end 202B, this may facilitate to provide an engagement member that may engage the inhalation aperture.

A storage compartment or reservoir 616 may be used to store vaporizable material for use with cartridge assembly 600. The storage compartment 616 may be enclosed by the outer housing sidewall 614. In the example shown, the storage compartment 616 may be parallel with the fluid conduit 604. That is, the fluid conduit 604 may define a passage that extends along a side of the storage compartment 616.

A heating element assembly 610 may be oriented with respect to the fluid conduit 604 in such a manner that the heating element assembly 610 may have a portion in a direct airstream fluid coupling when air flows within the fluid conduit 604 from the upstream inlet 604A at the first cartridge end 602A. The heating element assembly 610 may have a reservoir fluid end 610a and may have an airstream end 610b. In some embodiments the reservoir fluid end 610a and the airstream end 610b may be approximately parallel and in some embodiments, they may be at an angle to each other. The airstream end 610b may be disposed between the first or upstream section 658 and the third or downstream section 623 and may be within the second or intermediate section 626.

Referring to FIGS. 118 and 115 and 116, the reservoir fluid end 610a may be in fluid communication with the storage compartment or reservoir 616 and a heating element assembly cavity 616c may be formed within the heating element assembly 610 where the heating element assembly cavity 616c is open to the storage compartment or reservoir 616 at a cavity open end 616e and in fluid communication therewith. The heating element assembly cavity 616c may comprise an inner sidewall 616s and a cavity floor 616f where the inner sidewall 616s may be parallel with a heating element assembly outer sidewall 616o. The heating element assembly cavity 616c may extend from the reservoir fluid end 610a and may terminate at the cavity floor 616f. The inner sidewall 616s and the cavity floor 616f for enclosing of the heating element assembly cavity 616c having the cavity open end 616e. A heating element assembly flange 616z may be formed about the heating element assembly outer sidewall 616o and it may extend radially from the outer sidewall 616o by about 1 mm to 1.2 mm to 1.5 mm in some areas and may have a height of about 1 mm to about 1.5 mm to about 1.1 mm to about 0.9 mm.

A heating element assembly seal member 697 may be utilized that is manufactured from an elastomeric and deformable material that may form a frictional seal between the heating element assembly 610 and an interface member 624. The seal member 697 may be wrapped around an entire periphery of the heating element assembly flange 616z where a top side and a bottom side of the flange 616z are embedded within the seal member 597 with the cavity open end 616e still protrudes through a center of the seal member 697 for being exposed to the storage compartment or reservoir 616. The interface member 624 compresses the seal member 697 on both sides of the flange 616z with the flange 616z and with an inner surface 616s of the storage compartment 616 having a flat seal member interface area 616f that may surround the inner surface 616s of the storage compartment 616. Male snap fittings 624m may be provided as part of the interface member 624 that protrude past an outside surface and are for mating with snap fitting recesses 602f formed within the cartridge housing 602. When the male snap fittings 624m are engaged with the snap fitting recesses 602f the flat seal member interface area 616f compresses the heating element assembly flange 616z with the seal member 697 and may form a sealed storage compartment or reservoir 616 enclosing a inner volume of storage compartment 616. The inner volume may be about 0.5 ml or 0.75 ml or 1 ml.

A distance between the cavity floor 616f and the airstream end 610b may be about 1 mm or 0.6 mm or 0.8 mm or 0.5 mm. A distance between the inner sidewall 616s and the outer sidewall 616o may be about 1 mm or 0.8 mm or 0.6 mm or 1.1 mm. The distance between the cavity floor 616f and the airstream end 610b as well as a porosity thereof may define the wicking time. The heating element assembly 610 may be of a unitary construction and manufactured from a porous ceramic material and may have a 40-50% open porosity and with a tortuous pore structure and use pore sizes ranging from 1 to 100 microns, where more specifically it may have pore sizes of 10, 15, 30, 50, 60 and 100 microns.

As described hereinabove, the heating element assembly 610 may have a heating element inlaid within the heating element assembly 610 proximate the airstream end 610b or it may have a silk-screened heating element printed on its surface proximate the airstream end 610b.

As an inhalation takes place and air propagates through the cartridge assembly 600 and more specially through the fluid conduit 604 from the upstream inlet 604A to the downstream inlet 604B, the propagating air impacts the airstream end 610b at the intermediate section 626 and is deflected by the airstream end 610b towards the downstream section 623. This deflection of the propagating air may result in vapor to be mixed with the propagating air as well as for the propagating air to cool the heating element and may result in reduced condensation forming within the cartridge assembly as a result of the deflection of the propagating air. The propagating air impacting the airstream end 610b may serve to remove a majority of the formed vapor from this surface and to direct it into the third or downstream section 623.

In some embodiments an angle of the airstream end 610b with respect to the upstream section 658 may be about 20 degrees to about 10 degrees to about 45 degrees to about 60 degrees to about 90 degrees and it may be about 50 degrees. In some embodiments a wicking pad 639w may be provided along at least a portion of the downstream section 623 to collect condensation that may have formed along the downstream section 623.

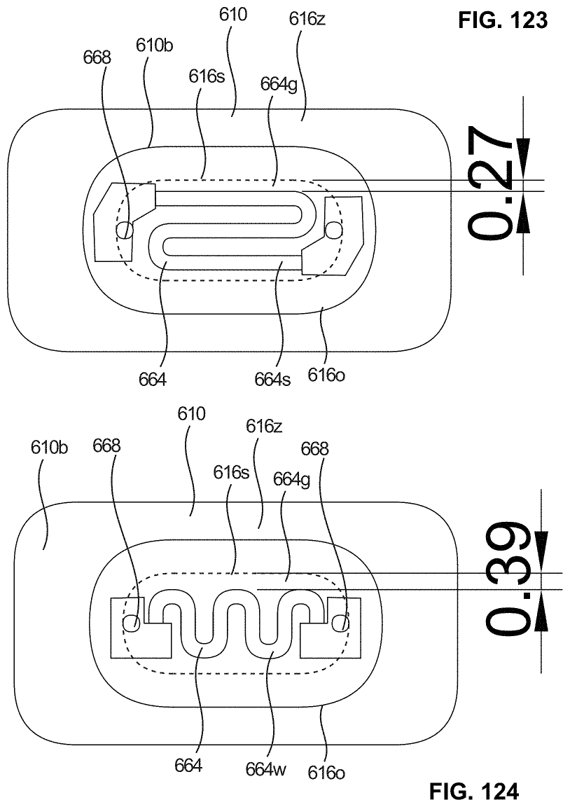

Referring to FIGS. 118, 119, 120 and 123 and 124, in some embodiments a heating element 664, which may be inlaid or silk screened. In some embodiments the heating element 664 may be in the form of a “S” as a S-type heating element 664s (FIGS. 119 and 123) or a “W” as W-type or wave type heating element 664w (FIGS. 120 and 124). Electrical couplings 668 may extend from the heating element assembly 610 where the these may couple with corresponding electrical contacts as part of the cartridge assembly 600 and with the with electrical couplings when the cartridge assembly 600 is inserted into the vaporization device.

Referring to FIGS. 123 and 124, the heating element 664 may be disposed in such an orientation on the airstream end 610b in such a manner that a heating element gap 664g (when viewed from the airstream end 610b) between traces of the heating element 664 and the inner sidewall 616s of the heating element assembly cavity 616c is about 0.27 mm or about 0.3 mm or about 0.39 mm or about 0.5 mm. This minimum gap may facilitate less carbonization of the material for vaporization during a heating process of the heating element. In some embodiments if this gap is too small, for example zero millimeters, then the material for vaporization will not wick in time through a too narrow gap and it may result in the heating element to reach a too high temperature and cause burning of the material for vaporization. A larger heating element gap 664g facilitates improved wicking through the heating element assembly 610 from the cavity floor 616f to the airstream end 610b where it has been observed by the inventor that when the heating element may overlap the inner sidewall 616s with a less than zero gap (horizontal gap as viewed from the airstream end 610b, this may cause carbonization. Optimally the heating element 664 is disposed equally at least with the heating element gap 664g when viewed from the airstream end 610b) within confines of the inner sidewall 616s (when viewed from the airstream end 610b).

The heating element assembly 610 may measure approximately L=7 mm×W=4 mm at the airstream end 610b and have a height of about H=6 mm and where the cavity 616c allows for vaporable material to flow into the cavity and to be substantially retained within the cavity 616c where in some embodiments the cavity 616c and its sidewalls are oriented at an angle with the third or downstream section 623, for example at an angle of 45 degrees, then vaporizable material may be retained with in the cavity when the cartridge assembly is oriented vertically or horizontally, resulting in vaporizable material to be substantially retained within the porous structure of the heating element assembly 610 proximate the heating element 664 between the cavity floor 616f and the airstream end 610b. Preferably about 5 mg to 6 mg to 8 mg of vaporizable material is retained between the cavity floor 616f and the airstream end 610b proximate the heating element 664. Retention of vaporizable material within this area may facilitate a faster re-wicking time.

A filling aperture 690 may be formed within the cartridge housing 602 proximate the second cartridge end 602B and a filling plug 690p may be used to seal the filling aperture 690 where the filling plug 690p may be first inserted in to the filling aperture 690 during cartridge assembly manufacturing and in a filling process, a needle may be used to pierce filling plug 690p with the needle exposed to the storage reservoir 616 and fill the cartridge assembly in an inverted manner whereby air contained within the storage reservoir 616 escapes from the storage reservoir 616 during the filling operation through the fluidly coupled porous heating element assembly 610 (filling in a non-inverted manner may result in the vaporizable material to plug pores of the heating element assembly and result in the cartridge assembly to leak during filling). Post filling, the needle may be removed and the filling plug 690p self-seals and the material for vaporization within the storage reservoir 616.

As used herein, the wording “and/or” is intended to represent an inclusive—or. That is, “X and/or Y” is intended to mean X or Y or both, for example. As a further example, “X, Y, and/or Z” is intended to mean X or Y or Z or any combination thereof.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

What I claim is:

1. A vaporizer device and system comprising:
providing a vaporizer device comprising a vaporizer body;
providing a control assembly substantially enclosed with the vaporizer device, the control assembly comprising a first wireless communication module and the control assembly comprising a memory circuit for storing a first SSID and a first password and for executing steps of entering a wireless provisioning mode through creating a web server using the control assembly and the first wireless communication module by providing a first access point functioning as a web server having the first SSID and the first password and first IP address;
provisioning the vaporizer device for wirelessly connect with a third wireless communication module as part of a router assembly having a third SSID and third password for and accessing a dosing data server having a dosing data server database through internet access by providing a second wireless communication module as part of a computing device having a display screen and a processing circuitry for executing a web browser and
connecting of the second wireless communication module to the first wireless communication module first access point functioning as the web server in an administrator mode by using the first SSID and the first password and the first IP address through the web server displaying a vaporizer device HTML page wirelessly provided by the web server,
wherein on the display screen the third SSID and third password is provided as input parameters to the displaying vaporizer device HTML page and the provided input is wirelessly provided to the control assembly,
enabling storing of the third SSID and third password within the memory circuit of the control assembly for enabling of the first wireless communication module and the control assembly to directly connect with the dosing data server database through the internet access.

2. A vaporizer device and system according to claim 1 comprising: providing a keypad electrically coupled with the control assembly and entering a key sequence on the keypad for enabling of the control assembly for functioning as the web server.

3. A vaporizer device and system according to claim 1 wherein the first IP address comprises 192.168.X.X.

4. A vaporizer device and system according to claim 1 wherein the vaporizer device comprises an elongated base extending from a first end to a second end, the elongated base including a pair of opposed sidewalls extending between the first end and the second end and a second end wall at the second end;
a mouthpiece formed at the second end of the base, the mouthpiece comprising an inhalation aperture through the second end wall;
an air intake manifold mounted to the base, the air intake manifold having a first manifold end and a second manifold end with a manifold fluid flow path defined therethrough, the air intake manifold comprising an ambient air input port disposed between the first manifold end and the second manifold end, the ambient air input port being exposed to an external environment;
a fluid flow sensor assembly fluidly coupled between first manifold end and a second manifold with the manifold fluid flow path, the fluid flow sensor assembly for generating a fluid flow signal in dependence upon a flow of air through the manifold fluid flow exceeding a predetermined flow threshold;
an elongated storage compartment, the storage compartment being configured to store a vaporizable material, the storage compartment comprising an inner storage volume wherein the vaporizable material is storable in the inner storage volume, the elongated storage compartment comprising a first end and a second end opposite the first end;
a heating element assembly disposed at the elongated storage compartment first end, the heating assembly comprising a heating element, wherein the heating element is thermally coupled with the heating element assembly, and wherein heating element assembly is in fluid communication with the inner storage volume for wicking of the vaporizable material into the heating element assembly; and
a fluid conduit extending parallel with the elongated storage compartment from the first end to the second end, the fluid conduit having a fluid conduit inlet proximate the elongated storage compartment first end and a fluid conduit outlet proximate the elongated storage compartment second end,
wherein the fluid conduit is in fluid communication with the heating element assembly and the fluid conduit inlet is fluidly connected to the air intake manifold and the fluid conduit outlet is fluidly connected to the mouthpiece, and a fluid flow path is defined between the ambient air input port and the inhalation aperture, the fluid flow path passing proximate the heating element assembly;
where the control assembly includes a vaporizer device memory circuit and is substantially enclosed with the vaporizer body and electrically coupled with the fluid flow sensor assembly and the heating element, the control assembly for reading from a memory circuit which is for storing at least a pulse width modulation profile therein where upon the fluid flow signal being generated the at least a pulse width modulation profile stored within the memory circuit for controllably applying electrical power with respect to time to the heating element based upon the least a pulse width modulation profile,
the heating element for heating of the heating element assembly and for creating an aerosol from the vaporizable material that is wicked into the heating element assembly and for the aerosol to flow into the fluid flow path and for the aerosol to mix together with the ambient air flow through the manifold fluid flow path for together to flow from the mouthpiece,
wherein upon inhalation from the mouthpiece an interaction is generated and also generating an interaction log by the control assembly, where for an interaction from a plurality of interaction with the vaporizer device a plurality of interaction parameters are stored are stored within the vaporizer device memory circuit.

5. A vaporizer device and system according to claim 1 comprising providing a mouthpiece as part of the vaporization device and generating an interaction through inhalation through the mouthpiece and generating an interaction log by the control assembly for storing of the interaction log within the vaporization device memory circuit wherein for each interaction with the vaporizer device by the user, there may be a plurality of interaction parameters generated.

6. A vaporizer device and system according to claim 5 wherein the interaction log comprises data relating to at least two of the following interaction parameters comprising a start use time and an end use time and a duration of use time and a an ambient temperature surrounding vaporizer device and a battery level of the vaporizer device and an actual inhalation profile achieve through inhalation from the mouthpiece and a selected dose size and a pulse width modulation profile applied to the heating element and a corresponding calibrated measured dose value and an angle at which the vaporizer device is being held in relation to ground and a user reported dose effectiveness.

7. A vaporizer device and system according to claim 6 comprising providing a keypad comprising a plurality of keys electrically coupled with the control assembly and post inhalation from the vaporization device for entering a key from the plurality of keys for receiving input from the user as the user reported dose effectiveness.

8. A vaporizer device and system according to claim 5 wherein the enabling of the first wireless communication module and the control assembly to directly connect with the dosing data server through the internet access comprises transferring of the interaction log stored within the vaporization device memory circuit to the dosing data server database as a stored interaction log.

9. A vaporizer device and system according to claim 8 wherein the interaction log stored within the vaporization device memory circuit comprises a first interaction and a second interaction where data corresponding to the first interaction is different than data corresponding to the second interaction.

10. A vaporizer device and system comprising:
providing a vaporizer device comprising a vaporizer body;
providing a control assembly substantially enclosed with the vaporizer device, the control assembly comprising a vaporization device memory circuit and a first wireless communication module and for storing a first SSID and a first password and a first IP address and for storing a third SSID and third password;
providing a mouthpiece as part of the vaporization device and generating an interaction through inhalation through the mouthpiece and generating an interaction log by the control assembly for storing of the interaction log within the vaporization device memory circuit wherein for each interaction with the vaporizer device by the user, there may be a plurality of interaction parameters generated;
wirelessly provisioning the vaporizer device using the first SSID and the first password for wirelessly connect with a third wireless communication module as part of a router assembly comprising the third SSID and the third password for and accessing a dosing data server having a dosing data server database through internet access;
enabling of the first wireless communication module and the control assembly to directly connect with the dosing data server through the internet access comprises transferring of the interaction log stored within the vaporization device memory circuit to the dosing data server database as a stored interaction log.

11. A vaporizer device and system according to claim 10 comprising:
providing a second wireless communication module as part of a computing device having a display screen and a processing circuitry for executing a web browser and
connecting of the second wireless communication module to the first wireless communication module first access point functioning as the web server in an administrator mode by using the first SSID and the first password and the first IP address through the web server displaying a vaporizer device HTML page wirelessly provided by the web server,
wherein on the display screen the third SSID and third password is provided as input parameters to the displaying vaporizer device HTML page and the provided input is wirelessly provided to the control assembly.

12. A vaporizer device and system according to claim 10 comprising:
providing a removable cartridge assembly comprising a heating element assembly comprising a heating element a cartridge memory module for storing therein at least parameters relating to a PWM profile for being applied to the heating element;
releasably electrically coupling of the cartridge memory module with the vaporization device memory circuit;
applying of the at least parameters relating to a PWM profile to the heating element;
storing of the of the at least parameters relating to a PWM profile applied to the heating element as at least an entry within the interaction log data stored within the vaporization device memory circuit.

13. A vaporizer device and system according to claim 10 comprising:
providing a progressive web application for being executed within the web browser and visually represented on display screen; and
receiving of stored interaction log from the dosing data server database;
visually representing of the interaction log data stored on the dosing data server database server on the display screen.

14. A vaporizer device and system according to claim 10 disconnecting of the first wireless communication module from the third wireless communication module and connecting the first wireless communication module to the second wireless communication module;
enabling of the vaporizer device control assembly to function as the web server;
displaying the vaporizer device HTML page;
production a visual indication of the interaction through inhalation from the mouthpiece through a progress indicator on the display screen.

15. A vaporizer device and system according to claim 11 wherein the first, second and third wireless communication modules comprise an 802.11x protocol and operate between 2.4 GHz and 5 GHz.

16. A vaporizer device and system according to claim 10 comprising providing an audio microphone and audio processing circuitry for having ability to record of audio post inhalation from the vaporization device from the user as the user reported dose effectiveness.
